(19) United States
(12) Patent Application Publication
(10) Pub. No.: US 2025/0073164 A1
(43) Pub. Date: Mar. 6, 2025

(54) IONIZABLE DISULFIDE LIPIDS AND LIPID NANOPARTICLES DERIVED THEREFROM

(71) Applicant: PROVIDENCE THERAPEUTICS HOLDINGS INC., Calgary (CA)

(72) Inventors: Rajesh Krishnan GOPALAKRISHNA PANICKER, Calgary (CA); Yury KARPOV, Calgary (CA); Kirstin OLSEN, Calgary (AB)

(21) Appl. No.: 18/722,759

(22) PCT Filed: Dec. 22, 2022
(86) PCT No.: PCT/CA2022/051889

§ 371 Date: Jun. 21, 2024

(30) Foreign Application Priority Data
Dec. 22, 2021 (CA) 3143650

(51) Int. Cl. A61K 9/127; A61K 9/51; A61K 39/00; A61K 39/39; C07C 323/52

(52) U.S. Cl. CPC A61K 9/1271; A61K 9/5123; A61K 39/39; C07C 323/52; A61K 2039/53; A61K 2039/55555

(57) ABSTRACT

Ionizable lipids with a disulfide moiety and lipid nanoparticles comprising said lipids are disclosed. In select preferred embodiments, the ionizable lipids have a structure of formulae 11-15. The lipid nanoparticles formed from the ionizable lipids can be used as delivery vehicles for medicinal small molecules, antibodies, nucleotides, and polypeptides. Such nanoparticles can be used as vaccines or the treatment of cancer.

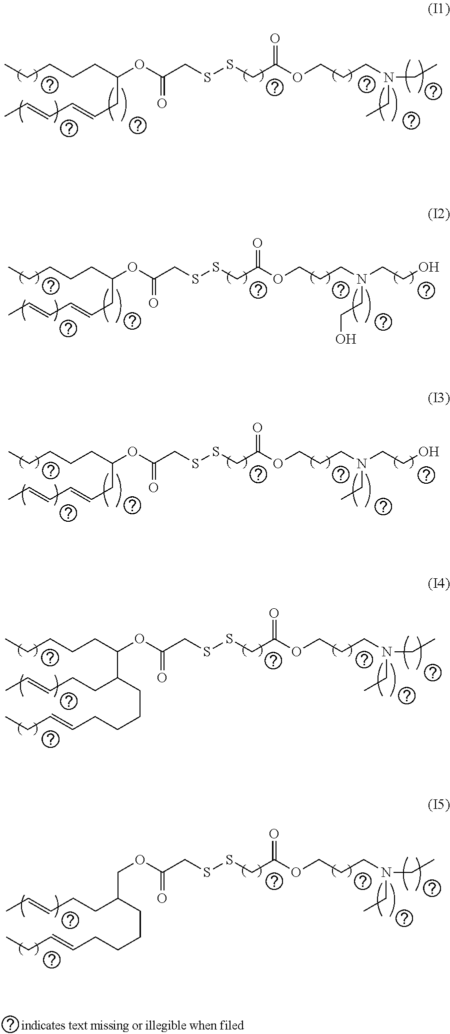

### FIELD OF THE DISCLOSURE

The present disclosure relates to disulfide lipids, and their use for preparing systems for encapsulating nucleic acid sequences, polypeptides or peptides. More particularly, the present disclosure relates to ionizable disulfide compounds useful to prepare lipids nanoparticles (LNPs).

### BACKGROUND

The development of systems, such as lipid nanoparticle delivery systems, for delivering therapeutics such as proteins, nucleic acid sequences, polypeptides or peptides to prevent or treat diseases has been increasing in the recent years. Lipid nanoparticles (LNPs) usually contain four ingredients: an ionizable lipid, a phospholipid, cholesterol and a PEGylated lipid. A major component of LNPs is the ionizable lipid. The phospholipid supports the formation of a lipid bilayer while cholesterol can stabilize the lipid bilayer. The PEGylated lipid, being amphiphilic, remains on the surface of LNPs to provide colloidal stability by steric shielding. Designing new ionizable lipids with suitable efficacy, stability and/or biodegradability to allow the preparation of LNPs is needed.

Proteins have been the standard for therapeutics but the use of nucleic acids as therapeutic modalities for a variety of diseases and therapeutic indications has gained in prominence over the past few years. Various companies have shown that nucleic acids (e.g., siRNA, mRNA, circular RNA, DNA, etc.) can be more effective when compared to protein-based therapies. There is a need for new delivery systems, such as new LNPs, for both nucleic acid and protein therapeutics. New LNPs can be obtained by designing suitable lipids.

### SUMMARY

The present disclosure provides lipid compounds, more particularly disulfide lipid compounds. Particles, such as nanoparticles, comprising the compounds, constructs comprising the nanoparticles and cargos, wherein the cargo can be a small molecule, an antibody, a polynucleotide, or a polypeptide, methods of using the particles/constructs, and methods of preparing the compounds, particles and constructs are also provided.

The details of various embodiments are set forth in the description below. Other features, objects and advantages will be apparent from the description, and from the claims.

### BRIEF DESCRIPTION OF THE DRAWINGS

FIG. 1 depicts a TEM-image of LNPs-100-01.

FIG. 2 depicts a TEM-image of LNPs-105-01.

FIG. 3 depicts a TEM-image of LNPs-109-01.

### DETAILED DESCRIPTION

### I. Introduction

The following description sets forth exemplary compounds, compositions, methods, parameters and the like. It should be recognized, however, that such description is not intended as a limitation on the scope of the present disclosure but is instead provided as a description of exemplary embodiments.

The delivery of a therapeutic agent to a subject is important for its therapeutic effects and usually it can be impeded by limited ability of the compound to reach targeted cells and tissues. Improvement of such therapeutic agents to enter the targeted cells of tissues by a variety of means of delivery is crucial. Nucleic acid therapy has emerged as the dominant method of treating various diseases and therapeutic indications given the versatility, lower immune response and higher potency as compared to traditional therapies. For example, nucleic acid therapy includes the use of small interfering (siRNA) to reduce the translation of messenger RNA (mRNA), mRNA as a way to produce a target of interest, circular RNA (oRNA) which can provide continuous production of a polypeptide or peptide or can be a sponge to compete with other RNA molecules, and viral vectors to provide a continuous production of a target of interest. However, some nucleic acids are unstable and easily degraded so they need to be formulated to prevent the degradation and to aid in the intracellular delivery of the nucleic acids.

The present invention relates to novel disulfide lipid compounds and compositions comprising the same, more particularly nanoparticles based on these disulfide compounds, capable of encapsulating a cargo such as a biologically active and therapeutic agent.

Examples of biologically active agents include but are not limited to: (1) proteins including immunoglobin proteins, (2) polynucleotides such as genomic DNA, cDNA, or mRNA, (3) antisense polynucleotides, and (4) low molecular weight compounds, whether synthetic or naturally occurring, such as the peptide hormones and antibiotics.

“Lipid” means an organic compound that comprises an ester of fatty acid and is characterized by being insoluble in water, but soluble in many organic solvents. Lipids are usually divided into at least three classes: (1) “simple lipids,” which include fats and oils as well as waxes; (2) “compound lipids,” which include phospholipids and glycolipids; and (3) “derived lipids” such as steroids.

“Lipid particle” or “lipid nanoparticle (LNP)” means a lipid formulation that can be used to deliver a cargo, such as a therapeutic nucleic acid (e.g., mRNA) to a target site of interest (e.g., cell, tissue, organ, and the like). In preferred embodiments, the lipid particle is a nucleic acid-lipid particle, which is typically formed from a cationic lipid, a non-cationic lipid (e.g., a phospholipid), a conjugated lipid that prevents aggregation of the particle (e.g., a PEG-lipid), and optionally cholesterol. Typically, the therapeutic nucleic acid (e.g., mRNA) may be encapsulated in the lipid portion of the particle, thereby protecting it from enzymatic degradation.

Lipid particles typically have a mean diameter of from 30 nm to 200 nm, from 40 nm to 180 nm, from 50 nm to 150 nm, from 60 nm to 130 nm, from 70 nm to 110 nm, from 70 nm to 100 nm, from 80 nm to 100 nm, from 90 nm to 100 nm, from 70 to 90 nm, from 80 nm to 90 nm, from 70 nm to 80 nm, or 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm or 200 nm and are substantially non-toxic. In addition, nucleic acids, when present in the lipid particles of the present invention, are resistant in aqueous solution to degradation with a nuclease.

### II. Lipids

The present disclosure provides compounds that are ionizable lipids, more particularly ionizable disulfide lipids. The ionizable lipids may be cationic lipids.

In some embodiments, compounds of the present disclosure comprise at least one disulfide bond (—S—S—). In some embodiments, compounds of the present disclosure further comprise at least two ester bonds (—CO—O— or —O—CO—). In some embodiments, compounds of the present disclosure further comprise at least one terminal amino group, wherein the amino group may be substituted with at least one lower alkyl group (e.g., C1-C3 alky groups), which may be further substituted. In some embodiments, the terminal amino group can be NH₂, a primary amino group, a secondary amino group, or a tertiary amino group. In some embodiments, the terminal amino group can be N(CH₃)₂, —N(CH₃)(CH₂CH₃), —N(CH₃)(CH₂CH₂OH), —N(CH₂CH₂OH)₂, or N((CH₂)₂O(CO)CH₃)₂.

### Formula (I)

In some embodiments, compounds of the present disclosure have a structure of Formula (I):

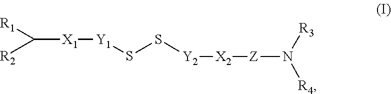

or a pharmaceutically acceptable salt thereof, wherein
X1 and X2 are independently an optionally substituted ester, amino, or amido or either group,
Y1 and Y2 are independently a bond or an optionally substituted C1-C10 alkyl group with any possible isomerism,
Z is an optionally substituted C1-C10 alkyl group with any possible isomerism,
R1 and R2 are independently an optionally substituted C8-C20 alkyl, C8-C20 alkenyl, or C8-C20 alkynyl group with any possible isomerism, and
R3 and R4 are independently H, an optionally substituted C1-C4 alkyl group, or an optionally substituted C1-C4 alkoxyl group.

In some embodiments, compounds of the present disclosure have a structure of Formula (I):

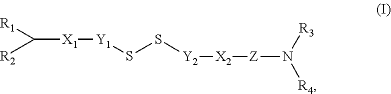

or a pharmaceutically acceptable salt thereof, wherein
X1 and X2 independently represent an ester bond —CO—O— or —O—CO—,
Y1 and Y2 are independently an optionally substituted linear or branched C1-C10 alkyl group,
Z is an optionally substituted linear or branched C1-C10 alkyl group,
R1 and R2 are independently a linear or branched C8-C20 alkyl, a linear or branched C8-C20 alkenyl, or a linear or branched C8-C20 alkynyl group, wherein the C8-C20 alkyl is optionally substituted with a linear or branched C2-C12 alkenyl group, and
R3 and R4 are independently an optionally substituted C1-C4 alkyl group.

In some embodiments, compounds of the present disclosure have a structure of Formula (I):

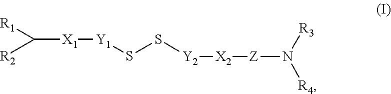

or a pharmaceutically acceptable salt thereof, wherein
X1 represents an ester bond —CO—O— and X2 represents an ester bond —O—CO—, or X1 represents an ester bond —O—CO— and X2 represents an ester bond —CO—O—,
Y1 and Y2 independently represent a linear or branched C1-C10 alkyl group,
Z is a linear or branched C1-C10 alkyl group,
R1 and R2 are different and independently represent a linear or branched C8-C20 alkyl, or a linear or branched C8-C20 alkenyl, wherein the C8-C20 alkyl is optionally substituted with a linear or branched C2-C12 alkenyl group, and
R3 and R4 are independently a C1-C4 alkyl group, wherein the C1-C4 alkyl group is optionally substituted with halogen, hydroxyl, acetoxy, alkoxycarbonyl, formyl, acyl, thiocarbonyl, alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, an aromatic moiety or an heteroaromatic moiety.

In some embodiments, compounds of the present disclosure have a structure of Formula (I):

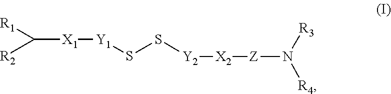

or a pharmaceutically acceptable salt thereof, wherein
X1 represents an ester bond —CO—O— and X2 represents an ester bond —O—CO—, or X1 represents an ester bond —O—CO— and X2 represents an ester bond —CO—O—,
Y1 and Y2 independently represent a linear or branched C1-C10 alkyl group,
Z is a linear or branched C1-C10 alkyl group,
R1 and R2 are different and independently represent a linear or branched C8-C20 alkyl, or a linear or branched C8-C20 alkenyl, wherein the C8-C20 alkyl is optionally substituted with a linear or branched C2-C12 alkenyl group, and
R3 and R4 are independently a C1-C4 alkyl group, wherein the C1-C4 alkyl group is optionally substituted with hydroxyl or acetoxy.

In some embodiments, compounds of the present disclosure have a structure of Formula (I):

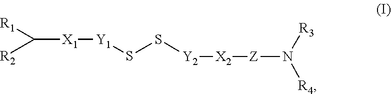

or a pharmaceutically acceptable salt thereof, wherein
X1 represents an ester bond —CO—O— and X2 represents an ester bond —O—CO—, or X1 represents an ester bond —O—CO— and X2 represents an ester bond —CO—O—,
Y1 and Y2 independently represent a linear or branched C1-C4 alkyl group,
Z is a linear or branched C1-C10 alkyl group,
R1 and R2 are different and independently represent a linear or branched C8-C20 alkyl, or a linear or branched C8-C20 alkenyl, wherein the C8-C20 alkyl is optionally substituted with a linear C2-C12 alkenyl group, and
R3 and R4 are independently a C1-C4 alkyl group, wherein the C1-C4 alkyl group is optionally substituted with hydroxyl or acetoxy.

In some embodiments, in the compounds of Formula (I) or the pharmaceutically acceptable salt thereof, Y1 and Y2 are identical.

In some embodiments, in the compounds of Formula (I) or the pharmaceutically acceptable salt thereof, Z is a linear or branched C1-C4 alkyl group.

In some embodiments, in the compounds of Formula (I) or the pharmaceutically acceptable salt thereof, R1 and R2 are different and independently represent a linear or branched C8-C18 alkyl, or C8-C18 alkenyl, wherein the C8-C18 alkyl is optionally substituted with a C6-C10 alkenyl group, the alkenyl groups independently comprising one or two double bonds.

In some embodiments, in the compounds of Formula (I) or the pharmaceutically acceptable salt thereof, R3 and R4 are independently a C1-C2 alkyl group, wherein the C1-C2 alkyl group is optionally substituted with hydroxyl or acetoxy.

In some embodiments, in the compounds of Formula (I) or the pharmaceutically acceptable salt thereof, R3 and R4 are identical.

In some embodiments, compounds of the present disclosure have a structure of Formula (I1):

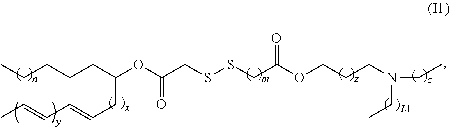

or a pharmaceutically acceptable salt thereof. n is a number from 4 to 16; x is a number from 1 to 15; y is a number from 1 to 8; m is a number from 1 to 10; z is a number from 0 to 8; and L1 and L2 are independently a number from 0 to 3.

In some embodiments, compounds of the present disclosure have a structure of Formula (I2):

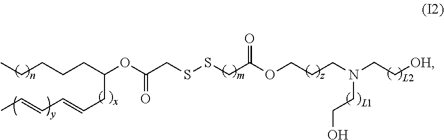

or a pharmaceutically acceptable salt thereof. n is a number from 4 to 16; x is a number from 1 to 15; y is a number from 1 to 8; m is a number from 1 to 10; z is a number from 0 to 8; and L1 and L2 are independently a number from 0 to 3.

In some embodiments, compounds of the present disclosure have a structure of Formula (I3):

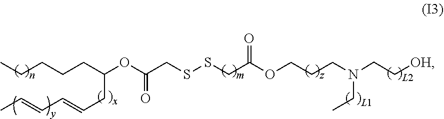

or a pharmaceutically acceptable salt thereof. n is a number from 4 to 16; x is a number from 1 to 15; y is a number from 1 to 8; m is a number from 1 to 10; z is a number from 0 to 8; and L1 and L2 are independently a number from 0 to 3.

In some embodiments, compounds of the present disclosure have a structure of Formula (I4):

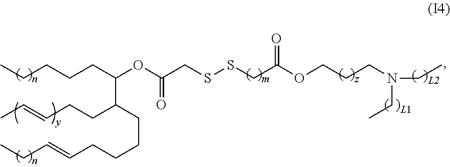

or a pharmaceutically acceptable salt thereof. n is a number from 4 to 16; y is a number from 1 to 8; m is a number from 1 to 10; z is a number from 0 to 8; and L1 and L2 are independently a number from 0 to 3.

In some embodiments, compounds of the present disclosure have a structure of Formula (I5):

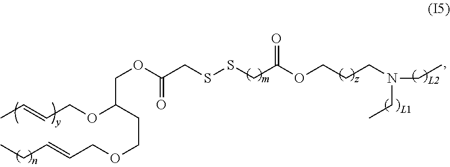

a pharmaceutically acceptable salt thereof. n is a number from 4 to 16; y is a number from 1 to 8; m is a number from 1 to 10; z is a number from 0 to 8; and L1 and L2 are independently a number from 0 to 3.

In some embodiments, compounds of the present disclosure can have a structure of Formula (I6):

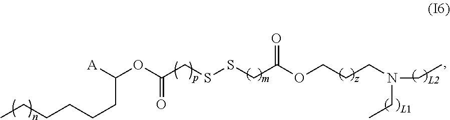

or a pharmaceutically acceptable salt thereof, wherein n is a number from 1 to 16; m and p are independently a number from 1 to 10; z is a number from 0 to 8; L1 and L2 are independently a number from 0 to 3; and A is a linear or branched C4-C20 alkyl, a linear or branched C4-C20 alkenyl, or a linear or branched C4-C20 alkynyl group.

In some embodiments, the compounds of Formula (I6), or the pharmaceutically acceptable salt thereof, are such that n is a number from 1 to 16; m and p are independently a number from 1 to 3; z is a number from 0 to 2; L1 and L2 are 0; and A is a linear or branched C5-C20 alkyl, a linear or branched C5-C20 alkenyl, or a linear or branched C5-C20 alkynyl group.

In some embodiments, the compound of Formula (I6) can have a structure of Formula (I6a) or (I6b), or a pharmaceutically acceptable salt thereof,

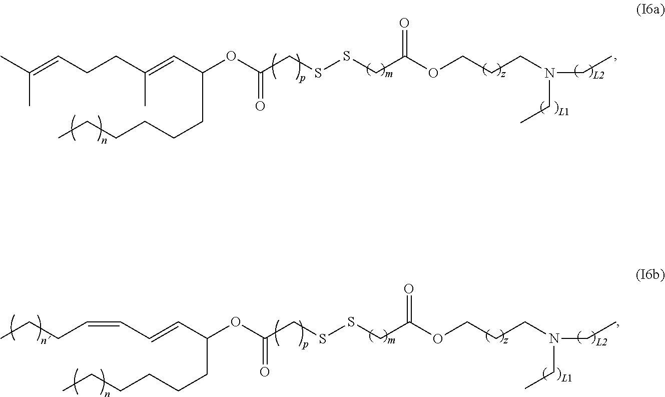

wherein m, n, p, z, L1 and L2 are as defined in claims 12 or 13, and n′ is a number from 1 to 14.

In some embodiments, compounds of the present disclosure can have a structure of Formula (I7):

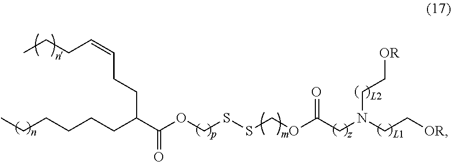

or a pharmaceutically acceptable salt thereof, wherein n and n′ are each independently a number from 1 to 16; m and p are each independently a number from 1 to 10; z is a number from 1 to 8; L1 and L2 are independently a number from 0 and 3; and R is H or —COCH₃.

In some embodiments, the compounds of Formula (I7) or the pharmaceutically acceptable salt thereof, are such that n and n′ are each independently a number from 1 to 16; m and p are each independently a number from 1 to 3; z is 1 or 2; L1 and L2 are 1; and R is H or —COCH₃.

In some embodiments, compounds of the present disclosure can have a structure of Formula (I8):

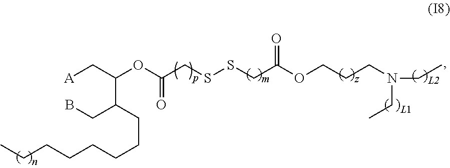

or a pharmaceutically acceptable salt thereof, wherein m and p are independently a number from 1 to 10; n is a number from 1 to 16; z is a number from 0 to 8; L1 and L2 are independently a number from 0 to 3; and A and B are independently a linear or branched C4-C20 alkyl, a linear or branched C4-C20 alkenyl, or a linear or branched C4-C20 alkynyl group.

In some embodiments, the compounds of Formula (I8), or the pharmaceutically acceptable salt thereof, are such that m and p are independently a number from 1 to 3; n is a number from 1 to 16; z is a number from 0 to 2; L1 and L2 are 0; A is a linear or branched C5-C20 alkyl, or a linear or branched C5-C20 alkenyl; and B is a linear or branched C5-C20 alkenyl group.

In some embodiments, the compounds of Formula (I8) can have a structure of Formula (I8a)

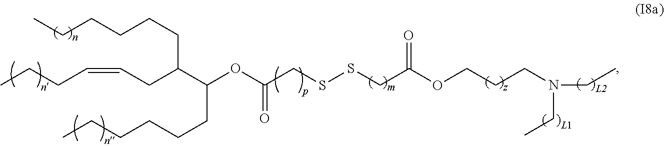

or a pharmaceutically acceptable salt thereof, wherein m, n, p, z, L1 and L2 are as defined in claims 17 or 18, n′ is a number from 1 to 14 and n″ is a number from 1 to 14.

In some embodiments, compounds of the present disclosure can have a structure of Formula (I9):

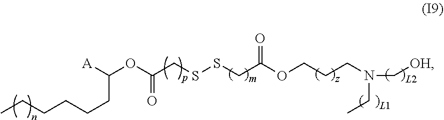

Formula (I9). (19), or a pharmaceutically acceptable salt thereof, and wherein n is a number from 1 to 16; m and p are independently a number from 1 to 10; z is a number from 0 to 8; L1 and L2 are independently a number from 0 to 3; and A is a linear or branched C4-C20 alkyl, a linear or branched C4-C20 alkenyl, or a linear or branched C4-C20 alkynyl group.

In some embodiments, the compounds of Formula (I9) or the pharmaceutically acceptable salt thereof, are such that n is a number from 1 to 16; m and p are independently a number from 1 to 3; z is a number from 0 to 2; L1 is 0; L2 is 1; and A is a linear or branched C5-C20 alkenyl group.

In some embodiments, compounds of the present disclosure, without being limited to, can be selected from the group consisting of Compounds 100, 101, 102, 103, 104, 105, 106, 107, 108, 109, 110, 111 and 112 of Table 1, or a pharmaceutically acceptable salt thereof.

TABLE 1
Non-Limiting Examples of Ionizable Lipids

| Structure | Name |
|---|---|
| 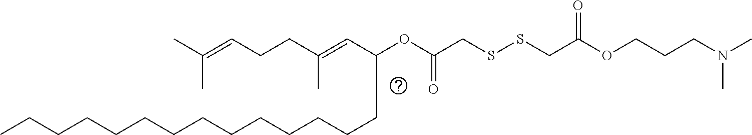 [SMILES: CCCCCCCCCCCCCCC(/C=C(\C)CCC=C(C)C)OC(=O)CSSCC(=O)OCCCN(C)C] | 100 |
| 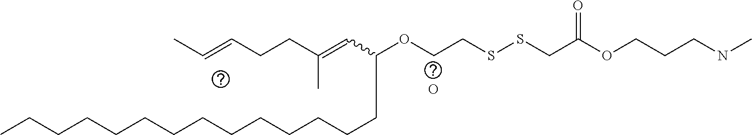 | 101 |
| 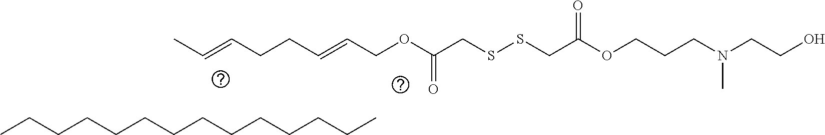 | 102 |
| 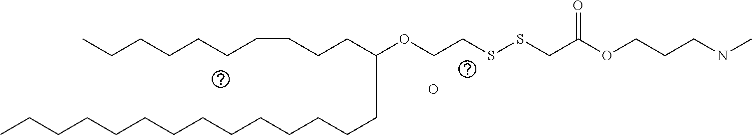 | 103 |
| 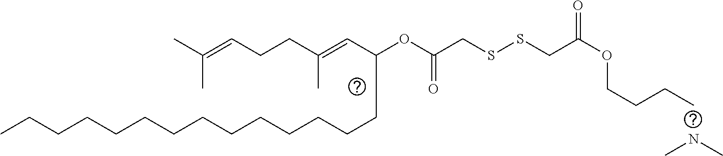 | 104 |
| 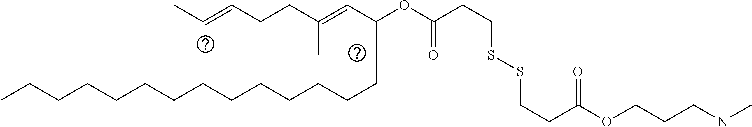 [SMILES: C/C=C/CC/C(C)=C/C(CCCCCCCCCCCCCC)OC(=O)CCSSCCC(=O)OCCCNC] | 105 |
| 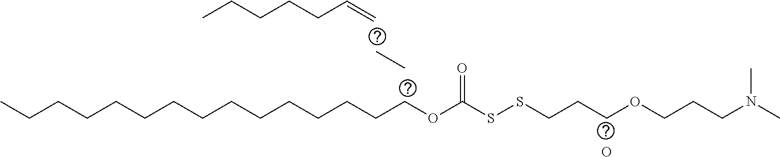 | 106 |
| 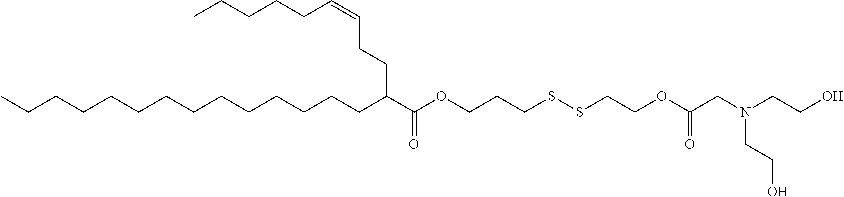 [SMILES: CCCCC/C=C\CCC(CCCCCCCCCCCCCC)C(=O)OCCCSSCCOC(=O)CN(CCO)CCO] | 107 |
| 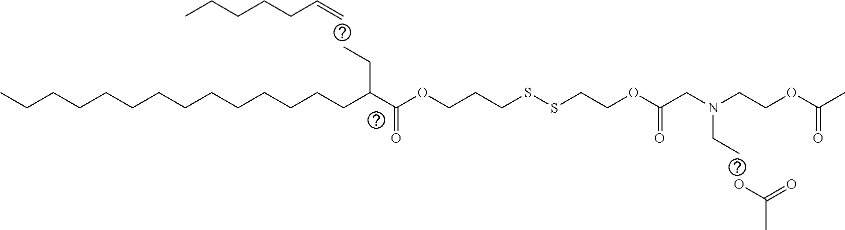 | 108 |
| 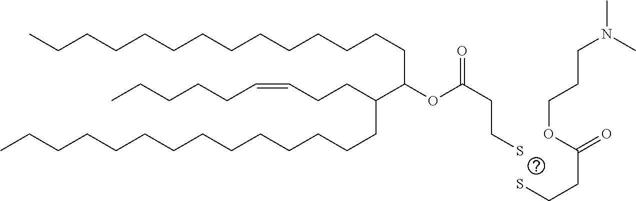 | 109 |
| 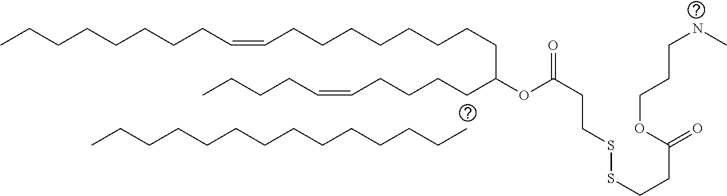 | 110 |
| 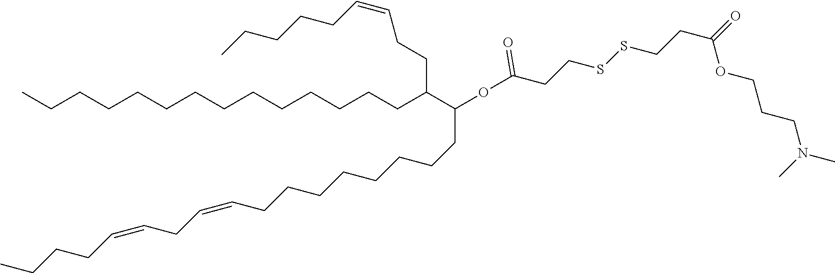 [SMILES: CCCC/C=C\C/C=C\CCCCCCCCC(OC(=O)CCSSCCC(=O)OCCCN(C)C)C(CC/C=C\CCCCC)CCCCCCCCCCCCCC] | 111 |
| 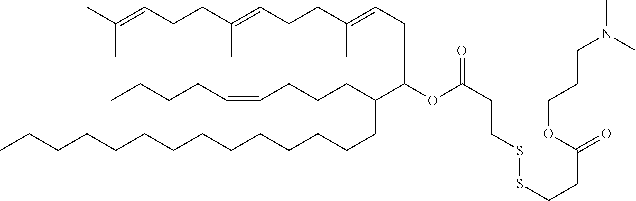 [SMILES: CCCC/C=C\CCCC(CCCCCCCCCCCCCC)C(C/C=C(\C)CC/C=C(\C)CCC=C(C)C)OC(=O)CCSSCCC(=O)OCCCN(C)C] | 112 |

The term “compound”, as used herein, is meant to embrace all stereoisomers, geometric isomers, tautomers, and isotopes of a depicted or described structure associated with the compound. When referring to compound features or substituents, the terms “optional” or “optionally” refer to a feature or substituent that may or may not occur. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

The compounds herein described may have asymmetric centers, geometric centers (e.g., double bond), or both. All chiral, diastereomeric, racemic forms and all geometric isomeric forms of a structure are intended, unless the specific stereochemistry or isomeric form is specifically indicated. Compounds of the present disclosure containing an asymmetrically substituted atom may be isolated in optically active or racemic forms. It is well known in the art how to prepare optically active forms, such as by resolution of racemic forms, by synthesis from optically active starting materials, or through use of chiral auxiliaries. Geometric isomers of olefins, C═N double bonds, or other types of double bonds may be present in the compounds described herein, and all such stable isomers are included in the present disclosure. Specifically, cis and trans geometric isomers of the compounds of the present disclosure may also exist and may be isolated as a mixture of isomers or as separated isomeric forms.

Compounds described herein also embrace tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds described herein also embrace all the isotopes of the atoms occurring in the intermediate or final compounds. “Isotopes” refers to atoms having the same atomic number but different mass numbers resulting from a different number of neutrons in the nuclei. Thus, by way of example, each individual hydrogen atom present in formula (200) may be present as a ¹H, ²H (deuterium) or ³H (tritium) atom, preferably ¹H or ²H. Similarly, by way of example, each individual carbon atom present in formula (200) may be present as a ¹²C, ¹³C or ¹⁴C atom, preferably ¹²C.

The compounds or structures and salts of the present disclosure can be prepared in combination with solvent or water molecules to form solvates and hydrates by routine methods.

### III. Delivery Vehicles

The present disclosure also provides delivery vehicles comprising a cargo or payload. As used herein, the term “cargo” or “payload” can refer to one or more molecules or structures encompassed in a delivery vehicle for delivery to or into a cell or tissue. Non-limiting examples of cargo can include a nucleic acid, a polypeptide, peptide, protein, a liposome, a label, a tag, a small chemical molecule, a large biological molecule, and any combinations or fragments thereof. In the originator constructs and benchmark constructs, the region of the construct which comprises or encodes the cargo or payload is referred to as the “cargo region” or the “payload region.”

In some embodiments, the delivery vehicle comprises at least one lipid of the present disclosure discussed in Section II, such as an ionizable lipid with a general structure of Formula (I), of any one of structures of Formula (I1), (I2), (I3), (I4), (I5), (I6), (I6a), (I6b), (I7), (I8), (I8a) or (I9), or one of the compounds in Table 1. Not willing to be bound to any theory, the disulfide bonds of the lipids cleave when the delivery vehicle goes to the targeted region thereby facilitating the release the cargo of the delivery vehicle. The length of the Y1, Y2, R1 and R2 groups in Formula (I) can also be adjusted to reach the desired zeta potential, particle size or membrane rigidity.

The total weight percentage of the lipid(s) of the present disclosure in the delivery vehicle is between about 10% to about 95%, such as between about 10% to about 20%, between about 21% to about 30%, between about 31% to about 40%, between about 41% to about 50%, between about 51% to about 60%, between about 61% to about 70%, between about 71% to about 80%, between about 81% to about 90%, or between about 91% to about 95%.

The total mole percentage of the lipid(s) in Table 1 in the delivery vehicle is between about 10% to about 95%, such as between about 10% to about 20%, between about 21% to about 30%, between about 31% to about 40%, between about 41% to about 50%, between about 51% to about 60%, between about 61% to about 70%, between about 71% to about 80%, between about 81% to about 90%, or between about 91% to about 95%.

In some embodiments, at least one lipid in the delivery vehicle has a structure of Formula (I).

The total weight percentage of the lipid(s) having a structure of Formula (I) in the delivery vehicle is between 10%-95%, such as between about 10% to about 20%, between about 21% to about 30%, between about 31% to about 40%, between about 41% to about 50%, between about 51% to about 60%, between about 61% to about 70%, between about 71% to about 80%, between about 81% to about 90%, or between about 91% to about 95%.

The total mole percentage of the lipid(s) having a structure of Formula (I) in the delivery vehicle is between 10%-95%, such as between about 10% to about 20%, between about 21% to about 30%, between about 31% to about 40%, between about 41% to about 50%, between about 51% to about 60%, between about 61% to about 70%, between about 71% to about 80%, between about 81% to about 90%, or between about 91% to about 95%.

In some embodiments, the delivery vehicle further comprises at lease additional lipid. Non-limiting examples include an additional cationic lipid, a neutral lipid, an anionic lipid, a helper lipid, a stealth lipid, or a polyethylene glycol (PEG) lipid.

“Helper lipids” are lipids that enhance transfection, such as transfection of the delivery vehicle including the payloads and cargos. The mechanism by which the helper lipid enhances transfection may include enhancing particle stability and/or enhancing membrane fusogenicity. Helper lipids include steroids and alkyl resorcinols. Helper lipids suitable for use in the present disclosure include, but are not limited to, cholesterol, 5-heptadecylresorcinol, and cholesterol hemisuccinate.

“Stealth lipids” are lipids that extend the length of time for which the delivery vehicle can exist in vivo (e.g. in the blood). Stealth lipids suitable for use in a lipid composition of the present disclosure include, but are not limited to, stealth lipids having a hydrophilic head group linked to a lipid moiety.

Non-limiting examples of cationic lipids suitable for use in the delivery vehicle of the present disclosure include, but are not limited to, N,N-dioleyl-N,N-dimethylammonium chloride (DODAC), N,N-distearyl-N,N-dimethylammonium bromide (DDAB), N-(1-(2,3-dioleoyloxy) propyl)-N,N,N-trimethylammonium chloride (DOTAP), 1,2-Dioleoyl-3-Dimethylammonium-propane (DODAP), N-(1-(2,3-dioleyloxy)propyl)-N,N,N-trimethylammonium chloride (DOTMA), 1,2-Dioleoylcarbamyl-3-Dimethylammonium-propane (DOCDAP), 1,2-Dilineoyl-3-Dimethylammonium-propane (DLINDAP), dilauryl(C12:0)trimethyl ammonium propane (DLTAP), Dioctadecylamidoglycyl spermine (DOGS), DC-Choi, Dioleoyloxy-N-[2-sperminecarboxamido)ethyl}-N,N-dimethyl-1-propanaminiumtrifluoroacetate (DOSPA), 1,2-Dimyristyloxypropyl-3-dimethyl-hydroxyethyl ammonium bromide (DMRIE), 3-Dimethylamino-2-(Cholest-5-en-3-beta-oxybutan-4-oxy)-1-(cis,cis-9,12-octadecadienoxy)propane (CLinDMA), N,N-dimethyl-2,3-dioleyloxy)propylamine (DODMA), 2-[5′-(cholest-5-en-3[beta]-oxy)-3′-oxapentoxy)-3-dimethyl-1-(cis,cis-9′,12′-octadecadienoxy) propane (CpLinDMA) and N,N-Dimethyl-3,4-dioleyloxybenzylamine (DMOBA), and 1,2-N,N′-Dioleylcarbamyl-3-dimethylaminopropane (DOcarbDAP).

Non-limiting example of neutral lipids suitable for use in the delivery vehicle of the present disclosure include a variety of neutral, uncharged or zwitterionic lipids. Examples of neutral phospholipids suitable for use in the present invention include, but are not limited to: 5-heptadecylbenzene-1,3-diol (resorcinol), dipalmitoylphosphatidylcholine (DPPC), distearoylphosphatidylcholine (DSPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-doeicosenyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof.

Non-limiting examples of anionic lipids suitable for use in the delivery vehicle of the present disclosure include, but are not limited to, phosphatidylglycerol, cardiolipin, diacylphosphatidylserine, diacylphosphatidic acid, N-dodecanoyl phosphatidyl ethanolamine, N-succinyl phosphatidylethanolamine, N-glutaryl phosphatidylethanolamine cholesterol hemisuccinate (CHEMS), and lysylphosphatidylglycerol.

In some embodiments, the weight ratio of the delivery vehicle (including all the lipids) and the payload is between about 100:1 to about 1:1, such as between about 100:1 to about 90:1, between about 89:1 to about 80:1, between about 79:1 to about 70:1, between about 69:1 to about 60:1, between about 59:1 to about 50:1, between about 49:1 to about 40:1, between about 39:1 to about 30:1, between about 29:1 to about 20:1, between about 19:1 to about 10:1, and between about 9:1 to about 1:1.

In some embodiments, the delivery vehicle further comprises an originator construct or a benchmark construct with at least one cargo or payload. The cargo or payload may be any DNA, RNA or polypeptide described herein.

In some embodiments, the delivery vehicle comprises an originator construct or a benchmark construct with at least one cargo or payload which is a coding RNA.

In some embodiments, the delivery vehicle comprises an originator construct or a benchmark construct with at least one cargo or payload which is a non-coding RNA.

In some embodiments, the delivery vehicle comprises an originator construct or a benchmark construct with at least one cargo or payload which is an oRNA.

In some embodiments, the delivery vehicle comprises an originator construct or a benchmark construct with at least one cargo or payload which is an mRNA.

In some embodiments, the at least one RNA compound is comprised of a functional RNA where the RNA results in at least one change in a cell, tissue, organ and/or organism. Said changes in state may include, but are not limited to, altering the expression level of a polypeptide, altering the translation level of a nucleic acid, altering the expression level of a nucleic acid, altering the amount of a polypeptide present in a cell, tissue, organ and/or organism, changing a genetic sequence of a cell, tissue, organ and/or organism, adding nucleic acids to a target genome, subtracting nucleic acids from a target genome, altering physiological activity in a cell, tissue, organ and/or organism or any combination thereof.

In some embodiments, the delivery vehicle comprises an originator construct or a benchmark construct with at least one cargo or payload which is DNA.

In some embodiments, the delivery vehicle comprises an originator construct or a benchmark construct with two cargos or payloads which are DNA. The DNA may be the same DNA or different DNA. As a non-limiting example, the DNA are the same. As a non-limiting example, the DNA are different. As a non-limiting example, the DNA are different but encode the same payload or cargo. As a non-limiting example, the DNA are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with three cargos or payloads which are DNA. The DNA may be the same DNA or different DNA. As a non-limiting example, the DNA are the same. As a non-limiting example, the DNA are different. As a non-limiting example, two DNA are the same and one is different. As a non-limiting example, the first DNA is different from the second and third DNA. As a non-limiting example, the first DNA, second DNA and third DNA are all different. As a non-limiting example, the first DNA is different from the second and third DNA but they all encode the same payload or cargo. As a non-limiting example, the first DNA is different from the second and third DNA but the second and third DNA encode the same payload or cargo.

In some embodiments, the delivery vehicle comprises an originator construct or a benchmark construct with at least one cargo or payload which is a polypeptide.

In some embodiments, the delivery vehicle comprises an originator construct or a benchmark construct with two cargos or payloads which are polypeptide. The polypeptide may be the same polypeptide or different polypeptide As a non-limiting example, the polypeptide are the same. As a non-limiting example, the polypeptide are different. As a non-limiting example, the polypeptides are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with three cargos or payloads which are polypeptide. The polypeptide may be the same polypeptide or different polypeptide. As a non-limiting example, the polypeptide are the same. As a non-limiting example, the polypeptide are different. As a non-limiting example, two polypeptide are the same and one is different. As a non-limiting example, the first polypeptide is different from the second and third polypeptide. As a non-limiting example, the first polypeptide, second polypeptide and third polypeptide are all different. As a non-limiting example, the first polypeptide is different from the second and third polypeptide but they all encode the same payload or cargo. As a non-limiting example, the first polypeptide is different from the second and third polypeptide but the second and third polypeptide encode the same payload or cargo.

In some embodiments, the delivery vehicle comprises an originator construct or a benchmark construct with at least one cargo or payload which is a peptide.

In some embodiments, the delivery vehicle comprises an originator construct or a benchmark construct with two cargos or payloads which are peptide. The peptide may be the same peptide or different peptide. As a non-limiting example, the peptide are the same. As a non-limiting example, the peptides are different. As a non-limiting example, the peptides are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with three cargos or payloads which are peptide. The peptide may be the same peptide or different peptide. As a non-limiting example, the peptides are the same. As a non-limiting example, the peptides are different. As a non-limiting example, two peptides are the same and one is different. As a non-limiting example, the first peptide is different from the second and third peptide. As a non-limiting example, the first peptide, second peptide and third peptide are all different. As a non-limiting example, the first peptide is different from the second and third peptide but they all encode the same payload or cargo. As a non-limiting example, the first peptide is different from the second and third peptide but the second and third peptide encode the same payload or cargo.

In some embodiments, the delivery vehicle comprises an originator construct or a benchmark construct with at least one cargo or payload which is RNA.

In some embodiments, the delivery vehicle comprises an originator construct or a benchmark construct with two cargos or payloads which are RNA. The RNA may be the same RNA or different RNA. As a non-limiting example, the RNAs are the same. As a non-limiting example, the RNAs are different. As a non-limiting example, the RNAs are different but encode the same payload or cargo. As a non-limiting example, the RNAs are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with three cargos or payloads which are RNA. The RNA may be the same RNA or different RNA. As a non-limiting example, the RNA are the same. As a non-limiting example, the RNA are different. As a non-limiting example, two RNA are the same and one is different. As a non-limiting example, the first RNA is different from the second and third RNA. As a non-limiting example, the first RNA, second RNA and third RNA are all different. As a non-limiting example, the first RNA is different from the second and third RNA but they all encode the same payload or cargo. As a non-limiting example, the first RNA is different from the second and third RNA but the second and third RNA encode the same payload or cargo.

In some embodiments, the delivery vehicle comprises an originator construct or a benchmark construct with two cargos or payloads where one is RNA and one is DNA. The RNA and DNA may encode the same peptide or polypeptide or may encode different peptides or polypeptides. As a non-limiting example, the RNA and DNA may encode the same peptide or polypeptide. As a non-limiting example, the RNA and DNA may encode different peptides or polypeptides. As a non-limiting example, the RNA and DNA are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with two cargos or payloads where one is RNA and one is a peptide. The RNA may encode the same peptide as the peptide cargo/payload the RNA may encode a different peptide. As a non-limiting example, the RNA encodes the same peptide. As a non-limiting example, the RNA encodes a different peptide. As a non-limiting example, the RNA and peptide are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with two cargos or payloads where one is RNA and one is a polypeptide. The RNA may encode the same polypeptide as the polypeptide cargo/payload the RNA may encode a different polypeptide. As a non-limiting example, the RNA encodes the same polypeptide. As a non-limiting example, the RNA encodes a different polypeptide. As a non-limiting example, the RNA and polypeptide are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with two cargos or payloads where one is DNA and one is a peptide. The DNA may encode the same peptide as the peptide cargo/payload the DNA may encode a different peptide. As a non-limiting example, the DNA encodes the same peptide. As a non-limiting example, the DNA encodes a different peptide. As a non-limiting example, the DNA and peptide are different pieces of a larger payload or cargo (e.g., heavy chain or light chain of an antibody) that can come together using natural systems or synthetic methods known in the art to produce a functional polypeptide (e.g., antibody).

In some embodiments, the delivery vehicle comprises an originator construct or a benchmark construct with two cargos or payloads where one is DNA and one is a polypeptide. The DNA may encode the same polypeptide as the polypeptide cargo/payload the DNA may encode a different polypeptide. As a non-limiting example, the DNA encodes the same polypeptide. As a non-limiting example, the DNA encodes a different polypeptide. As a non-limiting example, the DNA and polypeptide are different pieces of a larger payload or cargo (e.g., heavy chain or light chain of an antibody) that can come together using natural systems or synthetic methods known in the art to produce a functional polypeptide (e.g., antibody).

### Nanoparticles

In some embodiments, the delivery vehicle is a nanoparticle. The term “nanoparticle” as used herein refers to any particle ranging in size from 10-1000 nm. The nanoparticle may be 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 nm.

### Lipid Nanoparticles

In some embodiments, the nanoparticles may be a lipid nanoparticle (LNP). In general, LNPs can be characterized as small solid or semi-solid particles possessing an exterior lipid layer with a hydrophilic exterior surface that is exposed to the non-LNP environment, an interior space which may aqueous (vesicle like) or non-aqueous (micelle like), and at least one hydrophobic inter-membrane space. LNP membranes may be lamellar or non-lamellar and may be comprised of 1, 2, 3, 4, 5 or more layers. In some embodiments, LNPs may comprise a cargo or a payload into their interior space, into the inter membrane space, onto their exterior surface, or any combination thereof.

LNPs useful herein are known in the art and generally comprise cholesterol (aids in stability and promotes membrane fusion), a phospholipid (which provides structure to the LNP bilayer and also may aid in endosomal escape), a polyethylene glycol (PEG) derivative (which reduces LNP aggregation and “shields” the LNP from non-specific endocytosis by immune cells), and an ionizable lipid (complexes negatively charged RNA and enhances endosomal escape), which form the LNP-forming composition.

The components of the LNP may be selected based on the desired target, cargo, size, etc. As a non-limiting example, previous studies have shown that that polymeric nanoparticles made of low molecular weight polyamines and lipids can deliver nucleic acids to endothelial cells with high efficiency. (Dahlman, et al, In vivo endothelial siRNA delivery using polymeric nanoparticles with low molecular weight, Nat Nanotechnol. 2014 August; 9(8): 648-655; the contents of which is herein incorporated by reference in its entirety).

In some embodiments, compounds/lipids the present disclosure may be incorporated into lipid nanoparticles (LNPs). In some embodiments a lipid nanoparticle may be comprised of at least one cationic lipid, at least one non-cationic lipid, at least one sterol, at least one particle-activity-modifying-agent, or any combination thereof. In some embodiments a lipid nanoparticle may be comprised of at least one cationic lipid, at least one non-cationic lipid, at least one sterol, and at least one particle-activity-modifying-agent. In some embodiments, the LNP may be comprised of at least one cationic lipid, at least one non-cationic lipid, and at least one sterol. In some embodiments, the LNP may be comprised of at least one cationic lipid, at least one non-cationic lipid, and at least one particle-activity-modifying-agent. In some embodiments, the LNP may be comprised of at least one non-cationic lipid, at least one sterol, and at least one particle-activity-modifying-agent. In some embodiments, the LNP may be comprised of at least one cationic lipid and at least one non-cationic lipid. In some embodiments, the LNP may be comprised of at least one cationic lipid and at least one sterol. In some embodiments, the LNP may be comprised of at least one cationic lipid and at least one particle-activity-modifying-agent. In some embodiments, the LNP may be comprised of at least one non-cationic lipid and at least one sterol. In some embodiments, the LNP may be comprised of at least one non-cationic lipid and at least one particle-activity-modifying-agent. In some embodiments, the LNP may be comprised of at least one sterol and at least one particle-activity-modifying-agent. In some embodiments, the LNP may be comprised of at least one cationic lipid. In some embodiments, the LNP may be comprised of at least one non-cationic lipid. In some embodiments, a LNP may be comprised of a sterol. In some embodiments, the LNP may be comprised of a particle-activity-modifying-agent.

In some embodiments, the at least one cationic lipid may comprise any of at least one ionizable cationic lipid, at least one amino lipid, at least one saturated cationic lipid, at least one unsaturated cationic lipid, at least one zwitterionic lipid, at least one multivalent cationic lipid, or any combination thereof. In some embodiments, the LNP may be essentially devoid of the at least one cationic lipid. In some embodiments, the LNP may contain no amount of the at least one cationic lipid.

In some embodiments, at least one cationic lipid may be selected from, but not limited to, at least one of 1,3-Bis-(1,2-bis-tetradecyloxy-propyl-3-dimethylethoxyammoniumbromide)-propan-2-ol ((R)-PLC-2), 2-(Dinonylamino)ethan-1-ol (17-10), 2-(Didodecylamino)ethan-1-ol (17-11), 3-(Didodecylamino)propan-1-ol (17-12), 4-(Didodecylamino)butan-1-ol (17-13), 2-(Hexyl((9Z,12Z)-octadeca-9,12-dien-1-yl)amino)ethan-1-ol (17-2), 2-(Nonyl((9Z,12Z)-octadeca-9,12-dien-1-yl)amino)ethan-1-ol (17-3), 2-(Dodecyl((9Z,12Z)-octadeca-9,12-dien-1-yl)amino)ethan-1-ol (17-4), 2-(((9Z,12Z)-Octadeca-9,12-dien-1-yl)(tetradecyl)amino)ethan-1-ol (17-5), 2-(((9Z,12Z)-Octadeca-9,12-dien-1-yl)(octadecyl)amino)ethan-1-ol (17-6), 2-(Ditetradecylamino)ethan-1-ol (17-7), 2-(Di((Z)-octadec-9-en-1-yl)amino)ethan-1-ol (17-8), (9Z,12Z)—N-(2-Methoxyethyl)-N-((9Z,12Z)-octadeca-9,12-dien-1-yl)octadeca-9,12-dien-1-amine (17-9), N-Nonyl-N-(2-(piperazin-1-yl)ethyl)nonan-1-amine (19-1), N-Dodecyl-N-(2-(piperazin-1-yl)ethyl)dodecan-1-amine (19-2), (9Z,12Z)—N-((9Z,12Z)-Octadeca-9,12-dien-1-yl)-N-(2-(piperazin-1-yl)ethyl)octadeca-9,12-dien-1-amine (19-3), N-Dodecyl-N-(2-(4-methylpiperazin-1-yl)ethyl)dodecan-1-amine1ntermediate1:2-(Didodecylamino)ethan-1-ol (19-4), N-Dodecyl-N-(2-(4-(4-methoxybenzyl)piperazin-1-yl)ethyl)dodecan-1-amine (19-5), (9Z,12Z)—N-(2-(4-Dodecylpiperazin-1-yl)ethyl)-N-((9Z,12Z)-octadeca-9,12-dien-1-yl)octadeca-9,12-dien-1-amine (19-6), (3-((6Z,9Z,28Z,31Z)-heptatriaconta-6,9,28,31-tetraen-19-yloxy)-N,N-dimethylpropan-1-amine) (1-Bi 1), N-(2-(Didodecylamino)ethyl)-N-dodecylglycine (20-1), Dinonyl8,8′-((2-(dodecyl(2-hydroxyethyl)amino)ethyl)azanediyl)dioctanoate (20-10), 3-((2-(Ditetradecylamino)ethyl)(dodecyl)amino)propan-1-ol (20-11), 2-((2-(Ditetradecylamino)ethyl)(tetradecyl)amino)ethan-1-ol (20-12), 2-((2-(Di((9Z,12Z)-octadeca-9,12-dien-1-yl)amino)ethyl)(dodecyl)amino)ethan-1-ol (20-13), 2-((2-(Di((9Z,12Z)-octadeca-9,12-dien-1-yl)amino)ethyl)((9Z,12Z)-octadeca-9,12-dien-1-yl)amino)ethan-1-ol (20-14), 2-((2-(Didodecylamino)ethyl)(hexyl)amino)ethan-1-ol (20-15), 2-((2-(Dinonylamino)ethyl)(nonyl)amino)ethan-1-ol (20-16), 2-((2-(Didodecylamino)ethyl)(nonyl)amino)ethan-1-ol (20-17), 2-((2-(Dinonylamino)ethyl)(dodecyl)amino)ethan-1-ol (20-18), 2-((2-(Didodecylamino)ethyl)amino)ethan-1-ol (20-19), Pentyl6-(dodecyl(2-(dodecyl(2-hydroxyethyl)amino)ethyl)amino)hexanoate (20-2), 2-((2-(Didodecylamino)ethyl)(dodecyl)amino)ethan-1-ol (20-20), 3-((2-(Didodecylamino)ethyl)(dodecyl)amino)propan-1-ol (20-21), 4-((2-(Didodecylamino)ethyl)(dodecyl)amino)butan-1-ol (20-22), (Z)-2-((2-(Didodecylamino)ethyl)(dodec-6-en-1-yl)amino)ethan-1-ol (20-23), 2-((2-(Didodecylamino)ethyl)(tetradecyl)amino)ethan-1-ol (20-24), 2-((2-(Didodecylamino)ethyl)((9Z,12Z)-octadeca-9,12-dien-1-yl)amino)ethan-1-ol (20-25), Pentyl6-((2-(didodecylamino)ethyl)(2-hydroxyethyl)amino)hexanoate (20-3), Dipentyl6,6′-((2-(dodecyl(2-hydroxyethyl)amino)ethyl)azanediyl)dihexanoate (20-4), Diheptyl6,6′-((2-((6-(heptyloxy)-6-oxohexyl)(2hydroxyethyl)amino)ethyl)azanediyl)dihexanoate (20-5), Pentyl6-((2-(dinonylamino)ethyl)(2-hydroxyethyl)amino)hexanoate (20-6), Heptyl6-(dodecyl(2-(dodecyl(2-hydroxyethyl)amino)ethyl)amino)hexanoate (20-7), Nonyl8-((2-(didodecylamino)ethyl)(2-hydroxyethyl)amino)octanoate (20-8), Heptadecan-9-yl8-((2-(didodecylamino)ethyl)(2-hydroxyethyl)amino)octanoate (20-9), 1-(2,2-Di((9Z,12Z)-octadeca-9,12-dien-1-yl)cyclopropyl)-N,N-dimethylmethanamine (21-1), 3,3-Di((9Z,12Z)-octadeca-9,12-dien-1-yl)cyclobutyl4-(dimethylamino)butanoate (21-2), 3,3-Di((9Z,12Z)-octadeca-9,12-dien-1-yl)cyclopentyl3-(dimethylamino)propanoate (21-3), 3,3-Di((9Z,12Z)-octadeca-9,12-dien-1-yl)cyclopentyl4-(dimethylamino)butanoate (21-4), 1-(2,3-Di((8Z,11Z)-heptadeca-8,11-dien-1-yl)cyclopropyl)-N,N-dimethylmethanamine (21-6), Unknown (75-016B), poly{4-((2-(dimethylamino)ethyl)thio)tetrahydro-2H-pyran-2-one}-r-poly{4-(octylthio)tetrahydro-2H-pyran-2-one}(A7), (3aR5s,6aS)—N,N-dimethyl-2,2-di((9Z,12Z)-octadeca-9,12-dienyl)tetrahydro-3aH-cyclopentad1,3dioxol-5-amine (ALN100), (3aR,5s,6aS)—N,N-dimethyl-2,2-di((9Z,12Z)-octadeca-9,12-dienyl)tetrahydro-3aHcyclopenta[d][1,3]dioxol-5-amine (ALN1001), ((3aR,5s,6aS)—N,N-dimethyl-2,2-di((9Z,12Z)-octadeca-9,12-dienyl)tetrahydro-3aH-cyclopenta[d][1,3]dioxol-5-amine)) (ALNY-100), dimyristoyltrimethylammoniumpropane (Amino Lipid 6), Benzami7di7-dialkyl-carboxylicacid (BADACA), N,N-dihydroxyethylmethyl-N-2-(cholesteryloxycarbonylamino)ethylammoniumbromide (BHEM-Chol), N,N-bis-(2-hydroxyethyl)-N-methyl-N-(2-cholesteryloxycarbonylamino-ethyl)ammoniumbromide (BHEM-Chol1), 2-{4-[(33)-cholest-5-en-3-yloxy]butoxy}-iV?N-dimethyl-3-[(9Z,12Z)-octadeca-9!12-dien-1-yloxy]propan-1-amine (Butyl-CLinDMA), (2JR)-2-{4-[(3β)-cholest-5-en-3-yloxy]butoxy}-Λr\hat{ }dimethyl-3-[(9Z,12Z)-octadeca-9!12-dien-1-yloxyjpropan-1-amine (Butyl-CLinDMA (2R)), (25)-2-{4-[(3β)-cholest-5-en-3-yloxy]butoxy}-iVy/V-dimethyl-3-[(9Z,12Z)-octadeca-9,12-dien-1-yloxy]propan-1-amine (Butyl-CLinDMA (2S)), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-o! (C12-200), 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), Cholesteryl-succinyl Silane (C2), (9Z,9′Z,12Z,12′Z)-2-((4-(((3-(dimethylamino)propoxy)carbonyl)oxy)hexadecanoyl)oxy)propane-1,3-diylbis(octadeca-9,12-dienoate) (Cationic Lipid A2), 9Z,12Z)-3-((4,4-bis(octyloxy)butanoyl)oxy)-2-((((3-(diethylamino)propoxy)carbonyl)oxy)methyl)propyloctadeca-9,12-dienoate (Cationic Lipid A3), 1-(3-cholesteryl)-oxycarbonyl-aminomethylimidazole (CHIM), [(2-Morpholine-4-yl-ethylcarbamoyl)methyl]-carbamicacidcholesterylester (Chol-C3N—Mo2), [(2-Morpholine-4-yl-ethylcarbamoyl)-ethyl]-carbamicacidcholesterylesterChol-DMC3N—Mo2[1-Methyl-2-(2-morpholine-4-yl-ethylcarbamoyl)-propyl]-carbamicacidcholesterylester (Chol-C4N—Mo2), 1,17-bis(2-octylcyclopropyl)heptadecan-9-yl4-(dimethylamino)butanoate (CL), heptatriaconta-6,9,28,31-tetraen-19-yl-4-(dimethylamino)-butanoate (CL01), cholesteryl3-(dimethylamino)propanoate (CL06), cholesteryl2-(dimethylamino)acetate (CL08), N,N-dimethyl-2,3-bis(((9Z,12Z)-octadeca-9,12-dien-1-yl)oxy)propan-1-amine (CL-1), N-methyl-2-(((9Z,12Z)-octadeca-9,12-dien-1-yl)oxy)-N-(2-((((9Z,12Z)-octadeca-9,12-diene-1-yl)oxy)ethyl)ethan-1-amine (CL-11), (3R,4R)-3,4-bis(((Z)-hexadec-9-en-1-yl)oxy)-1-methylpyrrolidine(CompoundCL-12) (CL-12), 2-(Dimethylamino)-N-((6Z,9Z,28Z,31Z)-Heptatriaconta-6,9,28,31-tetraen-19-yl)acetamide (CL-13), 3-(Dimethylamino)propane-1,2-diyl(9Z,9′Z,12Z,12′Z)-bis(octadeca-9,12-dienoate) (CL-14), (9Z,12Z)-di((9Z,12Z)-octadeca-9,12-dien-1-yl)amine (CL-15), 7-Hydroxy7-(4-((1-methylpiperidine-4-carbonyl)oxy)butyl)tridecane-1,13-diyldidodecanoate (CL15B6), 7-Hydroxy7-(4-((1-methylpiperidine-4-carbonyl)oxy)butyl)tridecane-1,13-diylditetradecanoate (CL15C6), 7-Hydroxy7-(4-((1-methylpiperidine-4-carbonyl)oxy)butyl)tridecane-1,13-diyldipalmitate (CL15D6), 7-Hydroxy7-(4-((1-methylpiperidine-4-carbonyl)oxy)butyl)tridecane-1,13-diyldioleate (CL15H6), Bis(2-(((9Z,12Z)-octadeca-9,12-dien-1-yl)oxy)ethyl)amine (CL-16), (9Z,12Z)—N-Methyl-N-(2-(((9Z,12Z)-octadeca-9,12-dien-1-yl)oxy)ethyl)octadeca-9,12-dien-1-amine (CL-17), (9Z,12Z)—N-(3-(((9Z,12Z)-octadeca-9,12-dien-1-yl)oxy)propyl)octadeca-9,12-dien-1-amine (CL-18), (1-Methylpiperidin-3-yl)methyldi((11Z,14Z)-icosa-11,14-dien-1-yl)carbamate (CL-19), N-methyl-N,N-bis(2-((Z)-hexadec-9-enyloxy)ethyl)amine (CL-2), (13Z,16Z)—N,N-Dimethyl-4-((9Z,12Z)-octadeca-9,12-dien-1-yl)docosa-3,13,16-trien-1-amine (CL-20), (S)-2-Amino-3-hydroxy-N,N-bis(2-(((Z)-octadeca-9-en-1-yl)oxy)ethyl)propanamide (CL-21), C2:N,N-dihexadecyl-N′-(3-triethoxysilylpropyl)succinamide (CL3), trans-1-Methyl-3,4-bis((((Z)-octadec-9-en-1-yl)oxy)methyl)pyrrolidine (CL-3), trans-1-methylpyrrolidine-3,4-diyl)bis(methylene)(9Z,9′Z,12Z,12′Z)-bis(octadeca-9,12-dienoate) (CL-4), 7-(4-(Diisopropylamino)butyl)-7-hydroxytridecane-1,13-diylditetradecanoate (CL4C6), 7-(4-(Diisopropylamino)butyl)-7-hydroxytridecane-1,13-diyldipalmitate (CL4D6), 11-(4-(Diisopropylamino)butyl)-11-hydroxyhenicosane-1,21-diyldioleate (CL4H10), 7-(4-(Diisopropylamino)butyl)-7-hydroxytridecane-1,13-diyldioleate (CL4H6), 9-(4-(Diisopropylamino)butyl)-7-hydroxyheptadecane-1,17-diyldioleate (CL4H8), (6Z,9Z,28Z,31Z)-Heptatriaconta-6,9,28,31-tetraen-19-yl4-(dimethylamino)butanoate (CL-5), 2-(Dimethylamino)-N-(2-(((Z)-octadeca-9-en-1-yl)oxy)ethyl)-N-((9Z,12Z)-octadeca-9,12-diene-1-yl)acetamide (CL-53), 3-((2-(((Z)-octadeca-9-en-1-yl)oxy)ethyl)((9Z,12Z)-octadeca-9,12-dien-1-yl)amino)propane-1-All (CL-54), 1-Methyl-3,3-bis((((9Z,12Z)-octadeca-9,12-dien-1-yl)oxy)methyl)azetidine (CL-55), 1-Methyl-3,3-bis(2-(((9Z,12Z)-octadeca-9,12-dien-1-yl)oxy)ethyl)azetidine (CL-56), 1-Methyl-3,3-bis(2-(((9Z,12Z)-octadeca-9,12-dien-1-yl)oxy)propyl)azetidine (CL-57), 2-(3,3-di((9Z,12Z)-octadeca-9,12-dien-1-yl)azetidin-1-yl)ethan-1-ol (CL-58), 2-(3,3-di((9Z,12Z)-octadeca-9,12-dien-1-yl)azetidin-1-yl)propan-1-ol (CL-59), 3-(Di((9Z,12Z)-octadeca-9,12-dien-1-yl)amino)propan-1-o (CL-6), 3-(Dimethylamino)propyl3,3-di((9Z,12Z)-octadeca-9,12-dien-1-yl)azetidine-1-carboxylate (CL-60), 2-(Di((Z)-octadeca-9-en-1-yl)amino)ethane-1-ol (CL-61), 3-(Di((Z)-octadeca-9-en-1-yl)amino)propan-1-ol (CL-62), (11Z,14Z)-2-((Dimethylamino)methyl)-2-((9Z,12Z)-octadeca-9,12-dien-1-yl)icosa-11,14-dien-1-ol (CL-63), (11Z,14Z)-2-(Dimethylamino)-2-((9Z,12Z)-octadeca-9,12-dien-1-yl)icosa-11,14-dien-1-ol (CL-64), 3-(Dimethylamino)-2,2-bis((((9Z,12Z)-octadeca-9,12-dien-1-yl)oxy)methyl)propan-1-ol (CL-65), (9Z,12Z)—N-(2-(((Z)-Octadeca-9-en-1-yl)oxy)ethyl)octadeca-9,12-dien-1-amine (CL-7), 1-Methyl-3,3-di((9Z,12Z)-octadeca-9,12-dien-1-yl)azetidine (CL-8), N,2-Dimethyl-1,3-bis(((9Z,12Z)-octadeca-9,12-dien-1-yl)oxy)propan-2-amine (CL-9), 3-Dimethylamino-2-(Cholest-5-en-3B-oxybutan-4-oxy)-1-(cis,cis-9,12-octadecadienoxy)propane (CLinDMA), 2-[5′-(cholest-5-en-3-oxy)-3′-oxapentoxy)-3-dimethy-1-(cis,cis-9′,12′-octadecadienoxy)propane (CpLinDMA), cetyltrimethylammoniumbromide (CTAB), 1\hat{ }-Diarachidonyloxy-\hat{ }TV-dimethy\hat{ }-propyl-S-amine (DAraDMA), 0,0′-ditetradecanoyl-N—(α-trimethylammonioacetyl)diethanolaminechloride (DC-6-14), 3β-[N—(N′,N′-dimethylaminoethane)carbamoyl]cholesterol (DC-Chol), dimethyldioctadecylammonium (DDA), dimethyldioctadecylammoniumbromide (DDA), N,N-distearyl-N,N-dimethylammoniumbromide (DDAB), 1,2-Didocosahexaenyloxy-(7V,N-dimethyl)-propyl-3-amine (DDocDMA), N-(2-(dimethylamino)ethyl)-4,5-bis(dodecylthio)pentanamide (DEDPA), 3-Dimethylamino-2-(Cholest-5-en-33-oxypent-3-oxa-an-5-oxy)-1-(cis,cis-9,12-octadecadienoxy)propane (DEG-CLinDMA), 1,6-DileoylTriethylenetetramide (dio-TETA), N1,N19-bis((S,23E,25E,27E,29E)-16-((2E,4E,6E,8E)-3,7-dimethyl-9-(2,6,6-trimethylcyclo-hex-1-en-1-yl)nona-2,4,6,8-tetraenamido)-24,28-dimethyl-15,22-dioxo-30-(2,6,6-trimethylcyclohex-1-en-1-yl)-4,7,10-trioxa-14,21-diazatriaconta-23,25,27,29-tetraen-1-yl)-4,7,10,13,16-pentaoxanonadecane-1,19-diamide (diVA-PEG-diVA), DiLin-N-Methylpiperazine (DL-033), DiLin-N,N-DimethylGlycine (DL-036), Dioleyl-N,N-DimethylGlycine (DL-048), 3-((1,3-bis(((9Z,12Z)-octadeca-9,12-dienoyl)oxy)propan-2-yl)amino)propanoicacid (DLAPA), 1,2-dilinolenyloxy-3-dimethylaminopropane (DLenDMA), 1-Linoleoyl-2-linoleyloxy-3-dimethylaminopropane (DLin-2-DMAP), 3-(N,N-Dilinoleylamino)-1,2-propanediol (DLinAP), 1,2-N,N′-Dilinoleylcarbamyl-3-dimethylaminopropane (DLincarbDAP), 1,2-Dilinoleoylcarbamyl-3-dimethylaminopropane (DLinCDAP), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleoyl-3-dimethylaminopropane (DLinDAP), 1,2-DiLinoleyloxy-N,N-dimethylaminopropane (DLinDMA), 1,2-dilinoleyloxy-3-dimethylaminopropane (DLinDMA 1), 1,2-Dilinoleyloxo-3-(2-N,N-dimethylamino)ethoxypropane (DLin-EG-DMA), dilinoleoyl-4-aminobutyricacid (DLinFAB), 2,2-dilinoleyl-4-(2-dimethylaminoethyl)-[1,3]-dioxolane (DLin-K-C2-DMA), 2,2-Dilinoleyl-4-dimethylaminomethyl-[1,3]-dioxolane (DLin-K-DMA), 1,2-Dilinoleyoxy-3-morpholinopropane (DLin-MA), (6Z,9Z,28Z,31Z)-heptatriacont-6,9,28,31-tetraene-19-yl4-(dimethylamino)butanoate (DLin-MC3-DMA), 1,2-Dilinoleyloxy-3-(N-methylpiperazino)propane (DLinMPZ), 1,2-Dilinoleyloxy-3-(N-methylpiperazino)propane (DLin-MPZ), Dilinoleyloxy3-piperidinopropylamine (DLinPip), 1.2Dilinoleyloxy3-(3′-hydroxypiperidino)-propylamine (DLinPip-30H), 1,2Dilinoleyloxy3-(4′-hydroxypiperidino)-propylamine (DLinPip-40H), 1,2-Dilinoleyloxy-3-hydroxypropane (DLinPO), 1,2-Dilinoleylthio-3-dimethylaminopropane (DLin-S-DMA), 1,2-Dilinoleoyl-3-trimethylaminopropane (DLinTAP), 1,2-Dilinoleoyl-3-trimethylaminopropanechloridesalt (DLin-TAP.Cl), 1,2-Dilinoleyloxy-3-trimethylaminopropane (DLinTMA), 1,2-Dilinoleyloxy-3-trimethylaminopropanechloridesalt (DLin-TMA.Cl), 3-((1,3-bis(((9Z,12Z,15Z)-octadeca-9,12,15-trienoyl)oxy)propan-2-yl)amino)propanoicacid (DLLAPA), 1,2Dilinoleyloxy3-(N,NdimethyD-propylamme (DLmDEA), 1,2-Dilauroyl-sn-Glicero-3-Phosphoethanolamine (DLPE), 1,2-Dilauroyl-sn-Glicero-3-Glycerol (DLPG), N,N-Dimethyl-3,4-dioleyloxybenzylamine (DMOBA), dimyristoylphosphatidylserine (DMPS), N-[1-(2,3-dimyristyloxy)propyl]-N,N-dimethyl-N-(2-hydroxyethyl)ammoniumbromide (DMRIE), 1,2-Dimyristyloxypropyl-3-dimethyl-hydroxyethylammoniumbromide (DMRIE1), 1,2-dimyristoyl-3-trimethylammoniumpropane (DMTAP), 3-(N,N-Dioleylamino)-1,2-propanedio (DOAP), 3-((1,3-bis(oleoyloxy)propan-2-yl)amino)propanoicacid (DOAPA), 1,2-N,N′-dioleylcarbamyl-3-dimethylaminopropane (DOcarbDAP), 1,2-Dioleoylcarbamyl-3-Dimethylammonium-propane (DOCDAP), N,N-dioleyl-N,N-dimethylammoniumchloride (DODAC), 1,2-Dioleoyl-3-Dimethylammonium-propane (DODAP), N,N-dihydroxyethylN,N-dioctadecylammoniumchloride (DODEAC), N,N-dimethyl-2,3-dioleyloxypropylamine (DODMA), dioleoyl-4-aminobutyricacid (DOFAB), Dioctadecylamidoglycylspermine (DOGS), 1,2-Dioleoyl-3-methyl-(methoxycarbonyl-ethyl)ammonium-Propane (DOMCAP), 1,2-Dioleoyl-3-N-pyrrolidine-propane (DOP5P), 1,2-Dioleoyl-3-N-pyrridinium-propane,bromidesalt (DOP6P), 1,2-dioleoyl-3-dimethyl-hydroxyethylammoniumbromide (DORI), 1,2-dioleyloxypropyl-3-dimethyl-hydroxyethylammoniumbromide (DORIE), 1,2-dioleyloxypropyl-3-dimethyl-hydroxybutylammoniumbromide (DORIE-HB), 1,2-dioleyloxypropyl-3-dimetyl-hydroxypropylammoniumbromide (DORIE-HP), 1,2-dioleyloxypropyl-3-dimethyl-hydroxypentylammoniumbromide (DORIE-Hpe), 2,3-dioleyloxy-N-[2(spermine-carboxamido)ethyl]-N,N-dimethyl-1-propanaminiumtrifluoroacetate (DOSPA), 1,3-dioleoyloxy-2-(6-carboxy-spermyl)-propylamide (DOSPER), N-(1-(2,3-dioleoyloxy)propyl)-N,N,N-trimethylammoniumchloride (DOTAP), 1,2-dioleoyl-3-trimethylammonium-propane (DOTAP1), N-[5′-(2′,3′-dioleoyl)uridine]-N′,N′,N′-trimethylammoniumtosylate (DOTAU), 1-[2-(9(Z)-octadecenoyloxy)ethyl]-2-(8(Z)-heptadecenyl-3-(2-hydroxyethyl)imidazoliniumchloride (DOTIM), N-(1-(2,3-dioleyloxy)propyl)-N,N,N-trimethylammoniumchloride (DOTMA), dioleylphosphatidyluridinephosphatidylcholine (DOUPC), 1,2-Diphvtanyloxy-W.N-dimemyl)-butyl-4-amme (DPan-C2-DMA), 1,2-Diphytanyloxy-3-(iV,7V-dimethyl)-propylamine (DPanDMA), 2,3-bis(dodecylthio)propyl(2-(dimethylamino)ethyl)carbamate (DPDEC), dipalmitoyl-4-aminobutyricacid (DPFAB), 1,2-dipalmityloxypropyl-3-dimethyl-hydroxyethylammoniumbromide (DPRIE), 1,2-dipalmitoyl-3-trimethylammoniumpropane (DPTAP), 1-[2-(hexadecanoyloxy)ethyl]-2-pentadecyl-3-(2-hydroxyethyl)imidazoliniumchloride (DPTIM), 3-((1,3-bis(stearoyloxy)propan-2-yl)amino)propanoicacid (DSAPA), distearyldimethylammonium (DSDMA), 1,2-distearloxy-N,N-dimethylaminopropane (DSDMA1), 1,2-distearyloxypropyl-3-dimethyl-hydroxyethylammoniumbromide (DSRIE), 1,2-distearoyl-3-trimethylammoniumpropane (DSTAP), ditetradecyltrimethylammonium (DTDTMA), 1,2-dioleoyl-sn-glycero-3-ethylphosphocholine (EDOPC), N2-[N2,N5-bis(3-aminopropyl)-L-ormithyl]-N,N-dioctadecyl-L-glutaminetetrahydrotrifluoroacetate (GC33), Cholest-5-en-3-ol(3P)—,3-[(3-aminopropyl)[4-[(3-aminopropyl)amino]butyl]carbamate](GL67), glycerylmono-oleate (GMO), Guanidino-dialkyl-carboxylicacid (GUADACA), 2-(bis(2-(tetradecanoyloxy)ethyl)amino)-N-(2-hydroxyethyl)-N,N-dimethyl-2-oxoethan-aminiumbromide (HEDC), 2,2′-(tert-butoxycarbonylazanediyl)bis(ethane-2,1-diyl)ditetradecanoate (HEDC-BOC-TN), 1-(2-(((3S,10R,13R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yldisulfanyl)ethyl)guanidine (HGT4002), (15Z,18Z)—N,N-dimethyl-6-(9Z,12Z)-octadeca-9,12-dien-1-yl)tetracosa-15,18-dien-1-amine (HGT5000), (15Z,18Z)—N,N-dimethyl-6-((9Z,12Z)-octadeca-9,12-dien-1-yl)tetracosa-4,15,18-trien-1-amine (HGT5001), Histaminyl-Cholesterolhemisuccinat (HisChol), histidinylcholesterolhemisuccinate (Hist-Chol), HydroSoyPC (HSPC), imidazolecholesterolester (ICE), 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), N,N-di-n-lctradecyl,N-methyl-N-(2-guanidinyl)ethylammonium (Lipid 1), N,N-di-n-octadecyl,N-methyl-N-(2-guanidinyl)ethylammoniumchloride (Lipid 2), 3-((4,4-bis(octyloxy)butanoyl)oxy)-2-((((3-(diethylamino)propoxy)carbonyl)oxy)methyl)propyl(9Z,12Z)-octadeca-9,12-dienoate (Lipid A), (9Z,12Z)-3-((4,4-bis(octyloxy)butanoyl)oxy)-2-((((3-(diethylamino)propoxy)carbonyl)oxy)methyl)propyloctadeca-9,12-dienoate (Lipid A1), 2,2-Dilinoleyl-4-dimethylaminoethyl-[1,3]-dioxolane (Lipid A2), ((5-((dimethylamino)methyl)-1,3-phenylene)bis(oxy))bis(octane-8,1-diyl)bis(decanoate) (Lipid B), 2-((4-(((3-(dimethylamino)propoxy)carbonyl)oxy)hexadecanoyl)oxy)propane-1,3-diyl9Z,9′Z,12Z,12′Z)-bis(octadeca-9,12-dienoate) (Lipid C), 3-(((3-(dimethylamino)propoxy)carbonyl)oxy)-13-(octanoyloxy)tridecyl3-octylundecanoate (Lipid D), (6Z,16Z)-12-((Z)-dec-4-en-1-yl)docosa-6,16-dien-11-yl5-(dimethylamino)pentanoate (Lipid I), Dioctadecyl-(2-hydroxyl-3-propylamino)aminopolylysine (Lipid T), (3-((6Z,9Z,28Z,31Z)-heptatriaconta-6,9,28,31-tetraen-19-yloxy)-N,N-dimethylpropan-1-amine (MC3 Ether), described in U.S. Provisional Application No. 61/384,050 (MC3 Thioester), (4-((6Z,9Z,28Z,31Z)-heptatriaconta-6,9,28,31-tetraen-19-yloxy)-N,N-dimethylbutan-1-amine (MC4 Ether), 3-((2-(((9Z,12Z)-octadeca-9,12-dienoyl)oxy)ethyl)amino)propanoicacid (MLAPA), 3-((2-(((9Z,12Z,15Z)-octadeca-9,12,15-trienoyl)oxy)ethynamine)propanoicacid (MLLAPA), monomycolylglycerol (MMG), 3-((2-(oleoyloxy)ethyl)amino)propanoicacid (MOAPA), 4-(2-Aminoethyl)-Morpholino-Cholesterolhemisuccinat (MoChol), 1,2-Dioleoyl-3-N-morpholine-propane (MoDO), Methylpyridiyl-dialkyl-carboxylicacid (MPDACA), monopalmitoylphosphatidylcholin (MPPC), 3-((2-(stearoyloxy)ethyl)amino)propanoicacid (MSAPA), N1-[2-((1S)-1-[(3-aminopropyl)amino]-4-[di(3-amino-propyl)amino]butylcarboxamido)ethyl]-3,4-di[oleyloxy]-benzamide (MVL5), 2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(33)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), phosphatidylcholines (PC), 1,3-Bis-(1,2-bis-tetradecyloxy-propyl-3-dimemylethoxyammoniumbromide)-propane-2-ol (PCL-2), palmitoyi-oieoyl-nor-arginine (PONA), stearylamine (STA), 2-(((tert-Butyldimethylsilyl)oxy)methyl)-2-(hydroxymethyl)propane-1,3-diol (Synthesis Example 1 (A)), 3-((tert-Butyl(dimethyl)silyl)oxy)-2,2-bis(((9Z)-tetradec-9-enoyloxy)methyl)propyl(9Z)-tetradec-9-enoate (Synthesis Example 1 (B)), 3-Hydroxy-2,2-bis(((9Z)-tetradec-9-enoyloxy)methyl)propyl(9Z)-tetradec-9-enoate (Synthesis Example 1 (C)), 3-((4-(Dimethylamino)butanoyl)oxy)-2,2-bis(((9Z)-tetradec-9-enoyloxy)methyl)propyl(9Z)-tetradec-9-enoate (Synthesis Example 1 (D)), 3-(5-(bis(2-hydroxydodecyl)amino)pentan-2-yl)-6-(5-((2-hydroxydodecyl)(2-hydroxyundecyl)amino)pentan-2-yl)-1,4-dioxane-2,5-dione) (Target 24), trehalose6′6′-dibehenate (TDB), 1,1′-(2-(4-(2-((2-(bis(2hydroxydodecyl)amino)ethyl)(2-hydroxydodecyl)amino)ethyl)piperazin-1-yl)ethylazanediyl)didodecan-2-ol (Tech G1), 3-((1,3-bis(((9Z,12Z)-octadeca-9,12-dienoyl)oxy)-2-((((9Z,12Z)-octadeca-9,12-dienoyl)oxy)methyl)propan-2-yl)amino)propanoicacid (TLAPA), (1-(2,3-linoleyloxypropoxy)-2-(linoleyloxy)-(7V,Λ/-dimethyl)-propyl-3-amine) (TLinDMA), 3-((1,3-bis(((9Z.12Z.15Z)-octadeca-9.12.15-trienoyl)oxy)-2-((((9Z.12Z.15E)-octadeca-9,12,15-trienoyl)oxy)methyl)propan-2-yl)amino)propanoicacid (TLLAPA), N-(α-trimethylammonioacetyl)-didodecyl-D-glutamatechloride (TMAG), 3-((1,3-bis(((Z)-octadec-9-enoyl)oxy)-2-((((Z)-octadec-9-enoyl)oxy)methyl)propan-2-yl)amino)propanoicacid (TOAPA), 3-((1,3-bis(stearoyloxy)-2-((stearoyloxy)methyl)propan-2-yl)amino)propanoicacid (TSAPA), 1,N19-bis((16E,18E,20E,22E)-17,21-dimethyl-15-oxo-23-(2,6,6-trimethylcyclohex-1-en-1-yl)-4,7,10-trioxa-14-azatricosa-16,18,20,22-tetraen-1-yl)-4,7,10,13,16-pentaoxanonadecane-1,19-diamide (VA-PEG-VA), 2,2-Dilinoleyl-4-dimethylaminoethyl-[1,3]-dioxolane (XTC), disclosed in Non-Patent Literature 11 (YSK05), 1,2-di-7-linolenyloxy-N,N-dimethylaminopropane (γ-DLenDMA), a-D-Tocopherolhemisuccinoyl, (9Z,9,Z,12Z,12,Z)-2-((2-(((3-(dimethylamino)propoxy)carbonyl)oxy)tetradecanoyl)oxy)propane-1,3-diylbis(octadeca-9,12-dienoate), 2-(((13Z,16Z)-4-(((3-(diethylamino)propoxy)carbonyl)oxy)docosa-13,16-dienoyl)oxy)propane-1,3-diyldioctanoate, 2-(((13Z,16Z)-4-(((3-(dimethylamino)propoxy)carbonyl)oxy)docosa-13,16-dienoyl)oxy)propane-1,3-diyldioctanoate, 2-((4-(((3-(ethyl(methyl)amino)propoxy)carbonyl)oxy)hexadecanoyl)oxy)propane-1,3-diyldioctanoate, 2-((4-(((3-(ethyl(methyl)amino)propoxy)carbonyl)oxy)hexadecanoyl)oxy)propane-1,3-diylbis(decanoate), 2-((4-(((3-(diethylamino)propoxy)carbonyl)oxy)hexadecanoyl)oxy)propane-1,3-diylbis(decanoate), 2-(10-dodecyl-3-ethyl-8,14-dioxo-7,9,13-trioxa-3-azaicosan-20-yl)propane-1,3-diyldioctanoate, 2-(((4-(dimethylamino)butanoyl)oxy)methyl)-2-((octanoyloxy)methyl)propane-1,3-diyl(9Z,9′Z)bis-tetradec-9-enoate, (9Z,9′Z,12Z,12′Z)-2-(((1-(cyclopropylmethyl)piperidine-4-carbonyl)oxy)methyl)propane-1,3-diylbis(octadeca-9,12-dienoate), ((2-(((1-isopropylpiperidine-4-carbonyl)oxy)methyl)-1,4-phenylene)bis(oxy))bis(octane-8,1-diyl)bis(decanoate), 2-((4-(((3-(ethyl(methyl)amino)propoxy)carbonyl)oxy)hexadecanoyl)oxy)propane-1,3-diyldidodecanoate, 2-((4-(((3-(diethylamino)propoxy)carbonyl)oxy)hexadecanoyl)oxy)propane-1,3-diyldidodecanoate, 2-((4-(((3-(dimethylamino)propoxy)carbonyl)oxy)hexadecanoyl)oxy)propane-1,3-diyldidodecanoate, 2-((4-(((3-(ethyl(methyl)amino)propoxy)carbonyl)oxy)hexadecanoyl)oxy)propane-1,3-diylditetradecanoate, 2-((4-(((3-(dimethylamino)propoxy)carbonyl)oxy)hexadecanoyl)oxy)propane-1,3-diylditetradecanoate, 2-((4-(((3-(diethylamino)propoxy)carbonyl)oxy)hexadecanoyl)oxy)propane-1,3-diylditetradecanoate, (Z)-2-((4-(((3-(dimethylamino)propoxy)carbonyl)oxy)hexadecanoyl)oxy)propane-1,3-diyldioleate, (9Z,9,Z,12Z,12,Z,15Z,15,Z)-2-((4-(((3-(dimethylamino)propoxy)carbonyl)oxy)hexadecanoyl)oxy)propane-1,3-diylbis(octadeca-9,12,15-trienoate), (9Z,9,Z,12Z,12,Z)-2-((4-(((3-(diethylamino)propoxy)carbonyl)oxy)hexadecanoyl)oxy)propane-1,3-diylbis(octadeca-9,12-dienoate), (9Z,9,Z,12Z,12,Z)-2-((4-(((3-(dimethylamino)propoxy)carbonyl)oxy)hexadecanoyl)oxy)propane-1,3-diylbis(octadeca-9,12-dienoate), N,N,N-trimethyl-5-oxo-5-(3-((3-pentyloctanoyl)oxy)-2,2-bis(((3-pentyloctanoyl)oxy)methyl)propoxy)pentane-1-Aminiumiodide3-((5-(dimethylamino)pentanoyl)oxy)-2,2-bis(((3-pentyloctanoyl)oxy)methyl)propyl3-pentyloctanoate, 3-dimethylaminopropylcarbonate(9Z,12Z)-octacosa-19,22-dien-11-yl, 2-(((N,N-dimethyl-β-alanyl)oxy]methyl}-2-[(octanoyloxy)methyl)propane-1,3-diyl(9Z,9′Z)bis-tetradec-9-enoate, O′I,O1-(2-(7-dodecyl-14-methyl-3,9-dioxo-2,4,8,10-tetraoxa-14-azapentadecyl)propane-1,3-diyl)8-dimethyldioctanedioate, 8-dimethylO′I,01-(2-(((1-methylpyrrolidine-3-carbonyl)oxy)methyl)propane-1,3-diyl)dioctanedioate, 1-(3-((6,6-bis((2-propylpentyl)oxy)hexanoyl)oxy)-2-(((1,4-dimethylpiperidine-4-carbonyl)oxy)methyl)propyl)8-methyloctanedioate, (9Z,12Z)-5-(((3-(dimethylamino)propoxy)carbonyl)oxy)-7-octylpentadecyloctadeca-9,12-dienoate, 5-(((3-(dimethylamino)propoxy)carbonyl)oxy)-7-octylpentadecyloctanoate, 1-(3-((6,6-bis((2-propylpentyl)oxy)hexanoyl)oxy)-2-(((1,4-dimethylpiperidine-4-carbonyl)oxy)methyl)propyl)10-octyldecanedioate, 3-(((3-(dimethylamino)propoxy)carbonyl)oxy)-5-octyltridecyldecanoate, 1-(16-(((4,4-bis(octyloxy)butanoyl)oxy)methyl)-9-dodecyl-2-methyl-7,13-dioxo-6,8,12,14-tetraoxa-2-azaheptadecan-17-yl)8-methyloctanedioate, 3-((5-(dimethylamino)pentanoyl)oxy)-2,2-bis(((9Z)-tetradec-9-enoyloxy)methyl)propyl(9Z,12Z)-octadec-9,12-dienoate, 3-((5-(Dimethylamino)pentanoyl)oxy)-2,2-bis(((3-pentyloctanoyl)oxy)methyl)propyl3-pentyloctanoate, (9Z,9′Z,12Z,12′Z)-2-(((3-(diethylamino)propanoyl)oxy)methyl)propane-1,3-diylbis(octadeca-9,12-dienoate), ((2-(((4-(dimethylamino)butanoyl)oxy)methyl)-1,4-phenylene)bis(oxy))bis(octane-8,1-diyl)bis(decanoate), 1-(3-((4,4-bis(octyloxy)butanoyl)oxy)-2-(((1-methylpyrrolidine-3-carbonyl)oxy)methyl)propyl)8-methyloctanedioate, 3-((4,4-bis(octyloxy)butanoyl)oxy)-2-((palmitoyloxy)methyl)propyl1-methylpyrrolidine-3-carboxylate, 3-((4,4-bis(octyloxy)butanoyl)oxy)-2-((tetradecanoyloxy)methyl)propyl1-methylpyrrolidine-3-carboxylate, 3-(((3-(dimethylamino)propoxy)carbonyl)oxy)-13-(octanoyloxy)tridecyl9-pentyltetradecanoate, 3-((4,4-bis(octyloxy)butanoyl)oxy)-2-((dodecanoyloxy)methyl)propyl1-methylpyrrolidine-3-carboxylate, 3-(((3-(dimethylamino)propoxy)carbonyl)oxy)-13-hydroxytridecyl9-pentyltetradecanoate, 3-(((3-(dimethylamino)propoxy)carbonyl)oxy)-13-(octanoyloxy)tridecyl7-hexyltridecanoate, 2-(5-(3-((1-methylpyrrolidine-3-carbonyl)oxy)-2-((tetradecanoyloxy)methyl)propoxy)-5-oxopentyl)propane-1,3-diyldioctanoate, 3-(((3-(dimethylamino)propoxy)carbonyl)oxy)-13-(octanoyloxy)tridecyl5-heptyldodecanoate, 2-(5-(3-((1-methylpyrrolidine-3-carbonyl)oxy)-2-((palmitoyloxy)methyl)propoxy)-5-oxopentyl)propane-1,3-diyldioctanoate, 3-(((3-(dimethylamino)propoxy)carbonyl)oxy)-13-hydroxytridecyl5-heptyldodecanoate, 2-(((1-methylpyrrolidine-3-carbonyl)oxy)methyl)propane-1,3-diylbis(6,6-bis(octyloxy)hexanoate), (9Z,12Z)-3-(((3-dimethylamino)propoxy)carbonyl)oxy)-13-(octanoyloxy)tridecyloctadeca-9,12-dienoate, 3-((5-(dimethylamino)pentanoyl)oxy)-2,2-bis(((9Z)-tetradec-9-enoyloxy)methyl)propyl(9Z)-octadec-9-enoate, 2-(10-dodecyl-3-ethyl-8,14-dioxo-7,9,13-trioxa-3-azanonadecan-19-yl)propane-1,3-diyldioctanoate, ((2-(((1-methylpiperidine-4-carbonyl)oxy)methyl)-1,4-phenylene)bis(oxy))bis(octane-8,1-diyl)bis(decanoate), 2-(((3-(dimethylamino)propanoyl)oxy)methyl)propane-1,3-diylbis(4,4-bis(octyloxy)butanoate), (9Z,12Z)-2-(((11Z,14Z)-2-((3-(dimethylamino)propanoyl)oxy)icosa-11,14-dien-1-yl)oxy)ethyloctadeca-9,12-dienoate, 2-(((1,3-dimethylpyrrolidine-3-carbonyl)oxy)methyl)propane-1,3-diylbis(4,4-bis(octyloxy)butanoate), (13Z,16Z)-4-(((3-(dimethylamino)propoxy)carbonyl)oxy)docosa-13,16-dien-1-ylheptadecan-9-ylsuccinate, 2,2-bis(heptyloxy)ethyl3-((3-ethyl-10-((9Z,12Z)-octadeca-9,12-dien-1-yl)-8,15-dioxo-7,9,14-trioxa-3-azaheptadecan-17-yl)disulfanyl)propanoate, 2-(((1-methylpyrrolidine-3-carbonyl)oxy)methyl)propane-1,3-diylbis(4,4-bis(octyloxy)buta, 1-(3-((1,3-dimethylpyrrolidine-3-carbonyl)oxy)-2-(((9Z,12Z)-octadeca-9,12-dienoyloxy)methyl)propyl)₁₀-octyldecanedioate, (13Z,16Z)-4-(((3-(diethylamino)propoxy)carbonyl)oxy)docosa-13,16-dien-1-yl2,2-bis(heptyloxy)acetate, (13Z,16Z)-4-(((2-(dimethylamino)ethoxy)carbonyl)oxy)docosa-13,16-dien-1-yl2,2-bis(heptyloxy)acetate, Aceticacid(20,23R)-2-methyl-9-[(9Z,12Z)-octadeca-9,12-dien-1-yl]-7-oxo-6,8,11-trioxa-2-azanonacosa-20-En-23-yl3-(dimethylamino)propylcarbonate(11Z,14Z)-1-{[(9Z,12R)-12-hydroxyoctadec-9-en-1-yl], (12Z,15Z)-1-((((9Z,12Z)-octadeca-9,12-dien-1-yloxy)carbonyl)oxy)henicosa-12,15-dien-3-yl3-(dimethylamino)propanoate, (9Z,12Z)-3-((4,4-bis(octyloxy)butanoyl)oxy)-2-((((3-(dimethylamino)propyl)carbamoyl)oxy)methyl)propyloctadeca-9,12-dienoate, (12Z,15Z)-3-((4-(dimethylamino)butanoyl)oxy)henicosa-12,15-dien-1-yl9-pentyltetradecanoate, (9Z,12Z)-3-((4,4-bis(octyloxy)butanoyl)oxy)-2-(((((1,2,2,6,6-pentamethylpiperidin-4-yl)oxy)carbonyl)oxy)methyl)propyloctadeca-9,12-dienoate, (12Z,15Z)-3-((4-(dimethylamino)butanoyl)oxy)henicosa-12,15-dien-1-yl7-hexyltridecanoate, (9Z,12Z)-3-((4,4-bis(octyloxy)butanoyl)oxy)-2-(((((1-methylpiperidin-4-yl)methoxy)carbonyl)oxy)methyl)propyloctadeca-9,12-dienoate, (12Z,15Z)-3-((4-(dimethylamino)butanoyl)oxy)henicosa-12,15-dien-1-yl5-heptyldodecanoate, (9Z,12Z)-3-((4,4-bis(octyloxy)butanoyl)oxy)-2-(((((1-ethylpiperidin-4-yl)oxy)carbonyl)oxy)methyl)propyloctadeca-9,12-dienoate, (12Z,15Z)-3-((4-(dimethylamino)butanoyl)oxy)henicosa-12,15-dien-1-yl3-octylundecanoate,formatesalt, 3-((5-(dimethylamino)pentanoyl)oxy)-2,2-bis(((9Z)-tetradec-9-enoyloxy)methyl)propyl(9Z)-hexadec-9-enoate, (9Z,12Z)-3-((4,4-bis(octyloxy)butanoyl)oxy)-2-(((((1-methylazetidin-3-yl)oxy)carbonyl)oxy)methyl)propyloctadeca-9,12-dienoate, (9Z,12Z)-(12Z,15Z)-3-((3-(dimethylamino)propanoyl)oxy)henicosa-12,15-dien-1-yloctadeca-9,12-dienoate, 2-(((3-(diethylamino)propoxy)carbonyl)oxy)tetradecyl4,4-bis((2-ethylhexyl)oxy)butanoate, (9Z,12Z)-3-((4,4-bis(octyloxy)butanoyl)oxy)-2-(((((1-methylpiperidin-4-yl)oxy)carbonyl)oxy)methyl)propyloctadeca-9,12-dienoate, (9Z,12Z)-3-((4,4-bis(octyloxy)butanoyl)oxy)-2-(((((1-methylpyrrolidin-3-yl)oxy)carbonyl)oxy)methyl)propyloctadeca-9,12-dienoate, (9Z,12Z)-3-(((2-(dimethylamino)ethoxy)carbonyl)oxy)pentadecyloctadeca-9,12-dienoate, (9Z,12Z)-3-((4,4-bis(octyloxy)butanoyl)oxy)-2-((((3-(4-methylpiperazin-1-yl)propoxy)carbonyl)oxy)methyl)propyloctadeca-9,12-dienoate, 3-(Dimethylamino)propyltriacontan-11-ylcarbonateTriacontan-11-ol, (9Z,12Z)-3-((4,4-bis(octyloxy)butanoyl)oxy)-2-((((3-(pyrrolidin-1-yl)propoxy)carbonyl)oxy)methyl)propyloctadeca-9,12-dienoate, (9Z,12Z)-3-(((3-(ethyl(methyl)amino)propoxy)carbonyl)oxy)pentadecyloctadeca-9,12-dienoate, 3-((4,4-bis(octyloxy)butanoyl)oxy)-2-(((9Z,12Z)-octadeca-9,12-dienoyloxy)methyl)propyl4-((diethylamino)methyl)benzoate, (9Z,12Z)-3-(((3-(diethylamino)propoxy)carbonyl)oxy)pentadecyloctadeca-9,12-dienoate, 3-((4,4-bis(octyloxy)butanoyl)oxy)-2-(((9Z,12Z)-octadeca-9,12-dienoyloxy)methyl)propyl3-((dimethylamino)methyl)benzoate, (9Z,12Z)-3-(((3-(dimethylamino)propoxy)carbonyl)oxy)pentadecyloctadeca-9,12-dienoate, 3-((4,4-bis(octyloxy)butanoyl)oxy)-2-(((9Z,12Z)-octadeca-9,12-dienoyloxy)methyl)propyl1-methylpiperidine-3-carboxylate, 3-((4,4-bis(octyloxy)butanoyl)oxy)-2-(((9Z,12Z)-octadeca-9,12-dienoyloxy)methyl)propyl1-methylpiperidine-4-carboxylate, 3-((4,4-bis(octyloxy)butanoyl)oxy)-2-(((9Z,12Z)-octadeca-9,12-dienoyloxy)methyl)propyl1,4-dimethylpiperidine-4-carboxylate, 3-((4-(dimethylamino)butanoyl)oxy)-2,2-bis(((9Z)-tetradec-9-enoyloxy)methyl)propyl(9Z)-hexadec-9-enoate, 2-(10-dodecyl-3-ethyl-8,14-dioxo-7,9,13-trioxa-3-azahexadecan-16-yl)propane-1,3-diyldioctanoate, (9Z,9′Z,12Z,12′Z)-2-(((4-(piperidin-1-yl)butanoyl)oxy)methyl)propane-1,3-diylbis(octadeca-9,12-dienoate), 3-((4,4-bis(octyloxy)butanoyl)oxy)-2-(((9Z,12Z)-octadeca-9,12-dienoyloxy)methyl)propyl4-methylmorpholine-2-carboxylate, (2R)-3-((4,4-bis(octyloxy)butanoyl)oxy)-2-(((9Z,12Z)-octadeca-9,12-dienoyloxy)methyl)propyl1-methylpyrrolidine-2-carboxylate, (2S)-3-((4,4-bis(octyloxy)butanoyl)oxy)-2-(((9Z,12Z)-octadeca-9,12-dienoyloxy)methyl)propyl1-methylpyrrolidine-2-carboxylate, (9Z,9′Z,12Z,12′Z)-2-((((3-(diethylamino)propoxy)carbonyl)oxy)methyl)-2-(((9Z,12Z)-octadeca-9,12-dienoyloxy)methyl)propane-1,3-diylbis(octadeca-9,12-dienoate), (9Z,12Z)-3-((4,4-bis(octyloxy)butanoyl)oxy)-2-(((((1-ethylpiperidin-3-yl)methoxy)carbonyl)oxy)methyl)propyloctadeca-9,12-dienoate, 3-((4,4-bis(octyloxy)butanoyl)oxy)-2-(((9Z,12Z)-octadeca-9,12-dienoyloxy)methyl)propyl1-(cyclopropylmethyl)piperidine-4-carboxylate, 3-((4,4-bis(octyloxy)butanoyl)oxy)-2-(((9Z,12Z)-octadeca-9,12-dienoyloxy)methyl)propyl1-isopropylpiperidine-4-carboxylate, (9Z,12Z)-3-((4,4-bis(octyloxy)butanoyl)oxy)-2-(((3-(dimethylamino)propanoyl)oxy)methyl)propyloctadeca-9,12-dienoate, 4-(dimethylamino)butylcarbonate(6Z,9Z,26Z,29Z)-pentatriaconta-6,9,26,29-tetraen-18-yl, 3-((6-(dimethylamino)hexanoyl)oxy)-2,2-bis(((9Z)-tetradec-9-enoyloxy)methyl)propyl(9Z)-tetradec-9-enoate, 2,5-bis((9Z,12Z)-octadeca-9,12-dienyloxy)benzyl3-(dimethylamino)propylcarbonate, (9Z,9′Z,12Z,12′Z)-2-(((4-(pyrrolidin-1-yl)butanoyl)oxy)methyl)propane-1,3-diylbis(octadeca-9,12-dienoate), 3-(((3-(dimethylamino)propoxy)carbonyl)oxy)pentadecyl5-heptyldodecanoate, Aceticacid(7R,9Z)-18-({[3-(dimethylamino)propyloxy]carbonyl}oxy)octacosa-9-en-7-yl, 3-(((3-(dimethylamino)propoxy)carbonyl)oxy)pentadecyl9-pentyltetradecanoate, (9Z,12Z)-3-((6,6-bis(octyloxy)hexanoyl)oxy)-2-((((3-(diethylamino)propoxy)carbonyl)oxy)methyl)propyloctadeca-9,12-dienoate, 3-(((3-(dimethylamino)propoxy)carbonyl)oxy)pentadecyl7-hexyltridec-6-enoate, (9Z,12Z)-3-(2,2-bis(heptyloxy)acetoxy)-2-((((2-(dimethylamino)ethoxy)carbonyl)oxy)methyl)propyloctadeca-9,12-dienoate, 3-(((3-(dimethylamino)propoxy)carbonyl)oxy)pentadecyl3-octylundec-2-enoate, (9Z,12Z)-3-(((3-(diethylamino)propoxy)carbonyl)oxy)-2-(((5-heptyldodecanoyl)oxy)methyl)propyloctadeca-9,12-dienoate, 3-(((3-dimethylamino)propoxy)carbonyl)oxy)pentadecyl3octylundecanoate, (9Z,12Z)-3-(((3-(diethylamino)propoxy)carbonyl)oxy)-2-(((9-pentyltetradecanoyl)oxy)methyl)propyloctadeca-9,12-dienoate, Diaceticacid(7R,9Z,26Z,29R)-18-({[3-(dimethylamino)propoxy]carbonyl}oxy)pentatriaconta-9,26-diene-7,29-diyl, 3-(((3-(dimethylamino)propoxy)carbonyl)oxy)pentadecyl8,8-bis((2-propylpentyl)oxy)octanoate, (9Z,12Z)-3-(((3-(diethylamino)propoxy)carbonyl)oxy)-2-(((7-hexyltridecanoyl)oxy)methyl)propyloctadeca-9,12-dienoate, 3-(((3-(ethyl(methyl)amino)propoxy)carbonyl)oxy)pentadecyl8,8-bis((2-propylpentyl)oxy)octanoate, (9Z,12Z)-3-(((3-(diethylamino)propoxy)carbonyl)oxy)-2-(((3-octylundecanoyl)oxy)methyl)propyloctadeca-9,12-dienoate, 3-(((3-(diethylamino)propoxy)carbonyl)oxy)pentadecyl8,8-bis((2-propylpentyl)oxy)octanoate, 3-(((3-(diethylamino)propoxy)carbonyl)oxy)pentadecyl8,8-dibutoxyoctanoate, 3-((5-(dimethylamino)pentanoyl)oxy)-2,2-bis(((9Z)-tetradec-9-enoyloxy)methyl)propyl(9Z)-tetradec-9-enoate, 3-(Dimethylamino)propylcarbonate(6Z,9Z,26Z,29Z)-pentatriacontour-6,9,26,29-tetraen-18-yl, 2,5-bis((9Z,12Z)-octadeca-9,12-dien-1-yloxy)benzyl3-(dimethylamino)propanoate, (9Z,9′Z,12Z,12′Z)-2-(((3-(4-methylpiperazin-1-yl)propanoyl)oxy)methyl)propane-1,3-diylbis(octadeca-9,12-dienoate), 3-(((3-(diethylamino)propoxy)carbonyl)oxy)pentadecyl8,8-bis(octyloxy)octanoate, 3-(Dimethylamino)propyloctacosane-11-ylcarbonate, 2,4-bis((9Z,12Z)-octadeca-9,12-dienyloxy)benzyl4-(dimethylamino)butanoate, (9Z,12Z)-3-(((3-(diethylamino)propoxy)carbonyl)oxy)-2-(((2-heptylundecanoyl)oxy)methyl)propyloctadeca-9,12-dienoate, 3-(((3-(diethylamino)propoxy)carbonyl)oxy)pentadecyl6,6-bis((2-ethylhexyl)oxy)hexanoate, 2-((((3-(dimethylamino)propoxy)carbonyl)oxy)methyl)propane-1,3-diylbis(2-heptylundecanoate), 3-(((3-(diethylamino)propoxy)carbonyl)oxy)pentadecyl6,6-bis(hexyloxy)hexanoate, 4-methyl-2,5-bis((9Z,12Z)-octadeca-9,12-dien-1-yloxy)benzyl4-(dimethylamino)butanoate, 3-(((3-(diethylamino)propoxy)carbonyl)oxy)pentadecyl6,6-bis(octyloxy)hexanoate, 4-(dimethylamino)butyl4-methyl-2,5-bis((9Z,12Z)-octadeca-9,12-dienyloxy)benzylcarbonate, 3-(((3-(dimethylamino)propoxy)carbonyl)oxy)pendadecyl4,4-bis((2-propylpentyl)oxy)butanoate, 2-(12-dodecyl-3-ethyl-8,14-dioxo-7,9,13-trioxa-3-azaoctadecan-18-yl)propane-1,3-diyldioctanoate, 2-(5-oxo-5-((3-(((3-(piperidin-1-yl)propoxy)carbonyl)oxy)pentadecyl)oxy)pentyl)propane-1,3-diyldioctanoate, 3-(dimethylamino)propyl4-methyl-2,5-bis((9Z,12Z)-octadeca-9,12-dien-1-yloxy)benzylcarbonate, 3-(((3-(ethyl(methyl)amino)propoxy)carbonyl)oxy)pentadecyl4,4-bis((2-propylpentyl)oxy)butanoate, 2-(11-dodecyl-3-ethyl-9,15-dioxo-8,10,14-trioxa-3-azanonadecan-19-yl)propane-1,3-diyldioctanoate, 2-(10-dodecyl-3-ethyl-8,15-dioxo-7,9,14-trioxa-3-azanonadecan-19-yl)propane-1,3-diyldioctanoate, 2-(5-((4-((((1-methylpiperidin-4-yl)oxy)carbonyl)oxy)hexadecyl)oxy)-5-oxopentyl)propane-1,3-diyldioctanoate, 2-(5-((4-((((1-ethylpiperidin-3-yl)methoxy)carbonyl)oxy)hexadecyl)oxy)-5-oxopentyl)propane-1,3-diyldioctanoate, 2-(5-((4-(((((R)-1-methylpyrrolidin-3-yl)oxy)carbonyl)oxy)hexadecyl)oxy)-5-oxopentyl)propane-1,3-diyldioctanoate, 2-(5-((4-(((((S)-1-methylpyrrolidin-3-yl)oxy)carbonyl)oxy)hexadecyl)oxy)-5-oxopentyl)propane-1,3-diyldioctanoate, 2-(5-oxo-5-((4-(((S)-pyrrolidine-2-carbonyl)oxy)hexadecyl)oxy)pentyl)propane-1,3-diyldioctanoate, 2-(5-((4-((1,3-dimethylpyrrolidine-3-carbonyl)oxy)hexadecyl)oxy)-5-oxopentyl)propane-1,3-diyldioctanoate, 2-(5-((4-((1,4-dimethylpiperidine-4-carbonyl)oxy)hexadecyl)oxy)-5-oxopentyl)propane-1,3-diyldioctanoate, 4,4-bis(octyloxy)butyl(3-(diethylamino)propyl)pentadecane-1,3-diyldicarbonate, 3-(((3-(diethylamino)propoxy)carbonyl)oxy)pentadecyl4,4-bis((2-propylpentyl)oxy)butanoate, ((2-((((3-(diethylamino)propoxy)carbonyl)oxy)methyl)-1,4-phenylene)bis(oxy))bis(octane-8,1-diyl)bis(decanoate), 4,4-bis(octyloxy)butyl5-(((3-(diethylamino)propoxy)carbonyl)oxy)heptadecanoate, 6-((6,6-bis(octyloxy)hexanoyl)oxy)-4-(((3-(diethylamino)propoxy)carbonyl)oxy)hexyloctanoate, (12Z,15Z)-3-(((3-(diethylamino)propoxy)carbonyl)oxy)henicosa-12,15-dien-1-yl6,6-bis(octyloxy)hexanoate, 3-(((3-(diethylamino)propoxy)carbonyl)oxy)tridecyl6,6-bis(octyloxy)hexanoate, 3-(((3-(diethylamino)propoxy)carbonyl)oxy)undecyl6,6-bis(octyloxy)hexanoate, 3-(((3-(diethylamino)propoxy)carbonyl)oxy)pentadecyl5-(4,6-diheptyl-1,3-dioxan-2-yl)pentanoate, 3-((5-(diethylamino)pentanoyl)oxy)pentadecyl6,6-bis(octyloxy)hexanoate, 1-((6,6-bis(octyloxy)hexanoyl)oxy)pentadecan-3-yl1,4-dimethylpiperidine-4-carboxylate, 3-((3-(1-methylpiperidin-4-yl)propanoyl)oxy)pentadecyl6,6-bis(octyloxy)hexanoate, 1-((6,6-bis(octyloxy)hexanoyl)oxy)pentadecan-3-yl1,3-dimethylpyrrolidine-3-carboxylate, 3-(((3-(diethylamino)propoxy)carbonyl)oxy)pentadecyl4,4-bis((2-ethylhexyl)oxy)butanoate, 2-(((1,3-dimethylpyrrolidine-3-carbonyl)oxy)methyl)propane-1,3-diylbis(8-(octanoyloxy)octanoate), ((2-((((3-(dimethylamino)propoxy)carbonyl)oxy)methyl)-1,4-phenylene)bis(oxy))bis(octane-8,1-diyl)bis(decanoate), (2R)-1-((6,6-bis(octyloxy)hexanoyl)oxy)pentadecan-3-ylpyrrolidine-2-carboxylate, (2S)-1-((6,6-bis(octyloxy)hexanoyl)oxy)pentadecan-3-yl1-methylpyrrolidine-2-carboxylate, (2R)-1-((6,6-bis(octyloxy)hexanoyl)oxy)pentadecan-3-yl1-methylpyrrolidine-2-carboxylate, 3-(((3-(dimethylamino)propoxy)carbonyl)oxy)pentadecyl6,6-bis((3-ethylpentyl)oxy)hexanoate, 3-(((3-(dimethylamino)propoxy)carbonyl)oxy)pentadecyl6,6-bis((2-propylpentyl)oxy)hexanoate, 3-(((3-(diethylamino)propoxy)carbonyl)oxy)pentadecyl6,6-bis((2-propylpentyl)oxy)hexanoate, 3-(((2-(diethylamino)ethoxy)carbonyl)oxy)pentadecyl6,6-bis(octyloxy)hexanoate, 3-(((3-morpholinopropoxy)carbonyl)oxy)pentadecyl6,6-bis(octyloxy)hexanoate, 3-((((1-methylpiperidin-4-yl)methoxy)carbonyl)oxy)pentadecyl6,6-bis(octyloxy)hexanoate, 3-(((3-(4-methylpiperazin-1-yl)propoxy)carbonyl)oxy)pentadecyl6,6-bis(octyloxy)hexanoate, 3-(((3-(diethylamino)propoxy)carbonyl)oxy)pentadecyl4,4-bis(octyloxy)butanoate, 2-(((4-(dimethylamino)butanoyl)oxy)methyl)-2-((dodecanoyloxy)methyl)propane-1,3-diyl(9Z,9′Z)bis-tetradec-9-enoate, (9Z,9′Z,12Z,12′Z)-2-(((4-(dimethylamino)butanoyl)oxy)methyl)propane-1,3-diylbis(octadeca-9,12-dienoate), 3-(((4-(diethylamino)butoxy)carbonyl)oxy)pentadecyl6,6-bis(octyloxy)hexanoate, 3-(((3-(piperazin-1-yl)propoxy)carbonyl)oxy)pentadecyl6,6-bis(octyloxy)hexanoate, 3-(((3-piperidin-1-yl)propoxy)carbonyl)oxy)pentadecyl6.6-bis(octyloxy)hexanoate, 3-(((3-(dimethylamino)propoxy)carbonyl)oxy)pentadecyl4,4-bis(octyloxy)butanoate, (9Z,9′Z,12Z,12′Z)-2-(9-dodecyl-2-methyl-7,12-dioxo-6,8,13-trioxa-2-azatetradecan-14-yl)propane-1,3-diylbis(octadeca-9,12-dienoate), (9Z,12Z)-10-dodecyl-3-ethyl-14-(2-((9Z,12Z)-octadeca-9,12-dienoyloxy)ethyl)-8,13-dioxo-7,9-dioxa-3,14-diazahexadecan-16-yloctadeca-9,12-dienoate, 2-((2-(((3-(diethylamino)propoxy)carbonyl)oxy)tetradecanoyl)oxy)propane-1,3-diyldioctanoate, 2-(9-dodecyl-2-methyl-7,13-dioxo-6,8,12-trioxa-2-azanonadecan-19-yl)propane-1,3-diyldioctanoate, 2-((decanoyloxy)methyl)-2-(((4-(dimethylamino)butanoyl)oxy)methyl)propane-1,3-diyl(9Z,9′Z)bis-tetradec-9-enoate, (9Z,9′Z,12Z,12′Z)-2-(((3-morpholinopropanoyl)oxy)methyl)propane-1,3-diylbis(octadeca-9,12-dienoate), 3-(Dimethylamino)propylcarbonate(6Z,9Z,28Z,31Z)-heptatriaconta-6,9,28,31-tetraen-19-yl, 2,5-bis((9Z,12Z)-octadeca-9,12-dien-1-yloxy)benzyl4-(dimethylamino)butanoate, 2-(10-dodecyl-3-ethyl-8,14-dioxo-7,9,13-trioxa-3-azaoctadecan-18-yl)propane-1,3-diyldioctanoate, (9Z,9′Z,12Z,12′Z)-2-(((1,3-dimethylpyrrolidine-3-carbonyl)oxy)methyl)propane-1,3-diylbis(octadeca-9,12-dienoate), ((5-((dimethylamino)methyl)benzene-1,2,3-triyl)tris(oxy))tris(decane10,1-diyl)trioctanoate, 0′,0-(((5-((dimethylamino)methyl)-1,3-phenylene)bis(oxy))bis(propane-3,1-diyl))9-dioctyldinonanedioate, (9Z,12Z)-3-(3-((dimethylamino)methyl)-5-(3-((3-octylundecanoyl)oxy)propoxy)phenoxy)propyloctadeca-9,12-dienoate, ((((5-((dimethylamino)methyl)-1,3-phenylene)bis(oxy))bis(propane-3,1-diyl))bis(oxy))bis(4-oxobutane-4,1-diyl)bis(decanoate), (R)-4-(3-((R)-3,4-bis(octanoyloxy)butoxy)-5-((dimethylamino)methyl)phenoxy)butane-1,2-diyldioctanoate, (S)-4-(3-((S)-3,4-bis(octanoyloxy)butoxy)-5-((dimethylamino)methyl)phenoxy)butane-1,2-diyldioctanoate, (R)-4-(3-((S)-3,4-bis(octanoyloxy)butoxy)-5-((dimethylamino)methyl)phenoxy)butane-1,2-diyldioctanoate, 4,4′-((5-((dimethylamino)methyl)-1,3-phenylene)bis(oxy))bis(butane1,2-diyl)tetraoctanoate, didodecyl6,6′-((5-((dimethylamino)methyl)-1,3-phenylene)bis(oxy))dihexanoate, di((9Z,12Z)-octadeca-9,12-dien-1-yl)5,5′-((5-((dimethylamino)methyl)-1,3-phenylene)bis(oxy))dipentanoate, (((5-((dimethylamino)methyl)-1,3-phenylene)bis(methylene))bis(oxy))bis(6-oxohexane-6,1-diyl)bis(decanoate), (5-((dimethylamino)methyl)-1,3-phenylene)bis(methylene)bis(8-(octanoyloxy)octanoate), (5-((dimethylamino)methyl)-1,3-phenylene)bis(methylene)bis(10-(octanoyloxy)decanoate), (((5-((dimethylamino)methyl)-1,3-phenylene)bis(methylene))bis(oxy))bis(6-oxohexane-6,1-diyl)dioctanoate, (((5-((dimethylamino)methyl)-1,3-phenylene)bis(methylene))bis(oxy))bis(8-oxooctane-8,1-diyl)bis(decanoate), (9Z,9′Z,12Z,12′Z)-(((5-((dimethylamino)methyl)-1,3-phenylene)bis(methylene))bis(oxy))bis(4-oxobutane-4,1-diyl)bis(octadeca-9,12-dienoate), 0′,0-((5-((dimethylamino)methyl)-1,3-phenylene)bis(methylene))8-dinonyldioctanedioate, 0,0′-((5-((dimethylamino)methyl)-1,3-phenylene)bis(methylene))bis(10-(octanoyloxy)decyl)disuccinate, 0,0′-((5-((dimethylamino)methyl)-1,3-phenylene)bis(methylene))di((9Z,12Z)-octadeca-9,12-dien-1-yl)disuccinate, (9Z,9′Z,12Z,12′Z)-(5-((((3-(diethylamino)propoxy)carbonyl)oxy)methyl)-1,3-phenylene)bis(methylene)bis(octadeca-9,12-dienoate), (9Z,12Z)-4-(3-((dimethylarnino)methyl)-5-(4-(oleoyloxy)butoxy)phenoxy)butyloctadeca-9,12-dienoate, (9Z,9′Z,12Z,12′Z,15Z,15′Z)-((5-((dimethylamino)methyl)-1,3-phenylene)bis(oxy))bis(butane-4,1-diyl)bis(octadeca-9,12,15-trienoate), ((5-((dimethylamino)methyl)-1,3-phenylene)bis(oxy))bis(butane-4,1-diyl)ditetradecanoate, (Z)-((5-((dimethylamino)methyl)-1,3-phenylene)bis(oxy))bis(butane-4,1-diyl)dioleate, ((5-((dimethylamino)methyl)-1,3-phenylene)bis(oxy))bis(hexane-6,1-diyl)didodecanoate, (9Z,9′Z,12Z,12′Z)-((((5-((diethylamino)methyl)-1,3-phenylene)bis(oxy))bis(ethane-2,1-diyl))bis(oxy))bis(ethane-2,1-diyl)bis(octadeca-9,12-dienoate), didecyl8,8′-((5-((dimethylamino)methyl)-1,3-phenylene)bis(oxy))dioctanoate, ((5-((dimethylamino)methyl)-1,3-phenylene)bis(oxy))bis(propane-3,1-diyl)bis(3-octylundecanoate), (9Z.9′Z.12Z.12′Z)-((5-((diethylamino)methan-2-methyl-1.3-phenylene)bis(oxy))bis(butane-4,1-diyl)bis(octadeca-9,12-dienoate), ((5-((dimethylamino)methyl)-1,3-phenylene)bis(oxy))bis(octane-8,1-diyl)didodecanoate, ((5-((dimethylamino)methyl)-1,3-phenylene)bis(oxy))bis(octane-8,1-diyl)bis(decanoate), (9Z.9′Z.12Z.12′Z)-((5-((dimethylarsino)methan-2-methyl-1.3-phenylene)bis(oxy))bis(butane-4,1-diyl)bis(octadeca-9,12-dienoate), (8Z,8′Z)-((5-((dimethylamino)methyl)-1,3-phenylene)bis(oxy))bis(hexane-bis(dodec-8-enoate), (9Z,9′Z,12Z,12′Z)-((5-((3-hydroxyazetidine-1-yl)methyl)-1,3-phenylene)bis(oxy))bis(butane-4,1-diyl)bis(octadeca-9,12-dienoate), ((5-((dimethylamino)methyl)-1,3-phenylene)bis(oxy))bis(hexane-6,1-diyl)dioctanoate, ((5-((dimethylamino)methyl)-1,3-phenylene)bis(oxy))bis(hexane-6,1-diyl)bis(decanoate), (9Z.9′Z.12Z.12′Z)-((5-((dimethylamino)methan-1.3-phenylene)bis(oxy))bis(octane-8,1-diyl)bis(octadeca-9,12-dienoate), (9Z,9′Z,12Z,12′Z)-((5-((dimethylamino)methyl)-1,3-phenylene)bis(oxy))bis(hexane-6,1-diyl)bis(octadeca-9,12-dienoate), ((5-((dimethylamino)methyl)-1,3-phenylene)bis(oxy))bis(decane-10,1-diyl)dihexanoate, ((5-((dimethylamino)methyl)-1,3-phenylene)bis(oxy))bis(decane-10,1-diyl)dioctanoate, ((5-((dimethylamino)methyl)-1,3-phenylene)bis(oxy))bis(octane-8,1-diyl)dioctanoate, ((5-((dimethylamino)methyl)-1,3-phenylene)bis(oxy))bis(octane-8,1-diyl)dihexanoate, (9Z,9′Z,12Z,12′Z)-((5-((dimethylamino)methyl)-1,3-phenylene)bis(oxy))bis(ethane-2,1-diyl)bis(octadeca-9,12-dienoate), (9Z,9′Z,12Z,12′Z)-((5-((dimethylamino)methyl)-1,3-phenylene)bis(oxy))bis(propane-3,1-diyl)bis(octadeca-9,12-dienoate), (9Z,9′Z,12Z,12′Z)-((5-((dimethylamino)methyl)-1,3-phenylene)bis(oxy))bis(butane-4,1-diyl)bis(octadeca-9,12-dienoate), (5-((dimethylamino)methyl)-1,3-phenylene)bis(methylene)ditridecanoate, (9Z,9′Z,12Z,12′Z)-(5-((dimethylamino)methyl)-1,3-phenylene)bis(methylene)bis(octadeca-9,12-dienoate), (2,6-bis((9Z,12Z)-octadeca-9,12-dien-1-yloxy)pyridin-4-yl)methyl3-(dimethylamino)propanoate, (9Z,9′Z,12Z,12′Z)-5-(((3-(dimethylamino)propanoyl)oxy)methyl)-1,3-phenylenebis(octadeca-9,12-dienoate), 1-(3,5-bis((9Z,12Z)-octadeca-9,12-dien-1-yloxy)phenyl)-N,N-dimethylmethanamine, 3,5-bis((9Z,12Z)-octadeca-9,12-dien-1-yloxy)benzyl3-(dimethylamino)propanoate, 1-(3,5-bis(4,4-bis(octyloxy)butoxy)phenyl)-N,N-dimethylmethanamine, ((((5-((dimethylamino)methyl)-1,3-phenylene)bis(oxy))bis(butane-4,1-diyl))bis(oxy))bis(propane-3,2,1-triyl)tetraoctanoate, ((5-(((4-(dimethylamino)butanoyl)oxy)methyl)-1,3-phenylene)bis(oxy))bis(octane-8,1-diyl)bis(decanoate), ((5-(((3-(dimethylamino)propanoyl)oxy)methyl)-1,3-phenylene)bis(oxy))bis(octane-8,1-diyl)bis(decanoate), (9Z,9′Z,12Z,12′Z)-((5-(3-morpholinopropyl)-1,3-phenylene)bis(oxy))bis(butane4,1-diyl)bis(octadeca-9,12-dienoate), (9Z,9′Z,12Z,12′Z)-((5-(3-(dimethylamino)propyl)-1,3-phenylene)bis(oxy))bis(butane-4,1-diyl)bis(octadeca-9,12-dienoate), (9Z,9′Z,12Z,12′Z)-((5-(3-(piperidin-1-yl)propyl)-1,3-phenylene)bis(oxy))bis(butane-4,1-diyl)bis(octadeca-9,12-dienoate), (5-((dimethylamino)methyl)-1,3-phenylene)bis(methylene)bis(9-pentyltetradecanoate), (5-((dimethylamino)methyl)-1,3-phenylene)bis(methylene)bis(7-hexyltridecanoate), (5-((dimethylamino)methyl)-1,3-phenylene)bis(methylene)bis(5-heptyldodecanoate), ((5-((dimethylamino)methyl)-1,3-phenylene)bis(oxy))bis(butane-4,1-diyl)bis(3-octylundecanoate), ((5-((dimethylamino)methyl)-1,3-phenylene)bis(oxy))bis(butane-4,1-diyl)bis(5-heptyldodecanoate), ((5-((dimethylamino)methyl)-1,3-phenylene)bis(oxy))bis(butane-4,1-diyl)bis(9-pentyltetradecanoate), ((5-((dimethylamino)methyl)-1,3-phenylene)bis(oxy))bis(butane-4,1-diyl)bis(7-hexyltridecanoate), (9Z,9′Z,12Z,12′Z)-((5-(pyrrolidin-1-ylmethyl)-1,3-phenylene)bis(oxy))bis(butan4,1-diyl)bis(octadeca-9,12-dienoate), (((5-((dimethylamino)methyl)-1,3-phenylene)bis(oxy))bis(methylene))bis(propane-3,2,1-triyl)tetraoctanoate, (((5-((dimethylamino)methyl)-1,3-phenylene)bis(oxy))bis(butane-4,1-diyl))bis(propane-3,2,1-triyl)tetraoctanoate, (9Z.12Z)-4-(3-((dimethylamino)methan-5-(4-((3-octylundecanoyl)oxy)butoxy)phenoxy)butyloctadeca-9,12-dienoate, bis(1,3-bis(octanoyloxy)propan-2-yl)0,0′-((5-((dimethylamino)methyl)-1,3-phenylene)bis(methylene))disuccinate, (5-((dimethylamino)methyl)-1,3-phenylene)bis(methylene)bis(6-(((nonyloxy)carbonyl)oxy)hexanoate), 2-(3-(4-(5-((dimethylamino)methyl)-2-methyl-3-((9Z,12Z)-octadeca9,12-dien-1-yloxy)phenoxy)butoxy)-3-oxopropyl)propane-1,3-diyldihexanoate, 3-((dimethylamino)methyl)-5-(((8-(octanoyloxy)octanoyl)oxy)methyl)benzyl3-octylundecanoate, ((5-((diethylamino)methyl)benzene-1,2,3-triyl)tris(oxy))tris(decane-10,1-diyl)trioctanoate, 1-(3,5-bis((Z)-octadec-9-en-1-yloxy)phenyl)-N,N-dimethylmethanamine, N′-methyl-N′,N″.N″-tris((2E.6E)-3.7.11-trimethyldodeca-2.6.10-trien-1-vnpropane-1,3-diamine, 1,17-bis(2-((2-pentylcyclopropyl)methyl)cyclopropyl)heptadecan-9-yl4-(dimethylamino)butanoate, ethyl(7Z)-17-{[4-(dimethylamino)butanoyl]oxy}hexacos-7-enoate, (Z)-methyl6-(2-(dimethylamino)-3-(octadec-9-en-1-yloxy)propoxy)hexanoate, 2-(Didodecylamino)-1-(4-(N-(2-(dinonylamino)ethyl)-N-dodecylglycyl)piperazin-1-yl)ethan-1-one, 3-((3-(1-(3-((2-(Dinonylamino)ethyl)(nonyl)amino)propanoyl)piperidin-4-yl)propyl)(nonyl)amino)propylhexanoate, 3-((3-(4-(3-((2-(Dinonylamino)ethyl)(nonyl)amino)propanoyl)piperazin-1-yl)-3-oxopropyl)(nonyl)amino)propylhexanoate, 3-((2-(Dinonylamino)ethyl)(nonyl)amino)-1-(4-(3-(dinonylamino)propyl)piperidin-1-yl)propan-1-one, Pentyl4-((3-(1-(3-((2-(dinonylamino)ethyl)(nonyl)amino)propanoyl)piperidin-4-yl)propyl)(nonyl)amino)butano, Pentyl4-((2-(1-(N-(2-(dinonylamino)ethyl)-N-nonylglycyl)piperidin-4-yl)ethyl)(nonyl)amino)butanoate, Pentyl4-(((1-(N-(2-(dinonylamino)ethyl)-N-nonylglycyl)pyrrolidin-3-yl)methyl)(nonyl)amino)butanoate, Pentyl4-((2-(1-(N-(2-(dinonylamino)ethyl)-N-nonylglycyl)pyrrolidin-3-yl)ethyl)(nonyl)amino)butanoate, Pentyl4-((2-(1-(N-(2-(dinonylamino)ethyl)-N-nonylglycyl)piperidin-3-yl)ethyl)(nonyl)amino)butanoate, 2-(Didodecylamino)-1-(4-(N-(2-(dinonylamino)ethyl)-N-nonylglycyl)piperazin-1-yl)ethan-1-one, 2-((2-(Dinonylamino)ethyl)(nonyl)amino)-1-(3-(2-(dinonylamino)ethyl)piperidin-1-yl)ethan-1-one, Dipentyl4,4′-((2-(4-(N-(2-(dinonylamino)ethyl)-N-nonylglycyl)piperazin-1-yl)-2-oxoethyl)azanediyl)dibutyrate, Pentyl4-(nonyl(2-(4-(N-nonyl-N-(2-(nonyl(4-oxo-4-(pen1yloxy)buryl)amino)ethyl)glycyl)piperazin-1-yl)-2-oxoethyl)amino)butanoate, 2-((2-(Dinonylamino)ethyl)(nonyl)amino)-1-(3-((dinonylamino)methyl)pyrrolidin-1-yl)ethan-1-one, 2-((2-(Didodecylamino)ethyl)(dodecyl)amino)-1-(4-(dinonylglycyl)piperazin-1-yl)ethan-1-one, 2-((2-(Dinonylamino)ethyl)(nonyl)amino)-1-(3-(2-(dinonylamino)ethyl)pyrrolidin-1-yl)ethan-1-one, Pentyl4-((3-(4-(3-((2-(dinonylamino)ethyl)(nonyl)amino)propanoyl)piperazin-1-yl)-3-oxopropyl)(nonyl)amino)butanoate, 3-((2-(1-(N-(2-(Dinonylamino)ethyl)-N-nonylglycyl)piperidin-4-yl)ethyl)(nonyl)amino)propylhexanoate, Butyl5-((2-(1-(N-(2-(dinonylamino)ethyl)-N-nonylglycyl)piperidin-4-yl)ethyl)(nonyl)amino)pentanoate, 2-((2-(Didodecylamino)ethyl)(nonyl)amino)-1-(4-(dinonylglycyl)piperazin-1-yl)ethan-1-one, Propyl6-((2-(1-(N-(2-(dinonylamino)ethyl)-N-nonylglycyl)piperidin-4-yl)ethyl)(nonyl)amino)hexanoate, Ethyl7-((2-(1-(N-(2-(dinonylamino)ethyl)-N-nonylglycyl)piperidin-4-yl)ethyl)(nonyl)amino)heptanoate, Methyl8-((2-(1-(N-(2-(dinonylamino)ethyl)-N-nonylglycyl)piperidin-4-yl)ethyl)(nonyl)amino)octanoate, 3-((2-(4-(N-(2-(Dinonylamino)ethyl)-N-nonylglycyl)piperazin-1-yl)-2-oxoethyl)(nonyl)amino)propylhexanoate, Butyl5-((2-(4-(N-(2-(dinonylamino)ethyl)-N-nonylglycyl)piperazin-1-yl)-2-oxoethyl)(nonyl)amino)pentanoate, Propyl6-((2-(4-(N-(2-(dinonylamino)ethyl)-N-nonylglycyl)piperazin-2-oxoethyl)(nonyl)amino)hexanoate, Ethyl7-((2-(4-(N-(2-(dinonylamino)ethyl)-N-nonylglycyl)piperazin-1-yl)-2-oxoethyl)(nonyl)amino)heptanoate, 3-(Dinonylamino)-1-(4-(3-((2-(dinonylamino)ethyl)(nonyl)amino)propanoyl)piperazin-1-yl)propan-1-one, 2-((2-(Dinonylamino)ethyl)(nonyl)amino)-1-(4-(ditetradecylglycyl)piperazin-1-yl)ethan-1-one, 2-(Dinonylamino)-1-(4-(2-((2-(dinonylamino)ethyl)(nonyl)amino)ethyl)piperidin-1-yl)ethan-1-one, 2-(Dinonylamino)-1-(4-(N-(2-(dinonylamino)ethyl)-N-dodecylglycyl)piperazin-1-yl)ethan-1-one, 2-((2-(Dinonylamino)ethyl)(nonyl)amino)-1-(4-(2-(dinonylamino)ethyl)piperidin-1-yl)ethan-1-one, Methyl8-((2-(4-(dinonylglycyl)piperazin-1-yl)-2-oxoethyl)(2-((8-methoxy-8-oxooctyl)(nonyl)amino)ethyl)amino)octanoate, Methyl8-((2-(dinonylamino)ethyl)(2-(4-(dinonylglycyl)piperazin-1-yl)-2-oxoethyl)amino)octanoate, Methyl8-((2-((2-(4-(dinonylglycyl)piperazin-1-yl)-2-oxoethyl)(nonyl)amino)ethyl)(nonyl)amino)octanoate, Pentyl4-((2-(4-(N-(2-(dinonylamino)ethyl)-N-nonylglycyl)piperazin-2-oxoethyl)(nonyl)amino)butanoate, Methyl8-((2-(4-(N-(2-(dinonylamino)ethyl)-N-nonylglycyl)piperazin-1-yl)-2-oxoethyl)(nonyl)amino)octanoate, 2-((2-(Didodecylamino)ethyl)(dodecyl)amino)-1-(5-(dinonylglycyl)-2,5-diazabicyclo[2.2.1]heptan-2-yl)ethan-1-one3,2-(Dinonylamino)-1-(5-(N-(2-(dinonylamino)ethyl)-N-nonylglycyl)-2,5-diazabicyclo[2.2.1]heptan-2-yl)ethan-1-one, N1,N1,N2-Tri((9Z,12Z)-octadeca-9,12-dien-1-yl)-N2-(2-(piperazin-1-yl)ethyl)ethane-1,2-diamine, N1,N1,N2-Tri((Z)-octadec-9-en-1-yl)-N2-(2-(piperazin-1-yl)ethyl)ethane-1,2-diamine, 2-(Dinonylamino)-1-(4-(N-(2-(dinonylamino)ethyl)-N-nonylglycyl)piperazin-1-yl)ethan-1-one, N1,N1,N2-Tridodecyl-N2-(2-(piperazin-1-yl)ethyl)ethane-1,2-diamine, N1,N1,N2-Trinonyl-N2-(2-(piperazin-1-yl)ethyl)ethane-1,2-diamine, N1,N1,N2-Trihexyl-N2-(2-(piperazin-1-yl)ethyl)ethane-1,2-diamine, N1-(2-(4-(2-(Didodecylamino)ethyl)piperazin-1-yl)ethyl)-N1,N2,N2-tri((9Z,12Z)-octadeca-9,12-dien-1-yl)ethane-1,2-diamine, N1-(2-(4-(2-(Didodecylamino)ethyl)piperazin-1-yl)ethyl)-N1,N2,N2-tri((Z)-octadec-9-en-1-yl)ethane-1,2-diamine, N1-(2-(4-(2-(Ditetradecylamino)ethyl)piperazin-1-yl)ethyl)-N1,N2,N2-tritetradecylethane-1,2-diamine, N1-(2-(4-(2-(Didodecylamino)ethyl)piperazin-1-yl)ethyl)-N1,N2,N2-tritetradecylethane-1,2-diamine, N1-(2-(4-(2-(Dinonylamino)ethyl)piperazin-1-yl)ethyl)-N1,N2,N2-tritetradecylethane-1,2-diamine, 2-(Didodecylamino)-1-(4-(2-((2-(didodecylamino)ethyl)(dodecyl)amino)ethyl)piperazin-1-yl)ethan-1-one, N1-(2-(4-(2-(Di((9Z,12Z)-octadeca-9,12-dien-1-yl)amino)ethyl)piperazin-1-yl)ethyl)-N1,N2,N2-tridodecylethane-1,2-diamine, N1-(2-(4-(2-(Di((Z)-octadec-9-en-1-yl)amino)ethyl)piperazin-1-yl)ethyl)-N1,N2,N2-tridodecylethane-1,2-diamine, N1,N1,N2-Tridodecyl-N2-(2-(4-(2-(dodecyl((9Z,12Z)-octadeca-9,12-dien-1-yl)amino)ethyl)piperazin-1-yl)ethyl)ethane-1,2-diamine, N1-(2-(4-(2-(Ditetradecylamino)ethyl)piperazin-1-yl)ethyl)-N1,N2,N2-tridodecylethane-1,2-diamine, N1-(2-(4-(2-(Di((Z)-dodec-6-en-1-yl)amino)ethyl)piperazin-1-yl)ethyl)-N1,N2,N2-tridodecylethane-1,2-diamine, (Z)-N1-(2-(4-(2-(Dodec-6-en-1-yl(dodecyl)amino)ethyl)piperazin-1-yl)ethyl)-N,N2,N2-tridodecylethane-1,2-diamine, N1-(2-(4-(2-(Dinonylamino)ethyl)piperazin-1-yl)ethyl)-N1,N2,N2-tridodecylethane-1,2-diamine, N1-(2-(4-(2-(Dioctylamino)ethyl)piperazin-1-yl)ethyl)-N1,N2,N2-tridodecylethane-1,2-diamine, N1-(2-(4-(2-(Dihexylamino)ethyl)piperazin-1-yl)ethyl)-N1,N2,N2-tridodecylethan-1,2-diamine, N1-(2-(4-(2-(Ditetradecylamino)ethyl)piperazin-1-yl)ethyl)-N1,N2,N2-trinonylethane-1,2-diamine, 2-((2-(Didodecylamino)ethyl)(dodecyl)amino)-1-(4-(2-(didodecylamino)ethyl)piperazin-1-yl)ethan-1-one, N1-(2-(4-(2-(Didodecylamino)ethyl)piperazin-1-yl)ethyl)-N1,N2,N2-trinonylethane-1,2-diamine, N1-(2-(4-(2-(Dinonylamino)ethyl)piperazin-1-yl)ethyl)-N1,N2,N2-trinonylethane-1,2-diamine, N1-(2-(4-(2-(Didodecylamino)ethyl)piperazin-1-yl)ethyl)-N1,N2,N2-trihexylethane-1,2-diamine, Dimethyl12,12′-((2-(4-(2-((2-(didodecylamino)ethyl)(dodecyl)amino)ethyl)piperazin-1-yl)ethyl)azanediyl)didodecanoate, Methyl12-((2-(4-(2-((2-(didodecylamino)ethyl)(dodecyl)amino)ethyl)piperazin-1-yl)ethyl)(dodecyl)amino)dodecanoate, Dipentyl6,6′-((2-(4-(2-((2-(didodecylamino)ethyl)(dodecyl)amino)ethyl)piperazin-1-yl)ethyl)azanediyl)dihexanoate, Pentyl6-((2-(4-(2-((2-(ditetradecylamino)ethyl)(tetradecyl)amino)ethyl)piperazin-1-yl)ethyl)(dodecyl)amino)hexanoate, Pentyl6-((2-(4-(2-((2-(didodecylamino)ethyl)(dodecyl)amino)ethyl)piperazin-1-yl)ethyl)(dodecyl)amino)hexanoate, 2-(Didodecylamino)-1-(4-(N-(2-(didodecylamino)ethyl)-N-dodecylglycyl)piperazin-1-yl)ethan-1-one, 2-(Didodecylamino)-1-(4-(N-(2-(didodecylamino)ethyl)-N-nonylglycyl)piperazin-1-yl)ethan-1-one, 2-(Didodecylamino)-N-(2-(4-(2-(didodecylamino)ethyl)piperazin-1-yl)ethyl)-N-dodecylacetamide, ((2-((3,S′,4R)-3,4-dihydroxypyrrolidin-1-yl)acetyl)azanediyl)bis(ethane-2,1-diyl)(9Z,9′Z,12Z,12′Z)-bis(octadeca-9,12-dienoate), 2-amino-N,N-dihexadecyl-3-(1H-imidazol-5-yl)propanamide, (2-amino-N,N-dihexadecyl-3-(1H-imidazol-5-yl)propanamide, methyl(9Z)-19-[2-(dimethylamino)ethyl]heptacos-9-enoate, methyl8-(2-{9-[2-(dimethylamino)ethyl]octadecyl}cyclopropyl)octanoate, methyl(9Z)-19-[2-(dimethylamino)ethyl]octacos-9-enoate, ethyl8-(2-{ll-[(dimethylamino)methyl]heptadecyl}cyclopropyl)octanoate, ethyl8-(2-{ll-[(dimethylamino)methyl]octadecyl}cyclopropyl)octanoate, di((9Z,12Z)-octadeca-9,12-dien-1-yl)3-(((2-(dimethylamino)ethoxy)carbonyl)amino)pentanedioate, Heptyl6-((2-(1-(N-(2-(dinonylamino)ethyl)-N-nonylglycyl)piperidin-4-yl)ethyl)(tetradecyl)amino)hexanoate, ethyl8-(2-{ll-[(dimethylamino)methyl]nonadecyl}cyclopropyl)octanoate, Pentyl8-((2-(1-(N-(2-(dinonylamino)ethyl)-N-nonylglycyl)piperidin-4-yl)ethyl)(tetradecyl)amino)octanoate, ethyl8-(2-{ll-[(dimethylamino)methyl]icosyl}cyclopropyl)octanoate, ethyl8-(2-{9-[(dimethylamino)methyl]pentadecyl}cyclopropyl)octanoate, 3-((2-(1-(N-(2-(Dinonylamino)ethyl)-N-nonylglycyl)piperidin-4-yl)ethyl)(tetradecyl)amino)propyldecanoate, Heptyl6-((2-(4-(N-(2-(dinonylamino)ethyl)-N-nonylglycyl)piperazin-1-yl)-2-oxoethyl)(tetradecyl)amino)hexanoate, ethyl8-(2-{9-[(dimethylamino)methyl]hexadecyl}cyclopropyl)octanoate, Pentyl8-((2-(4-(N-(2-(dinonylamino)ethyl)-N-nonylglycyl)piperazin-2-oxoethyl)(tetradecyl)amino)octanoate, ethyl8-(2-{9-[(dimethylamino)methyl]heptadecyl}cyclopropyl)octanoate, methyl6-(2-(8-(2-(dimethylamino)-3-(nonyloxy)propoxy)octyl)cyclopropyl)hexanoate, methyl(9Z)-21-(dimethylamino)heptacos-9-enoate, methyl(9Z)-21-{[4-(dimethylamino)butanoyl]oxy}heptacos-9-enoate, (2R)—N,N-dimethyl-1-[(9Z,12Z)-octadeca-9,12-dien-1-yloxy]dodecan-2-amine, (15Z,18Z)—N,N-dimethyltetracoda-15,18-dien-5-amine, ethyl8-(2-{9-[(dimethylamino)methyl]octadecyl}cyclopropyl)octanoate, 3-((2-(4-(N-(2-(Dinonylamino)ethyl)-N-nonylglycyl)piperazin-1-yl)-2-oxoethyl)(tetradecyl)amino)propyldecanoate, ethyl4-(2-{ll-[(dimethylamino)methyl]icosyl}cyclopropyl)butanoate, ethyl8-(2-{7-[(dimethylamino)methyl]hexadecyl}cyclopropyl)octanoate, 3-((3-(1-(3-((2-(Dinonylamino)ethyl)(nonyl)amino)propanoyl)piperidin-4-yl)propyl)(nonyl)amino)propylhexanoate, ethyl6-(2-{9-[(dimethylamino)methyl]pentadecyl}cyclopropyl)hexanoate, 3-((3-(4-(3-((2-(Dinonylamino)ethyl)(nonyl)amino)propanoyl)piperazin-1-yl)-3-oxopropyl)(nonyl)amino)propylhexanoate, ethyl6-(2-{9-[(dimethylamino)methyl]hexadecyl}cyclopropyl)hexanoate, 3-((2-(Dinonylamino)ethyl)(nonyl)amino)-1-(4-(3-(dinonylamino)propyl)piperidin-1-yl)propan-1-one, Pentyl4-((3-(1-(3-((2-(dinonylamino)ethyl)(nonyl)amino)propanoyl)piperidin-4-yl)propyl)(nonyl)amino)buta\hat{ }, ethyl6-(2-{9-[(dimethylamino)methyl]heptadecyl}cyclopropyl)hexanoate, Pentyl4-((2-(1-(N-(2-(dinonylamino)ethyl)-N-nonylglycyl)piperidin-4-yl)ethyl)(nonyl)amino)butanoate, ethyl6-(2-{9-[(dimethylamino)methyl]octadecyl}cyclopropyl)hexanoate, Pentyl4-(((1-(N-(2-(dinonylamino)ethyl)-N-nonylglycyl)pyrrolidin-3-yl)methyl)(nonyl)amino)butanoate, ethyl(9Z)-21-[(dimethylamino)methyl]heptacos-9-enoate, Pentyl4-((2-(1-(N-(2-(dinonylamino)ethyl)-N-nonylglycyl)pyrrolidin-3-yl)ethyl)(nonyl)amino)butanoate, ethyl(9Z)-21-[(dimethylamino)methyl]octacos-9-enoate, ((2-((3,S′,4R)-3,4-dihydroxypyrrolidin-1-yl)acetyl)azanediyl)bis(ethane-2,1-diyl)(9Z,9′Z,12Z,12′Z)-bis(octadeca-9,12-dienoate), Pentyl4-((2-(1-(N-(2-(dinonylamino)ethyl)-N-nonylglycyl)piperidin-3-yl)ethyl)(nonyl)amino)butanoate, ethyl(9Z)-21-[(dimethylamino)methyl]nonacos-9-enoate, methyl6-(2-(8-(2-(dimethylamino)-3-(heptyloxy)propoxy)octyl)cyclopropyl)hexanoate, methyl(9Z)-21-{[4-(dimethylamino)butanoyl]oxy}octacos-9-enoate, methyl(9Z)-21-(dimethylamino)octacos-9-enoate, 2-(Didodecylamino)-1-(4-(N-(2-(dinonylamino)ethyl)-N-nonylglycyl)piperazin-1-yl)ethan-1-, (2S)—N.N-dimethyl-1-[(9Z,12Z)-octadeca-9,12-dien-1-yloxy]nonan-2-amine, (18Z,21Z)—N,N-dimethylheptacosa-18,21-dien-10-amine, ethyl(9Z)-21-[(dimethylamino)methyl]triacont-9-enoate, ethyl(9Z)-19-[(dimethylamino)methyl]pentacos-9-enoate, ethyl(9Z)-19-[(dimethylamino)methyl]hexacos-9-enoate, ethyl(9Z)-19-[(dimethylamino)methyl]heptacos-9-enoate, ethyl(9Z)-19-[(dimethylamino)methyl]octacos-9-enoate, ethyl(5Z)-17-[(dimethylamino)methyl]hexacos-5-enoate, ethyl(9Z)-17-[(dimethylamino)methyl]hexacos-9-enoate, 2-((2-(Dinonylamino)ethyl)(nonyl)amino)-1-(3-(2-(dinonylamino)ethyl)piperidin-1-yl)ethan-1-one, ethyl(7Z)-17-[(dimethylamino)methyl]tricos-7-enoate, Dipentyl4,4′-((2-(4-(N-(2-(dinonylamino)ethyl)-N-nonylglycyl)piperazin-1-yl)-2-oxoethyl)azanediyl)dibutyrate, Pentyl4-(nonyl(2-(4-(N-nonyl-N-(2-(nonyl(4-oxo-4-(pentyloxy)butyl)amino)ethyl)glycyl)piperazin-1-yl)-2-oxoethyl)amino)butanoate, ethyl(7Z)-17-[(dimethylamino)methyl]tetracos-7-enoate, ethyl(7Z)-17-[(dimethylamino)methyl]pentacos-7-enoate, 2-((2-(Dinonylamino)ethyl)(nonyl)amino)-1-(3-((dinonylamino)methyl)pyrrolidin-1-yl)ethan-1-one, trans-3-[(3}7-dimethyloctyl)oxy]-1-methyl-4˜[(9Z,12Z)-octadeca-9512-dien-1-yloxyjpyrrolidine, methyl6-(2-(8-(2-(dimethylamino)-3-(hexyloxy)propoxy)octyl)cyclopropyl)hexanoate, methyl(9Z)-21-{[4-(dimethylamino)butanoyl]oxy}nonacos-9-enoate, methyl(9Z)-21-(dimethylamino)nonacos-9-enoate, (2S)—N,N-dimethyl-1-[(9Z,12Z)-octadeca-9,12-dien-1-yloxy]tridecan-2-amine, (15Z,18Z)—N,N-dimethyltetracosa-15,18-dien-7-amine, ethyl(7Z)-17-[(dimethylamino)methyl]hexacos-7-enoate, 2-((2-(Dinonylamino)ethyl)(nonyl)amino)-1-(3-(2-(dinonylamino)ethyl)pyrrolidin-1-yl)ethan-1-one, methyl6-(2-{ll-[(dimethylamino)methyl]icosyl}cyclopropyl)hexanoate, methyl10-(2-{7-[(dimethylamino)methyl]hexadecyl}cyclopropyl)decanoate, methyl8-(2-{ll-[(dimethylamino)methyl]heptadecyl}cyclopropyl)octanoate, methyl8-(2-{ll-[(dimethylamino)methyl]octadecyl}cyclopropyl)octanoate, methyl8-(2-{ll-[(dimethylamino)methyl]nonadecyl}cyclopropyl)octanoate, methyl8-(2-{ll-[(dimethylamino)methyl]icosyl}cyclopropyl)octanoate, Pentyl4-((3-(4-(3-((2-(dinonylamino)ethyl)(nonyl)amino)propanoyl)piperazin-1-yl)-3-oxopropyl)(nonyl)amino)butanoate, methyl8-(2-{9-[(dimethylamino)methyl]pentadecyl}cyclopropyl)octanoate, methyl8-(2-{9-[(dimethylamino)methyl]hexadecyl}cyclopropyl)octanoate, 3-((2-(1-(N-(2-(Dinonylamino)ethyl)-N-nonylglycyl)piperidin-4-yl)ethyl)(nonyl)amino)propylhexanoate, methyl8-(2-{9-[(dimethylamino)methyl]heptadecyl}cyclopropyl)octanoate, methyl8-(2-(dimethylamino)-3-((6-((2-octylcyclopropyl)methoxy)-6-oxohexyl)oxy)propoxy)octanoate, Butyl5-((2-(1-(N-(2-(dinonylamino)ethyl)-N-nonylglycyl)piperidin-4-yl)ethyl)(nonyl)amino)pentanoate, trans-1-methyl-3-[(12Z)-octadec-12-en-1-yloxy]-4-(octyloxy)pyrrolidine, methyl(9Z)-21-{[4-(dimethylamino)butanoyl]oxy}triacont-9-enoate, methyl(9Z)-21-(dimethylamino)triacont-9-enoate, 2-((2-(Didodecylamino)ethyl)(nonyl)amino)-1-(4-(dinonylglycyl)piperazin-1-yl)ethan-1-oneStep1:MethylN-(2-(didodecylamino)ethyl)-N-nonylglycinate, 1-((2R,3S,5R)-3-(bis(hexadecyloxy)methoxy)-5-(5-methyl-2,4-dioxo-3,4-dihydropyrimidin-1(2H)-yl)tetrahydrofumethanesulfonate, (Z)-methyl16-(3-(decyloxy)-2-(dimethylamino)propoxy)hexadec-7-enoate, (2S)-1-[(9Z,12Z)-octadeca-9,12-dien-1-yloxy]nonan-2-amine, (14Z,17Z)—N,N-dimethyltricosane-14,17-dien-6-amine, Propyl6-((2-(1-(N-(2-(dinonylamino)ethyl)-N-nonylglycyl)piperidin-4-yl)ethyl)(nonyl)amino)hexanoate, methyl7-(2-(dimethylamino)-3-((6-((2-octylcyclopropyl)methoxy)-6-oxohexyl)oxy)propoxy)heptanoate, methyl(7Z)-19-[(dimethylamino)methyl]octacos-7-enoate, methyl(HZ)-19-[(dimethylamino)methyl]octacos-ll-enoate, Ethyl7-((2-(1-(N-(2-(dinonylamino)ethyl)-N-nonylglycyl)piperidin-4-yl)ethyl)(nonyl)amino)heptanoate, (2-octylcyclopropyl)methyl6-(2-(dimethylamino)-3-((5-methoxy-5-oxopentyl)oxy)propoxy)hexanoate, Methyl8-((2-(1-(N-(2-(dinonylamino)ethyl)-N-nonylglycyl)piperidin-4-yl)ethyl)(nonyl)amino)octanoate, methyl(9Z)-21-[(dimethylamino)methyl]heptacos-9-enoate, (2-octylcyclopropyl)methyl6-(2-(dimethylamino)-3-(4-methoxy-4-oxobutoxy)propoxy)hexanoate, methyl(9Z)-21-[(dimethylamino)methyl]octacos-9-enoate, 3-((2-(4-(N-(2-(Dinonylamino)ethyl)-N-nonylglycyl)piperazin-1-yl)-2-oxoethyl)(nonyl)amino)propylhexanoate, (Z)-methyl8-(2-(dimethylamino)-3-((6-oxo-6-(undec-2-en-1-yloxy)hexyl)oxy)propoxy)octanoate, methyl(9Z)-21-[(dimethylamino)methyl]nonacos-9-enoate, Butyl5-((2-(4-(N-(2-(dinonylamino)ethyl)-N-nonylglycyl)piperazin-1-yl)-2-oxoethyl)(nonyl)amino)pentanoate, (Z)-methyl7-(2-(dimethylamino)-3-((6-oxo-6-(undec-2-en-1-yloxy)hexyl)oxy)propoxy)heptanoate, Propyl6-((2-(4-(N-(2-(dinonylamino)ethyl)-N-nonylglycyl)piperazin-1-yl)-2-oxoethyl)(nonyl)amino)hexanoate, methyl(9Z)-21-[(dimethylamino)methyl]triacont-9-enoate, (Z)-undec-2-en-1-yl6-(2-(dimethylamino)-3-((5-methoxy-5-oxopentyl)oxy)propoxy)hexanoate, methyl(9Z)-19-[(dimethylamino)methyl]pentacos-9-enoate, Ethyl7-((2-(4-(N-(2-(dinonylamino)ethyl)-N-nonylglycyl)piperazin-1-yl)-2-oxoethyl)(nonyl)amino)heptanoate, (Z)-undec-2-en-1-yl6-(2-(dimethylamino)-3-(4-methoxy-4-oxobutoxy)propoxy)hexanoate, methyl6-(2-(dimethylamino)-3-((6-((2-octylcyclopropyl)methoxy)-6-oxohexyl)oxy)propoxy)hexanoate, methyl(9Z)-19-[(dimethylamino)methyl]hexacos-9-enoate, 3-(Dinonylamino)-1-(4-(3-((2-(dinonylamino)ethyl)(nonyl)amino)propanoyl)piperazin-1-yl)propan-1-one, methyl(9Z)-19-[(dimethylamino)methyl]heptacos-9-enoate, 2-((2-(Dinonylamino)ethyl)(nonyl)amino)-1-(4-(ditetradecylglycyl)piperazin-1-yl)ethan-1-one, (Z)-methyl6-(2-(dimethylamino)-3-((6-oxo-6-(undec-2-en-1-yloxy)hexyl)oxy)propoxy)hexanoate, methyl8-(2-(dimethylamino)-3-((8-(2-(6-methoxy-6-oxohexyl)cyclopropyl)octyl)oxy)propoxy)octanoate, methyl8-(2-{9-[(dimethylamino)methyl]octadecyl}cyclopropyl)octanoate, 2-(Dinonylamino)-1-(4-(2-((2-(dinonylamino)ethyl)(nonyl)amino)ethyl)piperidin-1-yl)ethan-1-one, trans-1-methyl-3-[(9Z)-octadec-9-en-1-yloxy]-4-(octyloxy)pyrrolidine, methyl(9Z)-19-{[4-(dimethylamino)butanoyl]oxy}pentacos-9-enoate, methyl(9Z)-19-(dimethylamino)pentacos-9-enoate, (Z)-methyl16-(2-(dimethylamino)-3-(nonyloxy)propoxy)hexadec-7-enoate, (2S)-1-[(9Z,12Z)-octadeca-9,12-dien-1-yloxy]decan-2-amine, (12Z,15Z)—N,N-dimethylhenicosan-12,15-dien-4-amine, methyl7-(2-(dimethylamino)-3-((8-(2-(6-methoxy-6-oxohexyl)cyclopropyl)octyl)oxy)propoxy)heptanoate, methyl(9Z)-19-[(dimethylamino)methyl]octacos-9-enoate, 2-((2-(Dinonylamino)ethyl)(nonyl)amino)-1-(4-(2-(dinonylamino)ethyl)piperidin-1-yl)ethan-1-one, Methyl8-((2-(4-(dinonylglycyl)piperazin-1-yl)-2-oxoethyl)(2-((8-methoxy-8-oxooctyl)(nonyl)amino)ethyl)amino)octanoate, methyl6-(2-(8-(2-(dimethylamino)-3-((5-methoxy-5-oxopentyl)oxy)propoxy)octyl)cyclopropyl)hexanoate, ethyl8-{2-[ll-(dimethylamino)heptadecyl]cyclopropyl}octanoate, Methyl8-((2-(dinonylamino)ethyl)(2-(4-(dinonylglycyl)piperazin-1-yl)-2-oxoethyl)amino)octanoate, methyl6-(2-(8-(2-(dimethylamino)-3-(4-methoxy-4-oxobutoxy)propoxy)octyl)cyclopropyl)hexanoate, ethyl8-{2-[ll-(dimethylamino)octadecyl]cyclopropyl}octanoate, Methyl8-((2-((2-(4-(dinonylglycyl)piperazin-1-yl)-2-oxoethyl)(nonyl)amino)ethyl)(nonyl)amino)octanoate, ethyl8-{2-[ll-(dimethylamino)nonadecyl]cyclopropyl}octanoate, (Z)-methyl16-(2-(dimethylamino)-3-((8-methoxy-8-oxooctyl)oxy)propoxy)hexadec-7-enoate, Pentyl4-((2-(4-(N-(2-(dinonylamino)ethyl)-N-nonylglycyl)piperazin-1-yl)-2-oxoethyl)(nonyl)amino)butanoate, ethyl8-{2-[ll-(dimethylamino)icosyl]cyclopropyl}octanoate, (Z)-methyl16-(2-(dimethylamino)-3-((7-methoxy-7-oxoheptyl)oxy)propoxy)hexadec-7-enoate, Methyl8-((2-(4-(N-(2-(dinonylamino)ethyl)-N-nonylglycyl)piperazin-1-yl)-2-oxoethyl)(nonyl)amino)octanoate, ethyl8-{2-[9-(dimethylamino)pentadecyl]cyclopropyl}octanoate, (Z)-methyl16-(2-(dimethylamino)-3-((5-methoxy-5-oxopentyl)oxy)propoxy)hexadec-7-enoate, (11E,20Z,23Z)—N,N-dimethylnonacosan-11,20,23-trien-10-amine, N,N-dimethyl-1-[(1S,2R)-2-octylcyclopropyl]pentadecan-8-amine, ethyl8-{2-[9-(dimethylamino)hexadecyl]cyclopropyl}octanoate, 2-((2-(Didodecylamino)ethyl)(dodecyl)amino)-1-(5-(dinonylglycyl)-2,5-diazabicyclo[2.2.1]heptan-2-yl)ethan-1-one3, (Z)-methyl16-(2-(dimethylamino)-3-(4-methoxy-4-oxobutoxy)propoxy)hexadec-7-enoate, methyl6-(2-(8-(2-(dimethylamino)-3-((6-methoxy-6-oxohexyl)oxy)propoxy)octyl)cyclopropyl)hexanoate, ethyl8-{2-[9-(dimethylamino)heptadecyl]cyclopropyl}octanoate, 2-(Dinonylamino)-1-(5-(N-(2-(dinonylamino)ethyl)-N-nonylglycyl)-2,5-diazabicyclo[2.2.1]heptan-2-yl)ethan-1-one, 1-[(1S,2R)-2-decylcyclopropyl]-N,N-dimethylpentadecane-6-amine, N1,N1,N2-Tri((9Z,12Z)-octadeca-9,12-dien-1-yl)-N2-(2-(piperazin-1-yl)ethyl)ethane-1,2-diamine, ethyl8-{2-[9-(dimethylamino)octadecyl]cyclopropyl}octanoate, 1-[(1R,2S)-2-heptylcyclopropyl]-N,N-dimethyloctadecan-9-amine, (Z)-methyl16-(2-(dimethylamino)-3-((6-methoxy-6-oxohexyl)oxy)propoxy)hexadec-7-enoate, N1,N1,N2-Tri((Z)-octadec-9-en-1-yl)-N2-(2-(piperazin-1-yl)ethyl)ethane-1,2-diamine, N,N-dimethyl-3-{7-[(1S,2R)-2-octylcyclopropyl]heptyl}dodecan-1-amine, methyl8-(2-(dimethylamino)-3-((8-(2-((2-pentylcyclopropyl)methyl)cyclopropyl)octyl)oxy)propoxy)octanoate, ethyl4-{2-[ll-(dimethylamino)icosyl]cyclopropyl}butanoate, trans-1-Methyl-3-[((9Z,12Z)-octadeca-9,12-dienyl)oxy]-4-octyloxy-pyrrolidine, methyl(9Z)-19-(dimethylamino)hexacos-9-enoate, methyl(9Z)-19-{[4-(dimethylamino)butanoyl]oxy}hexacos-9-enoate, (Z)-methyl16-(2-(dimethylamino)-3-(heptyloxy)propoxy)hexadec-7-enoate, (2R)-1-[(9Z,12Z)-octadeca-9,12-dien-1-yloxy]dodecan-2-amine, (13Z,16Z)—N,N-dimethyldocosa-13,16-dien-5-amine, N,N-dimethyl-1-[(1R,2S)-2-undecylcyclopropyl]tetradecan-5-amine, methyl7-(2-(dimethylamino)-3-((8-(2-((2-pentylcyclopropyl)methyl)cyclopropyl)octyl)oxy)propoxy)heptanoate, ethyl8-{2-[7-(dimethylamino)hexadecyl]cyclopropyl}octanoate, 2-(Didodecylamino)-N-dodecyl-N-(2-(piperazin-1-yl)ethyl)acetamide, N,N-dimethyl-1-[(1S,2R)-2-octylcyclopropyl]hexadecan-8-amine, N1-(2-(Piperazin-1-yl)ethyl)-N1,N2,N2-tritetradecylethane-1,2-diamine, methyl6-(2-(dimethylamino)-3-((8-(2-((2-pentylcyclopropyl)methyl)cyclopropyl)octyl)oxy)propoxy)hexanoate, ethyl6-{2-[9-(dimethylamino)pentadecyl]cyclopropyl}hexanoate, N,N-dimethyl-1-[(1S,2S)-2-{[(1R,2R)-2-pentylcyclopropyl]methyl}cyclopropyl]nonadecan-10-amine, NN1,N2-Tridodecyl-N2-(2-(piperazin-1-yl)ethyl)ethane-1,2-diamine, methyl5-(2-(dimethylamino)-3-((8-(2-((2-pentylcyclopropyl)methyl)cyclopropyl)octyl)oxy)propoxy)pentanoate, ethyl6-{2-[9-(dimethylamino)hexadecyl]cyclopropyl}hexanoate, N,N-dimethyl-21-[(1S,2R)-2-octylcyclopropyl]henicosan-10-amine, NNN2-Trinonyl-N2-(2-(piperazin-1-yl)ethyl)ethane-1,2-diamine, methyl4-(2-(dimethylamino)-3-((8-(2-((2-pentylcyclopropyl)methyl)cyclopropyl)octyl)oxy)propoxy)butanoate, ethyl6-{2-[9-(dimethylamino)heptadecyl]cyclopropyl}hexanoate, N,N-dimethyl-1-[(1S,2R)-2-octylcyclopropyl]nonadecan-10-amine, N1,N1,N2-Trihexyl-N2-(2-(piperazin-1-yl)ethyl)ethane-1,2-diamine, methyl8-(2-(dimethylamino)-3-((9Z,12Z)-octadeca-9,12-dien-1-yloxy)propoxy)octanoate, ethyl6-{2-[9-(dimethylamino)octadecyl]cyclopropyl}hexanoate, N1-(2-(4-(2-(Didodecylamino)ethyl)piperazin-1-yl)ethyl)-N1,N2,N2-tri((9Z,12Z)-octadeca-9,12-dien-1-yl)ethane-1,2-diamine, methyl7-(2-(dimethylamino)-3-((9Z,12Z)-octadeca-9,12-dien-1-yloxy)propoxy)heptanoate, ethyl(9Z)-21-(dimethylamino)heptacos-9-enoate, 1-[(1S,2R)-2-hexylcyclopropyl]-N,N-dimethylnonadecane-10-amine, 1-methyl18-[(2Z)-non-2-en-1-yl]9-{[4-(dimethylamino)butanoyl]oxy}octadecanedioate, N1-(2-(4-(2-(Didodecylamino)ethyl)piperazin-1-yl)ethyl)-N1,N2,N2-tri((Z)-octadec-9-en-1-yl)ethane-1,2-diamine, N,N-dimethyl-1-[(1S,2R)-2-octylcyclopropyl]heptadecan-8-amine, methyl6-(2-(dimethylamino)-3-((9Z,12Z)-octadeca-9,12-dien-1-yloxy)propoxy)hexanoate, ethyl(9Z)-21-(dimethylamino)octacos-9-enoate, dimethyl(9Z)-19-{[4-(dimethylamino)butanoyl]oxy}heptacos-9-enedioate, N1-(2-(4-(2-(Ditetradecylamino)ethyl)piperazin-1-yl)ethyl)-N1,N2,N2-tritetradecylethane-1,2-diamine, methyl5-(2-(dimethylamino)-3-((9Z,12Z)-octadeca-9,12-dien-1-yloxy)propoxy)pentanoate, ethyl8-{[4-(dimethylamino)butanoyl]oxy}-15-(2-octylcyclopropyl)pentadecanoate, ethyl(9Z)-21-(dimethylamino)nonacos-9-enoate, (13Z,16Z)—N,N-dimethyl-3-nonyldocosa-13,16-dien-1-amine, N1-(2-(4-(2-(Didodecylamino)ethyl)piperazin-1-yl)ethyl)-N1,N2,N2-tritetradecylethane-1,2-diamine, methyl9-{[4-(dimethylamino)butanoyl]oxy}-16-(2-octylcyclopropyl)hexadecanoate, methyl4-(2-(dimethylamino)-3-((9Z,12Z)-octadeca-9,12-dien-1-yloxy)propoxy)butanoate, ethyl(9Z)-21-(dimethylamino)triacont-9-enoate, (12Z,15Z)—N,N-dimethyl-2-nonylhenicosa-12,15-dien-1-amine, methyl8-(2-(dimethylamino)-3-((8-(2-octylcyclopropyl)octyl)oxy)propoxy)octanoate, ethyl(9Z)-19-(dimethylamino)pentacos-9-enoate, ethyl(18Z,21Z)-8-{[4-(dimethylamino)butanoyl]oxy}heptacosa-18,21-dienoate, (16Z)—N,N-dimethylpentacos-16-en-8-amine, methyl(9Z)-19-{[4-(dimethylamino)butanoyl]oxy}heptacos-9-enoate, methyl(9Z)-19-(dimethylamino)heptacos-9-enoate, 2-(Didodecylamino)-1-(4-(2-((2-(didodecylamino)ethyl)(dodecyl)amino)ethyl)piperazin-1-yl)ethan-1-one, (Z)-methyl16-(2-(dimethylamino)-3-(hexyloxy)propoxy)hexadec-7-enoate, (2S)-1-[(9Z,12Z)-octadeca-9,12-dien-1-yloxy]dodecan-2-amine, (16Z,19Z)—N,N-dimethylpentacosa˜16,19-dien-8-amine, N1-(2-(4-(2-(Dinonylamino)ethyl)piperazin-1-yl)ethyl)-N1,N2\hat{ }V2-tritetradecylethane-1,2-diamine, methyl7-(2-(dimethylamino)-3-((8-(2-octylcyclopropyl)octyl)oxy)propoxy)heptanoate, methyl(19Z,22Z)-9-{[4-(dimethylamino)butanoyl]oxy}octacosa-19,22-dienoate, ethyl(9Z)-19-(dimethylamino)hexacos-9-enoate, (22Z)—N,N-dimethylhentriacont-22-en-10-amine, N1-(2-(4-(2-(Di((Z)-octadec-9-en-1-yl)amino)ethyl)piperazin-1-yl)ethyl)-!\hat{ }\hat{ }-tridodecylethane-1,2-diamine, methyl5-(2-(dimethylamino)-3-((8-(2-octylcyclopropyl)octyl)oxy)propoxy)pentanoate, ethyl(9Z)-19-(dimethylamino)heptacos-9-enoate, (2-butylcyclopropyl)methyl12-{[4-(dimethylamino)butanoyl]oxy}henicosanoate, (20Z)—N,N-dimethylnonacos-20-en-10-amine, N1,N1,N2-Tridodecyl-N2-(2-(4-(2-(dodecyl((9Z,12Z)-octadeca-9,12-dien-1-yl)amino)ethyl)piperazin-1-yl)ethyl)ethane-1,2-diamine, methyl4-(2-(dimethylamino)-3-((8-(2-octylcyclopropyl)octyl)oxy)propoxy)butanoate, ethyl(9Z)-19-(dimethylamino)octacos-9-enoate, (2-octylcyclopropyl)methyl8-{[4-(dimethylamino)butanoyl]oxy}heptadecanoate, (24Z)—N,N-dimethyltritriacont-24-en-10-amine, N1-(2-(4-(2-(Ditetradecylamino)ethyl)piperazin-1-yl)ethyl)-N1,N2,N2-tridodecylethane-1,2-diamine, ethyl(5Z)-17-(dimethylamino)hexacos-5-enoate, (Z)-methyl8-(2-(dimethylamino)-3-(octadec-9-en-1-yloxy)propoxy)octanoate, (2Z)-hept-2-en-1-yl12-{[4-(dimethylamino)butanoyl]oxy}henicosanoate, (17Z)—N,N-dimethylnonacos-17-en-10-amine, N1-(2-(4-(2-(Di((Z)-dodec-6-en-1-yl)amino)ethyl)piperazin-1-yl)ethyl)-N1,N2,N2-tridodecylethane-1,2,-diamine, ethyl(9Z)-17-(dimethylamino)hexacos-9-enoate, (Z)-methyl7-(2-(dimethylamino)-3-(octadec-9-en-1-yloxy)propoxy)heptanoate, (2Z)-undec-2-en-1-yl8-{[4-(dimethylamino)butanoyl]oxy}heptadecanoate, (14Z)—N,N-dimethylnonacos-14-en-10-amine, ethyl(7Z)-17-(dimethylamino)tricos-7-enoate, (Z)-N1-(2-(4-(2-(Dodec-6-en-1-yl(dodecyl)amino)ethyl)piperazin-N!\hat{ }\hat{ }-tridodecylethane-1,2-diamine, (Z)-methyl5-(2-(dimethylamino)-3-(octadec-9-en-1-yloxy)propoxy)pentanoate, (2-hexylcyclopropyl)methyl10-{[4-(dimethylamino)butanoyl]oxy}nonadecanoate, (15Z)—N,N-dimethylheptacos-15-en-10-amine, ethyl(7Z)-17-(dimethylamino)tetracos-7-enoate, (Z)-methyl4-(2-(dimethylamino)-3-(octadec-9-en-1-yloxy)propoxy)butanoate, (2Z)-non-2-en-1-yl10-{[4-(dimethylamino)butanoyl]oxy}nonadecanoate, (20Z)—N,N-dimethylheptacos-20-en-10-amine, N1-(2-(4-(2-(Dioctylamino)ethyl)piperazin-1-yl)ethyl)-N1,N2\hat{ }V2-tridodecylethane-1,2-diamine, methyl6-(2-(dimethylamino)-3-((8-(2-octylcyclopropyl)octyl)oxy)propoxy)hexanoate, ethyl6-[2-(9-{[4-(dimethylamino)butanoyl]oxy}octadecyl)cyclopropyl]hexanoate, ethyl(7Z)-17-(dimethylamino)pentacos-7-enoate, 1-[(11Z,14Z)-1-nonylicosa-11,14-dien-1-yl]pyrrolidine, ethyl(7Z)-17-(dimethylamino)hexacos-7-enoate, (20Z,23Z)—N-ethyl-N-methylnonacosa-20,23-dien-10-amine, N,N-dimethylheptacosan-10-amine, methyl6-{2-[ll-(dimethylamino)icosyl]cyclopropyl}hexanoate, methyl6-[2-(ll-{[4-(dimethylamino)butanoyl]oxy}icosyl)cyclopropyl]hexanoate, (2-octylcyclopropyl)methyl6-(3-(decyloxy)-2-(dimethylamino)propoxy)hexanoate, methyl8-{2-[9-(dimethylamino)octadecyl]cyclopropyl}octanoate, methyl8-[2-(9-{[4-(dimethylamino)butanoyl]oxy}octadecyl)cyclopropyl]octanoate, methyl7-(2-(8-(2-(dimethylamino)-3-(octyloxy)propoxy)octyl)cyclopropyl)heptanoate, Heptadecan-9-yl8-((2-hydroxyethyl)(tetradecyl)amino)octanoateRepresentative, 2-((2-(Didodecylamino)ethyl)(dodecyl)amino)-1-(4-(2-(didodecylamino)ethyl)piperazin-1-yl)ethan-1-one, (2S)-1-[(9Z,12Z)-octadeca-9,12-dien-1-yloxy]undecan-2-amine, (17Z,20Z)—N,N-dimemylhexacosa-17,20-dien-9-amine, (18Z)-heptacos-18-en-10-yl4-(dimethylamino)butanoate, (2S)-1-({6-[3B))-cholest-5-en-3-yloxy]hexyl}oxy)-N,N-dimethyl-3-[(9Z)-octadec-9-en-1-yloxy]propan-2-amine, methyl10-{2-[7-(dimethylamino)hexadecyl]cyclopropyl}decanoate, methyl10-[2-(7-{[4-(dimethylamino)butanoyl]oxy}hexadecyl)cyclopropyl]decanoate, (2S)—N,N-dimethyl-1-({8-[(1R,2R)-2-{[(1S,2S)-2-pentylcyclopropyl]methyl}cyclopropyl]octyl}oxy)tridecan-2-amine, (2-octylcyclopropyl)methyl6-(2-(dimethylamino)-3-(nonyloxy)propoxy)hexanoate, (19Z,22Z)—N,N-dimethyloctacosa-19,22-dien-7-amine, 4-((N-(2-(Dinonylamino)ethyl)-N-nonylglycyl)oxy)pentan-2-yldinonylglycinate, 3-Hydroxybutan-2-ylN-(2-(dinonylamino)ethyl)-N-nonyl, Di(heptadecan-9-yl)8,8′-(26,28-dimethyl-ll,24,30,43-tetraoxo-10,25,29,44-tetraoxa-19,35-diazatripentacontane-19,35-diyl)dioctanoate, Di(heptadecan-9-yl)8,8′-(26,27-dimethyl-ll,24,29,42-tetraoxo-10,25,28,43-tetraoxa-19,34-diazadopentacontane-19,34-diyl)dioctanoate, Di(heptadecan-9-yl)8,8′-(ll,24,29,42-tetraoxo-10,25,28,43-tetraoxa-19,34-diazadopentacontane-19,34-diyl)dioctanoate, Di(heptadecan-9-yl)8,8′-((piperazine-1,4-diylbis(5-oxopentane-5,1-diyl))bis((8-(nonyloxy)-8-oxooctyl)azanediyl))dioctanoate, Di(heptadecan-9-yl)15,18-dimethyl-9,24-bis(8-(nonyloxy)-8-oxooctyl)-14,19-dioxo-9,15,18,24-tetraazadotriacontanedioate, Di(heptadecan-9-yl)15,19-dimethyl-9,25-bis(8-(nonyloxy)-8-oxooctyl)-14,20-dioxo-9,15,19,25-tetraazatritriacontanedioate, Di(heptadecan-9-yl)15,18-diethyl-9,24-bis(8-(nonyloxy)-8-oxooctyl)-14,19-dioxo-9,15,18,24-tetraazadotriacontanedioate, N,N-dimethyl-3-{[(9Z,12Z)-octadeca-9,12-dien-1-yloxy]methyl}dodecan-1-amine, methyl8-[2-(ll-{[4-(dimethylamino)butanoyl]oxy}octadecyl)cyclopropyl]octanoate, methyl8-{2-[ll-(dimethylamino)heptadecyl]cyclopropyl}octanoate(Compound18); Heptadecan-9-yl8-((2-hydroxyethyl)(8-(nonyloxy)-8-oxooctyl)amino)octanoate, (2-octylcyclopropyl)methyl6-(2-(dimethylamino)-3-(heptyloxy)propoxy)hexanoate, (17Z)—N,N-dimethylhexacos-17-en-9-amine, N1-(2-(4-(2-(Didodecylamino)ethyl)piperazin-1-yl)ethyl)-N1\hat{ }V2,N2-trihexylethane-1,2-diamine, N,N-dimethyl-2-{[(9Z,12Z)-octadeca-9,12-dien-1-yloxy]methyl}undecan-1-amine, methyl8-{2-[11-(dimethylamino)octadecyl]cyclopropyl}octanoate, (2-octylcyclopropyl)methyl6-(2-(dimethylamino)-3-(hexyloxy)propoxy)hexanoate, (18Z)—N,N-dimethylheptacos-18-en-10-amine, 2-((2-(Dinonylamino)ethyl)(nonyl)amino)ethyltetradecanoate, 2-((2-(Dinonylamino)ethyl)(nonyl)amino)ethylnonanoate, TetradecylN-(2-(dinonylamino)ethyl)-N-nonylglycinate, NonylN-(2-(dinonylamino)ethyl)-N-nonylglycinate, 4-(2-((2-(dinonylamino)ethyl)(nonyl)amino)acetamido)butylpentanoate, 1,l′-(Piperazine-1,4-diyl)bis(5-(didecylamino)pentan-1-one, 2-((2-(dinonylamino)ethyl)(nonyl)amino)-N-tetradecylacetamide, N-decyl-2-((2-(dinonylamino)ethyl)(nonyl)amino), N1-(3-(3-(dinonylamino)propoxy)propyl)-N1,N2,N2-trinonylethane-1,2-diamine, N1-(2-(dinonylamino)ethyl)-N\N8,N8-trinonyloctane-1,8-diamine, methyl8-[2-(ll-{[4-(dimethylamino)butanoyl]oxy}nonadecyl)cyclopropyl]octanoate, methyl8-{2-[ll-(dimethylamino)nonadecyl]cyclopropyl}octanoate, (Z)-undec-2-en-1-yl6-(3-(decyloxy)-2-(dimethylamino)propoxy)hexanoate, (2R,12Z,15Z)—N,N-dimethyl-1-(undecyloxy)henicosa-12,15-dien-2-amine, (21Z,24Z)—N,N-dimethyltriaconta-21,24-dien-9-amine, 2-(dinonylamino)-N-(4-(2-((2-(dinonylamino)ethyl)(nonyl)amino)-N-methylacetamido)butyl)-N-methylacetamide, 7,10-dimethyl-13,16-dinonyl-6,11-dioxo-4-tetradecyl-4,7,10,13,16-pentaazapentacosyldecanoate, 2-(dinonylamino)-N-(2-(2-((2-(dinonylamino)ethyl)(nonyl)amino)-N-ethylacetamido)ethyl)-N-ethylacetamide, 2-(dinonylamino)-N-(3-(2-((2-(dinonylamino)ethyl)(nonyl)amino)-N-methylacetamido)propyl)-N-methylacetamide, 2-((2-(di((Z)-non-3-en-1-yl)amino)ethyl)((Z)-non-3-en-1-yl)amino)-N-(2-(2-(dinonylamino)-N-methylacetamido)ethyl)-N-methylacetamide, 2-(dinonylamino)-N-(2-(2-((2-(dinonylamino)ethyl)(nonyl)amino)acetamido)ethyl)acetamide, Pentyl8,11-dimethyl-5,14,17-trinonyl-7,12-dioxo-5,8,11,14,17-pentaazahexacosanoate2-((2-(Dinonylamino)ethyl)(nonyl)anilino)-N-methyl-N-(2-(methylindeno)ethyl)acetami, 2-(Dinonylamino)-N-(2-(2-((2-(dinonylamino)ethyl)(nonyl)amino)-N-methylacetamido)ethyl)-N-methylacetamide2-(Dinonylamino)-N-methyl-N-(2-(methylamino)ethyl)acetamide, 2-((N-(2-(Dinonylamino)ethyl)-N-nonylglycyl)oxy)ethyldinonylglycinate2-Hydroxyethyldinonylglycinate, methyl8-[2-(ll-{[4-(dimethylamino)butanoyl]oxy}icosyl)cyclopropyl]octanoate, methyl8-{2-[ll-(dimethylamino)icosyl]cyclopropyl}octanoate, (Z)-undec-2-en-1-yl6-(2-(dimethylamino)-3-(nonyloxy)propoxy)hexanoate, (2R,12Z,15Z)-1-(hexadecyloxy)-N,N-dimethylhenicosa-12,15-dien-2-amine, (22Z,25Z)—N,N-dimethylhentriaconta-22,25-dien-10-amine, 1,1-(Piperazine-1,4-diyl)bis(4-(didecylamino)butan-1-one)fert-Butyl4-(didecylaminobutanoate, Heptyl5-(4-(N-(2-(dinonylamino)ethyl)-N-nonylglycyl)piperazin-1-yl)-5-oxopentanoate5-(Heptloxy)-5-oxopentanoicacid, Heptyl5-(4-(N-(2-(dinonylamino)ethyl)-N-nonylglycyl)piperazin-1-yl)-5-oxopentanoate5-(Heptloxy)-5-oxopentanoic, (Z)-4-((2-(4-(N-(2-(dinonylamino)ethyl)-N-nonylglycyl)piperazin-1-yl)-2-oxoethyl)(tetradecyl)amino)but-2-en-1-ylnonanoate(Z)-4-Hydroxybut-2-en-1-ylnonanoate, (Z)-3-((2-(4-(N-(2-(Dinonylamino)ethyl)-N-nonylglycyl)piperazin-1-yl)-2-oxoethyl)(tetradec-9-en-1-yl)amino)propy 1 decanoate(Z)-Tetradec-9-en-1-ylmethanesulfonate, methyl8-[2-(9-{[4-(dimethylamino)butanoyl]oxy}pentadecyl)cyclopropyl]octanoate, methyl8-{2-[9-(dimethylamino)pentadecyl]cyclopropyl}octanoate, (Z)-undec-2-en-1-yl6-(2-(dimethylamino)-3-(heptyloxy)propoxy)hexanoate, (2R,12Z,15Z)-1-(hexyloxy)-N,N-dimethylhenicosa-12,15-dien-2-amine, (16Z,19Z)—N,N-dimethylpentacosa-16,19-dien-6-amine, Methyl8-((2-(4-(N-(2-(Di((Z)-non-3-en-1-yl)amino)ethyl)-N—((Z)-non-3-en-1-yl)glycyl)piperazin-1-yl)-2-oxoethyl)(nonyl)amino)octanoatefert-Butyl4-(nonylglycyl)piperazine-1-carboxylate, 3-((2-(4-(N-(2-(Dinonylamino)ethyl)-N-nonylglycyl)piperazin-1-yl)-2-oxoethyl)(tetradecyl)amino)propyl(Z)-dec-3-enoate(Z)-Dec-3-en-1-ol, 2-((2-(Di((Z)-non-3-en-1-yl)amino)ethyl)((Z)-non-3-en-1-yl)amino)-1-(4-(dinonylglycyl)piperazin-1-yl)ethan-1-one(Z)-1-Bromonon-4-ene, 3-((2-(4-(N-(2-(Dinonylamino)ethyl)-N-nonylglycyl)piperazin-oxoethyl)(dodecyl)amino)propyloctanoatetot-Butyldodecylglycinate, S-Pentyl4-((2-(4-(N-(2-(dinonylamino)ethyl)-N-nonylglycyl)piperazin-1-yl)-2-oxoethyl)(nonyl)amino)butanethioate, 3-((2-(1-(N-(2-(Dinonylamino)ethyl)-N-nonylglycyl)piperidin-\hat{ }yl)ethyl)(nonyl)amino)propyl3-methylhexanoatefert-Butyl4-(2-((3-((3-methylhexanoyl)oxy)propyl)(nonyl)amino)ethyl)piperidine-1-, 3-((2-(1-(N-(2-(Dinonylamino)ethyl)-N-nonylglycyl)piperidin-4-yl)ethyl)(nonyl)amino)-2-methylpropylhexanoate, 3-((2-(4-(N-(2-(Dinonylamino)ethyl)-N-nonylglycyl)piperazin-oxoethyl)(nonyl)amino)propyl3-methylhexanoate, 3-((2-(4-(N-(2-(Dinonylamino)ethyl)-N-nonylglycyl)piperazin-oxoethyl)(nonyl)amino)-2-methylpropylhexanoate, methyl8-[2-(9-{[4-(dimethylamino)butanoyl]oxy}hexadecyl)cyclopropyl]octanoate, methyl8-{2-[9-(dimethylamino)hexadecyl]cyclopropyl}octanoate, (Z)-undec-2-en-1-yl6-(2-(dimethylamino)-3-(hexyloxy)propoxy)hexanoate, (2R,12Z,15Z)-1-(decyloxy)-N,N-dimethylhenicosa-12,15-dien-2-amine, (17Z,20Z)—N,N-dimethylhexacosa-17,20-dien-7-amine, 2-((2-(Dinonylamino)ethyl)(nonyl)amino)ethyl1-(dinonylglycyl)piperidine-4-carboxylate, 1-(2-(Dinonylamino)ethyl)4-(2-((2-(dinonylamino)ethyl)(nonyl)amino)ethyl)cyclohexane-1,4-dicarboxylate2-(Dinonylamino)ethan-1-ol, Methyl12-((2-(1-(N-(2-(dinonylamino)ethyl)-N-nonylglycyl)pyrrolidin-3-yl)ethyl)(tetradecyl)amino)dodecanoatefert-Butyl3-(2-((12-methoxy-12-oxododecyl)(tetradecyl)amino)ethyl)pyrrolidine-1-carboxylate, 3-((2-(1-(N-(2-(Dinonylamino)ethyl)-N-nonylglycyl)pyrrolidin-3-yl)ethyl)(tetradecyl)amino)propyldecanoateter/-Butyl3-(2-((3-(decanoyloxy)propyl)(tetradecyl)amino)ethyl)pyrrolidine-1-carboxylate, “Heptyl6-((2-(1-(N-(2-(dinonylamino)ethyl)-N-nonylglycyl)pyrrolidin-3-yl)ethyl)(tetradecyl)amino)hexanoatetot-Butyl3-(2-((6-(heptyloxy)-6-oxohexyl)(tetradecyl)amino)ethyl)pyrrolidine-1-carboxylate, Pentyl8-((2-(1-(N-(2-(dinonylamino)ethyl)-N-nonylglycyl)pyrrolidin-3-yl)ethyl)(tetradecyl)amino)octanoate/er/-Butyl3-(2-(tetradecylamino)ethyl)pyrrolidine-1-carboxylate, Methyl12-((2-(1-(N-(2-(dinonylamino)ethyl)-N-nonylglycyl)piperidin-3-yl)ethyl)(tetradecyl)amino)dodecanoate-Butyl3-(2-((12-methoxy-12-oxododecyl)(tetradecyl)amino)ethyl)piperidine-1-carboxylate, 3-((2-(1-(N-(2-(Dinonylamino)ethyl)-N-nonylglycyl)piperidin-3-yl)ethyl)(tetradecyl)amino)propyldecanoate, Heptyl6-((2-(1-(N-(2-(dinonylamino)ethyl)-N-nonylglycyl)piperidin-3-yl)ethyl)(tetradecyl)amino)hexanoate, Pentyl8-((2-(1-(N-(2-(dinonylamino)ethyl)-N-nonylglycyl)piperidin-3-yl)ethyl)(tetradecyl)amino)octanoate, Pentyl6-((2-(4-(2-((2-(didodecylamino)ethyl)(dodecyl)amino)ethyl)piperazin-1-yl)ethyl)(dodecyl)amino)hexanoateStep1:Pentyl6-bromohexanoate, methyl8-[2-(9-{[4-(dimethylamino)butanoyl]oxy}heptadecyl)cyclopropyl]octanoate, methyl8-{2-[9-(dimethylamino)heptadecyl]cyclopropyl}octanoate, (2S,12Z,15Z)—N,N-dimethyl-1-(octyloxy)henicosa-12,15-dien-2-amine, (2-octylcyclopropyl)methyl6-(2-(dimethylamino)-3-(octyloxy)propoxy)hexanoate, (18Z,21Z)—N,N-dimethylheptacosa-18,21-dien-8-amine, trans-1-methyl-3,4-bis(((Z)-hexadec-9-enoyloxy)methyl)pyrrolidine, (Z)-Non-2-en-1-yl4-((2-(4-(N-(2-(dinonylamino)ethyl)-N-nonylglycyl)piperazin-1-yl)-2-oxoethyl)(tetradecyl)amino)butanoate, trans-1-methyl-3,4-bis(((9Z,12Z)-octadeca-9,12-dienoyloxy)methyl)pyrrolidine, Methyl12-((2-(4-(N-(2-(dinonylamino)ethyl)-N-nonylglycyl)piperazin-1-yl)-2-oxoethyl)(tetradecyl)amino)dodecanoate, ethyl(7Z)-17-[2-(dimethylamino)ethyl]hexacos-7-enoate, trans-1-methyl-3,4-bis(((Z)-octadeca-9-enoyloxy)methyl)pyrrolidine, methyl6-(2-{]11-\hat{ }2-(dimethylamino)ethyl]icosyl}cyclopropyl)hexanoate, Methyl12-((2-(1-(N-(2-(dinonylamino)ethyl)-N-nonylglycyl)piperidin-4-yl)ethyl)(tetradecyl)amino)dodecanoate, methyl10-(2-V-\hat{ }2-(dimethylamino)ethyl]hexadecyl}cyclopropyl)decanoate, methyl8-(2-{111-; 2-(dimethylamino)ethyl]heptadecyl}cyclopropyl)octanoate, 2-(1-(N-(2-(Dinonylamino)ethyl)-N-nonylglycyl)piperidin-4-yl)ethyldinonylglycinatefert-Butyl4-(2-((dinonylglycyl)oxy)ethyl)piperidine-1-carboxylate, methyl8-(2-{lL1-; 2-(dimethylamino)ethyl]octadecyl}cyclopropyl)octanoate, methyl8-(2-{l11-\hat{ }2-(dimethylamino)ethyl]nonadecyl}cyclopropyl)octanoate, 1,-(piperazine-1,4-diyl)bis(2-(dinonylamino)ethan-1-one), methyl8-[2-{]11-\hat{ }2-(dimethylamino)ethyl]icosyl}cyclopropyl)octanoate, methyl8-(2-{9-[2-(dimethylamino)ethyl]pentadecyl}cyclopropyl)octanoate, methyl(7Z)-19-{[4-(dimethylamino)butanoyl]oxy}octacos-7-enoate, methyl(7Z)-19-(dimethylamino)octacos-7-enoate, cis-1-methyl-3-[(9Z,12Z)-octadeca-9,12-dien-1-yloxy]-4-(octyloxy)pyrrolidine, 2-(Didodecylamino)-1-(4-(N-(2-(didodecylamino)ethyl)-N-dodecylglycyl)piperazin-1-yl)ethan-1-one, (Z)-undec-2-en-1-yl6-(2-(dimethylamino)-3-(octyloxy)propoxy)hexanoate, (2SN,N-dimethyl-1-[(9Z,12Z)-octadeca-9,12-dien-1-yloxy]decan-2-amine(Compound11), (19Z,22Z)—N,N-dimeihyloctacosa-19,22-dien-9-amine, methyl8-(2-{9-[2-(dimethylamino)ethyl]hexadecyl}cyclopropyl)octanoate, 5-((2-(4-(N-(2-(Dinonylamino)ethyl)-N-nonylglycyl)piperazin-oxoethyl)(nonyl)amino)pentylmethylcarbonate, methyl8-(2-{9-[2-(dimethylamino)ethyl]heptadecyl}cyclopropyl)octanoate, methyl(7Z)-19-[2-(dimethylamino)ethyl]octacos-7-enoate, (Z)-Pent-2-en-1-yl4-((2-(4-(N-(2-(dinonylamino)ethyl)-N-nonylglycyl)piperazin-1-yl)-2-oxoethyl)(nonyl)amino)butanoate, methyl(1lZ)-19-[2-(dimethylamino)ethyl]octacos-11-enoate, methyl(9Z)-21-[2-(dimethylamino)ethyl]heptacos-9-enoate, methyl(9Z)-21-[2-(dimethylamino)ethyl]octacos-9-enoate, methyl(9Z)-21-[2-(dimethylamino)ethyl]nonacos-9-enoate, 2-(1-(N-(2-(Dinonylamino)ethyl)-N-nonylglycyl)pyrrolidin-3-yl)ethyldinonylglycinate, methyl(9Z)-21-[2-(dimethylamino)ethyl]triacont-9-enoate, (1-(N-(2-(Dinonylamino)ethyl)-N-nonylglycyl)pyrrolidin-3-yl)methyldinonylglycinate, methyl(9Z)-19-[2-(dimethylamino)ethyl]pentacos-9-enoate, methyl(9Z)-19-[2-(dimethylamino)ethyl]hexacos-9-enoate, methyl6-(2-(8-(3-(decyloxy)-2-(dimethylamino)propoxy)octyl)cyclopropyl)hexanoate, methyl(1lZ)-19-{[4-(dimethylamino)butanoyl]oxy}octacos-ll-enoate, methyl(1lZ)-19-(dimethylamino)octacos-ll-enoate, (2S)—N,N-dimethyl-1-[(9Z,12Z)-octadeca-9,12-dien-1-yloxy]dodecan-2-amine, (14Z,17Z)—N,N-dimethyltricosa-14,17-dien-4-amine, Methyldi((9Z,12Z)-octadeca-9,12-dienyl)amine, methyl(9Z)-19-{[4-(dimethylamino)butanoyl]oxy}octacos-9-enoate, methyl(9Z)-19-(dimethylamino)octacos-9-enoate, (Z)-methyl17-(2-(dimethylamino)-3-(octyloxy)propoxy)heptadec-8-enoate, (3R,4R)-3,4-bis((Z)-hexadec-9-enyloxy)-1-methylpyrrolidine, (2S)—N,N-dimethyl-1-[(9Z,12Z)-octadeca-9,12-dien-1-yloxy]undecan-2-amine, (20Z,23Z)-nonacosa-20,23-dien-10-yl4-(dimethylamino)butanoate, (20Z,23Z)—N,N-dimethylnonacosa-20,23-dien-10-amine, 3-((6Z,9Z,28Z,31Z)-heptatriaconta-6,9,28,31-tetraen-19-yloxy)-N,N-dimethylpropan-1-amine, 3-((6Z,9Z,28Z,31Z)-heptatriaconta-6,9,28,31-tetraen-19-yloxy)-N,N-dimethylpropan-1-amine, (6Z,9Z,28Z,31Z)-heptatriaconta-6,9,28,31-tetraen-19-yl4-(dimethylamino)butanoate), (6Z,16Z)-12-((Z)-dec-4-enyl)docosa-6,16-dien-1l-yl5-(dimethylamino)pentanoate, (6Z,16Z)-12-((Z)-dec-4-enyl)docosa-6,16-dien-ll-yl5-(dimethylamino)pentanoate, (6Z,16Z)-12-((Z)-dec-4-enyl)docosa-6,16-dien-11-yl5-(dimethylamino)pentanoate, L-arginine-alpha-(2,3-dilauryloxy)propylamide, L-lysine-alpha-(2,3-dilauryloxy)propylamide, 2,3-dioleyloxypropylamine, 2,3-distearyloxypropylamine, 2,3-dilauryloxypropylamine, dilinoleylmethyl4-(dimethylamino)propylether), dilinoleylmethyl4-(dimethylamino)butylether), and 2,2-dilinoleyl-4-(2-dimethylaminoethyl)-[1,3]-dioxolane.

In some embodiments, the at least one non-cationic lipid comprises at least one phospholipid, at least one fusogenic lipid, at least one anionic lipid, at least one helper lipid, at least one neutral lipid, or any combination thereof. In some embodiments, the LNP may be essentially devoid of the at least one non-cationic lipid. In some embodiments, the LNP may contain no amount of the at least one non-cationic lipid.

In some embodiments, at least one non-cationic lipid may be selected from, but is not limited to, at least one of 1,2-di-O-octadecenyl-sn-glycero-3-phosphocholine (18:0 Diether PC), DSPCbutwith3unsaturateddoublebondspertail (18:3 PC), Acylcarnosine (AC), 1-hexadecyl-sn-glycero-3-phosphocholine (C16 Lyso PC), N-oleoyl-SPM (C18:1), N-lignocerylSPM (C24:0), N-nervacylC (C24:1), carbamoyl]cholesterol (Cet-P), cholesterolhemisuccinate (CHEMS), cholesterol (Chol), Cholesterolhemidodecanedicarboxylicacid (Chol-C12), 12-Cholesteryloxycarbonylaminododecanoicacid (Chol-C13N), Cholesterolhemioxalate (Chol-C2), Cholesterolhemimalonate (Chol-C3), N-(Cholesteryl-oxycarbonyl)glycine (Chol-C3N), Cholesterolhemiglutarate (Chol-C5), Cholesterolhemiadipate (Chol-C6), Cholesterolhemipimelate (Chol-C7), Cholesterolhemisuberate (Chol-C8), Cardiolipid (CL), 1,2-bis(tricosa-10,12-diynoyl)-sn-glycero-3-phosphocholine (DC8-9PC), dicetylphosphate (DCP), dihexadecylphosphate (DCP1), 1,2-Dipalmitoylglycerol-3-hemisuccinate (DGSucc), short-chainbis-n-heptadecanoylphosphatidylcholine (DHPC), dihexadecoylphosphoethanolamine (DHPE), 1,2-dilinoleoyl-sn-glycero-3-phosphocholine (DLPC), 1,2-dilauroyl-sn-glycero-3-PE (DLPE), Dimyristoylglycerolhemisuccinate (DMGS), dimyristoylphosphatidylcholine (DMPC), dimyristoylphosphoethanolamine (DMPE), dimyristoylphosphatidylglycerol (DMPG), dioleyloxybenzylalcohol (DOBA), 1,2-dioleoylglycerol-3-hemisuccinate (DOGHEMS), N-[2˜(2-{2-[2-(2,3-Bis-octadec-9-enyloxy-propoxy)-ethoxy]-ethoxy}-ethoxy)-ethyl]-3-(3,4,5-1rihydroxy-6-hydroxymethyl-1etrahydro-pyran-2-ylsulfanyl)-propionamide (DOGP4αMan), dioleoylphosphatidylcholine (DOPC), dioleoylphosphatidylethanolamine (DOPE), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylcholine (DPPC), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoylphosphoethanolamineimidazole (DSPEI), 1,2-diundecanoyl-sn-glycero-phosphocholine (DUPC), eggphosphatidylcholine (EPC), N-histidinylcholesterolcarbamate (HCChol), histaminedistearoylglycerol (HDSG), N-histidinylcholesterolhemisuccinate (HistChol), 1,2-Dipalmitoylglycerol-hemisuccinate-Na-Histidinyl-Hemisuccinate (HistSuccDG), N-(5′-hydroxy-3′-oxypentyl)-10-12-pentacosadiynamide (h-Pegi-PCDA), 2-[1-hexyloxyethyl]-2-devinylpyropheophorbide-a (HPPH), hydrogenatedsoybeanphosphatidylcholine (HSPC), 1,2-Dipalmitoylglycerol-Oα-histidinyl-Nα-hemisuccinate (IsohistsuccDG), mannosializeddipalmitoylphosphatidylethanolamine (ManDOG), 1,2-Dioleoyl-sn-Glycero-3-Phosphoethanolamine-N-[4-(p-maleimidomethyl)cyclohexane-carboxamide](MCC-PE), 1,2-diphytanoyl-sn-glycero-3-phosphoethanolamine (ME 16.0 PE), 1-myristoyl-2-hydroxy-sn-glycero-phosphocholine (MHPC), athiol-reactivemaleimideheadgrouplipide.g.1,2-dioleoyl-sn-glycero-3-phosphoethanolamine-N-[4-(p-maleimidophenyl)but-yramid (MPB-PE), NervonicAcid (NA), sodiumcholate (NaChol), 1,2-dioleoyl-sn-glycero-3-[phosphoethanolamine-N-dodecanoyl (NC12-DOPE), Defined by synthesis example in WO2008042973A2 (ND98), “N-glutarylphosphatidylethanolamine(s) of Formula 1” (NG-PE), N-hydroxysulfosuccinimide (NHS-‘x’), “N˜(co)-dicarboxylicacid-derivatizedphosphatidylethanolaminesencompassedbyFormula1” (NωPE-‘x’), OleicAcid (OA), 1-oleoyl-2-cholesterylhemisuccinoyl-sn-glycero-3-phosphocholine (OChemsPC), phosphatidic acid (PA), phosphatidylethanolaminelipid (PE), PElipidconjugatedwithpolyethyleneglycol(PEG). One example of PEG-PEcanbepolyethyleneglycol-distearoylphosphatidylethanolaminelipid (PEG-PE), phosphatidylglycerol (PG), partiallyhydrogenatedsoyphosphatidylchloline (PHSPC), phosphatidylinositollipid (PI), phosphotidylinositol-4-phosphate (PIP), palmitoyloleoylphosphatidylcholine (POPC), phosphatidylethanolamine (POPE), palmitoyloleyolphosphatidylglycerol (POPG), phosphatidylserine (PS), lissaminerhodamineB-phosphatidylethanolaminelipid (Rh-PE), purified soy-derived mixture of phospholipids (SIOO), phosphatidylcholine (SM), 18-1-transPE,1-stearoyl-2-oleoyl-phosphatidyethanolamine (SOPE), soybean phosphatidylcholine (SPC), sphingomyelins (SPM), alpha.alpha′-trehalose6,6′-dibehenate (TDB), 1,2-dielaidoyl-sn-glycero-3-phophoethanolamine (transDOPE), ((23S,5R)-3-(bis(hexadecyloxy)methoxy)-5-(5-methyl-2,4-dioxo-3,4-dihydropyrimidin-1(2/−/)-yl)tetrahydrofuran-2-yl)methylmethylphosphate, 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, 16-O-monomethylPE,16-O-dimethylPE, and dioleylphosphatidylethanolamine.

In some embodiments, the LNP comprises an ionizable lipid or lipid-like material. As a non-limiting example, the ionizable lipid may be C12-200, CKK-E12, 5A2-SC8, BAMEA-016B, or 7C1. Other ionizable lipids are known in the art and are useful herein.

In some embodiments, the LNP comprises a phospholipid. As a non-limiting example, the phospholipid (helper) may be DOPE, DSPC, DOTAP, or DOTMA.

In some embodiments, the LNP comprises a PEG derivative. As a non-limiting example, the PEG derivative may be a lipid-anchored such as PEG is C14-PEG2000, C14-PEG1000, C14-PEG3000, C14-PEG5000, C12-PEG1000, C12-PEG2000, C12-PEG3000, C12-PEG5000, C16-PEG1000, C16-PEG2000, C16-PEG3000, C16-PEG5000, C18-PEG1000, C18-PEG2000, C18-PEG3000, or C18-PEG5000. In some embodiments, the PEG derivative is a cyclic PEG such as

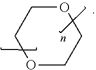

In some embodiments, the at least one sterol comprises at least one cholesterol or cholesterol derivative. In some embodiments, the LNP may be essentially devoid of an at least one sterol. In some embodiments, the LNP may contain no amount of the at least one sterol.

In some embodiments, the at least one particle-activity-modifying-agent comprises at least one component that reduced aggregation of particles, at least one component that decreases clearing of the LNP from circulation in a subject, at least component that increases the LNP's ability to traverse mucus layers, at least one component that decreases a subjects immune response to administration of the LNP, at least one component that modifies membrane fluidity of the LNP, at least one component that contributes to the stability of the LNP, or any combination thereof. In some embodiments, the LNP may be essentially devoid of the at least one particle-activity-modifying-agent. In some embodiments, the LNP may contain no amount of the at least one particle-activity-modifying-agent.

In some embodiments, the particle-activity-modifying-agent may be comprised of a polymer. In some embodiments, the polymer comprising the particle-activity-modifying-agent may be comprised of at least one polyethylene glycol (PEG), at least one polypropylene glycol (PPG), poly(2-oxazoline) (POZ), at least one polyamide (ATTA), at least one cationic polymer, or any combination thereof.

In some embodiments, the average molecular weight of the polymer moiety (e.g., PEG) may be between 500 and 20,000 daltons. In some embodiments, the molecular weight of the polymer may be about 500 to 20,000, 1,000 to 20,000, 1,500 to 20,000, 2,000 to 20,000, 2,500 to 20,000, 3,000 to 20,000, 3,500 to 20,000, 4,000 to 20,000, 4,500 to 20,000, 5,000 to 20,000, 5,500 to 20,000, 6,000 to 20,000, 6,500 to 20,000, 7,000 to 20,000, 7,500 to 20,000, 8,000 to 20,000, 8,500 to 20,000, 9,000 to 20,000, 9,500 to 20,000, 10,000 to 20,000, 10,500 to 20,000, 11,000 to 20,000, 11,500 to 20,000, 12,000 to 20,000, 12,500 to 20,000, 13,000 to 20,000, 13,500 to 20,000, 14,000 to 20,000, 14,500 to 20,000, 15,000 to 20,000, 15,500 to 20,000, 16,000 to 20,000, 16,500 to 20,000, 17,000 to 20,000, 17,500 to 20,000, 18,000 to 20,000, 18,500 to 20,000, 19,000 to 20,000, 19,500 to 20,000, 500 to 19,500, 1,000 to 19,500, 1,500 to 19,500, 2,000 to 19,500, 2,500 to 19,500, 3,000 to 19,500, 3,500 to 19,500, 4,000 to 19,500, 4,500 to 19,500, 5,000 to 19,500, 5,500 to 19,500, 6,000 to 19,500, 6,500 to 19,500, 7,000 to 19,500, 7,500 to 19,500, 8,000 to 19,500, 8,500 to 19,500, 9,000 to 19,500, 9,500 to 19,500, 10,000 to 19,500, 10,500 to 19,500, 11,000 to 19,500, 11,500 to 19,500, 12,000 to 19,500, 12,500 to 19,500, 13,000 to 19,500, 13,500 to 19,500, 14,000 to 19,500, 14,500 to 19,500, 15,000 to 19,500, 15,500 to 19,500, 16,000 to 19,500, 16,500 to 19,500, 17,000 to 19,500, 17,500 to 19,500, 18,000 to 19,500, 18,500 to 19,500, 19,000 to 19,500, 1,500 to 19,000, 2,000 to 19,000, 2,500 to 19,000, 3,000 to 19,000, 3,500 to 19,000, 4,000 to 19,000, 4,500 to 19,000, 5,000 to 19,000, 5,500 to 19,000, 6,000 to 19,000, 6,500 to 19,000, 7,000 to 19,000, 7,500 to 19,000, 8,000 to 19,000, 8,500 to 19,000, 9,000 to 19,000, 9,500 to 19,000, 10,000 to 19,000, 10,500 to 19,000, 11,000 to 19,000, 11,500 to 19,000, 12,000 to 19,000, 12,500 to 19,000, 13,000 to 19,000, 13,500 to 19,000, 14,000 to 19,000, 14,500 to 19,000, 15,000 to 19,000, 15,500 to 19,000, 16,000 to 19,000, 16,500 to 19,000, 17,000 to 19,000, 17,500 to 19,000, 18,000 to 19,000, 18,500 to 19,000, 1,500 to 18,500, 2,000 to 18,500, 2,500 to 18,500, 3,000 to 18,500, 3,500 to 18,500, 4,000 to 18,500, 4,500 to 18,500, 5,000 to 18,500, 5,500 to 18,500, 6,000 to 18,500, 6,500 to 18,500, 7,000 to 18,500, 7,500 to 18,500, 8,000 to 18,500, 8,500 to 18,500, 9,000 to 18,500, 9,500 to 18,500, 10,000 to 18,500, 10,500 to 18,500, 11,000 to 18,500, 11,500 to 18,500, 12,000 to 18,500, 12,500 to 18,500, 13,000 to 18,500, 13,500 to 18,500, 14,000 to 18,500, 14,500 to 18,500, 15,000 to 18,500, 15,500 to 18,500, 16,000 to 18,500, 16,500 to 18,500, 17,000 to 18,500, 17,500 to 18,500, 18,000 to 18,500, 1,500 to 18,000, 2,000 to 18,000, 2,500 to 18,000, 3,000 to 18,000, 3,500 to 18,000, 4,000 to 18,000, 4,500 to 18,000, 5,000 to 18,000, 5,500 to 18,000, 6,000 to 18,000, 6,500 to 18,000, 7,000 to 18,000, 7,500 to 18,000, 8,000 to 18,000, 8,500 to 18,000, 9,000 to 18,000, 9,500 to 18,000, 10,000 to 18,000, 10,500 to 18,000, 11,000 to 18,000, 11,500 to 18,000, 12,000 to 18,000, 12,500 to 18,000, 13,000 to 18,000, 13,500 to 18,000, 14,000 to 18,000, 14,500 to 18,000, 15,000 to 18,000, 15,500 to 18,000, 16,000 to 18,000, 16,500 to 18,000, 17,000 to 18,000, 17,500 to 18,000, 1,500 to 17,500, 2,000 to 17,500, 2,500 to 17,500, 3,000 to 17,500, 3,500 to 17,500, 4,000 to 17,500, 4,500 to 17,500, 5,000 to 17,500, 5,500 to 17,500, 6,000 to 17,500, 6,500 to 17,500, 7,000 to 17,500, 7,500 to 17,500, 8,000 to 17,500, 8,500 to 17,500, 9,000 to 17,500, 9,500 to 17,500, 10,000 to 17,500, 10,500 to 17,500, 11,000 to 17,500, 11,500 to 17,500, 12,000 to 17,500, 12,500 to 17,500, 13,000 to 17,500, 13,500 to 17,500, 14,000 to 17,500, 14,500 to 17,500, 15,000 to 17,500, 15,500 to 17,500, 16,000 to 17,500, 16,500 to 17,500, 17,000 to 17,500, 1,500 to 17,000, 2,000 to 17,000, 2,500 to 17,000, 3,000 to 17,000, 3,500 to 17,000, 4,000 to 17,000, 4,500 to 17,000, 5,000 to 17,000, 5,500 to 17,000, 6,000 to 17,000, 6,500 to 17,000, 7,000 to 17,000, 7,500 to 17,000, 8,000 to 17,000, 8,500 to 17,000, 9,000 to 17,000, 9,500 to 17,000, 10,000 to 17,000, 10,500 to 17,000, 11,000 to 17,000, 11,500 to 17,000, 12,000 to 17,000, 12,500 to 17,000, 13,000 to 17,000, 13,500 to 17,000, 14,000 to 17,000, 14,500 to 17,000, 15,000 to 17,000, 15,500 to 17,000, 16,000 to 17,000, 16,500 to 17,000, 1,500 to 16,500, 2,000 to 16,500, 2,500 to 16,500, 3,000 to 16,500, 3,500 to 16,500, 4,000 to 16,500, 4,500 to 16,500, 5,000 to 16,500, 5,500 to 16,500, 6,000 to 16,500, 6,500 to 16,500, 7,000 to 16,500, 7,500 to 16,500, 8,000 to 16,500, 8,500 to 16,500, 9,000 to 16,500, 9,500 to 16,500, 10,000 to 16,500, 10,500 to 16,500, 11,000 to 16,500, 11,500 to 16,500, 12,000 to 16,500, 12,500 to 16,500, 13,000 to 16,500, 13,500 to 16,500, 14,000 to 16,500, 14,500 to 16,500, 15,000 to 16,500, 15,500 to 16,500, 16,000 to 16,500, 1,500 to 16,000, 2,000 to 16,000, 2,500 to 16,000, 3,000 to 16,000, 3,500 to 16,000, 4,000 to 16,000, 4,500 to 16,000, 5,000 to 16,000, 5,500 to 16,000, 6,000 to 16,000, 6,500 to 16,000, 7,000 to 16,000, 7,500 to 16,000, 8,000 to 16,000, 8,500 to 16,000, 9,000 to 16,000, 9,500 to 16,000, 10,000 to 16,000, 10,500 to 16,000, 11,000 to 16,000, 11,500 to 16,000, 12,000 to 16,000, 12,500 to 16,000, 13,000 to 16,000, 13,500 to 16,000, 14,000 to 16,000, 14,500 to 16,000, 15,000 to 16,000, 15,500 to 16,000, 1,500 to 15,500, 2,000 to 15,500, 2,500 to 15,500, 3,000 to 15,500, 3,500 to 15,500, 4,000 to 15,500, 4,500 to 15,500, 5,000 to 15,500, 5,500 to 15,500, 6,000 to 15,500, 6,500 to 15,500, 7,000 to 15,500, 7,500 to 15,500, 8,000 to 15,500, 8,500 to 15,500, 9,000 to 15,500, 9,500 to 15,500, 10,000 to 15,500, 10,500 to 15,500, 11,000 to 15,500, 11,500 to 15,500, 12,000 to 15,500, 12,500 to 15,500, 13,000 to 15,500, 13,500 to 15,500, 14,000 to 15,500, 14,500 to 15,500, 15,000 to 15,500, 1,500 to 15,000, 2,000 to 15,000, 2,500 to 15,000, 3,000 to 15,000, 3,500 to 15,000, 4,000 to 15,000, 4,500 to 15,000, 5,000 to 15,000, 5,500 to 15,000, 6,000 to 15,000, 6,500 to 15,000, 7,000 to 15,000, 7,500 to 15,000, 8,000 to 15,000, 8,500 to 15,000, 9,000 to 15,000, 9,500 to 15,000, 10,000 to 15,000, 10,500 to 15,000, 11,000 to 15,000, 11,500 to 15,000, 12,000 to 15,000, 12,500 to 15,000, 13,000 to 15,000, 13,500 to 15,000, 14,000 to 15,000, 14,500 to 15,000, 1,500 to 14,500, 2,000 to 14,500, 2,500 to 14,500, 3,000 to 14,500, 3,500 to 14,500, 4,000 to 14,500, 4,500 to 14,500, 5,000 to 14,500, 5,500 to 14,500, 6,000 to 14,500, 6,500 to 14,500, 7,000 to 14,500, 7,500 to 14,500, 8,000 to 14,500, 8,500 to 14,500, 9,000 to 14,500, 9,500 to 14,500, 10,000 to 14,500, 10,500 to 14,500, 11,000 to 14,500, 11,500 to 14,500, 12,000 to 14,500, 12,500 to 14,500, 13,000 to 14,500, 13,500 to 14,500, 14,000 to 14,500, 1,500 to 14,000, 2,000 to 14,000, 2,500 to 14,000, 3,000 to 14,000, 3,500 to 14,000, 4,000 to 14,000, 4,500 to 14,000, 5,000 to 14,000, 5,500 to 14,000, 6,000 to 14,000, 6,500 to 14,000, 7,000 to 14,000, 7,500 to 14,000, 8,000 to 14,000, 8,500 to 14,000, 9,000 to 14,000, 9,500 to 14,000, 10,000 to 14,000, 10,500 to 14,000, 11,000 to 14,000, 11,500 to 14,000, 12,000 to 14,000, 12,500 to 14,000, 13,000 to 14,000, 13,500 to 14,000, 1,500 to 13,500, 2,000 to 13,500, 2,500 to 13,500, 3,000 to 13,500, 3,500 to 13,500, 4,000 to 13,500, 4,500 to 13,500, 5,000 to 13,500, 5,500 to 13,500, 6,000 to 13,500, 6,500 to 13,500, 7,000 to 13,500, 7,500 to 13,500, 8,000 to 13,500, 8,500 to 13,500, 9,000 to 13,500, 9,500 to 13,500, 10,000 to 13,500, 10,500 to 13,500, 11,000 to 13,500, 11,500 to 13,500, 12,000 to 13,500, 12,500 to 13,500, 13,000 to 13,500, 1,500 to 13,000, 2,000 to 13,000, 2,500 to 13,000, 3,000 to 13,000, 3,500 to 13,000, 4,000 to 13,000, 4,500 to 13,000, 5,000 to 13,000, 5,500 to 13,000, 6,000 to 13,000, 6,500 to 13,000, 7,000 to 13,000, 7,500 to 13,000, 8,000 to 13,000, 8,500 to 13,000, 9,000 to 13,000, 9,500 to 13,000, 10,000 to 13,000, 10,500 to 13,000, 11,000 to 13,000, 11,500 to 13,000, 12,000 to 13,000, 12,500 to 13,000, 1,500 to 12,500, 2,000 to 12,500, 2,500 to 12,500, 3,000 to 12,500, 3,500 to 12,500, 4,000 to 12,500, 4,500 to 12,500, 5,000 to 12,500, 5,500 to 12,500, 6,000 to 12,500, 6,500 to 12,500, 7,000 to 12,500, 7,500 to 12,500, 8,000 to 12,500, 8,500 to 12,500, 9,000 to 12,500, 9,500 to 12,500, 10,000 to 12,500, 10,500 to 12,500, 11,000 to 12,500, 11,500 to 12,500, 12,000 to 12,500, 1,500 to 12,000, 2,000 to 12,000, 2,500 to 12,000, 3,000 to 12,000, 3,500 to 12,000, 4,000 to 12,000, 4,500 to 12,000, 5,000 to 12,000, 5,500 to 12,000, 6,000 to 12,000, 6,500 to 12,000, 7,000 to 12,000, 7,500 to 12,000, 8,000 to 12,000, 8,500 to 12,000, 9,000 to 12,000, 9,500 to 12,000, 10,000 to 12,000, 10,500 to 12,000, 11,000 to 12,000, 11,500 to 12,000, 1,500 to 11,500, 2,000 to 11,500, 2,500 to 11,500, 3,000 to 11,500, 3,500 to 11,500, 4,000 to 11,500, 4,500 to 11,500, 5,000 to 11,500, 5,500 to 11,500, 6,000 to 11,500, 6,500 to 11,500, 7,000 to 11,500, 7,500 to 11,500, 8,000 to 11,500, 8,500 to 11,500, 9,000 to 11,500, 9,500 to 11,500, 10,000 to 11,500, 10,500 to 11,500, 11,000 to 11,500, 1,500 to 11,000, 2,000 to 11,000, 2,500 to 11,000, 3,000 to 11,000, 3,500 to 11,000, 4,000 to 11,000, 4,500 to 11,000, 5,000 to 11,000, 5,500 to 11,000, 6,000 to 11,000, 6,500 to 11,000, 7,000 to 11,000, 7,500 to 11,000, 8,000 to 11,000, 8,500 to 11,000, 9,000 to 11,000, 9,500 to 11,000, 10,000 to 11,000, 10,500 to 11,000, 1,500 to 10,500, 2,000 to 10,500, 2,500 to 10,500, 3,000 to 10,500, 3,500 to 10,500, 4,000 to 10,500, 4,500 to 10,500, 5,000 to 10,500, 5,500 to 10,500, 6,000 to 10,500, 6,500 to 10,500, 7,000 to 10,500, 7,500 to 10,500, 8,000 to 10,500, 8,500 to 10,500, 9,000 to 10,500, 9,500 to 10,500, 10,000 to 10,500, 1,500 to 10,000, 2,000 to 10,000, 2,500 to 10,000, 3,000 to 10,000, 3,500 to 10,000, 4,000 to 10,000, 4,500 to 10,000, 5,000 to 10,000, 5,500 to 10,000, 6,000 to 10,000, 6,500 to 10,000, 7,000 to 10,000, 7,500 to 10,000, 8,000 to 10,000, 8,500 to 10,000, 9,000 to 10,000, 9,500 to 10,000, 1,500 to 9,500, 2,000 to 9,500, 2,500 to 9,500, 3,000 to 9,500, 3,500 to 9,500, 4,000 to 9,500, 4,500 to 9,500, 5,000 to 9,500, 5,500 to 9,500, 6,000 to 9,500, 6,500 to 9,500, 7,000 to 9,500, 7,500 to 9,500, 8,000 to 9,500, 8,500 to 9,500, 9,000 to 9,500, 1,500 to 9,000, 2,000 to 9,000, 2,500 to 9,000, 3,000 to 9,000, 3,500 to 9,000, 4,000 to 9,000, 4,500 to 9,000, 5,000 to 9,000, 5,500 to 9,000, 6,000 to 9,000, 6,500 to 9,000, 7,000 to 9,000, 7,500 to 9,000, 8,000 to 9,000, 8,500 to 9,000, 1,500 to 8,500, 2,000 to 8,500, 2,500 to 8,500, 3,000 to 8,500, 3,500 to 8,500, 4,000 to 8,500, 4,500 to 8,500, 5,000 to 8,500, 5,500 to 8,500, 6,000 to 8,500, 6,500 to 8,500, 7,000 to 8,500, 7,500 to 8,500, 8,000 to 8,500, 1,500 to 8,000, 2,000 to 8,000, 2,500 to 8,000, 3,000 to 8,000, 3,500 to 8,000, 4,000 to 8,000, 4,500 to 8,000, 5,000 to 8,000, 5,500 to 8,000, 6,000 to 8,000, 6,500 to 8,000, 7,000 to 8,000, 7,500 to 8,000, 1,500 to 7,500, 2,000 to 7,500, 2,500 to 7,500, 3,000 to 7,500, 3,500 to 7,500, 4,000 to 7,500, 4,500 to 7,500, 5,000 to 7,500, 5,500 to 7,500, 6,000 to 7,500, 6,500 to 7,500, 7,000 to 7,500, 1,500 to 7,000, 2,000 to 7,000, 2,500 to 7,000, 3,000 to 7,000, 3,500 to 7,000, 4,000 to 7,000, 4,500 to 7,000, 5,000 to 7,000, 5,500 to 7,000, 6,000 to 7,000, 6,500 to 7,000, 1,500 to 6,500, 2,000 to 6,500, 2,500 to 6,500, 3,000 to 6,500, 3,500 to 6,500, 4,000 to 6,500, 4,500 to 6,500, 5,000 to 6,500, 5,500 to 6,500, 6,000 to 6,500, 1,500 to 6,000, 2,000 to 6,000, 2,500 to 6,000, 3,000 to 6,000, 3,500 to 6,000, 4,000 to 6,000, 4,500 to 6,000, 5,000 to 6,000, 5,500 to 6,000, 1,500 to 5,500, 2,000 to 5,500, 2,500 to 5,500, 3,000 to 5,500, 3,500 to 5,500, 4,000 to 5,500, 4,500 to 5,500, 5,000 to 5,500, 1,500 to 5,000, 2,000 to 5,000, 2,500 to 5,000, 3,000 to 5,000, 3,500 to 5,000, 4,000 to 5,000, 4,500 to 5,000, 1,500 to 4,500, 2,000 to 4,500, 2,500 to 4,500, 3,000 to 4,500, 3,500 to 4,500, 4,000 to 4,500, 1,500 to 4,000, 2,000 to 4,000, 2,500 to 4,000, 3,000 to 4,000, 3,500 to 4,000, 1,500 to 3,500, 2,000 to 3,500, 2,500 to 3,500, 3,000 to 3,500, 1,500 to 3,000, 2,000 to 3,000, 2,500 to 3,000, 1,500 to 2,500, 2,000 to 2,500, and 1,500 to 2,000 daltons.

In some embodiments the polymer (e.g., PEG) is conjugated to at least one lipid. In some embodiments the lipid conjugated to the polymer comprised of at least one neutral lipid, at least one phospholipid, at least one anionic lipid, at least one cationic lipid, at least one cholesterol, at least one cholesterol derivative, or any combination thereof.

In some embodiments, the lipid conjugated to the polymer may be selected from, but is not limited to, at least one of the cationic, non-cationic, or sterol lipids listed previously.

In some embodiments, the at least one PEG-lipid conjugate may be selected from, but is not limited to at least one of Siglec-1L-PEG-DSPE, R)-2,3-bis(octadecyloxy)propyl-1-(methoxypoly(ethyleneglycol)2000)propylcarbamate, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-cDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG(2k)-DMG, PEG DSPE C18, PEG DMPE C14, PEG DLPE C12, PEG Click DMG C14, PEG Click C12, PEG Click C10, N(Carbonyl-methoxypolyethyleneglycol-2000)-1,2-distearoyl-sn-glycero3-phosphoethanolamine, Myrj52, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, 1,2-distearoyl-sn-glycero-3-phosphoethanolamine-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSPE-PEG, DSG-PEGMA, DSG-PEG5000, DPPE-PEG-2K, DPPE-PEG, DPPE-mPEG2000, DPPE-mPEG, DPG-PEGMA, DOPE-PEG2000, DMPE-PEGMA, DMPE-PEG2000, DMPE-Peg, DMPE-mPEG2000, DMG-PEGMA, DMG-PEG2000, DMG-PEG, distearoyl-glycerol-polyethyleneglycol, C18PEG750, CI8PEG5000, CI8PEG3000, CI8PEG2000, CI6PEG2000, CI4PEG2000, C18-PEG5000, C18PEG, C16PEG, C16 mPEG (polyethylene glycol) 2000 Ceramide, C14-PEG-DSPE200, C14-PEG2000, C14PEG2000, C14-PEG 2000, C14-PEG, C14PEG, 14:0-PEG2KPE, 1,2-distearoyl-sn-glycero-3-phosphoethanolamine-PEG2000, (R)-2,3-bis(octadecyloxy)propyl-1-(methoxypoly(ethyleneglycol)2000)propylcarbamate, (PEG)-C-DOMG, PEG-C-DMA, and DSPE-PEG-X.

The amounts and ratios of LNP components may be varied by any amount dependent on the desired form, structure, function, cargo, target, or any combination thereof. The amount of each component may be expressed in various embodiments as percent of the total molar mass of all lipid or lipid conjugated components accounted for by the indicated component (mol %), The amount of each component may be expressed in various embodiments as the relative ratio of each component based on molar mass (Molar Ratio). The amount of each component may be expressed in various embodiments as the weight of each component used to formulate the LNP prior to fabrication (mg or equivalent). The amount of each component may be expressed in various embodiments by any other method known in the art. Any formulation given in one representation of component amounts (“units”) is expressly meant to encompass any formulation expressed in different units of component amounts, wherein those representations are effectively equivalent when converted into the same units. In some embodiments, “effectively equivalent” means two or more values within about 10% of one another.

In some embodiments, the LNP comprises at least one cationic lipid in an amount of about 0.1 to 100 mol %. In some embodiments, the LNP comprises at least one cationic lipid in an amount of about 20 to 60 mol %. In some embodiments, the LNP comprises at least one cationic lipid in an amount of about 50 to 85 mol %. In some embodiments, the LNP comprises at least one cationic lipid in an amount of less than about 20 mol %. In some embodiments, the LNP comprises at least one cationic lipid in an amount of more than about 60 mol % or about 85 mol %. In some embodiments, the LNP comprises at least one cationic lipid in an amount of about 95 mol % or less. In some embodiments, the LNP comprises a cationic lipid in an amount of less than or equal to about 95, 90, 85, 80, 75, 70, 65, 60, 55, 50, 45, 40, 35, 30, 25, 20, 15, 10, and 5 mol %. In some embodiments, the LNP comprises at least one cationic lipid in an amount of more than or equal to about 5, 10, 15, 20, 25, 30, 35, 40, 45, 50, 55, 60, 65, 70, 75, 80, 85, 90, and 95 mol %. In some embodiments, the LNP comprises at least one cationic lipid in an amount from about 20 to 30 mol %, 20 to 35 mol %, 20 to 40 mol %, 20 to 45 mol %, 20 to 50 mol %, 20 to 55 mol %, 20 to 60 mol %, 20 to 65 mol %, 20 to 70 mol %, 20 to 75 mol %, 20 to 80 mol %, 20 to 85 mol %, 20 to 90 mol %, 25 to 35 mol %, 25 to 40 mol %, 25 to 45 mol %, 25 to 50 mol %, 25 to 55 mol %, 25 to 60 mol %, 25 to 65 mol %, 25 to 70 mol %, 25 to 75 mol %, 25 to 80 mol %, 25 to 85 mol %, 25 to 90 mol %, 30 to 40 mol %, 30 to 45 mol %, 30 to 50 mol %, 30 to 55 mol %, 30 to 60 mol %, 30 to 65 mol %, 30 to 70 mol %, 30 to 75 mol %, 30 to 80 mol %, 30 to 85 mol %, 30 to 90 mol %, 35 to 40 mol %, 35 to 45 mol %, 35 to 50 mol %, 35 to 55 mol %, 35 to 60 mol %, 35 to 65 mol %, 35 to 70 mol %, 35 to 75 mol %, 35 to 80 mol %, 35 to 85 mol %, 35 to 90 mol %, 40 to 45 mol %, 40 to 50 mol %, 40 to 55 mol %, 40 to 60 mol %, 40 to 65 mol %, 40 to 70 mol %, 40 to 75 mol %, 40 to 80 mol %, 40 to 85 mol %, 40 to 90 mol %, 45 to 55 mol %, 45 to 60 mol %, 45 to 65 mol %, 45 to 70 mol %, 45 to 75 mol %, 45 to 80 mol %, 45 to 85 mol %, 45 to 90 mol %, 50 to 60 mol %, 50 to 65 mol %, 50 to 70 mol %, 50 to 75 mol %, 50 to 80 mol %, 50 to 85 mol %, 50 to 90 mol %, 55 to 65 mol %, 55 to 70 mol %, 55 to 75 mol %, 55 to 80 mol %, 55 to 85 mol %, 55 to 90 mol %, 60 to 70 mol %, 60 to 75 mol %, 60 to 80 mol %, 60 to 85 mol %, 60 to 90 mol %, 65 to 75 mol %, 65 to 80 mol %, 65 to 85 mol %, 65 to 90 mol %, 70 to 80 mol %, 70 to 85 mol %, 70 to 90 mol %, 75 to 85 mol %, 75 to 90 mol %, 80 to 90 mol % or 85 to 95 mol %.

In some embodiments, the LNP comprises at least one non-cationic lipid in an amount of about 0.1 to 100 mol %. In some embodiments, the LNP comprises at least one non-one cationic lipid in an amount of about 5 to 35 mol %. In some embodiments, the LNP comprises at least one cationic lipid in an amount of about 5 to 25 mol %. In some embodiments, the LNP comprises at least one non-cationic lipid in an amount of less than about 5 mol %. In some embodiments, the LNP comprises at least one non-cationic lipid in an amount of more than about 25 mol % or about 35 mol %. In some embodiments, the LNP comprises at least one non-cationic lipid in an amount of about 95 mol % or less. In some embodiments, the LNP comprises at least one non-cationic lipid in an amount of less than or equal to about 95, 90, 85, 80, 75, 70, 65, 60, 55, 50, 45, 40, 35, 30, 25, 20, 15, 10, and 5 mol %. In some embodiments, the LNP comprises at least one non-cationic lipid in an amount of more than or equal to about 5, 10, 15, 20, 25, 30, 35, 40, 45, 50, 55, 60, 65, 70, 75, 80, 85, 90, and 95 mol %. In some embodiments, the LNP comprises at least one non-cationic lipid in an amount from about 5 to 15 mol %, 5 to 25 mol %, 5 to 35 mol %, 5 to 45 mol %, 5 to 55 mol %, 10 to 20 mol %, 10 to 30 mol %, 10 to 40 mol %, 10 to 50 mol %, 15 to 25 mol %, 15 to 35 mol %, 15 to 45 mol %, 20 to 30 mol %, 20 to 40 mol %, 20 to 50 mol %, 25 to 35 mol %, 25 to 45 mol %, 30 to 40 mol %, 30 to 50 mol %, and 35 to 45 mol %.

In some embodiments, the LNP comprises at least one sterol in an amount of about 0.1 to 100 mol %. In some embodiments, the LNP comprises at least one sterol in an amount of about 20 to 45 mol %. In some embodiments, the LNP comprises at least one sterol in an amount of about 25 to 55 mol %. In some embodiments, the LNP comprises at least one sterol in an amount of less than about 20 mol %. In some embodiments, the LNP comprises at least one sterol in an amount of more than about 45 mol % or about 55 mol %. In some embodiments, the LNP comprises at least one sterol in an amount of about 95 mol % or less. In some embodiments, the LNP comprises at least one sterol in an amount of less than or equal to about 95, 90, 85, 80, 75, 70, 65, 60, 55, 50, 45, 40, 35, 30, 25, 20, 15, 10, and 5 mol %. In some embodiments, the LNP comprises at least one sterol in an amount of more than or equal to about 5, 10, 15, 20, 25, 30, 35, 40, 45, 50, 55, 60, 65, 70, 75, 80, 85, 90, and 95 mol %. In some embodiments, the LNP comprises at least one sterol in an amount from about 10 to 20 mol %, 10 to 30 mol %, 10 to 40 mol %, 10 to 50 mol %, 10 to 60 mol %, 15 to 25 mol %, 15 to 35 mol %, 15 to 45 mol %, 15 to 55 mol %, 15 to 65 mol %, 20 to 30 mol %, 20 to 40 mol %, 20 to 50 mol %, 20 to 60 mol %, 25 to 35 mol %, 25 to 45 mol %, 25 to 55 mol %, 25 to 65 mol %, 30 to 40 mol %, 30 to 50 mol %, 30 to 60 mol %, 35 to 45 mol %, 35 to 55 mol %, 35 to 65 mol %, 40 to 50 mol %, 40 to 60 mol %, 45 to 55 mol %, 45 to 65 mol %, 50 to 60 mol %, and 55 to 65 mol %.

In some embodiments, the LNP comprises at least one particle-activity-modifying-agent in an amount of about 0.1 to 100 mol %. In some embodiments, the LNP comprises at least one particle-activity-modifying-agent in an amount of about 0.5 to 15 mol %. In some embodiments, the LNP comprises at least one particle-activity-modifying-agent in an amount of about 15 to 40 mol %. In some embodiments, the LNP comprises at least one particle-activity-modifying-agent in an amount of less than about 0.1 mol %. In some embodiments, the LNP comprises at least one particle-activity-modifying-agent in an amount of more than about 15 mol % or about 40 mol %. In some embodiments, the LNP comprises at least one particle-activity-modifying-agent in an amount of about 95 mol % or less. In some embodiments, the LNP comprises at least one particle-activity-modifying-agent in an amount of less than or equal to about 95, 90, 85, 80, 75, 70, 65, 60, 55, 50, 45, 40, 35, 30, 25, 20, 15, 10, and 5 mol %. In some embodiments, the LNP comprises at least one particle-activity-modifying-agent in an amount of more than or equal to about 5, 10, 15, 20, 25, 30, 35, 40, 45, 50, 55, 60, 65, 70, 75, 80, 85, 90, and 95 mol %. In some embodiments, the LNP comprises at least one particle-activity-modifying-agent in an amount from about 0.1 to 1 mol %, 0.1 to 2 mol %, 0.1 to 3 mol %, 0.1 to 4 mol %, 0.1 to 5 mol %, 0.1 to 6 mol %, 0.1 to 7 mol %, 0.1 to 8 mol %, 0.1 to 9 mol %, 0.1 to 10 mol %, 0.1 to 15 mol %, 0.1 to 20 mol %, 0.1 to 25 mol %, 1 to 2 mol %, 1 to 3 mol %, 1 to 4 mol %, 1 to 5 mol %, 1 to 6 mol %, 1 to 7 mol %, 1 to 8 mol %, 1 to 9 mol %, 1 to 10 mol %, 1 to 15 mol %, 1 to 20 mol %, 1 to 25 mol %, 2 to 3 mol %, 2 to 4 mol %, 2 to 5 mol %, 2 to 6 mol %, 2 to 7 mol %, 2 to 8 mol %, 2 to 9 mol %, 2 to 10 mol %, 2 to 15 mol %, 2 to 25 mol %, 3 to 4 mol %, 3 to 5 mol %, 3 to 6 mol %, 3 to 7 mol %, 3 to 8 mol %, 3 to 9 mol %, 3 to 10 mol %, 3 to 15 mol %, 3 to 20 mol %, 3 to 25 mol %, 4 to 5 mol %, 4 to 6 mol %, 4 to 7 mol %, 4 to 8 mol %, 4 to 9 mol %, 4 to 10 mol %, 4 to 15 mol %, 4 to 20 mol %, 4 to 25 mol %, 5 to 10 mol %, 5 to 15 mol %, 5 to 20 mol %, 5 to 25 mol %, 10 to 15 mol %, 10 to 20 mol %, 10 to 25 mol %, 15 to 20 mol %, 15 to 25 mol %, and 20 to 25 mol %.

In some embodiments, the LNP is comprised of about 30-60 mol % of at least one cationic lipid, about 0-30 mol % of at least one non-cationic lipid (e.g., a phospholipid), about 18.5-48.5 mol % of at least one sterol (e.g., cholesterol), and about 0-10 mol % of at least one particle-activity-modifying-agent (e.g., a PEGylated lipid).

In some embodiments, the LNP is comprised of about 35-55 mol % of at least one cationic lipid, about 5-25 mol % of at least one non-cationic lipid (e.g., a phospholipid), about 30-40 mol % of at least one sterol (e.g., cholesterol), and about 0-10 mol % of at least one particle-activity-modifying-agent (e.g., a PEGylated lipid).

In some embodiments, the LNP is comprised of about 35-45 mol % of at least one cationic lipid, about 25-35 mol % of at least one non-cationic lipid (e.g., a phospholipid), about 20-30 mol % of at least one sterol (e.g., cholesterol), and about 0-10 mol % of at least one particle-activity-modifying-agent (e.g., a PEGylated lipid).

In some embodiments, the LNP is comprised of about 45-65 mol % of at least one cationic lipid, about 5-10 mol % of at least one non-cationic lipid (e.g., a phospholipid), about 25-40 mol % of at least one sterol (e.g., cholesterol), and about 0.5-10 mol % of at least one particle-activity-modifying-agent (e.g., a PEGylated lipid).

In some embodiments, the LNP is comprised of about 40-60 mol % of at least one cationic lipid, about 5-15 mol % of at least one non-cationic lipid (e.g., a phospholipid), about 35-45 mol % of at least one sterol (e.g., cholesterol), and about 0.5-3 mol % of at least one particle-activity-modifying-agent (e.g., a PEGylated lipid).

In some embodiments, the LNP is comprised of about 30-60 mol % of at least one cationic lipid, about 0-30 mol % of at least one non-cationic lipid (e.g., a phospholipid), about 15-50 mol % of at least one sterol (e.g., cholesterol), and about 0.01-10 mol % of at least one particle-activity-modifying-agent (e.g., a PEGylated lipid).

In some embodiments, the LNP is comprised of about 10-75 mol % of at least one cationic lipid, about 0.5-50 mol % of at least one non-cationic lipid (e.g., a phospholipid), about 5-60 mol % of at least one sterol (e.g., cholesterol), and about 0.1-20 mol % of at least one particle-activity-modifying-agent (e.g., a PEGylated lipid).

In some embodiments, the LNP is comprised of about 50-65 mol % of at least one cationic lipid, about 3-15 mol % of at least one non-cationic lipid (e.g., a phospholipid), about 30-40 mol % of at least one sterol (e.g., cholesterol), and about 0.5-2 mol % of at least one particle-activity-modifying-agent (e.g., a PEGylated lipid).

In some embodiments, the LNP is comprised of about 50-85 mol % of at least one cationic lipid, about 3-15 mol % of at least one non-cationic lipid (e.g., a phospholipid), about 30-40 mol % of at least one sterol (e.g., cholesterol), and about 0.5-2 mol % of at least one particle-activity-modifying-agent (e.g., a PEGylated lipid).

In some embodiments, the LNP is comprised of about 25-75 mol % of at least one cationic lipid, about 0.1-15 mol % of at least one non-cationic lipid (e.g., a phospholipid), about 5-50 mol % of at least one sterol (e.g., cholesterol), and about 0.5-20 mol % of at least one particle-activity-modifying-agent (e.g., a PEGylated lipid).

In some embodiments, the LNP is comprised of about 50-65 mol % of at least one cationic lipid, about 5-10 mol % of at least one non-cationic lipid (e.g., a phospholipid), about 25-35 mol % of at least one sterol (e.g., cholesterol), and about 5-10 mol % of at least one particle-activity-modifying-agent (e.g., a PEGylated lipid).

In some embodiments, the LNP is comprised of about 20-60 mol % of at least one cationic lipid, about 5-25 mol % of at least one non-cationic lipid (e.g., a phospholipid), about 25-55 mol % of at least one sterol (e.g., cholesterol), and about 0.5-15 mol % of at least one particle-activity-modifying-agent (e.g., a PEGylated lipid).

In some embodiments, the LNPs can be characterized by their shape. In some embodiments, the LNPs are essentially spherical. In some embodiments, the LNPs are essentially rod-shaped (i.e., cylindrical). In some embodiments, the LNPs are essentially disk shaped.

In some embodiments, the LNPs can be characterized by their size. In some embodiments, the size of an LNP can be defined as the diameter of its largest circular cross section, referred to herein simply as its diameter. In some embodiments the LNPs may have a diameter between 30 nm to about 150 nm. In some embodiments, the LNP may have diameters ranging between about 40 to 150 nm 50 to 150 nm, 60 to 150 nm, about 70 to 150 nm, or 80 to 150 nm, 90 to 150 nm, 100 to nm, 110 to 150 nm, 120 to 150 nm, 130 to 150 nm, 140 to 150 nm, 30 to 30 to 140 mol %, 40 to 140 mol %, 50 to 140 mol %, 60 to 140 mol %, 70 to 140 mol %, 80 to 140 mol %, 90 to 140 mol %, 100 to 140 mol %, 110 to 140 mol %, 120 to 140 mol %, 130 to 140 mol %, 140 to 140 mol %, 30 to 140 mol %, 40 to 130 mol %, 50 to 130 mol %, 60 to 130 mol %, 70 to 130 mol %, 80 to 130 mol %, 90 to 130 mol %, 100 to 130 mol %, 110 to 130 mol %, 120 to 130 mol %, 30 to 120 mol %, 40 to 120 mol %, 50 to 120 mol %, 60 to 120 mol %, 70 to 120 mol %, 80 to 120 mol %, 90 to 120 mol %, 100 to 120 mol %, 110 to 120 mol %, 30 to 110 mol %, 40 to 110 mol %, 50 to 110 mol %, 60 to 110 mol %, 70 to 110 mol %, 80 to 110 mol %, 90 to 110 mol %, 100 to 110 mol %, 30 to 100 mol %, 40 to 100 mol %, 50 to 100 mol %, 60 to 100 mol %, 70 to 100 mol %, 80 to 100 mol %, 90 to 100 mol %, 30 to 90 mol %, 40 to 90 mol %, 50 to 90 mol %, 60 to 90 mol %, 70 to 90 mol %, 80 to 90 mol %, 30 to 80 mol %, 40 to 80 mol %, 50 to 80 mol %, 60 to 80 mol %, 70 to 80 mol %, 30 to 70 mol %, 40 to 70 mol %, 50 to 70 mol %, 60 to 70 mol %, 30 to 60 mol %, 40 to 60 mol %, 50 to 60 mol %, 30 to 50 mol %, 40 to 50 mol %, and 30 to 40 mol %.

In some embodiments, a population of LNPs, such as those resulting from the same formulation, may be characterized by measuring the uniformity of size, shape, or mass of the particles in the population. uniformity may be expressed in some embodiments as the polydispersity index (PI) of the population. In some embodiments uniformity may be expressed in some embodiments as the disparity (D) of the population. The terms “polydispersity index” and “disparity” are understood herein to be equivalent and may be used interchangeably. In some embodiments, a population of LNPs resulting from a given formulation will have a PI of between about 0.1 and 1. In some embodiments, a population of LNPs resulting from a giving formulation will have a PI of less than about 1, less than about 0.5, less than about 0.4, less than about 0.3, less than about 0.2, less than about 0.1. In some embodiments, a population of LNPs resulting from a given formulation will have a PI of between about 0.1 to 1, 0.1 to 0.8, 0.1 to 0.6, 0.1 to 0.4, 0.1 to 0.2, 0.2 to 1, 0.2 to 0.8, 0.2 to 0.6, 0.2 to 0.4, 0.4 to 1, 0.4 to 0.8, 0.4 to 0.6, 0.6 to 1, 0.6 to 0.8, and 0.8 to 1.

In some embodiments, the LNP may fully or partially encapsulate a cargo. In some embodiments, essentially 0% of the cargo present in the final formulation is exposed to the environment outside of the LNP (i.e., the cargo is fully encapsulated. In some embodiments, the cargo is associated with the LNP but is at least partially exposed to the environment outside of the LNP. In some embodiments, the LNP may be characterized by the % of the cargo not exposed to the environment outside of the LNP, e.g., the encapsulation efficiency. For the sake of clarity, an encapsulation efficiency of about 100% refers to an LNP formulation where essentially all the cargo is fully encapsulated by the LNP, while an encapsulation rate of about 0% refers to an LNP where essential none of the cargo is encapsulated in the LNP, such as with an LNP where the cargo is bound to the external surface of the LNP. On some embodiments, an LNP may have an encapsulation efficiency of less than about 100%, less than about 95%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15% less than about 10%, or less than 5%. In some embodiments, an LNP may have an encapsulation efficiency of between about 90 to 100%, 80 to 100%, 70 to 100%, 60 to 100%, 50 to 100%, 40 to 100%, 30 to 100%, 20 to 100%, 10 to 100%, 80 to 90%, 70 to 90%, 60 to 90%, 50 to 90%, 40 to 90%, 30 to 90%, 20 to 90%, 10 to 90%, 70 to 80%, 60 to 80%, 50 to 80%, 40 to 80%, 30 to 80%, 20 to 80%, 10 to 80%, 60 to 70%, 50 to 70%, 40 to 70%, 30 to 70%, 20 to 70%, 10 to 70%, 40 to 50%, 30 to 50%, 20 to 50%, 10 to 50%, 30 to 40%, 20 to 40%, 10 to 40%, 20 to 30%, 10 to 30%, and 10 to 20%.

In some embodiments, a LNP may include at least one identifier moiety. Non-limiting examples of an identifier moiety include glycans, antibodies, peptides, small molecules, and any combination thereof. In some embodiments, the at least one targeting agent may be incorporated into the lipid membrane of the lipid-based nanoparticle. In some embodiments, the at least one targeting agent may be presented on the external surface of the nanoparticle. In some embodiments, the at least one targeting agent may be conjugated to a lipid-component of the nanoparticle. In some embodiments, the at least one targeting agent may be conjugated to a polymer component of the nanoparticle. In some embodiments, the at least one targeting agent may be anchored to the nanoparticle via hydrophobic ad hydrophilic interactions among the at least one targeting agent, the nanoparticle membrane, and the aqueous environments inside or outside the nanoparticle. In some embodiments, the at least one targeting agent is conjugated to a peptide/protein component of the nanoparticle membrane. In some embodiments, the at least one targeting agent is conjugated to a suitable linker moiety which is conjugated to a component of the nanoparticle membrane. In some embodiments, any combination of forces and bonds can result in the targeting agent being associated with the nanoparticle.

The LNPs described herein may be formed using techniques known in the art. As a non-limiting example, an organic solution containing the lipids is mixed together with an acidic aqueous solution containing the originator construct or benchmark construct in a microfluidic channel resulting in the formation of targeting system (delivery vehicle and the benchmark construct).

In some embodiments, each LNP formulation includes a benchmark construct having a uniquely identifiable nucleotide identifier sequence (e.g., barcode). The unique identifier sequence provides the ability to identify the specific LNP which produces the desired result. The LNP formulation may also differ in the LNP-forming composition used to generate the LNP. For example, the LNP-forming compositions can be varied in the molar amount and/or structure of the ionizable lipid, the molar amount and/or structure of the helper lipid, the molar amount/or structure of PEG, and/or the molar amount of cholesterol. Additionally, or alternatively, the LNP formulation may comprise benchmark constructs which differ in the coding sequence for the biologically active molecule. Additionally, or alternatively, the LNP formulation may comprise benchmark constructs which differ in the modifications made to the nucleic acid sequence.

In some embodiments, the lipid compositions described according to the respective molar ratios of the component lipids in the formulation. As a non-limiting example, the mol-% of the ionizable lipid may be from about 10 mol-% to about 80 mol-%. As a non-limiting example, the mol-% of the ionizable lipid may be from about 20 mol-% to about 70 mol-%. As a non-limiting example, the mol-% of the ionizable lipid may be from about 30 mol-% to about 60 mol-%. As a non-limiting example, the mol-% of the ionizable lipid may be from about 35 mol-% to about 55 mol-%. As a non-limiting example, the mol-% of the ionizable lipid may be from about 40 mol-% to about 50 mol-%. As a non-limiting example, the ionizable lipid mol-% of the transfer vehicle batch will be ±30%, ±25%, ±20%, ±15%, ±10%, ±5%, or ±2.5% of the target mol-%. In some embodiments, transfer vehicle variability between lots will be less than 15%, less than 10% or less than 5%.

In some embodiments, the mol-% of the helper lipid may be from about 1 mol-% to about 50 mol-%. In some embodiments, the mol-% of the helper lipid may be from about 2 mol-% to about 45 mol-%. In some embodiments, the mol-% of the helper lipid may be from about 3 mol-% to about 40 mol-%. In some embodiments, the mol-% of the helper lipid may be from about 4 mol-% to about 35 mol-%. In some embodiments, the mol-% of the helper lipid may be from about 5 mol-% to about 30 mol-%. In some embodiments, the mol-% of the helper lipid may be from about 10 mol-% to about 20 mol-%. In some embodiments, the helper lipid mol-% of the transfer vehicle batch will be ±30%, ±25%, ±20%, ±15%, ±10%, ±5%, or ±2.5% of the target mol-0.

In some embodiments, the mol-% of the structural lipid may be from about 10 mol-% to about 80 mol-%. In some embodiments, the mol-% of the structural lipid may be from about 20 mol-% to about 70 mol-%. In some embodiments, the mol-% of the structural lipid may be from about 30 mol-% to about 60 mol-%. In some embodiments, the mol-% of the structural lipid may be from about 35 mol-% to about 55 mol-%. In some embodiments, the mol-% of the structural lipid may be from about 40 mol-% to about 50 mol-%. In some embodiments, the structural lipid mol-% of the transfer vehicle batch will be ±30%, ±25%, ±20%, ±15%, ±10%, ±5%, or ±2.5% of the target mol-%.

In some embodiments, the mol-% of the PEG modified lipid may be from about 0.1 mol-% to about 10 mol-%. In some embodiments, the mol-% of the PEG modified lipid may be from about 0.2 mol-% to about 5 mol-%. In some embodiments, the mol-% of the PEG modified lipid may be from about 0.5 mol-% to about 3 mol-%. In some embodiments, the mol-% of the PEG modified lipid may be from about 1 mol-% to about 2 mol-%. In some embodiments, the mol-% of the PEG modified lipid may be about 1.5 mol-%. In some embodiments, the PEG modified lipid mol-% of the transfer vehicle batch will be ±30%, ±25%, ±20%, ±15%, ±10%, ±5%, or ±2.5% of the target mol-%.

In some embodiments, the delivery vehicle may be any of the lipid nanoparticles described in WO2021113777, the contents of which are herein incorporated by reference in their entirety.

In some embodiments, the delivery vehicle is a lipid nanoparticle which comprises any of the ionizable lipids (e.g., amine lipids), PEG lipids, non-cationic (helper) lipids, or structural lipids in WO2021113777, the contents of which are herein incorporated by reference in their entirety.

In some embodiments, a lipid nanoparticle formulation may be prepared by the methods described in International Publication Nos. WO2011127255 or WO2008103276, the contents of each of which is herein incorporated by reference in their entirety. In some embodiments, lipid nanoparticle formulations may be as described in International Publication No. WO2019131770, the contents of which is herein incorporated by reference in its entirety.

In some embodiments, a lipid nanoparticle formulation may be prepared by the methods described in International Publication No. WO2020237227, the contents of each of which is herein incorporated by reference in their entirety. In some embodiments, lipid nanoparticle formulations may be as described in International Publication No. WO2020237227, the contents of which is herein incorporated by reference in its entirety.

Described herein are polynucleotides (e.g., mRNAs), compositions, formulations, methods, and/or use of nucleic acid vaccines, specifically nucleic acid vaccines comprising polynucleotides encoding one or more antigen proteins, fragments or variants thereof of SARS-CoV-2 for the prevention, alleviation and/or treatment of COVID-19. The antigen protein may be a structural protein of SARS-CoV-2. The structural protein may be the spike(S) protein, the membrane(M) protein, the nucleocapsid(N) phosphoprotein or the envelope(E) protein.

In some embodiments, at least one component of the nucleic acid vaccine is a polynucleotide encoding at least one of the antigen proteins or the fragments or variants of the antigen proteins of SARS-CoV-2. The antigen protein may be a structural protein of SARS-CoV-2. The polynucleotide may be a RNA polynucleotide such as an mRNA polynucleotide.

In some embodiments, the nucleic acid vaccine includes at least one mRNA polynucleotide encoding at least one of the structural proteins or the fragments or variants of the structural proteins of SARS-CoV-2.

In some embodiments, the polynucleotide may be designed to encode one or more polypeptides of interest from SARS-CoV-2, or fragments or variants thereof. Such polypeptide of interest of SARS-CoV-2 may include, but is not limited to, whole polypeptides, a plurality of polypeptides or fragments of polypeptides or variants of polypeptides, which independently may be encoded by one or more regions or parts or the whole of a polynucleotide from SARS-CoV-2. As used herein, the term “polypeptides of interest” refer to any polypeptide which is selected to be encoded within, or whose function is affected by, the polynucleotides described herein. Any of the peptides or polypeptides described herein may be antigenic (also referred to as immunogenic).

As used herein, “polypeptide” means a polymer of amino acid residues (natural or unnatural) linked together most often by peptide bonds. The term, as used herein, refers to proteins, polypeptides, and peptides of any size, structure, or function, or origin. In some embodiments, the polypeptides of interest are antigens encoded by the polynucleotides as described herein.

In some embodiments, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides such as antibodies or insulin and may be associated or linked. Most commonly disulfide linkages are found in multichain polypeptides. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, or at least about 85%, more preferably at least about 90%, even more preferably at least about 95% identical (homologous) to a native or reference sequence.

In some embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gap and penalties introduced in the calculation.

By “homologs” as it applies to polypeptide sequences means the corresponding sequence of other species having substantial identity to a second sequence of a second species.

“Analogs”, as used herein, is meant to include polypeptide variants which differ by one or more amino acid alterations, e.g., substitutions, additions or deletions of amino acid residues that still maintain one or more of the properties of the parent or starting polypeptide.

In some embodiments, the present disclosure contemplates several types of compositions which are polypeptide based including variants and derivatives. These include substitutional, insertional, deletion and covalent variants and derivatives. The term “derivative” is used synonymously with the term “variant” but generally refers to a molecule that has been modified and/or changed in any way relative to a reference molecule or starting molecule.

For example, sequence tags or amino acids, such as one or more lysines, can be added to the peptide sequences described herein (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support.

“Substitutional variants” when referring to polypeptides are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of nonconservative substitutions include the substitution of a nonpolar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to polypeptides are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to polypeptides are those with one or more amino acids in the native or starting amino acid sequence removed. Ordinarily, deletional variants will have one or more amino acids deleted in a particular region of the molecule.

“Covalent derivatives” when referring to polypeptides include modifications of a native or starting protein with an organic proteinaceous or non-proteinaceous derivatizing agent, and/or post-translational modifications. Covalent modifications are traditionally introduced by reacting targeted amino acid residues of the protein with an organic derivatizing agent that is capable of reacting with selected side chains or terminal residues, or by harnessing mechanisms of post-translational modifications that function in selected recombinant hosT-cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

“Features” when referring to polypeptides are defined as distinct amino acid sequence-based components of a molecule. Features of the polypeptides encoded by the polynucleotides described herein include surface manifestations, local conformational shape, folds, loops, half-loops, domains, half-domains, sites, termini or any combination thereof.

As used herein when referring to polypeptides the term “surface manifestation” refers to a polypeptide-based component of a protein appearing on an outermost surface.

As used herein when referring to polypeptides the term “local conformational shape” means a polypeptide based structural manifestation of a protein which is located within a definable space of the protein.

As used herein when referring to polypeptides the term “fold” refers to the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to polypeptide conformation means a bend which alters the direction of the backbone of a peptide or polypeptide and may involve one, two, three or more amino acid residues.

As used herein when referring to polypeptides the term “loop” refers to a structural feature of a polypeptide which may serve to reverse the direction of the backbone of a peptide or polypeptide. Where the loop is found in a polypeptide and only alters the direction of the backbone, it may comprise four or more amino acid residues. Oliva et al. have identified at least 5 classes of protein loops (J. Mol Biol., 266 (4): 814-830; 1997). Loops may be open or closed. Closed loops or “cyclic” loops may comprise 2, 3, 4, 5, 6, 7, 8, 9, 10 or more amino acids between the bridging moieties. Such bridging moieties may comprise a cysteine-cysteine bridge (Cys-Cys) typical in polypeptides having disulfide bridges or alternatively bridging moieties may be non-protein based such as the dibromozylyl agents used herein.

As used herein when referring to polypeptides the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids).

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the term “half-domain” means a portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+1−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein, when referring to polypeptides the term “site” as it pertains to amino acid-based embodiments is used synonymously with “amino acid residue” and “amino acid side chain.” A site represents a position within a peptide or polypeptide that may be modified, manipulated, altered, derivatized or varied within the polypeptide-based molecules described herein.

As used herein the terms “termini” or “terminus” when referring to polypeptides refers to an extremity of a peptide or polypeptide. Such extremity is not limited only to the first or final site of the peptide or polypeptide but may include additional amino acids in the terminal regions. The polypeptide-based molecules described herein may be characterized as having both an N-terminus (terminated by an amino acid with a free amino group (NH2)) and a C-terminus (terminated by an amino acid with a free carboxyl group (COOH)). Proteins described herein are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide-based moiety such as an organic conjugate.

Once any of the features have been identified or defined as a desired component of a polypeptide to be encoded by a polynucleotide described herein, any of several manipulations and/or modifications of these features may be performed by moving, swapping, inverting, deleting, randomizing or duplicating. Furthermore, it is understood that manipulation of features may result in the same outcome as a modification to the molecules described herein. For example, a manipulation which involved deleting a domain would result in the alteration of the length of a molecule just as modification of a nucleic acid to encode less than a full-length molecule would.

In a polypeptide, the term “modification” refers to a modification as compared to the canonical set of 20 amino acids. The modifications may be various distinct modifications. In some embodiments, the regions may contain one, two, or more (optionally different) modifications.

Modifications and manipulations can be accomplished by methods known in the art such as, but not limited to, site directed mutagenesis or a priori incorporation during chemical synthesis. The resulting modified molecules may then be tested for activity using in vitro or in vivo assays such as those described herein or any other suitable screening assay known in the art.

In some embodiments, the polypeptides may comprise a consensus sequence which is discovered through rounds of experimentation. As used herein a “consensus” sequence is a single sequence which represents a collective population of sequences allowing for variability at one or more sites.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical to a reference protein. The protein fragment may contain 10, 20, 30, 40, 50, 60, 70, 80, 90, 100, or greater than 100 amino acids in length. In another example, any protein that includes a stretch of about 20, about 30, about 40, about 50, or about 100 amino acids, or more, which are about 40%, about 50%, about 60%, about 70%, about 80%, about 85%, about 90%, about 95%, or about 100% identical to any of the sequences described herein can be utilized in accordance with the nucleic acid vaccines described herein. In certain embodiments, a polypeptide to be utilized in accordance with the nucleic acid vaccines described herein includes 2, 3, 4, 5, 6, 7, 8, 9, 10, or more mutations as shown in any of the sequences provided or referenced herein.

As such, polynucleotides of the present disclosure encode peptides or polypeptides containing substitutions, insertions and/or additions, deletions and covalent modifications with respect to reference sequences, in particular the peptide or polypeptide sequences disclosed herein. The polynucleotides may also contain substitutions, insertions and/or additions, deletions and covalent modifications with respect to the polynucleotide reference sequences.

Reference molecules (polypeptides or polynucleotides) may share a certain identity with the designed molecules (polypeptides or polynucleotides). The term “identity” as known in the art, refers to a relationship between the sequences of two or more peptides, polypeptides or polynucleotides, as determined by comparing the sequences. In the art, identity also means the degree of sequence relatedness between them as determined by the number of matches between strings of two or more amino acid residues or nucleosides. Identity measures the percent of identical matches between the smaller of two or more sequences with gap alignments (if any) addressed by a particular mathematical model or computer program (e.g., “algorithms”). Identity of related peptides can be readily calculated by known methods. Such methods include, but are not limited to, those described in Computational Molecular Biology, Lesk, A. M., ed., Oxford University Press, N.Y., 1988; Biocomputing: Informatics and Genome Projects, Smith, D. W., ed., Academic Press, N.Y., 1993; Computer Analysis of Sequence Data, Part 1, Griffin, A. M., and Griffin, H. G., eds., Humana Press, N.J., 1994; Sequence Analysis in Molecular Biology, von Heinje, G., Academic Press, 1987; Sequence Analysis Primer, Gribskov, M. and Devereux, J., eds., M. Stockton Press, N.Y, 1991; and Carillo et al., SIAM J. Applied Math. 48: 1073; 1988).

In some embodiments, the encoded polypeptide variant may have the same or a similar activity as the reference polypeptide. Alternatively, the variant may have an altered activity (e.g., increased or decreased) relative to a reference polypeptide. Generally, variants of a particular polynucleotide or polypeptide described herein will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul et al., Gapped BLAST and PSLBLAST: a new generation of protein database search programs, Nucleic Acids Res. 1997, 25:3389-3402.) Other tools are described herein, specifically in the definition of “Identity.”

### IV. Cargo and Payloads

The present disclosure also provides compositions or constructs comprising the delivery vehicles of the present disclosure, wherein the delivery vehicles may comprise, encode or be conjugated to a cargo or payload to produce the constructs. In some embodiments, the cargo or payload is or encodes a biologically active molecule such as, but not limited to a therapeutic protein. As used herein, the term “biologically active” refers to a characteristic of any agent that has activity in a biological system, and particularly in an organism. For instance, an agent that, when administered to an organism, has a biological effect on that organism, is considered to be biologically active. In some embodiments, the cargo or payload is or encodes one or more prophylactically- or therapeutically-active proteins, polypeptides, or other factors. As a non-limiting example, the cargo or payload may be or encode an agent that enhances tumor killing activity such as, but not limited to, TRAIL or tumor necrosis factor (TNF), in a cancer. As another non-limiting example, the cargo or payload may be or encode an agent suitable for the treatment of conditions such as muscular dystrophy (e.g., cargo or payload is or encodes Dystrophin), cardiovascular disease (e.g., cargo or payload is or encodes SERCA2a, GATA4, Tbx5, Mef2C, Hand2, Myocd, etc.), neurodegenerative disease (e.g., cargo or payload is or encodes NGF, BDNF, GDNF, NT-3, etc.), chronic pain (e.g., cargo or payload is or encodes GlyRal), an enkephalin, or a glutamate decarboxylase (e.g., cargo or payload is or encodes GAD65, GAD67, or another isoform), lung disease (e.g., cargo or payload is or encodes CFTR), hemophilia (e.g., cargo or payload is or encodes Factor VIII or Factor IX), neoplasia (e.g., cargo or payload is or encodes PTEN, ATM, ATR, EGFR, ERBB2, ERBB3, ERBB4, Notch1, Notch2, Notch3, Notch4, AKT, AKT2, AKT3, HIF, HI Fla, HIF3a, Met, HRG, Bcl2, PPARalpha, PPAR gamma, WT1 (Wilms Tumor), FGF Receptor Family members (5 members: 1, 2, 3, 4, 5), CDKN2a, APC, RB (retinoblastoma), MEN1, VHL, BRCA1, BRCA2, AR (Androgen Receptor), TSG101, IGF, IGF Receptor, Igfl (4 variants), Igf2 (3 variants), Igfl Receptor, Igf2 Receptor, Bax, Bcl2, caspases family (9 members: 1, 2, 3, 4, 6, 7, 8, 9, 12), Kras, Ape), age-related macular degeneration (e.g., cargo or payload is or encodes Aber, Ccl2, Cc2, cp (ceruloplasmin), Timp3, cathepsin D, Vldlr), schizophrenia (e.g. Neuregulin (Nrgl), Erb4 (receptor for Neuregulin), Complexin-1 (Cp1x1), Tphl Tryptophan hydroxylase, Tph2 Tryptophan hydroxylase 2, Neurexin 1, GSK3, GSK3a, GSK3b, 5-HIT (Slc6a4), COMT, DRD (Drdla), SLC6A3, DAOA, DTNBPI, Dao (Daol)), trinucleotide repeat disorders (e.g., HTT (Huntington's Dx), SBMA/SMAXI/AR (Kennedy's Dx), FXN/X25 (Friedrich's Ataxia), ATX3 (Machado-Joseph's Dx), ATXNI and ATXN2 (spinocerebellar ataxias), DMPK (myotonic dystrophy), Atrophin-1 and Atnl(DRPLA Dx), CBP (Creb-BP-global instability), VLDLR (Alzheimer's), Atxn7, Atxn10), fragile X syndrome (e.g., cargo or payload is or encodes FMR2, FXRI, FXR2, mGLUR5), secretase related disorders (e.g., cargo or payload is or encodes APH-1 (alpha and beta), Presenilin (Psenl), nicastrin (Ncstn), PEN-2), ALS (e.g., cargo or payload is or encodes SOD1, ALS2, STEX, FUS, TARD BP, VEGF (VEGF-a, VEGF-b, VEGF-c)), autism (e.g., cargo or payload is or encodes Mecp2, BZRAP1, MDGA2, Sema5A, Neurexin 1), Alzheimer's disease (e.g., cargo or payload is or encodes E1, CHIP, UCH, UBB, Tau, LRP, PICALM, Clusterin, PS1, SORL1, CR1, Vldlr, Ubal, Uba3, CHIP28 (Aqpl, Aquaporin 1), Uchll, Uchl3, APP), inflammation (e.g., cargo or payload is or encodes IL-10, IL-1 (IL-Ia, IL-Ib), IL-13, IL-17 (IL-17a (CTLA8), IL-17b, IL-17c, IL-17d, IL-171), 11-23, Cx3crl, ptpn22, TNFa, NOD2/CARD15 for IBD, IL-6, IL-12 (IL-12a, IL-12b), CTLA4, Cx3cll), Parkinson's Disease (e.g., x-Synuclein, DJ-1, LRRK2, Parkin, PINK1), blood and coagulation disorders, such as, e.g., anemia, bare lymphocyte syndrome, bleeding disorders, hemophagocytic lymphohistiocytosis disorders, hemophilia A, hemophilia B, hemorrhagic disorders, leukocyte deficiencies and disorders, sickle cell anemia, and thalassemia (e.g., cargo or payload is or encodes CRAN1, CDA1, RPS19, DBA, PKLR, PK1, NT5C3, UMPH1, PSNI, RHAG, RH50A, NRAMP2, SPTB, ALAS2, ANH1, ASB, ABCB7, ABC7, ASAT, TAPBP, TPSN, TAP2, ABCB3, PSF2, RING11, MHC2TA, C2TA, RFX5, RFXAP, RFX5, TBXA2R, P2RX1, P2X1, HF1, CFH, HUS, MCFD2, FANCA, FAC A, FA1, FA, FA A, FAAP95, FAAP90, FLJ34064, FANCB, FANCC, FACC, BRCA2, FANCDI, FANCD2, FANCD, FACD, FAD, FANCE, FACE, FANCF, XRCC9, FANCG, BR1PI, BACH1, FANCJ, PHF9, FANCL, FANCM, KIAA1596, PRF1, HPLH2, UNC13D, MUNC13-4, HPLH3, HLH3, FHL3, F8, FSC, PI, ATT, F5, ITGB2, CD18, LCAMB, LAD, EIF2B1, EIF2BA, EIF2B2, EIF2B3, EIF2B5, LVWM, CACH, CLE, EIF2B4, HBB, HBA2, HBB, HBD, LCRB, HBA1), B-cell non-Hodgkin lymphoma or leukemia (e.g., cargo or payload is or encodes BCL7A, BCL7, ALI, TCL5, SCL, TAL2, FLT3, NBS1, NBS, ZNFN1AI, 1KI, LYF1, HOXD4, HOX4B, BCR, CML, PHL, ALL, ARNT, KRAS2, RASK2, GMPS, AFIO, ARHGEF12, LARG, KIAA0382, CALM, CLTH, CEBPA, CEBP, CHIC2, BTL, FLT3, KIT, PBT, LPP, NPMI, NUP214, D9S46E, CAN, CAIN, RUNXI, CBFA2, AML1, WHSC1LI, NSD3, FLT3, AF1Q, NPMI, NUMA1, ZNF145, PLZF, PML, MYL, STAT5B, AF1Q, CALM, CLTH, ARL11, ARLTS1, P2RX7, P2X7, BCR, CML, PHL, ALL, GRAF, NF1, VRNF, WSS, NFNS, PTPNII, PTP2C, SHP2, NS1, BCL2, CCND1, PRAD1, BCL1, TCRA, GATA1, GF1, ERYF1, NFE1, ABLI, NQO1, DIA4, NMOR1, NUP214, D9S46E, CAN, CAIN), inflammation and immune related diseases and disorders (e.g., cargo or payload is or encodes KIR3DL1, NKAT3, NKB1, AMB11, K1R3DS1, IFNG, CXCL12, TNFRSF6, APT1, FAS, CD95, ALPS1A, IL2RG, SCIDX1, SCIDX, IMD4, CCL5, SCYA5, D17S136E, TCP228, IL10, CSIF, CMKBR2, CCR2, CMKBR5, CCCKR5 (CCR5), CD3E, CD3G, AICDA, AID, HIGM2, TNFRSF5, CD40, UNG, DGU, HIGM4, TNFSFS, CD40LG, HIGM1, IGM, FOXP3, IPEX, AIID, XPID, PIDX, TNFRSF14B, TACI), inflammation (e.g., cargo or payload is or encodes IL-10, IL-1 (IL-IA, IL-IB), IL-13, IL-17 (IL-17a (CTLA8), IL-17b, IL-17c, IL-17d, IL-171), 11-23, Cx3crl, ptpn22, TNFa, NOD2/CARD15 for IBD, IL-6, IL-12 (IL-12a, IL-12b), CTLA4, Cx3cII), JAK3, JAKL, DCLREIC, ARTEMIS, SCIDA, RAG1, RAG2, ADA, PTPRC, CD45, LCA, IL7R, CD3D, T3D, IL2RG, SCIDXI, SCIDX, IMD4), metabolic, liver, kidney and protein diseases and disorders (e.g., cargo or payload is or encodes TTR, PALB, APOA1, APP, AAA, CVAP, ADI, GSN, FGA, LYZ, TTR, PALB, KRT18, KRT8, CIRH1A, NAIC, TEX292, KIAA1988, CFTR, ABCC7, CF, MRP7, SLC2A2, GLUT2, G6PC, G6PT, G6PT1, GAA, LAMP2, LAMPB, AGL, GDE, GBE1, GYS2, PYGL, PFKM, TCF1, HNF1A, MODY3, SCOD1, SCOl, CTNNB1, PDGFRL, PDGRL, PRLTS, AX1NI, AXIN, CTNNB1, TP53, P53, LFS1, IGF2R, MPRI, MET, CASP8, MCH5, UMOD, HNFJ, FJHN, MCKD2, ADMCKD2, PAH, PKU1, QDPR, DHPR, PTS, FCYT, PKHD1, ARPKD, PKD1, PKD2, PKD4, PKDTS, PRKCSH, G19P1, PCLD, SEC63), muscular/skeletal diseases and disorders (e.g., cargo or payload is or encodes DMD, BMD, MYF6, LMNA, LMN1, EMD2, FPLD, CMDIA, HGPS, LGMDIB, LMNA, LMNI, EMD2, FPLD, CMDIA, FSHMD1A, FSHD1A, FKRP, MDC1C, LGMD2I, LAMA2, LAMM, LARGE, KIAA0609, MDC1D, FCMD, TTID, MYOT, CAPN3, CANP3, DYSF, LGMD2B, SGCG, LGMD2C, DMDA1, SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDCIC, LGMD21, TTN, CMD1G, TMD, LGMD2J, POMT1, CAV3, LGMD1C, SEPN1, SELN, RSMD1, PLEC1, PLTN, EBS1, LRP5, BMND1, LRP7, LR3, OPPG, VBCH2, CLCN7, CLC7, OPTA2, OSTMI, GL, TCIRG1, TIRC7, OC116, OPTB1, VAPB, VAPC, ALS8, SMN1, SMA1, SMA2, SMA3, SMA4, BSCL2, SPG17, GARS, SMAD1, CMT2D, HEXB, IGHMBP2, SMUBP2, CATF1, SMARD1), neurological and neuronal diseases and disorders (e.g., cargo or payload is or encodes SOD1, ALS2, STEX, FUS, TARDBP, VEGF (VEGF-a, VEGF-b, VEGF-c), APP, AAA, CVAP, ADI, APOE, AD2, PSEN2, AD4, STM2, APBB2, FE65LI, NOS3, PLAU, URK, ACE, DCPI, ACEI, MPO, PAC1PI, PAXIPIL, PTIP, A2M, BLMH, BMH, PSEN1, AD3, Mecp2, BZRAP1, MDGA2, Sema5A, Neurexin 1, GLOl, MECP2, RTT, PPMX, MRX16, MRX79, NLGN3, NLGN4, KIAA1260, AUTSX2, FMR2, FXR1, FXR2, mGLUR5, HD, IT15, PRNP, PRIP, JPH3, JP3, HDL2, TBP, SCA17, NR4A2, NURR1, NOT, TINUR, SNCAIP, TBP, SCA17, SNCA, NACP, PARK1, PARK4, DJI, PARK7, LRRK2, PARK8, PINK1, PARK6, UCHL1, PARK5, SNCA, NACP, PARK1, PARK4, PRKN, PARK2, PDJ, DBH, NDUFV2, MECP2, RTT, PPMX, MRX16, MRX79, CDKL5, STK9, MECP2, RTT, PPMX, MRX16, MRX79, x-Synuclein, DJ-1, Neuregulin-1 (Nrgl), Erb4 (receptor for Neuregulin), Complexin-1 (Cplx1), Tphl Tryptophan hydroxylase, Tph2, Tryptophan hydroxylase 2, Neurexin 1, GSK3, GSK3a, GSK3b, 5-HTT (Slc6a4), CONT, DRD (Drdla), SLC6A, DAOA, DTNBP1, Dao (Daol), APH-l(alpha and beta), Presenilin (Psenl), Nicastrin, (Ncstn), PEN-2, Nosl, Parpl, Natl, Nat2, HTT, SBMA/SMAX1/AR, FXN/X25, ATX3, TXN, ATXN2, DMPK, Atrophin-1, Atnl, CBP, VLDLR, Atxn7, and AtxnlO), and ocular diseases and disorders (e.g., Aber, Ccl2, Cc2, cp (ceruloplasmin), Timp3, cathepsin-D, Vldlr, Ccr2, CRYAA, CRYA1, CRYBB2, CRYB2, PITX3, BFSP2, CP49, CP47, CRYAA, CRYAI, PAX6, AN2, MGDA, CRYBA1, CRYB1, CRYGC, CRYG3, CCL, LIM2, MP19, CRYGD, CRYG4, BFSP2, CP49, CP47, HSF4, CTM, HSF4, CTM, MIP, AQPO, CRYAB, CRYA2, CTPP2, CRYBB1, CRYGD, CRYG4, CRYBB2, CRYB2, CRYGC, CRYG3, CCL, CRYAA, CRYAI, GJA8, CX50, CAE1, GJA3, CX46, CZP3, CAE3, CCM1, CAM, KRIT1, APOA1, TGFBI, CSD2, CDGG1, CSD, BIGH3, CDG2, TACSTD2, TROP2, M1SI, VSX1, RINX, PPCD, PPD, KTCN, COL8A2, FECD, PPCD2, PIP5K3, CFD, KERA, CNA2, MYOC, TIGR, GLCIA, JO AG, GPOA, OPTN, GLC1E, FIP2, HYPL, NRP, CYP1BI, GLC3A, OPA1, NTG, NPG, CYP1BI, GLC3A, CRB1, RP12, CRX, CORD2, CRD, RPGRIPI, LCA6, CORD9, RPE65, RP20, AIPL1, LCA4, GUCY2D, GUC2D, LCA1, CORD6, RDH12, LCA3, ELOVL4, ADMD, STGD2, STGD3, RDS, RP7, PRPH2, PRPH, AVMD, AOFMD, and VMD2).

In some embodiments, the cargo or payload is or encodes a factor that can affect the differentiation of a cell. As a non-limiting example, the expression of one or more of Oct4, Klf4, Sox2, c-Myc, L-Myc, dominant-negative p53, Nanog, Glisl, Lin28, TFIID, mir-302/367, or other miRNAs can cause the cell to become an induced pluripotent stem (iPS) cell.

In some embodiments, the cargo or payload is or encodes a factor for transdifferentiating cells. Non-limiting examples of factors include: one or more of GATA4, Tbx5, Mef2C, Myocd, Hand2, SRF, Mespl, SMARCD3 for cardiomyocytes; Ascii, Nurrl, LmxlA, Bm2, Mytll, NeuroDl, FoxA2 for neural cells; and Hnf4a, Foxa1, Foxa2 or Foxa3 for hepatic cells.

### Polypeptides, Proteins and Peptides

The delivery vehicles of the present disclosure may comprise, encode or be conjugated to a cargo or payload which is a polypeptide, protein or peptide. As used herein, the term “polypeptide” generally refers to polymers of amino acids linked by peptide bonds and embraces “protein and “peptides.” Polypeptides for the present disclosure include all polypeptides, proteins and/or peptides known in the art. Non-limiting categories of polypeptides include antigens, antibodies, antibody fragments, cytokines, peptides, hormones, enzymes, oxidants, antioxidants, synthetic polypeptides, and chimeric polypeptides.

As used herein, the term “peptide” generally refers to shorter polypeptides of about 50 amino acids or less. Peptides with only two amino acids may be referred to as “dipeptides.” Peptides with only three amino acids may be referred to as “tripeptides.” Polypeptides generally refer to polypeptides with from about 4 to about 50 amino acids. Peptides may be obtained via any method known to those skilled in the art. In some embodiments, peptides may be expressed in culture. In some embodiments, peptides may be obtained via chemical synthesis (e.g. solid phase peptide synthesis).

In some embodiments, the delivery vehicles of the present disclosure may comprise, encode or be conjugated to a cargo or payload which is a simple protein which upon hydrolysis yields the amino acids and occasionally small carbohydrate compounds. Non-limiting examples of simple proteins include albumins, albuminoids, globulins, glutelins, histones and protamines.

In some embodiments, the delivery vehicles of the present disclosure may comprise, encode or be conjugated to a cargo or payload which is a conjugated protein which may be a simple protein associated with a non-protein. Non-limiting examples of conjugated proteins include, glycoproteins, hemoglobins, lecithoproteins, nucleoproteins, and phosphoproteins.

In some embodiments, the delivery vehicles of the present disclosure may comprise, encode or be conjugated to a cargo or payload which is a derived protein which is a protein that is derived from a simple or conjugated protein by chemical or physical means. Non-limiting examples of derived proteins include denatured proteins and peptides.

In some embodiments, the polypeptide, protein or peptide may be unmodified.

In some embodiments, the polypeptide, protein or peptide may be modified. Types of modifications include, but are not limited to, Phosphorylation, Glycosylation, Acetylation, Ubiquitylation/Sumoylation, Methylation, Palmitoylation, Quinone, Amidation, Myristoylation, Pyrrolidone carboxylic acid, Hydroxylation, Phosphopantetheine, Prenylation, GPI anchoring, Oxidation, ADP-ribosylation, Sulfation, S-nitrosylation, Citrullination, Nitration, Gamma-carboxyglutamic acid, Formylation, Hypusine, Topaquinone (TPQ), Bromination, Lysine topaquinone (LTQ), Tryptophan tryptophylquinone (TTQ), Iodination, and Cysteine tryptophylquinone (CTQ). In some aspects, the polypeptide, protein or peptide may be modified by a post-transcriptional modification which can affect its structure, subcellular localization, and/or function.

In some embodiments, the polypeptide, protein or peptide may be modified using phosphorylation. Phosphorylation, or the addition of a phosphate group to serine, threonine, or tyrosine residues, is one of most common forms of protein modification. Protein phosphorylation plays an important role in fine tuning the signal in the intracellular signaling cascades.

In some embodiments, the polypeptide, protein or peptide may be modified using ubiquitination which is the covalent attachment of ubiquitin to target proteins. Ubiquitination-mediated protein turnover has been shown to play a role in driving the cell cycle as well as in protein-degradation-independent intracellular signaling pathways.

In some embodiments, the polypeptide, protein or peptide may be modified using acetylation and methylation which can play a role in regulating gene expression. As a non-limiting example, the acetylation and methylation could mediate the formation of chromatin domains (e.g., euchromatin and heterochromatin) which could have an impact on mediating gene silencing.

In some embodiments, the polypeptide, protein or peptide may be modified using glycosylation. Glycosylation is the attachment of one of a large number of glycan groups and is a modification that occurs in about half of all proteins and plays a role in biological processes including, but not limited to, embryonic development, cell division, and regulation of protein structure. The two main types of protein glycosylation are N-glycosylation and O-glycosylation. For N-glycosylation the glycan is attached to an asparagine and for O-glycosylation the glycan is attached to a serine or threonine.

In some embodiments, the polypeptide, protein or peptide may be modified using Sumoylation. Sumoylation is the addition of SUMOs (small ubiquitin-like modifiers) to proteins and is a post-translational modification similar to ubiquitination.

### Antibodies

As used herein, the term “antibody” is referred to in the broadest sense and specifically covers various embodiments including, but not limited to monoclonal antibodies, polyclonal antibodies, multispecific antibodies (e.g. bispecific antibodies formed from at least two intact antibodies), and antibody fragments (e.g., diabodies) so long as they exhibit a desired biological activity (e.g., “functional”). Antibodies are primarily amino acid based molecules which are monomeric or multimeric polypeptides which comprise at least one amino acid region derived from a known or parental antibody sequence and at least one amino acid region derived from a non-antibody sequence. The antibodies may comprise one or more modifications (including, but not limited to the addition of sugar moieties, fluorescent moieties, chemical tags, etc.). For the purposes herein, an “antibody” may comprise a heavy and light variable domain as well as an Fc region.

The cargo or payload may comprise or may encode polypeptides that form one or more functional antibodies.

In some embodiments, the cargo or payload may comprise or may encode polypeptides that form or function as any antibody including, but not limited to, antibodies that are known in the art and/or antibodies that are commercially available which may be therapeutic, diagnostic, or for research purposes. Additionally, the cargo or payload may comprise or may encode fragments of such antibodies or antibodies such as, but not limited to, variable domains or complementarity determining regions (CDRs).

As used herein, the term “native antibody” refers to an usually heterotetrameric glycoprotein of about 150,000 Daltons, composed of two identical light (L) chains and two identical heavy (H) chains. Genes encoding antibody heavy and light chains are known and segments making up each have been well characterized and described (Matsuda, F. et al., 1998. The Journal of Experimental Medicine. 188(11); 2151-62 and Li, A. et al., 2004. Blood. 103(12: 4602-9, the content of each of which are herein incorporated by reference in their entirety). Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. As used herein, the term “light chain” refers to a component of an antibody from any vertebrate species assigned to one of two clearly distinct types, called kappa and lambda based on amino acid sequences of constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2.

As used herein, the term “variable domain” refers to specific antibody domains found on both the antibody heavy and light chains that differ extensively in sequence among antibodies and are used in the binding and specificity of each particular antibody for its particular antigen. Variable domains comprise hypervariable regions. As used herein, the term “hypervariable region” refers to a region within a variable domain comprising amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody comprising a structure that is complimentary to its target antigen or epitope. Other portions of the variable domain, not interacting with the antigen, are referred to as framework (FW) regions. The antigen-binding site (also known as the antigen combining site or paratope) comprises the amino acid residues necessary to interact with a particular antigen. The exact residues making up the antigen-binding site are typically elucidated by co-crystallography with bound antigen, however computational assessments can also be used based on comparisons with other antibodies (Strohl, W. R. Therapeutic Antibody Engineering. Woodhead Publishing, Philadelphia PA. 2012. Ch. 3, p 47-54, the contents of which are herein incorporated by reference in their entirety). Determining residues making up CDRs may include the use of numbering schemes including, but not limited to, those taught by Kabat [Wu, T. T. et al., 1970, JEM, 132(2):211-50 and Johnson, G. et al., 2000, Nucleic Acids Res. 28(1): 214-8, the contents of each of which are herein incorporated by reference in their entirety], Chothia [Chothia and Lesk, J. Mol. Biol. 196, 901 (1987), Chothia et al., Nature 342, 877 (1989) and Al-Lazikani, B. et al., 1997, J. Mol. Biol. 273(4):927-48, the contents of each of which are herein incorporated by reference in their entirety], Lefranc (Lefranc, M. P. et al., 2005, Immunome Res. 1:3) and Honegger (Honegger, A. and Pluckthun, A. 2001. J. Mol. Biol. 309(3):657-70, the contents of which are herein incorporated by reference in their entirety).

V_{H} and V_{L} domains each have three CDRs. V_{L} CDRs are referred to herein as CDR-L1, CDR-L2 and CDR-L3, in order of occurrence when moving from N- to C-terminus along the variable domain polypeptide. V_{H} CDRs are referred to herein as CDR-H1, CDR-H2, and CDR-H3, in order of occurrence when moving from N- to C-terminus along the variable domain polypeptide. Each of CDRs have favored canonical structures with the exception of the CDR-H3, which comprises amino acid sequences that may be highly variable in sequence and length between antibodies resulting in a variety of three-dimensional structures in antigen-binding domains. In some cases, CDR-H3s may be analyzed among a panel of related antibodies to assess antibody diversity.

Various methods of determining CDR sequences are known in the art and may be applied to known antibody sequences. The system described by Kabat, also referred to as “numbered according to Kabat,” “Kabat numbering,” “Kabat definitions,” and “Kabat labeling,” provides an unambiguous residue numbering system applicable to any variable domain of an antibody, and provides precise residue boundaries defining the three CDRs of each chain. (Kabat et al., Sequences of Proteins of Immunological Interest, National Institutes of Health, Bethesda, Md. (1987) and (1991), the contents of which are incorporated by reference in their entirety). Kabat CDRs and comprise about residues 24-34 (CDR1), 50-56 (CDR2) and 89-97 (CDR3) in the light chain variable domain, and 31-35 (CDR1), 50-65 (CDR2) and 95-102 (CDR3) in the heavy chain variable domain. Chothia and coworkers found that certain sub-portions within Kabat CDRs adopt nearly identical peptide backbone conformations, despite having great diversity at the level of amino acid sequence. (Chothia et al. (1987) J. Mol. Biol. 196: 901-917; and Chothia et al. (1989) Nature 342: 877-883, the contents of each of which is herein incorporated by reference in its entirety). These CDRs can be referred to as “Chothia CDRs,” “Chothia numbering,” or “numbered according to Chothia,” and comprise about residues 24-34 (CDR1), 50-56 (CDR2) and 89-97 (CDR3) in the light chain variable domain, and 26-32 (CDR1), 52-56 (CDR2) and 95-102 (CDR3) in the heavy chain variable domain. Mol. Biol. 196:901-917 (1987). The system described by MacCallum, also referred to as “numbered according to MacCallum,” or “MacCallum numbering” comprises about residues 30-36 (CDR1), 46-55 (CDR2) and 89-96 (CDR3) in the light chain variable domain, and 30-35 (CDR1), 47-58 (CDR2) and 93-101 (CDR3) in the heavy chain variable domain. (MacCallum et al. ((1996) J. Mol. Biol. 262(5):732-745), the contents of which is herein incorporated by reference in its entirety). The system described by AbM, also referred to as “numbering according to AbM,” or “AbM numbering” comprises about residues 24-34 (CDR1), 50-56 (CDR2) and 89-97 (CDR3) in the light chain variable domain, and 26-35 (CDR1), 50-58 (CDR2) and 95-102 (CDR3) in the heavy chain variable domain. The IMGT (INTERNATIONAL IMMUNOGENETICS INFORMATION SYSTEM) numbering of variable regions can also be used, which is the numbering of the residues in an immunoglobulin variable heavy or light chain according to the methods of the IIMGT (Lefranc, M. P., “The IMGT unique numbering for immunoglobulins, T cell Receptors and Ig-like domains”, The Immunologist, 7, 132-136 (1999), and is herein incorporated by reference in its entirety by reference). As used herein, “IMGT sequence numbering” or “numbered according to IMTG,” refers to numbering of the sequence encoding a variable region according to the IMGT. For the heavy chain variable domain, when numbered according to IMGT, the hypervariable region ranges from amino acid positions 27 to 38 for CDR1, amino acid positions 56 to 65 for CDR2, and amino acid positions 105 to 117 for CDR3. For the light chain variable domain, when numbered according to IMGT, the hypervariable region ranges from amino acid positions 27 to 38 for CDR1, amino acid positions 56 to 65 for CDR2, and amino acid positions 105 to 117 for CDR3.

In some embodiments, the cargo or payload may comprise or may encode antibodies which have been produced using methods known in the art such as, but are not limited to immunization and display technologies (e.g., phage display, yeast display, and ribosomal display), hybridoma technology, heavy and light chain variable region cDNA sequences selected from hybridomas or from other sources,

In some embodiments, the cargo or payload may comprise or may encode antibodies which were developed using any naturally occurring or synthetic antigen. As used herein, an “antigen” is an entity which induces or evokes an immune response in an organism. An immune response is characterized by the reaction of the cells, tissues and/or organs of an organism to the presence of a foreign entity. Such an immune response typically leads to the production by the organism of one or more antibodies against the foreign entity, e.g., antigen or a portion of the antigen. As used herein, “antigens” also refer to binding partners for specific antibodies or binding agents in a display library.

As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen

The modifier “monoclonal” indicates the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

As used herein, the term “humanized antibody” refers to a chimeric antibody comprising a minimal portion from one or more non-human (e.g., murine) antibody source(s) with the remainder derived from one or more human immunoglobulin sources. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and/or capacity.

In some embodiments, the cargo or payload may comprise or may encode antibody mimetics. As used herein, the term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold. In some embodiments, antibody mimetics may be those known in the art including, but are not limited to affibody molecules, affilins, affitins, anticalins, avimers, Centyrins, DARPINS™, fynomers, Kunitz domains, and domain peptides. In other embodiments, antibody mimetics may include one or more non-peptide regions.

### Antibody Fragments and Variants

In some embodiments, the cargo or payload may comprise or may encode antibody fragments which comprise antigen binding regions from full-length antibodies. Non-limiting examples of antibody fragments include Fab, Fab′, F(ab′)₂, and Fv fragments, diabodies, linear antibodies, single-chain antibody molecules, and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)₂ fragment that has two antigen-binding sites and is still capable of cross-linking antigen. Compounds and/or compositions of the present invention may comprise one or more of these fragments.

In some embodiments, the Fc region may be a modified Fc region wherein the Fc region may have a single amino acid substitution as compared to the corresponding sequence for the wild-type Fc region, wherein the single amino acid substitution yields an Fc region with preferred properties to those of the wild-type Fc region. Non-limiting examples of Fc properties that may be altered by the single amino acid substitution include bind properties or response to pH conditions

As used herein, the term “Fv” refers to an antibody fragment comprising the minimum fragment on an antibody needed to form a complete antigen binding site. These regions consist of a dimer of one heavy chain and one light chain variable domain in tight, non-covalent association. Fv fragments can be generated by proteolytic cleavage, but are largely unstable. Recombinant methods are known in the art for generating stable Fv fragments, typically through insertion of a flexible linker between the light chain variable domain and the heavy chain variable domain to form a single chain Fv (scFv) or through the introduction of a disulfide bridge between heavy and light chain variable domains.

As used herein, the term “single chain Fv” or “scFv” refers to a fusion protein of V_{H} and V_{L} antibody domains, wherein these domains are linked together into a single polypeptide chain by a flexible peptide linker. In some embodiments, the Fv polypeptide linker enables the scFv to form the desired structure for antigen binding. In some embodiments, scFvs are utilized in conjunction with phage display, yeast display or other display methods where they may be expressed in association with a surface member (e.g. phage coat protein) and used in the identification of high affinity peptides for a given antigen.

As used herein, the term “antibody variant” refers to a modified antibody (in relation to a native or starting antibody) or a biomolecule resembling a native or starting antibody in structure and/or function (e.g., an antibody mimetic). Antibody variants may be altered in their amino acid sequence, composition, or structure as compared to a native antibody. Antibody variants may include, but are not limited to, antibodies with altered isotypes (e.g., IgA, IgD, IgE, IgG₁, IgG₂, IgG₃, IgG₄, or IgM), humanized variants, optimized variants, multispecific antibody variants (e.g., bispecific variants), and antibody fragments.

### Multispecific Antibodies

In some embodiments, the cargo or payload may be or may encode antibodies that bind more than one epitope. As used herein, the terms “multibody” or “multispecific antibody” refer to an antibody wherein two or more variable regions bind to different epitopes. The epitopes may be on the same or different targets. In certain embodiments, a multispecific antibody is a “bispecific antibody,” which recognizes two different epitopes on the same or different antigens.

In some embodiments, multi-specific antibodies may be prepared by the methods used by BIOATLA® and described in International Patent publication WO201109726, the contents of which are herein incorporated by reference in their entirety. First a library of homologous, naturally occurring antibodies is generated by any method known in the art (i.e., mammalian cell surface display), then screened by FACSAria or another screening method, for multi-specific antibodies that specifically bind to two or more target antigens. In some embodiments, the identified multi-specific antibodies are further evolved by any method known in the art, to produce a set of modified multi-specific antibodies. These modified multi-specific antibodies are screened for binding to the target antigens. In some embodiments, the multi-specific antibody may be further optimized by screening the evolved modified multi-specific antibodies for optimized or desired characteristics.

In some embodiments, multi-specific antibodies may be prepared by the methods used by BIOATLA® and described in Unites States Publication No. US20150252119, the contents of which are herein incorporated by reference in their entirety. In one approach, the variable domains of two parent antibodies, wherein the parent antibodies are monoclonal antibodies are evolved using any method known in the art in a manner that allows a single light chain to functionally complement heavy chains of two different parent antibodies. Another approach requires evolving the heavy chain of a single parent antibody to recognize a second target antigen. A third approach involves evolving the light chain of a parent antibody so as to recognize a second target antigen. Methods for polypeptide evolution are described in International Publication WO2012009026, the contents of which are herein incorporated by reference in their entirety, and include as non-limiting examples, Comprehensive Positional Evolution (CPE), Combinatorial Protein Synthesis (CPS), Comprehensive Positional Insertion (CPI), Comprehensive Positional Deletion (CPD), or any combination thereof. The Fc region of the multi-specific antibodies described in United States Publication No. US20150252119 may be created using a knob-in-hole approach, or any other method that allows the Fc domain to form heterodimers. The resultant multi-specific antibodies may be further evolved for improved characteristics or properties such as binding affinity for the target antigen.

### Bispecific Antibodies

In some embodiments, the cargo or payload may be or may encode bispecific antibodies. As used herein, the term “bispecific antibody” refers to an antibody capable of binding two different antigens. Such antibodies typically comprise regions from at least two different antibodies. Such antibodies typically comprise antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigen.

In some cases, the cargo or payload may be or may encode bispecific antibodies comprising antigen-binding regions from two different anti-tau antibodies. For example, such bispecific antibodies may comprise binding regions from two different antibodies

Bispecific antibody frameworks may include any of those described in Riethmuller, G., 2012. Cancer Immunity. 12:12-18; Marvin, J. S. et al., 2005. Acta Pharmacologica Sinica. 26(6):649-58; and Schaefer, W. et al., 2011. PNAS. 108(27):11187-92, the contents of each of which are herein incorporated by reference in their entirety.

New generations of BsMAb, called “trifunctional bispecific” antibodies, have been developed. These consist of two heavy and two light chains, one each from two different antibodies, where the two Fab regions (the arms) are directed against two antigens, and the Fc region (the foot) comprises the two heavy chains and forms the third binding site.

Of the two paratopes that form the tops of the variable domains of a bispecific antibody, one can be directed against a target antigen and the other against a T-lymphocyte antigen like CD3. In the case of trifunctional antibodies, the Fc region may additionally bind to a cell that expresses Fc receptors, like a macrophage, a natural killer (NK) cell or a dendritic cell. In sum, the targeted cell is connected to one or two cells of the immune system, which subsequently destroy it.

Other types of bispecific antibodies have been designed to overcome certain problems, such as short half-life, immunogenicity and side-effects caused by cytokine liberation. They include chemically linked Fabs, consisting only of the Fab regions, and various types of bivalent and trivalent single-chain variable fragments (scFvs), fusion proteins mimicking the variable domains of two antibodies. The furthest developed of these newer formats are the bi-specific T-cell engagers (BiTEs) and mAb2's, antibodies engineered to contain an Fcab antigen-binding fragment instead of the Fc constant region.

Using molecular genetics, two scFvs can be engineered in tandem into a single polypeptide, separated by a linker domain, called a “tandem scFv” (tascFv). TascFvs have been found to be poorly soluble and require refolding when produced in bacteria, or they may be manufactured in mammalian cell culture systems, which avoids refolding requirements but may result in poor yields. Construction of a tascFv with genes for two different scFvs yields a “bispecific single-chain variable fragments” (bis-scFvs). Only two tascFvs have been developed clinically by commercial firms; both are bispecific agents in active early phase development by Micromet for oncologic indications, and are described as “Bispecific T-cell Engagers (BiTE).” Blinatumomab is an anti-CD19/anti-CD3 bispecific tascFv that potentiates T-cell responses to B-cell non-Hodgkin lymphoma in Phase 2. MT110 is an anti-EP-CAM/anti-CD3 bispecific tascFv that potentiates T-cell responses to solid tumors in Phase 1. Bispecific, tetravalent “TandAbs” are also being researched by Affimed.

In some embodiments, the cargo or payload may be or may encode antibodies comprising a single antigen-binding domain. These molecules are extremely small, with molecular weights approximately one-tenth of those observed for full-sized mAbs. Further antibodies may include “nanobodies” derived from the antigen-binding variable heavy chain regions (V_{HH}S) of heavy chain antibodies found in camels and llamas, which lack light chains.

Disclosed and claimed in PCT Publication WO2014144573 (the contents of which are herein incorporated by reference in its entirety) to Memorial Sloan-Kettering Cancer Center are multimerization technologies for making dimeric multispecific binding agents (e.g., fusion proteins comprising antibody components) with improved properties over multispecific binding agents without the capability of dimerization.

In some cases, the cargo or payload may be or may encode tetravalent bispecific antibodies (TetBiAbs as disclosed and claimed in PCT Publication WO2014144357, the contents of which are herein incorporated in its entirety). TetBiAbs feature a second pair of Fab fragments with a second antigen specificity attached to the C-terminus of an antibody, thus providing a molecule that is bivalent for each of the two antigen specificities. The tetravalent antibody is produced by genetic engineering methods, by linking an antibody heavy chain covalently to a Fab light chain, which associates with its cognate, co-expressed Fab heavy chain.

In some aspects, the cargo or payload may be or may encode biosynthetic antibodies as described in U.S. Pat. No. 5,091,513 (the contents of which are herein incorporated by reference in their entirety). Such antibody may include one or more sequences of amino acids constituting a region which behaves as a biosynthetic antibody binding site (BABS). The sites comprise 1) non-covalently associated or disulfide bonded synthetic V_{H} and V_{L} dimers, 2) V_{H}-V_{L} or V_{L}-V_{H} single chains wherein the V_{H} and V_{L} are attached by a polypeptide linker, or 3) individuals V_{H} or V_{L} domains. The binding domains comprise linked CDR and FR regions, which may be derived from separate immunoglobulins. The biosynthetic antibodies may also include other polypeptide sequences which function, e.g., as an enzyme, toxin, binding site, or site of attachment to an immobilization media or radioactive atom. Methods are disclosed for producing the biosynthetic antibodies, for designing BABS having any specificity that can be elicited by in vivo generation of antibody, and for producing analogs thereof.

In some embodiments, the cargo or payload may be or may encode antibodies with antibody acceptor frameworks taught in U.S. Pat. No. 8,399,625. Such antibody acceptor frameworks may be particularly well suited accepting CDRs from an antibody of interest. In some cases, CDRs from anti-tau antibodies known in the art or developed according to the methods presented herein may be used.

### Miniaturized Antibody

In some embodiments, the cargo or payload may be or may encode a “miniaturized” antibody. Among the best examples of mAb miniaturization are the small modular immunopharmaceuticals (SMIPs) from Trubion Pharmaceuticals. These molecules, which can be monovalent or bivalent, are recombinant single-chain molecules containing one V_{L}, one V_{H} antigen-binding domain, and one or two constant “effector” domains, all connected by linker domains. Presumably, such a molecule might offer the advantages of increased tissue or tumor penetration claimed by fragments while retaining the immune effector functions conferred by constant domains. At least three “miniaturized” SMIPs have entered clinical development. TRU-015, an anti-CD20 SMIP developed in collaboration with Wyeth, is the most advanced project, having progressed to Phase 2 for rheumatoid arthritis (RA). Earlier attempts in systemic lupus erythrematosus (SLE) and B cell lymphomas were ultimately discontinued. Trubion and Facet Biotechnology are collaborating in the development of TRU-016, an anti-CD37 SMIP, for the treatment of CLL and other lymphoid neoplasias, a project that has reached Phase 2. Wyeth has licensed the anti-CD20 SMIP SBI-087 for the treatment of autoimmune diseases, including RA, SLE, and possibly multiple sclerosis, although these projects remain in the earliest stages of clinical testing.

### Diabodies

In some embodiments, the cargo or payload may be or may encode diabodies. As used herein, the term “diabody” refers to a small antibody fragment with two antigen-binding sites. Diabodies comprise a heavy chain variable domain V_{H} connected to a light chain variable domain V_{L} in the same polypeptide chain. By using a linker that is too short to allow pairing between the two domains on the same chain, the domains are forced to pair with the complementary domains of another chain and create two antigen-binding sites.

Diabodies are functional bispecific single-chain antibodies (bscAb). These bivalent antigen-binding molecules are composed of non-covalent dimers of scFvs, and can be produced in mammalian cells using recombinant methods. (See, e.g., Mack et al, Proc. Natl. Acad. Sci., 92: 7021-7025, 1995). Few diabodies have entered clinical development. An iodine-123-labeled diabody version of the anti-CEA chimeric antibody cT84.66 has been evaluated for pre-surgical immunoscintigraphic detection of colorectal cancer in a study sponsored by the Beckman Research Institute of the City of Hope (Clinicaltrials.gov NCT00647153).

### Unibody

In some embodiments, the cargo or payload may be or may encode a “unibody,” in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fc region to ensure stability and half-life in vivo. This configuration may minimize the risk of immune activation or oncogenic growth, as IgG4 interacts poorly with FcRs and monovalent unibodies fail to promote intracellular signaling complex formation. These contentions are, however, largely supported by laboratory, rather than clinical, evidence. Other antibodies may be “miniaturized” antibodies, which are compacted 100 kDa antibodies.

### Intrabodies

In some embodiments, the cargo or payload may be or may encode intrabodies. The term “intrabody” refers to a form of antibody that is not secreted from a cell in which it is produced, but instead targets one or more intracellular proteins. Intrabodies may be used to affect a multitude of cellular processes including, but not limited to intracellular trafficking, transcription, translation, metabolic processes, proliferative signaling, and cell division. In some embodiments, methods of the present invention may include intrabody-based therapies. In some such embodiments, variable domain sequences and/or CDR sequences disclosed herein may be incorporated into one or more constructs for intrabody-based therapy. For example, intrabodies may target one or more glycated intracellular proteins or may modulate the interaction between one or more glycated intracellular proteins and an alternative protein.

More than two decades ago, intracellular antibodies against intracellular targets were first described (Biocca, Neuberger and Cattaneo EMBO J. 9: 101-108, 1990, the contents of which are herein incorporated by reference in their entirety). The intracellular expression of intrabodies in different compartments of mammalian cells allows blocking or modulation of the function of endogenous molecules (Biocca, et al., EMBO J. 9: 101-108, 1990; Colby et al., Proc. Natl. Acad. Sci. U.S.A. 101: 17616-21, 2004, the contents of which are herein incorporated by reference in their entirety). Intrabodies can alter protein folding, protein-protein, protein-DNA, protein-RNA interactions and protein modification. They can induce a phenotypic knockout and work as neutralizing agents by direct binding to the target antigen, by diverting its intracellular trafficking or by inhibiting its association with binding partners. They have been largely employed as research tools and are emerging as therapeutic molecules for the treatment of human diseases such as viral pathologies, cancer and misfolding diseases. The fast-growing bio-market of recombinant antibodies provides intrabodies with enhanced binding specificity, stability, and solubility, together with lower immunogenicity, for their use in therapy.

In some embodiments, intrabodies have advantages over interfering RNA (iRNA); for example, iRNA has been shown to exert multiple non-specific effects, whereas intrabodies have been shown to have high specificity and affinity to target antigens. Furthermore, as proteins, intrabodies possess a much longer active half-life than iRNA. Thus, when the active half-life of the intracellular target molecule is long, gene silencing through iRNA may be slow to yield an effect, whereas the effects of intrabody expression can be almost instantaneous. Lastly, it is possible to design intrabodies to block certain binding interactions of a particular target molecule, while sparing others.

Intrabodies are often single chain variable fragments (scFvs) expressed from a recombinant nucleic acid molecule and engineered to be retained intracellularly (e.g., retained in the cytoplasm, endoplasmic reticulum, or periplasm). Intrabodies may be used, for example, to ablate the function of a protein to which the intrabody binds. The expression of intrabodies may also be regulated through the use of inducible promoters in the nucleic acid expression vector comprising the intrabody. Intrabodies may be produced for use in the viral genomes of the invention using methods known in the art, such as those disclosed and reviewed in: Marasco et al., 1993 Proc. Natl. Acad. Sci. USA, 90: 7889-7893; Chen et al., 1994, Hum. Gene Ther. 5:595-601; Chen et al., 1994, Proc. Natl. Acad. Sci. USA, 91: 5932-5936; Maciejewski et al., 1995, Nature Med., 1: 667-673; Marasco, 1995, Immunotech, 1: 1-19; Mhashilkar, et al., 1995, EMBO J. 14: 1542-51; Chen et al., 1996, Hum. Gene Therap., 7: 1515-1525; Marasco, Gene Ther. 4:11-15, 1997; Rondon and Marasco, 1997, Annu. Rev. Microbiol. 51:257-283; Cohen, et al., 1998, Oncogene 17:2445-56; Proba et al., 1998, J. Mol. Biol. 275:245-253; Cohen et al., 1998, Oncogene 17:2445-2456; Hassanzadeh, et al., 1998, FEBS Lett. 437:81-6; Richardson et al., 1998, Gene Ther. 5:635-44; Ohage and Steipe, 1999, J. Mol. Biol. 291:1119-1128; Ohage et al., 1999, J. Mol. Biol. 291:1129-1134; Wirtz and Steipe, 1999, Protein Sci. 8:2245-2250; Zhu et al., 1999, J. Immunol. Methods 231:207-222; Arafat et al., 2000, Cancer Gene Ther. 7:1250-6; der Maur et al., 2002, J. Biol. Chem. 277:45075-85; Mhashilkar et al., 2002, Gene Ther. 9:307-19; and Wheeler et al., 2003, FASEB J. 17: 1733-5; and references cited therein). In particular, a CCR5 intrabody has been produced by Steinberger et al., 2000, Proc. Natl. Acad. Sci. USA 97:805-810). See generally Marasco, WA, 1998, “Intrabodies: Basic Research and Clinical Gene Therapy Applications” Springer: New York; and for a review of scFvs, see Pluckthun in “The Pharmacology of Monoclonal Antibodies,” 1994, vol. 113, Rosenburg and Moore eds. Springer-Verlag, New York, pp. 269-315; the contents of each of which are each incorporated by reference in their entireties.

Sequences from donor antibodies may be used to develop intrabodies. Intrabodies are often recombinantly expressed as single domain fragments such as isolated V_{H} and V_{L} domains or as a single chain variable fragment (scFv) antibody within the cell. For example, intrabodies are often expressed as a single polypeptide to form a single chain antibody comprising the variable domains of the heavy and light chains joined by a flexible linker polypeptide. Intrabodies typically lack disulfide bonds and are capable of modulating the expression or activity of target genes through their specific binding activity. Single chain antibodies can also be expressed as a single chain variable region fragment joined to the light chain constant region.

As is known in the art, an intrabody can be engineered into recombinant polynucleotide vectors to encode sub-cellular trafficking signals at its N or C terminus to allow expression at high concentrations in the sub-cellular compartments where a target protein is located. For example, intrabodies targeted to the endoplasmic reticulum (ER) are engineered to incorporate a leader peptide and, optionally, a C-terminal ER retention signal. Intrabodies intended to exert activity in the nucleus are engineered to include a nuclear localization signal. Lipid moieties are joined to intrabodies in order to tether the intrabody to the cytosolic side of the plasma membrane. Intrabodies can also be targeted to exert function in the cytosol. For example, cytosolic intrabodies are used to sequester factors within the cytosol, thereby preventing them from being transported to their natural cellular destination.

There are certain technical challenges with intrabody expression. In particular, protein conformational folding and structural stability of the newly-synthesized intrabody within the cell is affected by reducing conditions of the intracellular environment.

Intrabodies of the invention may be promising therapeutic agents for the treatment of misfolding diseases, including Tauopathies, prion diseases, Alzheimer's, Parkinson's, and Huntington's, because of their virtually infinite ability to specifically recognize the different conformations of a protein, including pathological isoforms, and because they can be targeted to the potential sites of aggregation (both intra- and extracellular sites). These molecules can work as neutralizing agents against amyloidogenic proteins by preventing their aggregation, and/or as molecular shunters of intracellular traffic by rerouting the protein from its potential aggregation site.

### Maxibodies

In some embodiments, the cargo or payload may be or may encode a maxibody (bivalent scFV fused to the amino terminus of the Fc (CH2-CH3 domains) of IgG.

### Chimeric Antigen Receptors (CARs)

In some embodiments, the cargo or payload may be or may encode a chimeric antigen receptors (CARs) which when transduced into immune cells (e.g., T cells and NK cells), can re-direct the immune cells against the target (e.g., a tumor cell) which expresses a molecule recognized by the extracellular target moiety of the CAR.

As used herein, the term “chimeric antigen receptor (CAR)” refers to a synthetic receptor that mimics TCR on the surface of T cells. In general, a CAR is composed of an extracellular targeting domain, a transmembrane domain/region and an intracellular signaling/activation domain. In a standard CAR receptor, the components: the extracellular targeting domain, transmembrane domain and intracellular signaling/activation domain, are linearly constructed as a single fusion protein. The extracellular region comprises a targeting domain/moiety (e.g., a scFv) that recognizes a specific tumor antigen or other tumor cell-surface molecules. The intracellular region may contain a signaling domain of TCR complex (e.g., the signal region of CD3ζ), and/or one or more costimulatory signaling domains, such as those from CD28, 4-1BB (CD137) and OX-40 (CD134). For example, a “first-generation CAR” only has the CD3ζ signaling domain, whereas in an effort to augment T-cell persistence and proliferation, costimulatory intracellular domains are added, giving rise to second generation CARs having a CD3ζ signal domain plus one costimulatory signaling domain, and third generation CARs having CD3ζ signal domain plus two or more costimulatory signaling domains. A CAR, when expressed by a T cell, endows the T cell with antigen specificity determined by the extracellular targeting moiety of the CAR. In some aspects you could add one or more elements such as homing and suicide genes to develop a more competent and safer architecture of CAR (so called the fourth generation CAR).

In some embodiments, the extracellular targeting domain is joined through the hinge (also called space domain or spacer) and transmembrane regions to an intracellular signaling domain. The hinge connects the extracellular targeting domain to the transmembrane domain which transverses the cell membrane and connects to the intracellular signaling domain. The hinge may need to be varied to optimize the potency of CAR transformed cells toward cancer cells due to the size of the target protein where the targeting moiety binds, and the size and affinity of the targeting domain itself. Upon recognition and binding of the targeting moiety to the target cell, the intracellular signaling domain leads to an activation signal to the CAR T cell, which is further amplified by the “second signal” from one or more intracellular costimulatory domains. The CAR T cell, once activated, can destroy the target cell.

In some embodiments, the CAR may be split into two parts, each part is linked a dimerizing domain, such that an input that triggers the dimerization promotes assembly of the intact functional receptor. Wu and Lim reported a split CAR in which the extracellular CD19 binding domain and the intracellular signaling element are separated and linked to the FKBP domain and the FRB* (T2089L mutant of FKBP-rapamycin binding) domain that heterodimerize in the presence of the rapamycin analog AP21967. The split receptor is assembled in the presence of AP21967 and together with the specific antigen binding, activates T cells (Wu et al., Science, 2015, 625(6258): aab4077, the contents of which are herein incorporated by reference in its entirety).

In some embodiments, the CAR may be designed as an inducible CAR which has an incorporation of a Tet-On inducible system to a CD19 CAR construct. The CD19 CAR is activated only in the presence of doxycycline (Dox). Sakemura reported that Tet-CD19 CAR T cells in the presence of Dox were equivalently cytotoxic against CD19+ cell lines and had equivalent cytokine production and proliferation upon CD19 stimulation, compared with conventional CD19 CAR T cells (Sakemura et al., Cancer Immuno. Res., 2016, Jun. 21, Epub; the contents of which is herein incorporated by reference in its entirety). The dual systems provide more flexibility to turn-on and off of the CAR expression in transduced T cells.

In some embodiments, the cargo or payload may be or may encode a first generation CAR, or a second generation CAR, or a third generation CAR, or a fourth generation CAR. In some embodiments, the cargo or payload may be or may encode a full CAR construct composed of the extracellular domain, the hinge and transmembrane domain and the intracellular signaling region. In other embodiments, the cargo or payload may be or may encode a component of the full CAR construct including an extracellular targeting moiety, a hinge region, a transmembrane domain, an intracellular signaling domain, one or more co-stimulatory domain, and other additional elements that improve CAR architecture and functionality including but not limited to a leader sequence, a homing element and a safety switch, or the combination of such components.

In some embodiments, the cargo or payload may be or may encode a tunable CARs. The reversible on-off switch mechanism allows management of acute toxicity caused by excessive CAR-T cell expansion. The ligand conferred regulation of the CAR may be effective in offsetting tumor escape induced by antigen loss, avoiding functional exhaustion caused by tonic signaling due to chronic antigen exposure and improving the persistence of CAR expressing cells in vivo. The tunable CAR may be utilized to down regulate CAR expression to limit on target on tissue toxicity caused by tumor lysis syndrome. Down regulating the expression of the CARs following anti-tumor efficacy may prevent (1) On target off tumor toxicity caused by antigen expression in normal tissue. (2) antigen independent activation in vivo.

### Extracellular Targeting Domain/Moiety

In some embodiments, the extracellular target moiety of a CAR may be any agent that recognizes and binds to a given target molecule, for example, a neoantigen on tumor cells, with high specificity and affinity. The target moiety may be an antibody and variants thereof that specifically binds to a target molecule on tumor cells, or a peptide aptamer selected from a random sequence pool based on its ability to bind to the target molecule on tumor cells, or a variant or fragment thereof that can bind to the target molecule on tumor cells, or an antigen recognition domain from native T-cell receptor (TCR) (e.g. CD4 extracellular domain to recognize HIV infected cells), or exotic recognition components such as a linked cytokine that leads to recognition of target cells bearing the cytokine receptor, or a natural ligand of a receptor.

In some embodiments, the targeting domain of a CAR may be a Ig NAR, a Fab fragment, a Fab′ fragment, a F(ab)′2 fragment, a F(ab)′3 fragment, Fv, a single chain variable fragment (scFv), a bis-scFv, a (scFv)₂, a minibody, a diabody, a triabody, a tetrabody, a disulfide stabilized Fv protein (dsFv), a unibody, a nanobody, or an antigen binding region derived from an antibody that specifically recognizes a target molecule, for example a tumor specific antigen (TSA). In one embodiment, the targeting moiety is a scFv antibody. The scFv domain, when it is expressed on the surface of a CAR T cell and subsequently binds to a target protein on a cancer cell, is able to maintain the CAR T cell in proximity to the cancer cell and to trigger the activation of the T cell. A scFv can be generated using routine recombinant DNA technology techniques and is discussed in the present invention.

In some embodiments, the targeting moiety of a CAR construct may be an aptamer such as a peptide aptamer that specifically binds to a target molecule of interest. The peptide aptamer may be selected from a random sequence pool based on its ability to bind to the target molecule of interest.

In some embodiments, the targeting moiety of a CAR construct may be a natural ligand of the target molecule, or a variant and/or fragment thereof capable of binding the target molecule. In some aspects, the targeting moiety of a CAR may be a receptor of the target molecule, for example, a full length human CD27, as a CD70 receptor, may be fused in frame to the signaling domain of CD3 ζ forming a CD27 chimeric receptor as an immunotherapeutic agent for CD70-positive malignancies.

In some embodiments, the targeting moiety of a CAR may recognize a tumor specific antigen (TSA), for example a cancer neoantigen which is restrictedly expressed on tumor cells.

As non-limiting examples, the CAR of the present invention may comprise the extracellular targeting domain capable of binding to a tumor specific antigen selected from 5T4, 707-AP, A33, AFP (□-fetoprotein), AKAP-4 (A kinase anchor protein 4), ALK, α5β1-integrin, androgen receptor, annexin II, alpha-actinin-4, ART-4, Bi, B7H3, B7H4, BAGE (B melanoma antigen), BCMA, BCR-ABL fusion protein, beta-catenin, BKT-antigen, BTAA, CA-I (carbonic anhydrase I), CA50 (cancer antigen 50), CA125, CA15-3, CA195, CA242, calretinin, CAIX (carbonic anhydrase), CAMEL (cytotoxic T-lymphocyte recognized antigen on melanoma), CAM43, CAP-1, Caspase-8/m, CD4, CD5, CD7, CD19, CD20, CD22, CD23, CD25, CD27/m, CD28, CD30, CD33, CD34, CD36, CD38, CD40/CD154, CD41, CD44v6, CD44v7/8, CD45, CD49f, CD56, CD68KP1, CD74, CD79a/CD79b, CD103, CD123, CD133, CD138, CD171, cdc27/m, CDK4 (cyclin dependent kinase 4), CDKN2A, CDS, CEA (carcinoembryonic antigen), CEACAM5, CEACAM6, chromogranin, c-Met, c-Myc, coa-1, CSAp, CT7, CT10, cyclophilin B, cyclin B1, cytoplasmic tyrosine kinases, cytokeratin, DAM-10, DAM-6, dek-can fusion protein, desmin, DEPDC1 (DEP domain containing 1), E2A-PRL, EBNA, EGF-R (epidermal growth factor receptor), EGP-1(epithelial glycoprotein-1) (TROP-2), EGP-2, EGP-40, EGFR (epidermal growth factor receptor), EGFRvIII, EF-2, ELF2M, EMMPRIN, EpCAM (epithelial cell adhesion molecule), EphA2, Epstein Barr virus antigens, Erb (ErbB1; ErbB3; ErbB4), ETA (epithelial tumor antigen), ETV6-AML1 fusion protein, FAP (fibroblast activation protein), FBP (folate-binding protein), FGF-5, folate receptor, FOS related antigen 1, fucosyl GM1, G250, GAGE (GAGE-1; GAGE-2), galectin, GD2 (ganglioside), GD3, GFAP (glial fibrillary acidic protein), GM2 (oncofetal antigen-immunogenic-1; OFA-1-1), GnT-V, Gp100, H4-RET, HAGE (helicase antigen), HER-2/neu, HIFs (hypoxia inducible factors), HIF-1, HIF-2, HLA-A2, HLA-A*0201-R170I, HLA-A11, HMWMAA, Hom/Mel-40, HSP70-2M (Heat shock protein 70), HST-2, HTgp-175, hTERT (or hTRT), human papillomavirus-E6/human papillomavirus-E7 and E6, iCE (immune-capture EIA), IGF-1R, IGH-IGK, IL-2R, IL-5, ILK (integrin-linked kinase), IMP3 (insulin-like growth factor II mRNA-binding protein 3), IRF4 (interferon regulatory factor 4), KDR (kinase insert domain receptor), KIAA0205, KRAB-zinc finger protein (KID)-3; KID31, KSA (17-1A), K-ras, LAGE, LCK, LDLR/FUT (LDLR-fucosyltransferaseAS fusion protein), LeY (Lewis Y), MAD-CT-1, MAGE (tyrosinase, melanoma-associated antigen) (MAGE-1; MAGE-3), melan-A tumor antigen (MART), MART-2/Ski, MC1R (melanocortin 1 receptor), MDM2, mesothelin, MPHOSPH1, MSA(muscle-specific actin), mTOR (mammalian targets of rapamycin), MUC-1, MUC-2, MUM-1 (melanoma associated antigen (mutated) 1), MUM-2, MUM-3, Myosin/m, MYL-RAR, NA88-A, N-acetylglucosaminyltransferase, neo-PAP, NF-KB (nuclear factor-kappa B), neurofilament, NSE (neuron-specific enolase), Notch receptors, NuMa, N-Ras, NY—BR-1, NY—CO-1, NY-ESO-1, Oncostatin M, OS-9, OY-TES1, p53 mutants, p190 minor bcr-abl, p15(58), pl85erbB2, pl80erbB-3, PAGE (prostate associated gene), PAP (prostatic acid phosphatase), PAX3, PAX5, PDGFR (platelet derived growth factor receptor), cytochrome P450 involved in piperidine and pyrrolidine utilization (PIPA), Pml-RAR alpha fusion protein, PR-3 (proteinase 3), PSA (prostate specific antigen), PSM, PSMA (Prostate stem cell antigen), PRAME (preferentially expressed antigen of melanoma), PTPRK, RAGE (renal tumor antigen), Raf (A-Raf, B-Raf and C-Raf), Ras, receptor tyrosine kinases, RCAS1, RGSS, ROR1 (receptor tyrosine kinase-like orphan receptor 1), RU1, RU2, SAGE, SART-1, SART-3, SCP-1, SDCCAG16, SP-17 (sperm protein 17), src-family, SSX (synovial sarcoma X breakpoint)-1, SSX-2(HOM-MEL-40), SSX-3, SSX-4, SSX-5, STAT-3, STAT-5, STAT-6, STEAD, STn, survivin, syk-ZAP70, TA-90 (Mac-2 binding protein\cyclophilin C-associated protein), TAAL6, TACSTD1 (tumor associated calcium signal transducer 1), TACSTD2, TAG-72-4, TAGE, TARP (T cell receptor gamma alternate reading frame protein), TEL/AML1 fusion protein, TEM1, TEM8 (endosialin or CD248), TGFβ, TIE2, TLP, TMPRSS2 ETS fusion gene, TNF-receptor (TNF-α receptor, TNF-β receptor; or TNF-γ receptor), transferrin receptor, TPS, TRP-1 (tyrosine related protein 1), TRP-2, TRP-2/INT2, TSP-180, VEGF receptor, WNT, WT-1 (Wilm's tumor antigen) and XAGE.

In some embodiments, the cargo or payload may be or may encode a CAR which comprises a universal immune receptor which has a targeting moiety capable of binding to a labelled antigen.

In some embodiments, the cargo or payload may be or may encode a CAR which comprises a targeting moiety capable of binding to a pathogen antigen.

In some embodiments, the cargo or payload may be or may encode a CAR which comprises a targeting moiety capable of binding to non-protein molecules such as tumor-associated glycolipids and carbohydrates.

In some embodiments, the cargo or payload may be or may encode a CAR which comprises a targeting moiety capable of binding to a component within the tumor microenvironment including proteins expressed in various tumor stroma cells including tumor associated macrophages (TAMs), immature monocytes, immature dendritic cells, immunosuppressive CD4+CD25+ regulatory T cells (Treg) and MDSCs.

In some embodiments, the cargo or payload may be or may encode a CAR which comprises a targeting moiety capable of binding to a cell surface adhesion molecule, a surface molecule of an inflammatory cell that appears in an autoimmune disease, or a TCR causing autoimmunity. As non-limiting examples, the targeting moiety of the present invention may be a scFv antibody that recognizes a tumor specific antigen (TSA), for example scFvs of antibodies SS, SS1 and HN1 that specifically recognize and bind to human mesothelin, scFv of antibody of GD2, a CD19 antigen binding domain, a NKG2D ligand binding domain, human anti-mesothelin scFvs, an anti-CS1 binding agent, an anti-BCMA binding domain, anti-CD19 scFv antibody, GFR alpha 4 antigen binding fragments, anti-CLL-1 (C-type lectin-like molecule 1) binding domains, CD33 binding domains, a GPC3 (glypican-3) binding domain, a GFR alpha4 (Glycosyl-phosphatidylinositol (GPI)-linked GDNF family α-receptor 4 cell-surface receptor) binding domain, CD123 binding domains, an anti-ROR1 antibody or fragments thereof, scFvs specific to GPC-3, scFv for CSPG4, and scFv for folate receptor alpha.

### Intracellular Signaling Domains

The intracellular domain of a CAR fusion polypeptide, after binding to its target molecule, transmits a signal to the immune effector cell, activating at least one of the normal effector functions of immune effector cells, including cytolytic activity (e.g., cytokine secretion) or helper activity. Therefore, the intracellular domain comprises an “intracellular signaling domain” of a T cell receptor (TCR).

In some aspects, the entire intracellular signaling domain can be employed. In other aspects, a truncated portion of the intracellular signaling domain may be used in place of the intact chain as long as it transduces the effector function signal.

In some embodiments, the intracellular signaling domain may contain signaling motifs which are known as immunoreceptor tyrosine-based activation motifs (ITAMs). Examples of ITAM containing cytoplasmic signaling sequences include those derived from TCR CD3zeta, FcR gamma, FcR beta, CD3 gamma, CD3 delta, CD3 epsilon, CD5, CD22, CD79a, CD79b, and CD66d. In one example, the intracellular signaling domain is a CD3 zeta (CD3ζ) signaling domain.

In some embodiments, the intracellular region further comprises one or more costimulatory signaling domains which provide additional signals to the immune effector cells. These costimulatory signaling domains, in combination with the signaling domain can further improve expansion, activation, memory, persistence, and tumor-eradicating efficiency of CAR engineered immune cells (e.g., CAR T cells). In some cases, the costimulatory signaling region contains 1, 2, 3, or 4 cytoplasmic domains of one or more intracellular signaling and/or costimulatory molecules. The costimulatory signaling domain may be the intracellular/cytoplasmic domain of a costimulatory molecule, including but not limited to CD2, CD7, CD27, CD28, 4-1BB (CD137), OX40 (CD134), CD30, CD40, ICOS (CD278), GITR (glucocorticoid-induced tumor necrosis factor receptor), LFA-1 (lymphocyte function-associated antigen-1), LIGHT, NKG2C, B7-H3. In one example, the costimulatory signaling domain is derived from the cytoplasmic domain of CD28. In another example, the costimulatory signaling domain is derived from the cytoplasmic domain of 4-1BB (CD137). In another example, the co-stimulatory signaling domain may be an intracellular domain of GITR as taught in U.S. Pat. No. 9,175,308; the contents of which are incorporated herein by reference in its entirety.

In some embodiments, the intracellular region may comprise a functional signaling domain from a protein selected from the group consisting of an MHC class I molecule, a TNF receptor protein, an immunoglobulin-like protein, a cytokine receptor, an integrin, a signaling lymphocytic activation protein (SLAM) such as CD48, CD229, 2B4, CD84, NTB-A, CRACC, BLAME, CD2F-10, SLAMF6, SLAMF7, an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), SLAMF7, NKp80 (KLRF1), NKp44, NKp30, NKp46, CD19, CD4, CD8alpha, CD8beta, IL2R beta, IL2R gamma, IL7R alpha, IL-15Ra, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, NKD2C SLP76, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, CD270 (HVEM), GADS, SLP-76, PAG/Cbp, CD19a, a ligand that specifically binds with CD83, DAP 10, TRIM, ZAP70, Killer immunoglobulin receptors (KIRs) such as KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, KIR3DL3, and KIR2DP1; lectin related NK cell receptors such as Ly49, Ly49A, and Ly49C.

In some embodiments, the intracellular signaling domain of the present invention may contain signaling domains derived from JAK-STAT. In other embodiments, the intracellular signaling domain of the present invention may contain signaling domains derived from DAP-12 (Death associated protein 12) (Topfer et al., Immunol., 2015, 194: 3201-3212; and Wang et al., Cancer Immunol., 2015, 3: 815-826). DAP-12 is a key signal transduction receptor in NK cells. The activating signals mediated by DAP-12 play important roles in triggering NK cell cytotoxicity responses toward certain tumor cells and virally infected cells. The cytoplasmic domain of DAP12 contains an Immunoreceptor Tyrosine-based Activation Motif (ITAM). Accordingly, a CAR containing a DAP12-derived signaling domain may be used for adoptive transfer of NK cells.

### Transmembrane Domains

In some embodiments, the CAR may comprise a transmembrane domain. As used herein, the term “Transmembrane domain (TM)” refers broadly to an amino acid sequence of about 15 residues in length which spans the plasma membrane. The transmembrane domain may include at least 20, 21, 22, 23, 24, 25, 26, 27, 28, 29, 30, 31, 32, 33, 34, 35, 36, 37, 38, 39, 40, 41, 42, 43, 44, or 45 amino acid residues and spans the plasma membrane. In some embodiments, the transmembrane domain may be derived either from a natural or from a synthetic source. The transmembrane domain of a CAR may be derived from any naturally membrane-bound or transmembrane protein. For example, the transmembrane region may be derived from (i.e. comprise at least the transmembrane region(s) of) the alpha, beta or zeta chain of the T-cell receptor, CD3 epsilon, CD4, CD5, CD8, CD8a, CD9, CD16, CD22, CD33, CD28, CD37, CD45, CD64, CD80, CD86, CD134, CD137, CD152, or CD154.

Alternatively, the transmembrane domain of the present invention may be synthetic. In some aspects, the synthetic sequence may comprise predominantly hydrophobic residues such as leucine and valine.

In some embodiments, the transmembrane domain may be selected from the group consisting of a CD8a transmembrane domain, a CD4 transmembrane domain, a CD 28 transmembrane domain, a CTLA-4 transmembrane domain, a PD-1 transmembrane domain, and a human IgG4 Fc region.

In some embodiments, the CAR may comprise an optional hinge region (also called spacer). A hinge sequence is a short sequence of amino acids that facilitates flexibility of the extracellular targeting domain that moves the target binding domain away from the effector cell surface to enable proper cell/cell contact, target binding and effector cell activation. The hinge sequence may be positioned between the targeting moiety and the transmembrane domain. The hinge sequence can be any suitable sequence derived or obtained from any suitable molecule. The hinge sequence may be derived from all or part of an immunoglobulin (e.g., IgG1, IgG2, IgG3, IgG4) hinge region, i.e., the sequence that falls between the CHI and CH2 domains of an immunoglobulin, e.g., an IgG4 Fc hinge, the extracellular regions of type 1 membrane proteins such as CD8a CD4, CD28 and CD7, which may be a wild-type sequence or a derivative. Some hinge regions include an immunoglobulin CH3 domain or both a CH3 domain and a CH2 domain. In certain embodiments, the hinge region may be modified from an IgG1, IgG2, IgG3, or IgG4 that includes one or more amino acid residues, for example, 1, 2, 3, 4 or 5 residues, substituted with an amino acid residue different from that present in an unmodified hinge.

In some embodiments, the CAR may comprise one or more linkers between any of the domains of the CAR. The linker may be between 1-30 amino acids long. In this regard, the linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. In other embodiments, the linker may be flexible.

In some embodiments, the components including the targeting moiety, transmembrane domain and intracellular signaling domains may be constructed in a single fusion polypeptide. The fusion polypeptide may be the payload of an effector module of the invention.

In some embodiments, the cargo or payload may be or may encode a CD19 specific CAR targeting different B cell malignancies and HER2-specific CAR targeting sarcoma, glioblastoma, and advanced Her2-positive lung malignancy. Tandem CAR (TanCAR)

In some embodiments, the CAR may be a tandem chimeric antigen receptor (TanCAR) which is able to target two, three, four, or more tumor specific antigens. In some aspects, The CAR is a bispecific TanCAR including two targeting domains which recognize two different TSAs on tumor cells. The bispecific TanCAR may be further defined as comprising an extracellular region comprising a targeting domain (e.g., an antigen recognition domain) specific for a first tumor antigen and a targeting domain (e.g., an antigen recognition domain) specific for a second tumor antigen. In other aspects, the CAR is a multispecific TanCAR that includes three or more targeting domains configured in a tandem arrangement. The space between the targeting domains in the TanCAR may be between about 5 and about 30 amino acids in length, for example, 6, 7, 8, 9, 10, 11, 12, 13, 14, 15, 16, 17, 18, 19, 20, 21, 22, 23, 24, 25, 26, 27, 28, 29 and 30 amino acids.

### Split CAR

In some embodiments, the CAR components including the targeting moiety, transmembrane domain and intracellular signaling domains may be split into two or more parts such that it is dependent on multiple inputs that promote assembly of the intact functional receptor. As a non-limiting example, the split CAR consists of two parts that assemble in a small molecule-dependent manner; one part of the receptor features an extracellular antigen binding domain (e.g. scFv) and the other part has the intracellular signaling domains, such as the CD3ζ intracellular domain.

In other aspects, the split parts of the CAR system can be further modified to increase signal. As a non-limiting example, the second part of cytoplasmic fragment may be anchored to the plasma membrane by incorporating a transmembrane domain (e.g., CD8α transmembrane domain) to the construct. An additional extracellular domain may also be added to the second part of the CAR system, for instance an extracellular domain that mediates homo-dimerization. These modifications may increase receptor output activity, i.e., T cell activation.

In some embodiments, the two parts of the split CAR system contain heterodimerization domains that conditionally interact upon binding of a heterodimerizing small molecule. As such, the receptor components are assembled in the presence of the small molecule, to form an intact system which can then be activated by antigen engagement. Any known heterodimerizing components can be incorporated into a split CAR system. Other small molecule dependent heterodimerization domains may also be used, including, but not limited to, gibberellin-induced dimerization system (GID1-GAI), trimethoprim-SLF induced ecDHFR and FKBP dimerization and ABA (abscisic acid) induced dimerization of PP2C and PYL domains. The dual regulation using inducible assembly (e.g., ligand dependent dimerization) and degradation (e.g., destabilizing domain induced CAR degradation) of the split CAR system may provide more flexibility to control the activity of the CAR modified T cells.

### Switchable CAR

In some embodiments, the CAR may be a switchable CAR which is a controllable CARs that can be transiently switched on in response to a stimulus (e.g. a small molecule). In this CAR design, a system is directly integrated in the hinge domain that separate the scFv domain from the cell membrane domain in the CAR. Such system is possible to split or combine different key functions of a CAR such as activation and costimulation within different chains of a receptor complex, mimicking the complexity of the TCR native architecture. This integrated system can switch the scFv and antigen interaction between on/off states controlled by the absence/presence of the stimulus.

### Reversible CAR

In some embodiments, the CAR may be a reversible CAR system. In this CAR architecture, a LID domain (ligand-induced degradation) is incorporated into the CAR system. The CAR can be temporarily down-regulated by adding a ligand of the LID domain.

Inhibitory CAR (iCAR)

In some embodiments, the CAR may be inhibitory CARs. Inhibitory CAR (iCAR) refers to a bispecific CAR design wherein a negative signal is used to enhance the tumor specificity and limit normal tissue toxicity. This design incorporates a second CAR having a surface antigen recognition domain combined with an inhibitory signal domain to limit T cell responsiveness even with concurrent engagement of an activating receptor. This antigen recognition domain is directed towards a normal tissue specific antigen such that the T cell can be activated in the presence of first target protein, but if the second protein that binds to the iCAR is present, the T cell activation is inhibited.

As a non-limiting example, iCARs against Prostate specific membrane antigen (PMSA) based on CTLA4 and PD1 inhibitory domains demonstrated the ability to selectively limit cytokine secretion, cytotoxicity and proliferation induced by T cell activation.

### Chimeric Switch Receptor

In some embodiments, the cargo or payload may be or may encode a chimeric switch receptors which can switch a negative signal to a positive signal. As used herein, the term “chimeric switch receptor” refers to a fusion protein comprising a first extracellular domain and a second transmembrane and intracellular domain, wherein the first domain includes a negative signal region and the second domain includes a positive intracellular signaling region. In some aspects, the fusion protein is a chimeric switch receptor that contains the extracellular domain of an inhibitory receptor on T cell fused to the transmembrane and cytoplasmic domain of a co-stimulatory receptor. This chimeric switch receptor may convert a T cell inhibitory signal into a T cell stimulatory signal.

As a non-limiting example, the chimeric switch receptor may comprise the extracellular domain of PD-1 fused to the transmembrane and cytoplasmic domain of CD28. In some aspects, Extracellular domains of other inhibitory receptors such as CTLA-4, LAG-3, TIM-3, KIRs and BTLA may also be fused to the transmembrane and cytoplasmic domain derived from costimulatory receptors such as CD28, 4-1BB, CD27, OX40, CD40, GTIR and ICOS.

In some embodiments, chimeric switch receptors may include recombinant receptors comprising the extracellular cytokine-binding domain of an inhibitory cytokine receptor (e.g., IL-13 receptor α (IL-13Rα1), IL-10R, and IL-4Rα) fused to an intracellular signaling domain of a stimulatory cytokine receptor such as IL-2R (IL-2R□, IL-2Rβ and IL-2Rgamma) and IL-7Rα. One example of such chimeric cytokine receptor is a recombinant receptor containing the cytokine-binding extracellular domain of IL-4Ra linked to the intracellular signaling domain of IL-7Ra.

In some embodiments, the chimeric switch receptor may be a chimeric TGFβ receptor. The chimeric TGFβ receptor may comprise an extracellular domain derived from a TGFβ receptor such as TGFβ receptor 1, TGFβ receptor 2, TGFβ receptor 3, or any other TGFβ receptor or variant thereof; and a non-TGFβ receptor intracellular domain. The non-TGFβ receptor intracellular domain may be the intracellular domain or fragment thereof derived from TLR1, TLR2, TLR3, TLR4, TLR5, TLR6, TLR7, TLR8, TLR9, TLR10, CD28, 4-1BB (CD137), OX40 (CD134), CD3zeta, CD40, CD27, or a combination thereof.

### Activation-Conditional CAR

In some embodiments, the cargo or payload may be or may encode an activation-conditional chimeric antigen receptor, which is only expressed in an activated immune cell. The expression of the CAR may be coupled to activation conditional control region which refers to one or more nucleic acid sequences that induce the transcription and/or expression of a sequence e.g., a CAR under its control. Such activation conditional control regions may be promoters of genes that are upregulated during the activation of the immune effector cell e.g. 1L2 promoter or NFAT binding sites.

CAR Targeting to Tumor Cells with Specific Proteoglycan Markers

In some embodiments, the cargo or payload may be or may encode a CAR that targets specific types of cancer cells. Human cancer cells and metastasis may express unique and otherwise abnormal proteoglycans, such as polysaccharide chains (e.g., chondroitin sulfate (CS), dermatan sulfate (DS or CSB), heparan sulfate (HS) and heparin). Accordingly, the CAR may be fused with a binding moiety that recognizes cancer associated proteoglycans. In one example, a CAR may be fused with VAR2CSA polypeptide (VAR2-CAR) that binds with high affinity to a specific type of chondroitin sulfate A (CSA) attached to proteoglycans. The extracellular ScFv portion of the CAR may be substituted with VAR2CSA variants comprising at least the minimal CSA binding domain, generating CARs specific to chondroitin sulfate A (CSA) modifications. Alternatively, the CAR may be fused with a split-protein binding system to generate a spy-CAR, in which the scFv portion of the CAR is substituted with one portion of a split-protein binding system such as SpyTag and Spy-catcher and the cancer-recognition molecules (e.g. scFv and or VAR2-CSA) are attached to the CAR through the split-protein binding system.

### Nucleic Acids

The delivery vehicles of the present disclosure may comprise a payload region (which may also be referred to as a cargo region) which is a nucleic acid. The term “nucleic acid,” in its broadest sense, includes any compound and/or substance that comprise a polymer of nucleotides which may be referred to as polynucleotides. Exemplary nucleic acids or polynucleotides include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs) or hybrids thereof.

In some embodiments, the payload region comprises nucleic acid sequences encoding more than one cargo or payload.

In some embodiments, the payload region may be or encode a coding nucleic acid sequence.

In some embodiments, the payload region may be or encode a non-coding nucleic acid sequence.

In some embodiments, the payload region may be or encode both a coding and a non-coding nucleic acid sequence.

### DNA

Deoxyribonucleic acid (DNA) is a molecule that carries genetic information for all living things and consists of two strands that wind around one another to form a shape known as a double helix. Each strand has a backbone made of alternating sugar (deoxyribose) and phosphate groups. Attached to each sugar is one of four bases: adenine (A), cytosine (C), guanine (G), and thymine (T). The two strands are held together by bonds between adenine and thymine or cytosine and guanine. The sequence of the bases along the backbones serves as instructions for assembling protein and RNA molecules.

In some embodiments, the payload region may be or encode a coding DNA.

In some embodiments, the payload region may be or encode a non-coding DNA.

In some embodiments, the payload region may be or encode both a coding and a non-coding DNA.

In some embodiments, the DNA may be modified. Types of modifications include, but are not limited to, methylation, acetylation, phosphorylation, ubiquitination, and sumoylation.

### Vectors

In some embodiments, the originator constructs and/or benchmark constructs described herein can be or be encoded by vectors such as plasmids or viral vectors. In some embodiments, the originator constructs and/or benchmark constructs are or are encoded by viral vectors. Viral vectors may be, but are not limited to, Herpesvirus (HSV) vectors, retroviral vectors, adenoviral vectors, adeno-associated viral (AAV) vectors, lentiviral vectors, and the like. In some embodiments, the viral vectors are AAV vectors. In some embodiments, the viral vectors are lentiviral vectors. In some embodiments, the viral vectors are retroviral vectors. In some embodiments, the viral vectors are adenoviral vectors.

### Adeno-Associated Viral (AAVs) Vectors

Viruses of the Parvoviridae family are small non-enveloped icosahedral capsid viruses characterized by a single stranded DNA genome. Parvoviridae family viruses consist of two subfamilies: Parvovirinae, which infect vertebrates, and Densovirinae, which infect invertebrates. Due to its relatively simple structure, easily manipulated using standard molecular biology techniques, this virus family is useful as a biological tool. The genome of the virus may be modified to contain a minimum of components for the assembly of a functional recombinant virus, or viral particle, which is loaded with or engineered to express or deliver a desired payload, which may be delivered to a target cell, tissue, organ, or organism.

The Parvoviridae family comprises the Dependovirus genus which includes adeno-associated viruses (AAV) capable of replication in vertebrate hosts including, but not limited to, human, primate, bovine, canine, equine, and ovine species.

The AAV vector genome is a linear, single-stranded DNA (ssDNA) molecule approximately 5,000 nucleotides (nts) in length. The AAV vector genome can comprise a payload region and at least one inverted terminal repeat (ITR) or ITR region. ITRs traditionally flank the coding nucleotide sequences for the non-structural proteins (encoded by Rep genes) and the structural proteins (encoded by capsid genes or Cap genes). While not wishing to be bound by theory, an AAV vector genome typically comprises two ITR sequences. The AAV vector genome comprises a characteristic T-shaped hairpin structure defined by the self-complementary terminal 145 nucleotides of the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

In addition to the encoded heterologous payload, AAV vector genomes may comprise, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entirety.

In some embodiments, the AAV vector genome comprises at least one control element which provides for the replication, transcription, and translation of a coding sequence encoded therein. Not all of the control elements need always be present as long as the coding sequence is capable of being replicated, transcribed, and/or translated in an appropriate host cell. Non-limiting examples of expression control elements include sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

AAV vector genomes of the present invention may be produced recombinantly and may be based on adeno-associated virus (AAV) parent or reference sequences. As used herein, a “vector genome” is any molecule or moiety which transports, transduces, or otherwise acts as a carrier of a heterologous molecule such as the nucleic acids described herein.

In addition to single stranded AAV vector genomes (e.g., ssAAVs), the present invention also provides for self-complementary AAV (scAAVs) vector genomes. scAAV vector genomes contain DNA strands which anneal together to form double stranded DNA. By skipping second strand synthesis, scAAVs allow for rapid expression in the cell.

In some embodiments, the AAV vector genome is an scAAV.

In some embodiments, the AAV vector genome is an ssAAV.

In some embodiments, the AAV vector genome may be part of an AAV particles where the serotype of the capsid may be, but is not limited to, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr—B7.3, AAV CBr—B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, PHP.B, PUPA, G2B-26, G2B-13, TH1.1-32, and/or TH1.1-35 and variants thereof.

### Inverted Terminal Repeats (ITRs)

In some embodiments, the AAV vector genomes may comprise at least one ITR region and a payload region. In some embodiments, the vector genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into vector genomes of the invention may be comprised of naturally occurring polynucleotide sequences or recombinantly derived polynucleotide sequences.

The ITRs may be derived from the same serotype as the capsid or a derivative thereof. The ITR may be of a different serotype than the capsid. In some embodiments, the AAV particle has more than one ITR. In a non-limiting example, the AAV particle has a vector genome comprising two ITRs. In some embodiments, the ITRs are of the same serotype as one another. In another embodiment, the ITRs are of different serotypes. Non-limiting examples include zero, one or both of the ITRs having the same serotype as the capsid. In some embodiments both ITRs of the vector genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In some embodiments, the ITRs are 140-142 nucleotides in length. Non-limiting examples of ITR length are 102, 140, 141, 142, 145 nucleotides in length, and those having at least 95% identity thereto.

### Promoters

In some embodiments, the payload region of the vector genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety). Non-limiting examples of elements to enhance the transgene target specificity and expression include promoters, endogenous miRNAs, post-transcriptional regulatory elements (PREs), polyadenylation (PolyA) signal sequences and upstream enhancers (USEs), CMV enhancers and introns.

In some embodiments, the promoter is efficient when it drives expression of the polypeptide(s) encoded in the payload region of the vector genome of the AAV particle.

In some embodiments, the promoter is deemed to be efficient when it drives expression in the cell being targeted.

In some embodiments, the promoter drives expression of the payload for a period of time in targeted tissues. Expression driven by a promoter may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years, or 5-10 years.

In some embodiment, the promoter drives expression of the payload for at least 1 month, 2 months, 3 months, 4 months, 5 months, 6 months, 7 months, 8 months, 9 months, 10 months, 11 months, 1 year, 2 years, 3 years 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years.

Promoters may be naturally occurring or non-naturally occurring. Non-limiting examples of promoters include viral promoters, plant promoters and mammalian promoters. In some embodiments, the promoters may be human promoters. In some embodiments, the promoter may be truncated.

Promoters which drive or promote expression in most tissues include, but are not limited to, human elongation factor 1α-subunit (EF1α), cytomegalovirus (CMV) immediate-early enhancer and/or promoter, chicken β-actin (CBA) and its derivative CAG, R glucuronidase (GUSB), or ubiquitin C (UBC). Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of muscle-specific promoters include mammalian muscle creatine kinase (MCK) promoter, mammalian desmin (DES) promoter, mammalian troponin I (TNNI2) promoter, and mammalian skeletal alpha-actin (ASKA) promoter (see, e.g. U.S. Patent Publication US20110212529, the contents of which are herein incorporated by reference in their entirety)

Non-limiting examples of tissue-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca²⁺/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter.

In some embodiments, the promoter may be less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800, or 700-800.

In some embodiments, the promoter may be a combination of two or more components of the same or different starting or parental promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or more than 800. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. In some embodiments, the promoter is a combination of a 382 nucleotide CMV-enhancer sequence and a 260 nucleotide CBA-promoter sequence.

In some embodiments, the vector genome comprises a ubiquitous promoter. Non-limiting examples of ubiquitous promoters include CMV, CBA (including derivatives CAG, CBh, etc.), EF-1α, PGK, UBC, GUSB (hGBp), and UCOE (promoter of HNRPA2B1-CBX3).

In some embodiments, the promoter is not cell specific.

In some embodiments, the vector genome comprises an engineered promoter.

In some embodiments, the vector genome comprises a promoter from a naturally expressed protein.

### Untranslated Regions (UTRs)

By definition, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs may be engineered into UTRs to enhance the stability and protein production. As a non-limiting example, a 5′ UTR from mRNA normally expressed in the liver (e.g., albumin, serum amyloid A, Apolipoprotein A/B/E, transferrin, alpha fetoprotein, erythropoietin, or Factor VIII) may be used in the vector genomes of the AAV particles of the invention to enhance expression in hepatic cell lines or liver.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In some embodiments, the 5′UTR in the vector genome includes a Kozak sequence.

In some embodiments, the 5′UTR in the vector genome does not include a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-a, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, e.g., payload regions of vector genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some embodiments, the 3′ UTR of the vector genome may include an oligo(dT) sequence for templated addition of a poly-A tail.

In some embodiments, the vector genome may include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence comprises a “seed” region, i.e., a sequence in the region of positions 2-8 of the mature microRNA, which sequence has perfect Watson-Crick complementarity to the miRNA target sequence of the nucleic acid.

In some embodiments, the vector genome may be engineered to include, alter or remove at least one miRNA binding site, sequence, or seed region.

Any UTR from any gene known in the art may be incorporated into the vector genome of the AAV particle. These UTRs, or portions thereof, may be placed in the same orientation as in the gene from which they were selected or they may be altered in orientation or location. In some embodiments, the UTR used in the vector genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In some embodiments, the vector genome of the AAV particle comprises at least one artificial UTRs which is not a variant of a wild-type UTR.

In some embodiments, the vector genome of the AAV particle comprises UTRs which have been selected from a family of transcripts whose proteins share a common function, structure, feature or property.

### Polyadenylation Sequence

In some embodiments, the vector genome comprises at least one polyadenylation sequence between the 3′ end of the payload coding sequence and the 5′ end of the 3′ITR.

In some embodiments, the polyadenylation (poly-A) sequence may range from absent to about 500 nucleotides in length. The polyadenylation sequence may be, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, and 500 nucleotides in length.

In some embodiments, the polyadenylation sequence is 50-100 nucleotides in length. In some embodiments, the polyadenylation sequence is 50-150 nucleotides in length. In some embodiments, the polyadenylation sequence is 50-160 nucleotides in length. In some embodiments, the polyadenylation sequence is 50-200 nucleotides in length. In some embodiments, the polyadenylation sequence is 60-100 nucleotides in length. In some embodiments, the polyadenylation sequence is 60-150 nucleotides in length. In some embodiments, the polyadenylation sequence is 60-160 nucleotides in length. In some embodiments, the polyadenylation sequence is 60-200 nucleotides in length. In some embodiments, the polyadenylation sequence is 70-100 nucleotides in length. In some embodiments, the polyadenylation sequence is 70-150 nucleotides in length. In some embodiments, the polyadenylation sequence is 70-160 nucleotides in length. In some embodiments, the polyadenylation sequence is 70-200 nucleotides in length. In some embodiments, the polyadenylation sequence is 80-100 nucleotides in length. In some embodiments, the polyadenylation sequence is 80-150 nucleotides in length. In some embodiments, the polyadenylation sequence is 80-160 nucleotides in length. In some embodiments, the polyadenylation sequence is 80-200 nucleotides in length. In some embodiments, the polyadenylation sequence is 90-100 nucleotides in length. In some embodiments, the polyadenylation sequence is 90-150 nucleotides in length. In some embodiments, the polyadenylation sequence is 90-160 nucleotides in length. In some embodiments, the polyadenylation sequence is 90-200 nucleotides in length.

### Linkers

Vector genomes may be engineered with one or more spacer or linker regions to separate coding or non-coding regions.

In some embodiments, the payload region of the vector genome may optionally encode one or more linker sequences. In some cases, the linker may be a peptide linker that may be used to connect the polypeptides encoded by the payload region (i.e., light and heavy antibody chains during expression). Some peptide linkers may be cleaved after expression to separate heavy and light chain domains, allowing assembly of mature antibodies or antibody fragments. Linker cleavage may be enzymatic. In some cases, linkers comprise an enzymatic cleavage site to facilitate intracellular or extracellular cleavage. Some payload regions encode linkers that interrupt polypeptide synthesis during translation of the linker sequence from an mRNA transcript. Such linkers may facilitate the translation of separate protein domains from a single transcript. In some cases, two or more linkers are encoded by a payload region of the vector genome.

Internal ribosomal entry site (IRES) is a nucleotide sequence (>500 nucleotides) that allows for initiation of translation in the middle of an mRNA sequence (Kim, J. H. et al., 2011. PLoS One 6(4): e18556; the contents of which are herein incorporated by reference in its entirety). Use of an IRES sequence ensures co-expression of genes before and after the IRES, though the sequence following the IRES may be transcribed and translated at lower levels than the sequence preceding the IRES sequence.

2A peptides are small “self-cleaving” peptides (18-22 amino acids) derived from viruses such as foot-and-mouth disease virus (F2A), porcine teschovirus-1 (P2A), Thosea asigna virus (T2A), or equine rhinitis A virus (E2A). The 2A designation refers specifically to a region of picornavirus polyproteins that lead to a ribosomal skip at the glycyl-prolyl bond in the C-terminus of the 2A peptide (Kim, J. H. et al., 2011. PLoS One 6(4): e18556; the contents of which are herein incorporated by reference in its entirety). This skip results in a cleavage between the 2A peptide and its immediate downstream peptide. As opposed to IRES linkers, 2A peptides generate stoichiometric expression of proteins flanking the 2A peptide and their shorter length can be advantageous in generating viral expression vectors.

Some payload regions encode linkers comprising furin cleavage sites. Furin is a calcium dependent serine endoprotease that cleaves proteins just downstream of a basic amino acid target sequence (Arg-X-(Arg/Lys)-Arg) (Thomas, G., 2002. Nature Reviews Molecular Cell Biology 3(10): 753-66; the contents of which are herein incorporated by reference in its entirety). Furin is enriched in the trans-golgi network where it is involved in processing cellular precursor proteins. Furin also plays a role in activating a number of pathogens. This activity can be taken advantage of for expression of polypeptides of the invention.

In some embodiments, the payload region may encode one or more linkers comprising cathepsin, matrix metalloproteinases or legumain cleavage sites. Such linkers are described e.g. by Cizeau and Macdonald in International Publication No. WO2008052322, the contents of which are herein incorporated in their entirety. Cathepsins are a family of proteases with unique mechanisms to cleave specific proteins. Cathepsin B is a cysteine protease and cathepsin D is an aspartyl protease. Matrix metalloproteinases are a family of calcium-dependent and zinc-containing endopeptidases. Legumain is an enzyme catalyzing the hydrolysis of (-Asn-Xaa-) bonds of proteins and small molecule substrates.

In some embodiments, payload regions may encode linkers that are not cleaved. Such linkers may include a simple amino acid sequence, such as a glycine rich sequence. In some cases, linkers may comprise flexible peptide linkers comprising glycine and serine residues. The linker may comprise flexible peptide linkers of different lengths, e.g., (G4S)ₙ, where n=1-10 ((Gly-Gly-Gly-Gly-Ser)ₙ) and the length of the encoded linker varies between 5 and 50 amino acids. In a non-limiting example, the linker may be (G4S)₅ (Gly-Gly-Gly-Gly-Ser)5. These flexible linkers are small and without side chains so they tend not to influence secondary protein structure while providing a flexible linker between antibody segments (George, R. A., et al., 2002. Protein Engineering 15(11): 871-9; Huston, J. S. et al., 1988. PNAS 85:5879-83; and Shan, D. et al., 1999. Journal of Immunology. 162(11):6589-95; the contents of each of which are herein incorporated by reference in their entirety). Furthermore, the polarity of the serine residues improves solubility and prevents aggregation problems.

In some embodiments, payload regions of the invention may encode small and unbranched serine-rich peptide linkers, such as those described by Huston et al. in U.S. Pat. No. 5,525,491, the contents of which are herein incorporated in their entirety. Polypeptides encoded by the payload region of the invention, linked by serine-rich linkers, have increased solubility.

In some embodiments, payload regions of the invention may encode artificial linkers, such as those described by Whitlow and Filpula in U.S. Pat. No. 5,856,456 and Ladner et al. in U.S. Pat. No. 4,946,778, the contents of each of which are herein incorporated by their entirety.

### Introns

In some embodiments, the payload region comprises at least one element to enhance the expression such as one or more introns or portions thereof. Non-limiting examples of introns include, MVM (67-97 bps), FIX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In some embodiments, the intron or intron portion may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400,410,420,430,440, 450, 460, 470, 480, 490 or 500. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500.

### Lentiviral Vectors

Lentiviral vectors are a type of retrovirus that can infect both dividing and nondividing cells because their viral shell can pass through the intact membrane of the nucleus of the target cell. Lentiviral vectors have the ability to deliver transgenes in tissues that had long appeared irremediably refractory to stable genetic manipulation. Lentivectors have also opened fresh perspectives for the genetic treatment of a wide array of hereditary as well as acquired disorders, and a real proposal for their clinical use seems imminent.

### RNA

Ribonucleic acid (RNA) is a molecule that is made up of nucleotides, which are ribose sugars attached to nitrogenous bases and phosphate groups. The nitrogenous bases include adenine (A), guanine (G), uracil (U), and cytosine (C). Generally, RNA mostly exists in the single-stranded form but can also exists double-stranded in certain circumstances. The length, form and structure of RNA is diverse depending on the purpose of the RNA. For example, the length of an RNA can vary from a short sequence (e.g., siRNA) to a long sequences (e.g., lncRNA), can be linear (e.g., mRNA) or circular (e.g., oRNA), and can either be a coding (e.g., mRNA) or a non-coding (e.g., lncRNA) sequence.

In some embodiments, the payload region may be or encode a coding RNA.

In some embodiments, the payload region may be or encode a non-coding RNA.

In some embodiments, the payload region may be or encode both a coding and a non-coding RNA.

In some embodiments, the payload region comprises nucleic acid sequences encoding more than one cargo or payload.

In some embodiments, the payload region comprises a nucleic acid sequence to enhance the expression of a gene. As a non-limiting example, the nucleic acid sequence is a messenger RNA (mRNA). As another non-limiting example, the nucleic acid sequence is a circular RNA (oRNA).

In some embodiments, the payload region comprises a nucleic acid sequence to reduce or inhibit the expression of a gene. As a non-limiting example, the nucleic acid sequence is a small interfering RNA (siRNA) or a microRNA (miRNA)

Messenger RNA (mRNA)

In some embodiments, the originator constructs and/or benchmark constructs may be mRNA. As used herein, the term “messenger RNA” (mRNA) refers to any polynucleotide which encodes a target of interest and which is capable of being translated to produce the encoded target of interest in vitro, in vivo, in situ or ex vivo.

Generally, an mRNA molecule comprises at least a coding region, a 5′ untranslated region (UTR), a 3′ UTR, a 5′ cap and a poly-A tail. In some aspects, one or more structural and/or chemical modifications or alterations may be included in the RNA which can reduce the innate immune response of a cell in which the mRNA is introduced. As used herein, a “structural” feature or modification is one in which two or more linked nucleotides are inserted, deleted, duplicated, inverted or randomized in a nucleic acid without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”.

Generally, the shortest length of a region of the originator constructs and/or benchmark constructs can be the length of a nucleic acid sequence that is sufficient to encode for a dipeptide, a tripeptide, a tetrapeptide, a pentapeptide, a hexapeptide, a heptapeptide, an octapeptide, a nonapeptide, or a decapeptide. In another embodiment, the length may be sufficient to encode a peptide of 2-30 amino acids, e.g. 5-30, 10-30, 2-25, 5-25, 10-25, or 10-20 amino acids. The length may be sufficient to encode for a peptide of at least 11, 12, 13, 14, 15, 17, 20, 25 or 30 amino acids, or a peptide that is no longer than 40 amino acids, e.g. no longer than 35, 30, 25, 20, 17, 15, 14, 13, 12, 11 or 10 amino acids.

Generally, the length of the region of the mRNA encoding a target of interest is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides).

In some embodiments, the mRNA includes from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

In some embodiments, the region or regions flanking the region encoding the target of interest may range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides).

In some embodiments, the mRNA comprises a tailing sequence which can range from absent to 500 nucleotides in length (e.g., at least 60, 70, 80, 90, 120, 140, 160, 180, 200, 250, 300, 350, 400, 450, or 500 nucleotides). Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional.

In some embodiments, the mRNA comprises a capping sequence which comprises a single cap or a series of nucleotides forming the cap. The capping sequence may be from 1 to 10, e.g. 2-9, 3-8, 4-7, 1-5, 5-10, or at least 2, or 10 or fewer nucleotides in length. In some embodiments, the caping sequence is absent.

In some embodiments, the mRNA comprises a region comprising a start codon. The region comprising the start codon may range from 3 to 40, e.g., 5-30, 10-20, 15, or at least 4, or 30 or fewer nucleotides in length.

In some embodiments, the mRNA comprises a region comprising a stop codon. The region comprising the stop codon may range from 3 to 40, e.g., 5-30, 10-20, 15, or at least 4, or 30 or fewer nucleotides in length.

In some embodiments, the mRNA comprises a region comprising a restriction sequence. The region comprising the restriction sequence may range from 3 to 40, e.g., 5-30, 10-20, 15, or at least 4, or 30 or fewer nucleotides in length.

### Untranslated Regions (UTRs)

In some embodiments, the mRNA comprises at least one untranslated region (UTR) which flanks the region encoding the target of interest. UTRs are transcribed by not translated.

The 5′ UTR starts at the transcription start site and continues to the start codon but does not include the start codon; whereas, the 3 'UTR starts immediately following the stop codon and continues until the transcriptional termination signal. While not wishing to be bound by theory, the UTRs may have a regulatory role in terms of translation and stability of the nucleic acid.

Natural 5′ UTRs usually include features which have a role in translation initiation as they tend to include Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

3′ UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo. Introduction, removal or modification of 3′ UTR AU rich elements (AREs) can be used to modulate the stability of mRNA. For example, one or more copies of an ARE can be introduced to make mRNA less stable and thereby curtail translation and decrease production of the resultant protein. Alternatively, AREs can be identified and removed or mutated to increase the intracellular stability and thus increase translation and production of the resultant protein.

In some embodiments, the introduction of features often expressed in genes of target organs the stability and protein production of the mRNA can be enhanced in a specific organ and/or tissue. As a non-limiting example, the feature can be a UTR. As another example, the feature can be introns or portions of introns sequences.

### 5′ Capping

The 5′ cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. The ribose sugars of the terminal and/or ante terminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

Modifications to mRNA may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap.

Additional modified guanosine nucleotides may be used such as a-methyl-phosphonate and seleno-phosphate nucleotides.

Additional modifications include, but are not limited to, 2′-O-methylation of the ribose sugars of 5′-terminal and/or 5′-ante terminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/or linked to a nucleic acid molecule.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which may equivalently be designated 3′ O-Me-m7G(5′)ppp(5′)G). The 3′-0 atom of the other, unmodified, guanine becomes linked to the 5′-terminal nucleotide of the capped nucleic acid molecule (e.g. an mRNA). The N7- and 3′-O-methylated guanine provides the terminal moiety of the capped nucleic acid molecule (e.g. mRNA).

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

While cap analogs allow for the concomitant capping of a nucleic acid molecule in an in vitro transcription reaction, up to 20% of transcripts can remain uncapped. This, as well as the structural differences of a cap analog from an endogenous 5′-cap structures of nucleic acids produced by the endogenous, cellular transcription machinery, may lead to reduced translational competency and reduced cellular stability.

mRNA may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′ cap structures are those which, among other things, have enhanced binding of cap binding proteins, increased half-life, reduced susceptibility to 5′ endonucleases and/or reduced 5′ decapping, as compared to synthetic 5′ cap structures known in the art (or to a wild-type, natural or physiological 5′ cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2′-O-methyl. Such a structure is termed the Capl structure. This cap results in a higher translational-competency and cellular stability and a reduced activation of cellular pro-inflammatory cytokines, as compared, e.g., to other 5′ cap analog structures known in the art. Cap structures include, but are not limited to, 7 mG(5*)ppp(5*)N,pN2p (cap 0), 7 mG(5*)ppp(5*)NlmpNp (cap 1), and 7 mG(5*)-ppp(5′)NlmpN2mp (cap 2).

In some embodiments, the 5′ terminal caps may include endogenous caps or cap analogs.

In some embodiments, a 5′ terminal cap may comprise a guanine analog. Useful guanine analogs include, but are not limited to, inosine, N1-methyl-guanosine, 2′ fluoro-guanosine, 7-deaza-guanosine, 8-oxo-guanosine, 2-amino-guanosine, LNA-guanosine, and 2-azido-guanosine.

### IRES Sequences

In some embodiments, the mRNA may contain an internal ribosome entry site (IRES). First identified as a feature Picorna virus RNA, IRES plays an important role in initiating protein synthesis in absence of the 5′ cap structure. An IRES may act as the sole ribosome binding site, or may serve as one of multiple ribosome binding sites of an mRNA. An mRNA that contains more than one functional ribosome binding site may encode several peptides or polypeptides that are translated independently by the ribosomes. Non-limiting examples of IRES sequences that can be used include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

### Poly-A Tails

During RNA processing, a long chain of adenine nucleotides (poly-A tail) may be added to a polynucleotide such as an mRNA molecules in order to increase stability. Immediately after transcription, the 3′ end of the transcript may be cleaved to free a 3′ hydroxyl. Then poly-A polymerase adds a chain of adenine nucleotides to the R A. The process, called polyadenylation, adds a poly-A tail of a certain length.

In some embodiments, the length of a poly-A tail is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the mRNA includes a poly-A tail from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall mRNA. This design may be based on the length of the region coding for a target of interest, the length of a particular feature or region (such as a flanking region), or based on the length of the ultimate product expressed from the mRNA.

In this context the poly-A tail may be 10, 20, 30, 40, 50, 60, 70, 80, 90, or 100% greater in length than the mRNA or feature thereof. The poly-A tail may also be designed as a fraction of mRNA to which it belongs. In this context, the poly-A tail may be 10, 20, 30, 40, 50, 60, 70, 80, or 90% or more of the total length of the construct or the total length of the construct minus the poly-A tail. Further, engineered binding sites and conjugation of mRNA for poly-A binding protein may enhance expression.

Additionally, multiple distinct mRNA may be linked together to the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the mRNA are designed to include a polyA-G Quartet. The G-quartet is a cyclic hydrogen bonded array of four guanine nucleotides that can be formed by G-rich sequences in both DNA and RNA. In this embodiment, the G-quartet is incorporated at the end of the poly-A tail.

### Stop Codons

In some embodiments, the mRNA may include one stop codon. In some embodiments, the mRNA may include two stop codons. In some embodiments, the mRNA may include three stop codons. In some embodiments, the mRNA may include at least one stop codon. In some embodiments, the mRNA may include at least two stop codons. In some embodiments, the mRNA may include at least three stop codons. As non-limiting examples, the stop codon may be selected from TGA, TAA and TAG.

In some embodiments, the mRNA includes the stop codon TGA and one additional stop codon. In a further embodiment the addition stop codon may be TAA.

Circular RNA (oRNA)

In some embodiments, the originator construct and/or the benchmark construct is a circular RNA (oRNA). As used herein, the terms “oRNA” or “circular RNA” are used interchangeably and can refer to a RNA that forms a circular structure through covalent or non-covalent bonds.

In some embodiments, the oRNA may be non-immunogenic in a mammal (e.g., a human, non-human primate, rabbit, rat, and mouse).

In some embodiments, the oRNA may be capable of replicating or replicates in a cell from an aquaculture animal (e.g., fish, crabs, shrimp, oysters etc.), a mammalian cell, a cell from a pet or zoo animal (e.g., cats, dogs, lizards, birds, lions, tigers and bears etc.), a cell from a farm or working animal (e.g., horses, cows, pigs, chickens etc.), a human cell, cultured cells, primary cells or cell lines, stem cells, progenitor cells, differentiated cells, germ cells, cancer cells (e.g., tumorigenic, metastatic), non-tumorigenic cells (e.g., normal cells), fetal cells, embryonic cells, adult cells, mitotic cells, non-mitotic cells, or any combination thereof.

In some embodiments, the oRNA has a half-life of at least that of a linear counterpart. In some embodiments, the oRNA has a half-life that is increased over that of a linear counterpart. In some embodiments, the half-life is increased by about 5%, 10%, 15%, 20%, 25%, 30%, 35%, 40%, 45%, 50%, or greater. In some embodiments, the oRNA has a half-life or persistence in a cell for at least about 1 hour to about 30 days, or at least about 2 hours, 6 hours, 12 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In some embodiments, the oRNA has a half-life or persistence in a cell for no more than about 10 mins to about 7 days, or no more than about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 24 hours (1 day), 36 hours (1.5 days), 48 hours (2 days), 60 hours (2.5 days), 72 hours (3 days), 4 days, 5 days, 6 days, or 7 days.

In some embodiments, the oRNA has a half-life or persistence in a cell while the cell is dividing. In some embodiments, the oRNA has a half-life or persistence in a cell post division. In certain embodiments, the oRNA has a half-life or persistence in a dividing cell for greater than about 10 minutes to about 30 days, or at least about 10 minutes, 15 minutes, 30 minutes, 45 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween.

In some embodiments, the oRNA modulates a cellular function, e.g., transiently or long term. In certain embodiments, the cellular function is stably altered, such as a modulation that persists for at least about 1 hour to about 30 days, or at least about 2 hours, 6 hours, 12 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer. In certain embodiments, the cellular function is transiently altered, e.g., such as a modulation that persists for no more than about 30 mins to about 7 days, or no more than about 30 minutes, 45 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours (1 day), 36 hours (1.5 days), 48 hours (2 days), 60 hours (2.5 days), 72 hours (3 days), 4 days, 5 days, 6 days, or 7 days.

In some embodiments, the oRNA is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the oRNA may be of a sufficient size to accommodate a binding site for a ribosome.

In some embodiments, the maximum size of the oRNA may be limited by the ability of packaging and delivering the RNA to a target. In some embodiments, the size of the oRNA is a length sufficient to encode polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 400 nucleotides, at least 300 nucleotides, at least 200 nucleotides, at least 100 nucleotides may be useful.

In some embodiments, the oRNA comprises one or more elements described elsewhere herein. In some embodiments, the elements may be separated from one another by a spacer sequence or linker. In some embodiments, the elements may be separated from one another by 1 nucleotide, 2 nucleotides, about 5 nucleotides, about 10 nucleotides, about 15 nucleotides, about 20 nucleotides, about 30 nucleotides, about 40 nucleotides, about 50 nucleotides, about 60 nucleotides, about 80 nucleotides, about 100 nucleotides, about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, about 1000 nucleotides, up to about 1 kb, at least about 1000 nucleotides.

In some embodiments, one or more elements are contiguous with one another, e.g., lacking a spacer element.

In some embodiments, one or more elements is conformationally flexible. In some embodiments, the conformational flexibility is due to the sequence being substantially free of a secondary structure.

In some embodiments, the oRNA comprises a secondary or tertiary structure that accommodates a binding site for a ribosome, translation, or rolling circle translation.

In some embodiments, the oRNA comprises particular sequence characteristics. For example, the oRNA may comprise a particular nucleotide composition. In some such embodiments, the oRNA may include one or more purine rich regions (adenine or guanosine). In some such embodiments, the oRNA may include one or more purine rich regions (adenine or guanosine). In some embodiments, the oRNA may include one or more AU rich regions or elements (AREs). In some embodiments, the oRNA may include one or more adenine rich regions.

In some embodiments, the oRNA comprises one or more modifications described elsewhere herein.

In some embodiments, the oRNA comprises one or more expression sequences and is configured for persistent expression in a cell of a subject in vivo. In some embodiments, the oRNA is configured such that expression of the one or more expression sequences in the cell at a later time point is equal to or higher than an earlier time point. In such embodiments, the expression of the one or more expression sequences can be either maintained at a relatively stable level or can increase over time. The expression of the expression sequences can be relatively stable for an extended period of time. For instance, in some cases, the expression of the one or more expression sequences in the cell over a time period of at least 7, 8, 9, 10, 12, 14, 16, 18, 20, 22, 23 or more days does not decrease by 50%, 45%, 40%, 35%, 30%, 25%, 20%, 15%, 10%, or 5%. In some cases, in some cases, the expression of the one or more expression sequences in the cell is maintained at a level that does not vary by more than 50%, 45%, 40%, 35%, 30%, 25%, 20%, 15%, 10%, or 5% for at least 7, 8, 9, 10, 12, 14, 16, 18, 20, 22, 23 or more days.

### Regulatory Elements

In some embodiments, the oRNA comprises a regulatory element. As used herein, a “regulatory element” is a sequence that modifies expression of an expression sequence. The regulatory element may include a sequence that is located adjacent to a payload or cargo region. The regulatory element may be operatively linked operatively to a payload or cargo region.

In some embodiments, a regulatory element may increase an amount of payload or cargo expressed as compared to an amount expressed when no regulatory element exists. As a non-limiting example, one regulatory element can increase an amount of payloads or cargos expressed for multiple payload or cargo sequences attached in tandem.

In some embodiments, a regulatory element may comprise a sequence to selectively initiates or activates translation of a payload or cargo.

In some embodiments, a regulatory element may comprise a sequence to initiate degradation of the oRNA or the payload or cargo. Non-limiting examples of the sequence to initiate degradation includes, but is not limited to, riboswitch aptazymes and miRNA binding sites.

In some embodiments, a regulatory element can modulate translation of the payload or cargo in the oRNA. The modulation can create an increase (enhancer) or decrease (suppressor) in the payload or cargo. The regulatory element may be located adjacent to the payload or cargo (e.g., on one side or both sides of the payload or cargo).

In some embodiments, a translation initiation sequence functions as a regulatory element. In some embodiments, the translation initiation sequence comprises an AUG/ATG codon. In some embodiments, a translation initiation sequence comprises any eukaryotic start codon such as, but not limited to, AUG/ATG, CUG/CTG, GUG/GTG, UUG/TTG, ACG, AUC/ATC, AUU, AAG, AUA/ATA, or AGG. In some embodiments, a translation initiation sequence comprises a Kozak sequence. In some embodiments, translation begins at an alternative translation initiation sequence, e.g., translation initiation sequence other than AUG/ATG codon, under selective conditions, e.g., stress induced conditions. As a non-limiting example, the translation of the circular polyribonucleotide may begin at alternative translation initiation sequence, such as ACG. As another non-limiting example, the circular polyribonucleotide translation may begin at alternative translation initiation sequence, CUG/CTG. As another non-limiting example, the translation may begin at alternative translation initiation sequence, GUG/GTG. As yet another non-limiting example, the translation may begin at a repeat-associated non-AUG (RAN) sequence, such as an alternative translation initiation sequence that includes short stretches of repetitive RNA e.g. CGG, GGGGCC, CAG, CTG.

### Masking Agents

Masking any of the nucleotides flanking a codon that initiates translation may be used to alter the position of translation initiation, translation efficiency, length and/or structure of the oRNA. In some embodiments, a masking agent may be used near the start codon or alternative start codon in order to mask or hide the codon to reduce the probability of translation initiation at the masked start codon or alternative start codon. Non-limiting examples of masking agents include antisense locked nucleic acids (LNA) oligonucleotides and exon junction complexes (EJCs). In some embodiments, a masking agent may be used to mask a start codon of the oRNA in order to increase the likelihood that translation will initiate at an alternative start codon.

### Translation Initiation Sequence

In some embodiments, the oRNA encodes a polypeptide or peptide and may comprise a translation initiation sequence. The translation initiation sequence may comprise, but is not limited to a start codon, a non-coding start codon, a Kozak sequence or a Shine-Dalgarno sequence. The translation initiation sequence may be located adjacent to the payload or cargo (e.g., on one side or both sides of the payload or cargo).

In some embodiments, the translation initiation sequence provides conformational flexibility to the oRNA. In some embodiments, the translation initiation sequence is within a substantially single stranded region of the oRNA.

The oRNA may include more than 1 start codon such as, but not limited to, at least 2, at least 3, at least 4, at least 5, at least 6, at least 7, at least 8, at least 9, at least 10, at least 11, at least 12, at least 13, at least 14, at least 15 or more than 15 start codons. Translation may initiate on the first start codon or may initiate downstream of the first start codon.

In some embodiments, the oRNA may initiate at a codon which is not the first start codon, e.g., AUG. Translation of the circular polyribonucleotide may initiate at an alternative translation initiation sequence, such as, but not limited to, ACG, AGG, AAG, CUG/CTG, GUG/GTG, AUA/ATA, AUU/ATT, UUG/TTG. In some embodiments, translation begins at an alternative translation initiation sequence under selective conditions, e.g., stress induced conditions. As a non-limiting example, the translation of the oRNA may begin at alternative translation initiation sequence, such as ACG. As another non-limiting example, the oRNA translation may begin at alternative translation initiation sequence, CUG/CTG. As yet another non-limiting example, the oRNA translation may begin at alternative translation initiation sequence, GTG/GUG. As yet another non-limiting example, the oRNA may begin translation at a repeat-associated non-AUG (RAN) sequence, such as an alternative translation initiation sequence that includes short stretches of repetitive RNA e.g. CGG, GGGGCC, CAG, CTG.

### IRES Sequences

In some embodiments, the oRNA described herein comprises an internal ribosome entry site (IRES) element capable of engaging an eukaryotic ribosome. In some embodiments, the IRES element is at least about 5 nucleotides, at least about 8 nucleotides, at least about 9 nucleotides, at least about 10 nucleotides, at least about 15 nucleotides, at least about 20 nucleotides, at least about 25 nucleotides, at least about 30 nucleotides, at least about 40 nucleotides, at least about 50 nucleotides, at least about 100 nucleotides, at least about 200 nucleotides, at least about 250 nucleotides, at least about 350 nucleotides, or at least about 500 nucleotides. In one embodiment, the IRES element is derived from the DNA of an organism including, but not limited to, a virus, a mammal, and a Drosophila. Such viral DNA may be derived from, but is not limited to, picornavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some embodiments, the IRES element is at least partially derived from a virus, for instance, it can be derived from a viral IRES element, such as ABPV_IGRpred, AEV, ALPV_IGRpred, BQCV_IGRpred, BVDV1_1-385, BVDV1_29-391, CrPV_5NCR, CrPV_IGR, crTMV_IREScp, crTMV_IRESmp75, crTMV_IRESmp228, crTMV_IREScp, crTMV_IREScp, CSFV, CVB3, DCV_IGR, EMCV-R, EoPV_5NTR, ERAV 245-961, ERBV 162-920, EV71_1-748, FeLV-Notch2, FMDV type_C, GBV-A, GBV-B, GBV-C, gypsy_env, gypsyD5, gypsyD2, HAV_HM175, HCV type_1a, HiPV_IGRpred, HIV-1, HoCV1_IGRpred, HRV-2, IAPV_IGRpred, idefix, KBV_IGRpred, LINE-1_ORF1_-101_to_-1, LINE-1_ORF1-302_to_-202, LINE-1_ORF2-138_to_-86, LINE-1_ORF1_-44 to_-1, PSIV_IGR, PV_type1_Mahoney, PV_type3_Leon, REV-A, RhPV_5NCR, RhPV_IGR, SINV1_IGRpred, SV40_661-830, TMEV, TMV_UI_IRESmp228, TRV_5NTR, TrV_IGR, or TSV_IGR. In some embodiments, the IRES element is at least partially derived from a cellular IRES, such as AML1/RUNX1, Antp-D, Antp-DE, Antp-CDE, Apaf-1, Apaf-1, AQP4, AT1R_var1, AT1R_var2, AT1R_var3, AT1R_var4, BAG1_p36delta236 nt, BAG1_p36, BCL2, BiP_-222_-3, c-IAP1_285-1399, c-IAP1_1313-1462, c-jun, c-myc, Cat-1224, CCND1, DAPS, eIF4G, eIF4GI-ext, eIF4GII, eIF4GII-long, ELG1, ELH, FGF1A, FMR1, Gtx-133-141, Gtx-1-166, Gtx-1-120, Gtx-1-196, hairless, HAP4, HIF1a, hSNM1, Hsp101, hsp70, hsp70, Hsp90, IGF2_leader2, Kv1.4_1.2, L-myc, LamB1_-335_-1, LEF1, MNT_75-267, MNT_36-160, MTG8a, MYB, MYT2_997-1152, n-MYC, NDST1, NDST2, NDST3, NDST4L, NDST4S, NRF_-653_-17, NtHSF1, ODC1, p27kip1, 03_128-269, PDGF2/c-sis, Pim-1, PITSLRE_p58, Rbm3, reaper, Scamper, TFIID, TIF4631, Ubx_1-966, Ubx_373-961, UNR, Ure2, UtrA, VEGF-A-133-1, XIAP_5-464, XIAP 305-466, or YAP1.

### Termination Element

In some embodiments, the oRNA includes one or more cargo or payload sequences (also referred to as expression sequences) and each cargo or payload sequence may or may not have a termination element.

In some embodiments, the oRNA includes one or more cargo or payload sequences and the sequences lack a termination element, such that the oRNA is continuously translated. Exclusion of a termination element may result in rolling circle translation or continuous expression of the encoded peptides or polypeptides as the ribosome will not stalling or fall-off. In such an embodiment, rolling circle translation expresses a continuous expression through each cargo or payload sequence.

In some embodiments, one or more cargo or payload sequences in the oRNA comprise a termination element.

In some embodiments, not all of the cargo or payload sequences in the oRNA comprise a termination element. In such instances, the cargo or payload may fall off the ribosome when the ribosome encounters the termination element and terminates translation. In some embodiments, translation is terminated while at least one region of the ribosome remains in contact with the oRNA.

### Rolling Circle Translation

In some embodiments, once translation of the oRNA is initiated, the ribosome bound to the oRNA does not disengage from the oRNA before finishing at least one round of translation of the oRNA. In some embodiments, the oRNA as described herein is competent for rolling circle translation. In some embodiments, during rolling circle translation, once translation of the oRNA is initiated, the ribosome bound to the oRNA does not disengage from the oRNA before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 10.sup.5 rounds, or at least 10.sup.6 rounds of translation of the oRNA.

In some embodiments, the rolling circle translation of the oRNA leads to generation of polypeptide that is translated from more than one round of translation of the oRNA. In some embodiments, the oRNA comprises a stagger element, and rolling circle translation of the oRNA leads to generation of polypeptide product that is generated from a single round of translation or less than a single round of translation of the oRNA.

### Circularization

In one embodiment, a linear RNA may be cyclized, or concatemerized. In some embodiments, the linear RNA may be cyclized in vitro prior to formulation and/or delivery. In some embodiments, the linear RNA may be cyclized within a cell.

In some embodiments, the mechanism of cyclization or concatemerization may occur through at least 3 different routes: 1) chemical, 2) enzymatic, and 3) ribozyme catalyzed. The newly formed 5′-/3′-linkage may be intramolecular or intermolecular.

In the first route, the 5′-end and the 3′-end of the nucleic acid contain chemically reactive groups that, when close together, form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a synthetic mRNA molecule will undergo a nucleophilic attack on the 5′-NHS-ester moiety forming a new 5′-/3′-amide bond.

In the second route, T4 RNA ligase may be used to enzymatically link a 5′-phosphorylated nucleic acid molecule to the 3′-hydroxyl group of a nucleic acid forming a new phosphorodiester linkage. In an example reaction, \hat{ }g of a nucleic acid molecule is incubated at 37° C. for 1 hour with 1-10 units of T4 RNA ligase (New England Biolabs, Ipswich, MA) according to the manufacturer's protocol. The ligation reaction may occur in the presence of a split oligonucleotide capable of base-pairing with both the 5′- and 3′-region in juxtaposition to assist the enzymatic ligation reaction.

In the third route, either the 5′- or 3′-end of the cDNA template encodes a ligase ribozyme sequence such that during in vitro transcription, the resultant nucleic acid molecule can contain an active ribozyme sequence capable of ligating the 5′-end of a nucleic acid molecule to the 3′-end of a nucleic acid molecule. The ligase ribozyme may be derived from the Group I Intron, Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In some embodiments, the oRNA is made via circularization of a linear RNA.

### Extracellular Circularization

In some embodiments, the linear RNA is cyclized, or concatemerized using a chemical method to form an oRNA. In some chemical methods, the 5′-end and the 3′-end of the nucleic acid (e.g., a linear RNA) includes chemically reactive groups that, when close together, may form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a linear RNA will undergo a nucleophilic attack on the 5′-NHS-ester moiety forming a new 5′-/3′-amide bond.

In one embodiment, a DNA or RNA ligase may be used to enzymatically link a 5′-phosphorylated nucleic acid molecule (e.g., a linear RNA) to the 3′-hydroxyl group of a nucleic acid (e.g., a linear nucleic acid) forming a new phosphorodiester linkage. In an example reaction, a linear RNA is incubated at 37C for 1 hour with 1-10 units of T4 RNA ligase according to the manufacturer's protocol. The ligation reaction may occur in the presence of a linear nucleic acid capable of base-pairing with both the 5′- and 3′-region in juxtaposition to assist the enzymatic ligation reaction. In one embodiment, the ligation is splint ligation where a single stranded polynucleotide (splint), like a single stranded RNA, can be designed to hybridize with both termini of a linear RNA, so that the two termini can be juxtaposed upon hybridization with the single-stranded splint. Splint ligase can thus catalyze the ligation of the juxtaposed two termini of the linear RNA, generating an oRNA.

In one embodiment, a DNA or RNA ligase may be used in the synthesis of the oRNA. As a non-limiting example, the ligase may be a circ ligase or circular ligase.

In one embodiment, either the 5′- or 3′-end of the linear RNA can encode a ligase ribozyme sequence such that during in vitro transcription, the resultant linear RNA includes an active ribozyme sequence capable of ligating the 5′-end of the linear RNA to the 3′-end of the linear RNA. The ligase ribozyme may be derived from the Group I Intron, Hepatitis Delta Virus, Hairpin ribozyme or may be selected by SELEX (systematic evolution of ligands by exponential enrichment).

In one embodiment, a linear RNA may be cyclized or concatemerized by using at least one non-nucleic acid moiety. In one aspect, the at least one non-nucleic acid moiety may react with regions or features near the 5′ terminus and/or near the 3′ terminus of the linear RNA in order to cyclize or concatermerize the linear RNA. In another aspect, the at least one non-nucleic acid moiety may be located in or linked to or near the 5′ terminus and/or the 3′ terminus of the linear RNA. The non-nucleic acid moieties contemplated may be homologous or heterologous. As a non-limiting example, the non-nucleic acid moiety may be a linkage such as a hydrophobic linkage, ionic linkage, a biodegradable linkage and/or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In one embodiment, a linear RNA may be cyclized or concatemerized due to a non-nucleic acid moiety that causes an attraction between atoms, molecular surfaces at, near or linked to the 5′ and 3′ ends of the linear RNA. As a non-limiting example, one or more linear RNA may be cyclized or concatemerized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In one embodiment, the linear RNA may comprise a ribozyme RNA sequence near the 5′ terminus and near the 3′ terminus. The ribozyme RNA sequence may covalently link to a peptide when the sequence is exposed to the remainder of the ribozyme. In one aspect, the peptides covalently linked to the ribozyme RNA sequence near the 5′ terminus and the 3′ terminus may associate with each other causing a linear RNA to cyclize or concatemerize. In another aspect, the peptides covalently linked to the ribozyme RNA near the 5′ terminus and the 3′ terminus may cause the linear RNA to cyclize or concatemerize after being subjected to ligated using various methods known in the art such as, but not limited to, protein ligation.

In some embodiments, the linear RNA may include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). Alternately, converting the 5′ triphosphate of the linear RNA into a 5′ monophosphate may occur by a two-step reaction comprising: (a) contacting the 5′ nucleotide of the linear RNA with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, RNA may be circularized using the methods described in WO2017222911 and WO2016197121, the contents of each of which are herein incorporated by reference in their entirety.

In some embodiments, RNA may be circularized, for example, by backsplicing of a non-mammalian exogenous intron or splint ligation of the 5′ and 3′ ends of a linear RNA. In one embodiment, the circular RNA is produced from a recombinant nucleic acid encoding the target RNA to be made circular. As a non-limiting example, the method comprises: a) producing a recombinant nucleic acid encoding the target RNA to be made circular, wherein the recombinant nucleic acid comprises in 5′ to 3′ order: i) a 3′ portion of an exogenous intron comprising a 3′ splice site, ii) a nucleic acid sequence encoding the target RNA, and iii) a 5′ portion of an exogenous intron comprising a 5′ splice site; b) performing transcription, whereby RNA is produced from the recombinant nucleic acid; and c) performing splicing of the RNA, whereby the RNA circularizes to produce a oRNA.

While not wishing to be bound by theory, circular RNAs generated with exogenous introns are recognized by the immune system as “non-self” and trigger an innate immune response. On the other hand, circular RNAs generated with endogenous introns are recognized by the immune system as “self” and generally do not provoke an innate immune response, even if carrying an exon comprising foreign RNA.

Accordingly, circular RNAs can be generated with either an endogenous or exogenous intron to control immunological self/nonself discrimination as desired. Numerous intron sequences from a wide variety of organisms and viruses are known and include sequences derived from genes encoding proteins, ribosomal RNA (rRNA), or transfer RNA (tRNA).

Circular RNAs can be produced from linear RNAs in a number of ways. In some embodiments, circular RNAs are produced from a linear RNA by backsplicing of a downstream 5′ splice site (splice donor) to an upstream 3′ splice site (splice acceptor). Circular RNAs can be generated in this manner by any nonmammalian splicing method. For example, linear RNAs containing various types of introns, including self-splicing group I introns, self-splicing group II introns, spliceosomal introns, and tRNA introns can be circularized. In particular, group I and group II introns have the advantage that they can be readily used for production of circular RNAs in vitro as well as in vivo because of their ability to undergo self-splicing due to their autocatalytic ribozyme activity.

In some embodiments, circular RNAs can be produced in vitro from a linear RNA by chemical or enzymatic ligation of the 5′ and 3′ ends of the RNA. Chemical ligation can be performed, for example, using cyanogen bromide (BrCN) or ethyl-3-(3′-dimethylaminopropyl) carbodiimide (EDC) for activation of a nucleotide phosphomonoester group to allow phosphodiester bond formation. See e.g., Sokolova (1988) FEBS Lett 232: 153-155; Dolinnaya et al. (1991) Nucleic Acids Res., 19:3067-3072; Fedorova (1996) Nucleosides Nucleotides Nucleic Acids 15: 1 137-1 147; herein incorporated by reference. Alternatively, enzymatic ligation can be used to circularize RNA. Exemplary ligases that can be used include T4 DNA ligase (T4 Dnl), T4 RNA ligase 1 (T4 Rnl 1), and T4 RNA ligase 2 (T4 Rnl 2).

In some embodiments, splint ligation using an oligonucleotide splint that hybridizes with the two ends of a linear RNA can be used to bring the ends of the linear RNA together for ligation. Hybridization of the splint, which can be either a DNA or a RNA, orientates the 5′-phosphate and 3′-OH of the RNA ends for ligation. Subsequent ligation can be performed using either chemical or enzymatic techniques, as described above. Enzymatic ligation can be performed, for example, with T4 DNA ligase (DNA splint required), T4 RNA ligase 1 (RNA splint required) or T4 RNA ligase 2 (DNA or RNA splint). Chemical ligation, such as with BrCN or EDC, in some cases is more efficient than enzymatic ligation if the structure of the hybridized splint-RNA complex interferes with enzymatic activity.

In some embodiments, the oRNA may further comprise an internal ribosome entry site (IRES) operably linked to an RNA sequence encoding a polypeptide. Inclusion of an IRES permits the translation of one or more open reading frames from a circular RNA. The IRES element attracts a eukaryotic ribosomal translation initiation complex and promotes translation initiation. See, e.g., Kaufman et al., Nuc. Acids Res. (1991) 19:4485-4490; Gurtu et al, Biochem. Biophys. Res. Comm. (1996) 229:295-298; Rees et al, BioTechniques (1996) 20: 102-110; Kobayashi et al., BioTechniques (1996) 21:399-402; and Mosser et al, BioTechniques 1997 22 150-161).

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization efficiency of the circularization methods provided herein is at least about 40%.

### Splicing Element

In some embodiment, the oRNA includes at least one splicing element. The splicing element can be a complete splicing element that can mediate splicing of the oRNA or the spicing element can be a residual splicing element from a completed splicing event. For instance, in some cases, a splicing element of a linear RNA can mediate a splicing event that results in circularization of the linear RNA, thereby the resultant oRNA comprises a residual splicing element from such splicing-mediated circularization event. In some cases, the residual splicing element is not able to mediate any splicing. In other cases, the residual splicing element can still mediate splicing under certain circumstances. In some embodiments, the splicing element is adjacent to at least one expression sequence. In some embodiments, the oRNA includes a splicing element adjacent each expression sequence. In some embodiments, the splicing element is on one or both sides of each expression sequence, leading to separation of the expression products, e.g., peptide(s) and or polypeptide(s).

In some embodiments, the oRNA includes an internal splicing element that when replicated the spliced ends are joined together. Some examples may include miniature introns (<100 nt) with splice site sequences and short inverted repeats (30-40 nt) such as AluSq2, AluJr, and AluSz, inverted sequences in flanking introns, Alu elements in flanking introns, and motifs found in (suptable4 enriched motifs) cis-sequence elements proximal to backsplice events such as sequences in the 200 bp preceding (upstream of) or following (downstream from) a backsplice site with flanking exons. In some embodiments, the oRNA includes at least one repetitive nucleotide sequence described elsewhere herein as an internal splicing element. In such embodiments, the repetitive nucleotide sequence may include repeated sequences from the Alu family of introns.

In some embodiments, the oRNA may include canonical splice sites that flank head-to-tail junctions of the oRNA.

In some embodiments, the oRNA may include a bulge-helix-bulge motif, comprising a 4-base pair stem flanked by two 3-nucleotide bulges. Cleavage occurs at a site in the bulge region, generating characteristic fragments with terminal 5′-hydroxyl group and 2′,3′-cyclic phosphate. Circularization proceeds by nucleophilic attack of the 5′—OH group onto the 2′,3′-cyclic phosphate of the same molecule forming a 3′,5′-phosphodiester bridge.

In some embodiments, the oRNA may include a sequence that mediates self-ligation. Non-limiting examples of sequences that can mediate self-ligation include a self-circularizing intron, e.g., a 5′ and 3′ slice junction, or a self-circularizing catalytic intron such as a Group I, Group II or Group III Introns. Non-limiting examples of group I intron self-splicing sequences may include self-splicing permuted intron-exon sequences derived from T4 bacteriophage gene td, and the intervening sequence (IVS) rRNA of Tetrahymena.

### Other Circularization Methods

In some embodiments, linear RNA may include complementary sequences, including either repetitive or nonrepetitive nucleic acid sequences within individual introns or across flanking introns. In some embodiments, the oRNA includes a repetitive nucleic acid sequence. In some embodiments, the repetitive nucleotide sequence includes poly CA or poly UG sequences. In some embodiments, the oRNA includes at least one repetitive nucleic acid sequence that hybridizes to a complementary repetitive nucleic acid sequence in another segment of the oRNA, with the hybridized segment forming an internal double strand. In some embodiments, repetitive nucleic acid sequences and complementary repetitive nucleic acid sequences from two separate oRNA that hybridize to generate a single oRNA, with the hybridized segments forming internal double strands. In some embodiments, the complementary sequences are found at the 5′ and 3′ ends of the linear RNA. In some embodiments, the complementary sequences include about 3, 4, 5,6,7,8,9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the oRNA. Such methods may include, but are not limited to click chemistry (e.g., alkyne and azide based methods, or clickable bases), olefin metathesis, phosphoramidate ligation, hemiaminal-imine crosslinking, base modification, and any combination thereof.

In some embodiments, enzymatic methods of circularization may be used to generate the oRNA. In some embodiments, a ligation enzyme, e.g., DNA or RNA ligase, may be used to generate a template of the oRNA or complement, a complementary strand of the oRNA, or the oRNA.

Small Interfering RNAs (siRNAs)

In some embodiments, the payload region may be or encode an RNA interference (RNAi) sequence which can be used to reduce or inhibit the expression of a gene. RNAi (also known as post-transcriptional gene silencing (PTGS), quelling, or co-suppression) is a post-transcriptional gene silencing process in which RNA molecules, in a sequence specific manner, reduce or inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2 nucleotide 3′ overhangs and that match the nucleic acid sequence of the target gene. These short RNA species may be naturally produced in vivo by Dicer-mediated cleavage of larger dsRNAs and they are functional in mammalian cells.

Naturally expressed small RNA molecules, named microRNAs (miRNAs), elicit gene silencing by regulating the expression of mRNAs. The miRNAs-containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA-mediated down-regulation of gene expression may be caused by cleavage of the target mRNAs, translational inhibition of the target mRNAs, or mRNA decay. miRNA targeting sequences are usually located in the 3′-UTR of the target mRNAs. A single miRNA may target more than 100 transcripts from various genes, and one mRNA may be targeted by different miRNAs.

siRNA duplexes or dsRNA targeting a specific mRNA may be designed and synthesized in vitro and introduced into cells for activating RNAi processes. It has been previously shown that 21-nucleotide siRNA duplexes (termed small interfering RNAs) were capable of effecting potent and specific gene knockdown without inducing immune response in mammalian cells. Now post-transcriptional gene silencing by siRNAs has quickly emerged as a powerful tool for genetic analysis in mammalian cells and has the potential to produce novel therapeutics.

In vitro synthetized siRNA sequences may be introduced into cells in order to activate RNAi. An exogenous siRNA duplex, when it is introduced into cells, similar to the endogenous dsRNAs, can be assembled to form the RNA Induced Silencing Complex (RISC), a multiunit complex that interacts with RNA sequences that are complementary to one of the two strands of the siRNA duplex (i.e., the antisense strand). During the process, the sense strand (or passenger strand) of the siRNA is lost from the complex, while the antisense strand (or guide strand) of the siRNA is matched with its complementary RNA. In particular, the targets of siRNA containing RISC complexes are mRNAs presenting a perfect sequence complementarity. Then, siRNA mediated gene silencing occurs by cleaving, releasing and degrading the target.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g. antisense strand RNA or antisense oligonucleotides). In many cases, it requires higher concentration of the ss-siRNA to achieve the effective gene silencing potency of the corresponding duplex.

Design and Sequences of siRNA Duplexes

Some guidelines for designing siRNAs have been proposed in the art. These guidelines generally recommend generating a 19-nucleotide duplexed region, symmetric 2-3 nucleotide 3′ overhangs, 5′-phosphate and 3′-hydroxyl groups targeting a region in the gene to be silenced. Other rules that may govern siRNA sequence preference include, but are not limited to, (i) A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such consideration, together with the specific sequence of a target gene, highly effective siRNA constructs essential for suppressing mammalian target gene expression may be readily designed.

In some embodiments, siRNA constructs (e.g., siRNA duplexes or encoded dsRNA) that target a specific gene are designed. Such siRNA constructs can specifically, suppress gene expression and protein production. In some aspects, the siRNA constructs are designed and used to selectively “knock out” gene variants in cells, i.e., mutated transcripts that are identified in patients or that are the cause of various diseases and/or disorders. In some aspects, the siRNA constructs are designed and used to selectively “knock down” variants of the gene in cells. In other aspects, the siRNA constructs are able to inhibit or suppress both the wild type and mutated versions of the gene.

In some embodiments, an siRNA sequence comprises a sense strand and a complementary antisense strand in which both strands are hybridized together to form a duplex structure. The antisense strand has sufficient complementarity to the mRNA sequence to direct target-specific RNAi, i.e., the siRNA sequence has a sequence sufficient to trigger the destruction of the target mRNA by the RNAi machinery or process.

In some embodiments, an siRNA sequence comprises a sense strand and a complementary antisense strand in which both strands are hybridized together to form a duplex structure and where the start site of the hybridization to the mRNA is between nucleotide 100 and 10,000 on the mRNA sequence. As a non-limiting example, the start site may be between nucleotide 100-150, 150-200, 200-250, 250-300, 300-350, 350-400, 400-450, 450-500, 500-550, 550-600, 600-650, 650-700, 700-70, 750-800, 800-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1900-1950, 1950-2000, 2000-2050, 2050-2100, 2100-2150, 2150-2200, 2200-2250, 2250-2300, 2300-2350, 2350-2400, 2400-2450, 2450-2500, 2500-2550, 2550-2600, 2600-2650, 2650-2700, 2700-2750, 2750-2800, 2800-2850, 2850-2900, 2900-2950, 2950-3000, 3000-3050, 3050-3100, 3100-3150, 3150-3200, 3200-3250, 3250-3300, 3300-3350, 3350-3400, 3400-3450, 3450-3500, 3500-3550, 3550-3600, 3600-3650, 3650-3700, 3700-3750, 3750-3800, 3800-3850, 3850-3900, 3900-3950, 3950-4000, 4000-4050, 4050-4100, 4100-4150, 4150-4200, 4200-4250, 4250-4300, 4300-4350, 4350-4400, 4400-4450, 4450-4500, 4500-4550, 4550-4600, 4600-4650, 4650-4700, 4700-4750, 4750-4800, 4800-4850, 4850-4900, 4900-4950, 4950-5000, 5000-5050, 5050-5100, 5100-5150, 5150-5200, 5200-5250, 5250-5300, 5300-5350, 5350-5400, 5400-5450, 5450-5500, 5500-5550, 5550-5600, 5600-5650, 5650-5700, 5700-5750, 5750-5800, 5800-5850, 5850-5900, 5900-5950, 5950-6000, 6000-6050, 6050-6100, 6100-6150, 6150-6200, 6200-6250, 6250-6300, 6300-6350, 6350-6400, 6400-6450, 6450-6500, 6500-6550, 6550-6600, 6600-6650, 6650-6700, 6700-6750, 6750-6800, 6800-6850, 6850-6900, 6900-6950, 6950-7000, 7000-7050, 7050-7100, 7100-7150, 7150-7200, 7200-7250, 7250-7300, 7300-7350, 7350-7400, 7400-7450, 7450-7500, 7500-7550, 7550-7600, 7600-7650, 7650-7700, 7700-7750, 7750-7800, 7800-7850, 7850-7900, 7900-7950, 7950-8000, 8000-8050, 8050-8100, 8100-8150, 8150-8200, 8200-8250, 8250-8300, 8300-8350, 8350-8400, 8400-8450, 8450-8500, 8500-8550, 8550-8600, 8600-8650, 8650-8700, 8700-8750, 8750-8800, 8800-8850, 8850-8900, 8900-8950, 8950-9000, 9000-9050, 9050-9100, 9100-9150, 9150-9200, 9200-9250, 9250-9300, 9300-9350, 9350-9400, 9400-9450, 9450-9500, 9500-9550, 9550-9600, 9600-9650, 9650-9700, 9700-9750, 9750-9800, 9800-9850, 9850-9900, 9900-9950, 9950-10000 on the mRNA sequence.

In some embodiments, the antisense strand and target mRNA sequences have 100% complementary. The antisense strand may be complementary to any part of the target mRNA sequence.

In other embodiments, the antisense strand and target mRNA sequences comprise at least one mismatch. As a non-limiting example, the antisense strand and the target mRNA sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementarity.

In some embodiments, the siRNA sequence has a length from about 10-50 or more nucleotides, i.e., each strand comprising 10-50 nucleotides (or nucleotide analogs). Preferably, the siRNA sequence has a length from about 15-30, e.g., 15, 16, 17, 18, 19, 20, 21, 22, 23, 24, 25, 26, 27, 28, 29, or 30 nucleotides in each strand, wherein one of the strands is sufficiently complementarity to a target region. In some embodiments, the siRNA sequence has a length from about 19 to 25, 19 to 24 or 19 to 21 nucleotides.

In some embodiments, the siRNA sequences can be synthetic RNA duplexes comprising about 19 nucleotides to about 25 nucleotides, and two overhanging nucleotides at the 3′-end. In some aspects, the siRNA constructs may be unmodified RNA molecules. In other aspects, the siRNA constructs may contain at least one modified nucleotide, such as base, sugar or backbone modifications.

In some embodiments, the siRNA sequences can be encoded in plasmid vectors, viral vectors or other nucleic acid expression vectors for delivery to a cell. DNA expression plasmids can be used to stably express the siRNA duplexes or dsRNA in cells and achieve long-term inhibition of the target gene expression. In one aspect, the sense and antisense strands of a siRNA duplex are typically linked by a short spacer sequence leading to the expression of a stem-loop structure termed short hairpin RNA (shRNA). The hairpin is recognized and cleaved by Dicer, thus generating mature siRNA constructs.

In some embodiments, the sense and antisense strands of a siRNA duplex may be linked by a short spacer sequence, which may optionally be linked to additional flanking sequence, leading to the expression of a flanking arm-stem-loop structure termed primary microRNA (pri-miRNA). The pri-miRNA may be recognized and cleaved by Drosha and Dicer, and thus generate mature siRNA constructs.

In some embodiments, the siRNA duplexes or encoded dsRNA suppress (or degrade) target mRNA. Accordingly, the siRNA duplexes or encoded dsRNA can be used to substantially inhibit gene expression in a cell. In some aspects, the inhibition of gene expression refers to an inhibition by at least about 20%, preferably by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, preferably by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the siRNA constructs comprise a miRNA seed match for the target located in the guide strand. In another embodiment, the siRNA constructs comprise a miRNA seed match for the target located in the passenger strand. In yet another embodiment, the siRNA duplexes or encoded dsRNA targeting gene do not comprise a seed match for the target located in the guide or passenger strand.

In some embodiments, the siRNA duplexes or encoded dsRNA targeting the gene may have almost no significant full-length off targets for the guide strand. In another embodiment, the siRNA duplexes or encoded dsRNA targeting the gene may have almost no significant full-length off target effects for the passenger strand. The siRNA duplexes or encoded dsRNA targeting the gene may have less than 1%, 2%, 3%, 4%, 5%, 6%, 7%, 8%, 9%, 10%, 11%, 12%, 13%, 14%, 15%, 20%, 25%, 30%, 35%, 40%, 45%, 50%, 1-5%, 2-6%, 3-7%, 4-8%, 5-9%, 5-10%, 6-10%, 5-15%, 5-20%, 5-25% 5-30%, 10-20%, 10-30%, 10-40%, 10-50%, 15-30%, 15-40%, 15-45%, 20-40%, 20-50%, 25-50%, 30-40%, 30-50%, 35-50%, 40-50%, 45-50% full-length off target effects for the passenger strand. In yet another embodiment, the siRNA duplexes or encoded dsRNA targeting the gene may have almost no significant full-length off targets for the guide strand or the passenger strand. The siRNA duplexes or encoded dsRNA targeting the gene may have less than 1%, 2%, 3%, 4%, 5%, 6%, 7%, 8%, 9%, 10%, 11%, 12%, 13%, 14%, 15%, 20%, 25%, 30%, 35%, 40%, 45%, 50%, 1-5%, 2-6%, 3-7%, 4-8%, 5-9%, 5-10%, 6-10%, 5-15%, 5-20%, 5-25% 5-30%, 10-20%, 10-30%, 10-40%, 10-50%, 15-30%, 15-40%, 15-45%, 20-40%, 20-50%, 25-50%, 30-40%, 30-50%, 35-50%, 40-50%, 45-50% full-length off target effects for the guide or passenger strand.

In some embodiments, the siRNA duplexes or encoded dsRNA targeting the gene may have high activity in vitro. In another embodiment, the siRNA constructs may have low activity in vitro. In yet another embodiment, the siRNA duplexes or dsRNA targeting the gene may have high guide strand activity and low passenger strand activity in vitro.

In some embodiments, the siRNA constructs have a high guide strand activity and low passenger strand activity in vitro. The target knock-down (KD) by the guide strand may be at least 40%, 50%, 60%, 65%, 70%, 75%, 80%, 85%, 90%, 95%, 99%, 99.5% or 100%. The target knock-down by the guide strand may be 40-50%, 45-50%, 50-55%, 50-60%, 60-65%, 60-70%, 60-75%, 60-80%, 60-85%, 60-90%, 60-95%, 60-99%, 60-99.5%, 60-100%, 65-70%, 65-75%, 65-80%, 65-85%, 65-90%, 65-95%, 65-99%, 65-99.5%, 65-100%, 70-75%, 70-80%, 70-85%, 70-90%, 70-95%, 70-99%, 70-99.5%, 70-100%, 75-80%, 75-85%, 75-90%, 75-95%, 75-99%, 75-99.5%, 75-100%, 80-85%, 80-90%, 80-95%, 80-99%, 80-99.5%, 80-100%, 85-90%, 85-95%, 85-99%, 85-99.5%, 85-100%, 90-95%, 90-99%, 90-99.5%, 90-100%, 95-99%, 95-99.5%, 95-100%, 99-99.5%, 99-100% or 99.5-100%. As a non-limiting example, the target knock-down (KD) by the guide strand is greater than 70%. As a non-limiting example, the target knock-down (KD) by the guide strand is greater than 60%.

In some embodiments, the guide to passenger (G:P) (also referred to as the antisense to sense) strand ratio expressed is 1:10, 1:9, 1:8, 1:7, 1:6, 1:5, 1:4, 1:3, 1:2, 1; 1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. The guide to passenger ratio refers to the ratio of the guide strands to the passenger strands after the intracellular processing of the pri-microRNA. For example, a 80:20 guide-to-passenger ratio would have 8 guide strands to every 2 passenger strands processed from the precursor. As a non-limiting example, the guide-to-passenger strand ratio is 8:2 in vitro. As a non-limiting example, the guide-to-passenger strand ratio is 8:2 in vivo. As a non-limiting example, the guide-to-passenger strand ratio is 9:1 in vitro. As a non-limiting example, the guide-to-passenger strand ratio is 9:1 in vivo.

In some embodiments, the guide to passenger (G:P) (also referred to as the antisense to sense) strand ratio expressed is greater than 1. In some embodiments, the guide to passenger (G:P) (also referred to as the antisense to sense) strand ratio expressed is greater than 2. In some embodiments, the guide to passenger (G:P) (also referred to as the antisense to sense) strand ratio expressed is greater than 5. In some embodiments, the guide to passenger (G:P) (also referred to as the antisense to sense) strand ratio expressed is greater than 10. In some embodiments, the guide to passenger (G:P) (also referred to as the antisense to sense) strand ratio expressed is greater than 20. In some embodiments, the guide to passenger (G:P) (also referred to as the antisense to sense) strand ratio expressed is greater than 50. In some embodiments, the guide to passenger (G:P) (also referred to as the antisense to sense) strand ratio expressed is at least 3:1. In some embodiments, the guide to passenger (G:P) (also referred to as the antisense to sense) strand ratio expressed is at least 5:1. In some embodiments, the guide to passenger (G:P) (also referred to as the antisense to sense) strand ratio expressed is at least 10:1. In some embodiments, the guide to passenger (G:P) (also referred to as the antisense to sense) strand ratio expressed is at least 20:1. In some embodiments, the guide to passenger (G:P) (also referred to as the antisense to sense) strand ratio expressed is at least 50:1.

In some embodiments, the passenger to guide (P:G) (also referred to as the sense to antisense) strand ratio expressed is 1:10, 1:9, 1:8, 1:7, 1:6, 1:5, 1:4, 1:3, 1:2, 1; 1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. The passenger to guide ratio refers to the ratio of the passenger strands to the guide strands after the excision of the guide strand. For example, a 80:20 passenger to guide ratio would have 8 passenger strands to every 2 guide strands processed from the precursor. As a non-limiting example, the passenger-to-guide strand ratio is 80:20 in vitro. As a non-limiting example, the passenger-to-guide strand ratio is 80:20 in vivo. As a non-limiting example, the passenger-to-guide strand ratio is 8:2 in vitro. As a non-limiting example, the passenger-to-guide strand ratio is 8:2 in vivo. As a non-limiting example, the passenger-to-guide strand ratio is 9:1 in vitro. As a non-limiting example, the passenger-to-guide strand ratio is 9:1 in vivo.

In some embodiments, the passenger to guide (P:G) (also referred to as the sense to antisense) strand ratio expressed is greater than 1. In some embodiments, the passenger to guide (P:G) (also referred to as the sense to antisense) strand ratio expressed is greater than 2. In some embodiments, the passenger to guide (P:G) (also referred to as the sense to antisense) strand ratio expressed is greater than 5. In some embodiments, the passenger to guide (P:G) (also referred to as the sense to antisense) strand ratio expressed is greater than 10. In some embodiments, the passenger to guide (P:G) (also referred to as the sense to antisense) strand ratio expressed is greater than 20. In some embodiments, the passenger to guide (P:G) (also referred to as the sense to antisense) strand ratio expressed is greater than 50. In some embodiments, the passenger to guide (P:G) (also referred to as the sense to antisense) strand ratio expressed is at least 3:1. In some embodiments, the passenger to guide (P:G) (also referred to as the sense to antisense) strand ratio expressed is at least 5:1. In some embodiments, the passenger to guide (P:G) (also referred to as the sense to antisense) strand ratio expressed is at least 10:1. In some embodiments, the passenger to guide (P:G) (also referred to as the sense to antisense) strand ratio expressed is at least 20:1. In some embodiments, the passenger to guide (P:G) (also referred to as the sense to antisense) strand ratio expressed is at least 50:1.

In some embodiments, a passenger-guide strand duplex is considered effective when the pri- or pre-microRNAs demonstrate, but methods known in the art and described herein, greater than 2-fold guide to passenger strand ratio when processing is measured. As a non-limiting examples, the pri- or pre-microRNAs demonstrate great than 2-fold, 3-fold, 4-fold, 5-fold, 6-fold, 7-fold, 8-fold, 9-fold, 10-fold, 11-fold, 12-fold, 13-fold, 14-fold, 15-fold, or 2 to 5-fold, 2 to 10-fold, 2 to 15-fold, 3 to 5-fold, 3 to 10-fold, 3 to 15-fold, 4 to 5-fold, 4 to 10-fold, 4 to 15-fold, 5 to 10-fold, 5 to 15-fold, 6 to 10-fold, 6 to 15-fold, 7 to 10-fold, 7 to 15-fold, 8 to 10-fold, 8 to 15-fold, 9 to 10-fold, 9 to 15-fold, 10 to 15-fold, 11 to 15-fold, 12 to 15-fold, 13 to 15-fold, or 14 to 15-fold guide to passenger strand ratio when processing is measured.

In some embodiments, the vector genome encoding the dsRNA comprises a sequence which is at least 60%, 65%, 70%, 75%, 80%, 85%, 90%, 95%, 99% or more than 99% of the full length of the construct. As a non-limiting example, the vector genome comprises a sequence which is at least 80% of the full length sequence of the construct.

In some embodiments, the siRNA constructs may be used to silence a wild type or mutant gene by targeting at least one exon on the sequence.

siRNA Modification

In some embodiments, the siRNA constructs, when not delivered as a precursor or DNA, may be chemically modified to modulate some features of RNA molecules, such as, but not limited to, increasing the stability of siRNAs in vivo. The chemically modified siRNA constructs can be used in human therapeutic applications, and are improved without compromising the RNAi activity of the siRNA constructs. As a non-limiting example, the siRNA constructs modified at both the 3′ and the 5′ end of both the sense strand and the antisense strand.

In some embodiments, the modified nucleotides may be on just the sense strand.

In some embodiments, the modified nucleotides may be on just the antisense strand.

In some embodiments, the modified nucleotides may be in both the sense and antisense strands.

In some embodiments, the chemically modified nucleotide does not affect the ability of the antisense strand to pair with the target mRNA sequence.

microRNA (miR) Scaffolds

In some embodiments, the siRNA constructs may be encoded in a polynucleotide sequence which also comprises a microRNA (miR) scaffold construct. As used herein a “microRNA (miR) scaffold construct” is a framework or starting molecule that forms the sequence or structural basis against which to design or make a subsequent molecule.

In some embodiments, the miR scaffold construct comprises at least one 5′ flanking region. As a non-limiting example, the 5′ flanking region may comprise a 5′ flanking sequence which may be of any length and may be derived in whole or in part from wild type microRNA sequence or be a completely artificial sequence.

In some embodiments, the miR scaffold construct comprises at least one 3′ flanking region. As a non-limiting example, the 3′ flanking region may comprise a 3′ flanking sequence which may be of any length and may be derived in whole or in part from wild type microRNA sequence or be a completely artificial sequence.

In some embodiments, the miR scaffold construct comprises at least one loop motif region. As a non-limiting example, the loop motif region may comprise a sequence which may be of any length.

In some embodiments, the miR scaffold construct comprises a 5′ flanking region, a loop motif region and/or a 3′ flanking region.

In some embodiment, at least one payload (e.g., siRNA, miRNA or other RNAi agent described herein) may be encoded by a polynucleotide which may also comprise at least one miR scaffold construct. The miR scaffold construct may comprise a 5′ flanking sequence which may be of any length and may be derived in whole or in part from wild type microRNA sequence or be completely artificial. The 3′ flanking sequence may mirror the 5′ flanking sequence and/or a 3′ flanking sequence in size and origin. Either flanking sequence may be absent. The 3′ flanking sequence may optionally contain one or more CNNC motifs, where “N” represents any nucleotide.

In some embodiments, the 5′ arm of the stem loop structure of the polynucleotide comprising or encoding the miR scaffold construct comprises a sequence encoding a sense sequence.

In some embodiments, the 3′ arm of the stem loop of the polynucleotide comprising or encoding the miR scaffold construct comprises a sequence encoding an antisense sequence. The antisense sequence, in some instances, comprises a “G” nucleotide at the 5′ most end.

In some embodiments, the sense sequence may reside on the 3′ arm while the antisense sequence resides on the 5′ arm of the stem of the stem loop structure of the polynucleotide comprising or encoding the miR scaffold construct.

In some embodiments, the sense and antisense sequences may be completely complementary across a substantial portion of their length. In other embodiments the sense sequence and antisense sequence may be at least 70, 80, 90, 95 or 99% complementarity across independently at least 50, 60, 70, 80, 85, 90, 95, or 99% of the length of the strands.

Neither the identity of the sense sequence nor the homology of the antisense sequence need to be 100% complementarity to the target sequence.

In some embodiments, separating the sense and antisense sequence of the stem loop structure of the polynucleotide is a loop sequence (also known as a loop motif, linker or linker motif). The loop sequence may be of any length, between 4-30 nucleotides, between 4-20 nucleotides, between 4-15 nucleotides, between 5-15 nucleotides, between 6-12 nucleotides, 6 nucleotides, 7 nucleotides, 8 nucleotides, 9 nucleotides, 10 nucleotides, 11 nucleotides, 12 nucleotides, 13 nucleotides, 14 nucleotides, and/or 15 nucleotides.

In some embodiments, the loop sequence comprises a nucleic acid sequence encoding at least one UGUG motif. In some embodiments, the nucleic acid sequence encoding the UGUG motif is located at the 5′ terminus of the loop sequence.

In some embodiments, spacer regions may be present in the polynucleotide to separate one or more modules (e.g., 5′ flanking region, loop motif region, 3′ flanking region, sense sequence, antisense sequence) from one another. There may be one or more such spacer regions present.

In some embodiments, a spacer region of between 8-20, i.e., 8, 9, 10, 11, 12, 13, 14, 15, 16, 17, 18, 19, or 20 nucleotides may be present between the sense sequence and a flanking region sequence.

In some embodiments, the length of the spacer region is 13 nucleotides and is located between the 5′ terminus of the sense sequence and the 3′ terminus of the flanking sequence. In some embodiments, a spacer is of sufficient length to form approximately one helical turn of the sequence.

In some embodiments, a spacer region of between 8-20, i.e., 8, 9, 10, 11, 12, 13, 14, 15, 16, 17, 18, 19, or 20 nucleotides may be present between the antisense sequence and a flanking sequence.

In some embodiments, the spacer sequence is between 10-13, i.e., 10, 11, 12 or 13 nucleotides and is located between the 3′ terminus of the antisense sequence and the 5′ terminus of a flanking sequence. In some embodiments, a spacer is of sufficient length to form approximately one helical turn of the sequence.

In some embodiment, the polynucleotide comprises in the 5′ to 3′ direction, a 5′ flanking sequence, a 5′ arm, a loop motif, a 3′ arm and a 3′ flanking sequence. As a non-limiting example, the 5′ arm may comprise a sense sequence and the 3′ arm comprises the antisense sequence. In another non-limiting example, the 5′ arm comprises the antisense sequence and the 3′ arm comprises the sense sequence.

In some embodiments, the 5′ arm, payload (e.g., sense and/or antisense sequence), loop motif and/or 3′ arm sequence may be altered (e.g., substituting 1 or more nucleotides, adding nucleotides and/or deleting nucleotides). The alteration may cause a beneficial change in the function of the construct (e.g., increase knock-down of the target sequence, reduce degradation of the construct, reduce off target effect, increase efficiency of the payload, and reduce degradation of the payload).

In some embodiments, the miR scaffold construct of the polynucleotides is aligned in order to have the rate of excision of the guide strand be greater than the rate of excision of the passenger strand. The rate of excision of the guide or passenger strand may be, independently, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99%. As a non-limiting example, the rate of excision of the guide strand is at least 80%. As another non-limiting example, the rate of excision of the guide strand is at least 90%.

In some embodiments, the rate of excision of the guide strand is greater than the rate of excision of the passenger strand. In one aspect, the rate of excision of the guide strand may be at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99% greater than the passenger strand.

In some embodiments, the efficiency of excision of the guide strand is at least 60%, 65%, 70%, 75%, 80%, 85%, 90%, 95%, 99% or more than 99%. As a non-limiting example, the efficiency of the excision of the guide strand is greater than 80%.

In some embodiments, the efficiency of the excision of the guide strand is greater than the excision of the passenger strand from the miR scaffold construct. The excision of the guide strand may be 2, 3, 4, 5, 6, 7, 8, 9, 10 or more than 10 times more efficient than the excision of the passenger strand from the miR scaffold construct.

In some embodiments, the miR scaffold construct comprises a dual-function targeting polynucleotide. As used herein, a “dual-function targeting” polynucleotide is a polynucleotide where both the guide and passenger strands knock down the same target or the guide and passenger strands knock down different targets.

In some embodiments, the miR scaffold construct of the polynucleotides described herein may comprise a 5′ flanking region, a loop motif region and a 3′ flanking region.

In some embodiments, the polynucleotide is designed using at least one of the following properties: loop variant, seed mismatch/bulge/wobble variant, stem mismatch, loop variant and vassal stem mismatch variant, seed mismatch and basal stem mismatch variant, stem mismatch and basal stem mismatch variant, seed wobble and basal stem wobble variant, or a stem sequence variant.

In some embodiments, the miR scaffold construct may be a natural pri-miRNA scaffold.

In some embodiments, the selection of a miR scaffold construct is determined by a method of comparing polynucleotides in pri-miRNA.

In some embodiments, the selection of a miR scaffold construct is determined by a method of comparing polynucleotides in natural pri-miRNA and synthetic pri-miRNA.

Transfer RNA (tRNA)

Transfer RNAs (tRNAs) are RNA molecules that translate mRNA into proteins. tRNA include a cloverleaf structure that comprise a 3′ acceptor site, 5′ terminal phosphate, D arm, T arm, and anticodon arm. The main purpose of a tRNA is to carry amino acids on its 3′ acceptor site to a ribosome complex with the help of aminoacyl-tRNA synthetases which are enzymes that load the appropriate amino acid onto a free tRNA to synthesize proteins. Once an amino acid is bound to tRNA, the tRNA is considered an aminoacyl-tRNA. The type of amino acid on a tRNA is dependent on the mRNA codon. The anticodon arm of the tRNA is the site of the anticodon, which is complementary to an mRNA codon and dictates which amino acid to carry. tRNAs are also known to have a role in the regulation of apoptosis by acting as a cytochrome c scavenger.

In some embodiments, the originator construct and/or the benchmark construct comprises or encodes a tRNA.

Ribosomal RNA (rRNA)

Ribosomal RNAs (rRNAs) are RNA which form ribosomes. Ribosomes are essential to protein synthesis and contain a large and small ribosomal subunit. In prokaryotes, a small 30S and large 50S ribosomal subunit make up a 70S ribosome. In eukaryotes, the 40S and 60S subunit form an 80S ribosome. In order to bind aminoacyl-tRNAs and link amino acids together to create polypeptides, the ribosome contains 3 sites: an exit site (E), a peptidyl site (P), and acceptor site (A).

In some embodiments, the originator construct and/or the benchmark construct comprises or encodes a rRNA.

microRNA (miRNA)

microRNAs (or miRNA) are 19-25 nucleotide long noncoding RNAs that bind to the 3′UTR of nucleic acid molecules and down-regulate gene expression either by reducing nucleic acid molecule stability or by inhibiting translation. The originator constructs and/or benchmark constructs may comprise one or more microRNA target sequences, microRNA sequences, or microRNA seeds.

A microRNA sequence comprises a “seed” region, i.e., a sequence in the region of positions 2-8 of the mature microRNA, which sequence has perfect Watson-Crick complementarity to the miRNA target sequence. A microRNA seed may comprise positions 2-8 or 2-7 of the mature microRNA. In some embodiments, a microRNA seed may comprise 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may comprise 6 nucleotides (e.g., nucleotides 2-7 of the mature microRNA), wherein the seed-complementary site in the corresponding miRNA target is flanked by an adenine (A) opposed to microRNA position 1. The bases of the microRNA seed have complete complementarity with the target sequence. By engineering microRNA target sequences into the 3′ UTR of the mRNA one can target the molecule for degradation or reduced translation, provided the microRNA in question is available. This process will reduce the hazard of off target effects upon nucleic acid molecule delivery.

As used herein, the term “microRNA site” refers to a microRNA target site or a microRNA recognition site, or any nucleotide sequence to which a microRNA binds or associates. It should be understood that “binding” may follow traditional Watson-Crick hybridization rules or may reflect any stable association of the microRNA with the target sequence at or adjacent to the microRNA site.

Non-limiting examples of tissues where microRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126). MicroRNA can also regulate complex biological processes such as angiogenesis (miR-132).

For example, if the nucleic acid molecule is an mRNA and is not intended to be delivered to the liver but ends up there, then miR-122, a microRNA abundant in liver, can inhibit the expression of the gene of interest if one or multiple target sites of miR-122 are engineered into the 3′ UTR of the mRNA. Introduction of one or multiple binding sites for different microRNA can be engineered to further decrease the longevity, stability, and protein translation of a mRNA.

Conversely, microRNA binding sites can be engineered out of (i.e. removed from) sequences in which they naturally occur in order to increase protein expression in specific tissues. For example, miR-122 binding sites may be removed to improve protein expression in the liver. Regulation of expression in multiple tissues can be accomplished through introduction or removal or one or several microRNA binding sites.

### Long Non-Coding RNA (LncRNA)

Long non-coding RNAs (lncRNAs) are regulatory RNA molecules that do not code for proteins but influence a vast array of biological processes. The lncRNA designation is generally restricted to non-coding transcripts longer than about 200 nucleotides. The length designation differentiates lncRNA from small regulatory RNAs such as short interfering RNA (siRNA) and micro RNA (miRNA). In vertebrates, the number of lncRNA species is thought to greatly exceed the number of protein-coding species. It is also thought that lncRNAs drive biologic complexity observed in vertebrates compared to invertebrates. Evidence of this complexity is seen in many cellular compartments of a vertebrate organism such as the T lymphocyte compartment of the adaptive immune system. Differences in expression and function of lncRNA can be major contributors to human disease.

In some embodiments, the originator constructs and/or the benchmark constructs comprise lncRNAs.

### RNA Modifications

In some aspects, the originator constructs or benchmark constructs may contain one or more modified nucleotides such as, but not limited to, sugar modified nucleotides, nucleobase modifications and/or backbone modifications. In some aspects, the originator constructs or benchmark constructs may contain combined modifications, for example, combined nucleobase and backbone modifications.

In some embodiments, the modified nucleotide may be a sugar-modified nucleotide. Sugar modified nucleotides include, but are not limited to 2′-fluoro, 2′-amino and 2′-thio modified ribonucleotides, e.g. 2′-fluoro modified ribonucleotides. Modified nucleotides may be modified on the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

In some embodiments, the modified nucleotide may be a nucleobase-modified nucleotide.

In some embodiments, the modified nucleotide may be a backbone-modified nucleotide. In some embodiments, the originator constructs or benchmark constructs may further comprise other modifications on the backbone. A normal “backbone”, as used herein, refers to the repeating alternating sugar-phosphate sequences in a DNA or RNA molecule. The deoxyribose/ribose sugars are joined at both the 3-hydroxyl and 5′-hydroxyl groups to phosphate groups in ester links, also known as “phosphodiester” bonds/linker (PO linkage). The PO backbones may be modified as “phosphorothioate backbone (PS linkage). In some cases, the natural phosphodiester bonds may be replaced by amide bonds but the four atoms between two sugar units are kept. Such amide modifications can facilitate the solid phase synthesis of oligonucleotides and increase the thermodynamic stability of a duplex formed with siRNA complement.

Modified bases refer to nucleotide bases such as, but not limited to, adenine, guanine, cytosine, thymine, uracil, xanthine, inosine, and queuosine that have been modified by the replacement or addition of one or more atoms or groups. Some examples of modifications on the nucleobase moieties include, but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides.

The originator constructs and/or benchmark constructs may include one or more substitutions, insertions and/or additions, deletions, and covalent modifications with respect to reference sequences, in particular, the parent RNA, are included within the scope of this invention.

In some embodiments, the originator constructs and/or benchmark constructs includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197) In some embodiments, the first isolated nucleic acid comprises messenger RNA (mRNA). In some embodiments, the originator constructs and/or benchmark constructs comprise at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The originator constructs and/or benchmark constructs may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the originator constructs and/or benchmark constructs includes at least one N(6)methyladenosine (m6A) modification to increase translation efficiency. In some embodiments, the N(6)methyladenosine (m6A) modification can reduce immunogenicity of the originator constructs and/or benchmark constructs.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

In some embodiments, chemical modifications to the RNA may enhance immune evasion. The RNA may be synthesized and/or modified by methods well established in the art, such as those described in “Current protocols in nucleic acid chemistry,” Beaucage, S. L. et al. (Eds.), John Wiley & Sons, Inc., New York, N.Y., USA, which is hereby incorporated herein by reference. Modifications include, for example, end modifications, e.g., 5′ end modifications (phosphorylation (mono-, di- and tri-), conjugation, inverted linkages, etc.), 3′ end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. The modified ribonucleotide bases may also include 5-methylcytidine and pseudouridine. In some embodiments, base modifications may modulate expression, immune response, stability, subcellular localization, to name a few functional effects, of the RNA. In some embodiments, the modification includes a bi-orthogonal nucleotides, e.g., an unnatural base. See for example, Kimoto et al, Chem Commun (Camb), 2017, 53:12309, DOI: 10.1039/c7cc06661a, which is hereby incorporated by reference.

In some embodiments, sugar modifications (e.g., at the 2′ position or 4′ position) or replacement of the sugar one or more RNA may, as well as backbone modifications, include modification or replacement of the phosphodiester linkages. Specific examples of modifications include modified backbones or no natural internucleoside linkages such as internucleoside modifications, including modification or replacement of the phosphodiester linkages. RNA having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, the RNA will include ribonucleotides with a phosphorus atom in its internucleoside backbone.

Modified RNA backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the RNA may be negatively or positively charged.

The modified nucleotides can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The a-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked to the RNA is expected to reduce the innate immune response through weaker binding/activation of cellular innate immune molecules.

In specific embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the RNA may include one or more cytotoxic nucleosides. For example, cytotoxic nucleosides may be incorporated into RNA, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the RNA sequence includes or comprises natural nucleosides (e.g., adenosine, guanosine, cytidine, uridine), nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), and/or modified phosphate groups (e.g., phosphorothioates and 5′-N-phosphoramidite linkages). In one embodiment, the RNA sequence includes or comprises incorporates pseudouridine (y). In another embodiment, the RNA sequence includes or comprises 5-methylcytosine (m5C).

The RNA may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the RNA, or in a given predetermined sequence region thereof. In some embodiments, the RNA includes a pseudouridine. In some embodiments, the RNA includes an inosine, which may aid in the immune system characterizing the RNA as endogenous versus viral RNAs. The incorporation of inosine may also mediate improved RNA stability/reduced degradation.

In some embodiments, all nucleotides in the RNA (or in a given sequence region thereof) are modified. In some embodiments, the modification may include an m6A, which may augment expression, an inosine, which may attenuate an immune response, pseudouridine, which may increase RNA stability, or translational readthrough (stagger element), an m5C, which may increase stability, and a 2,2,7-trimethylguanosine, which aids subcellular translocation (e.g., nuclear localization).

Different sugar modifications, nucleotide modifications, and/or internucleoside linkages (e.g., backbone structures) may exist at various positions in the RNA. One of ordinary skill in the art will appreciate that the nucleotide analogs or other modification(s) may be located at any position(s) of the RNA, such that the function of the RNA is not substantially decreased. A modification may also be a non-coding region modification. The RNA may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

### Codon Optimization

A nucleotide sequence of the originator construct and/or benchmark construct may be codon optimized. Codon optimization methods are known in the art and may be useful in efforts to achieve one or more of several goals. These goals include to match codon frequencies in target and host organisms to ensure proper folding, bias GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g. glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the mRNA. Codon optimization tools, algorithms and services are known in the art, non-limiting examples include services from GeneArt (Life Technologies), DNA2.0 (Menlo Park CA) and/or proprietary methods. In some embodiments, the ORF sequence is optimized using optimization algorithms.

### V. Pharmaceutical Composition and Route of Administration

### Pharmaceutical Compositions and Formulations

The delivery vehicles comprising payloads can be formulated using one or more excipients to: (1) increase stability; (2) increase cell transfection or transduction; (3) permit the sustained or delayed expression of the payload; (4) alter the biodistribution (e.g., target the viral particle to specific tissues or cell types); (5) increase the translation of encoded protein; (6) alter the release profile of encoded protein; and/or (7) allow for regulatable expression of the cargo and/or payload.

Formulations can include, without limitation, saline, liposomes, lipid nanoparticles, polymers, peptides, proteins, cells transfected with viral vectors (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients. As used herein, the phrase “active ingredient” generally refers either to an originator construct or benchmark construct with a payload region or cargo or payload as described herein.

Formulations of the delivery vehicles comprising payloads and pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, or at least 80% (w/w) active ingredient.

### Routes of Administration

The delivery vehicles comprising payloads and/or pharmaceutical compositions described herein may be administered by any delivery route which results in a therapeutically effective outcome. In some embodiments, delivery vehicles comprising payloads and/or pharmaceutical compositions described herein may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### VI. Methods of Use

One aspect of the present disclosure provides methods of using lipids and/or delivery vehicles of the present disclosure. In some embodiments, the delivery vehicles comprising payloads described herein may be used as a therapeutic to diagnose, prevent, treat and/or manage disease, disorders and conditions, or as a diagnostic. The therapeutic may be used in personalized medicine, immuno-oncology, cancer, vaccines, gene editing (e.g., CRISPR).

In some embodiments, the methods of use can be assessed using any endpoint indicating a benefit to the subject, including, without limitation, (1) inhibition, to some extent, of disease progression, including stabilization, slowing down and complete arrest; (2) reduction in the number of disease episodes and/or symptoms; (3) inhibition (i.e., reduction, slowing down or complete stopping) of a disease cell infiltration into adjacent peripheral organs and/or tissues; (4) inhibition (i.e. reduction, slowing down or complete stopping) of disease spread; (5) decrease of an autoimmune condition; (6) favorable change in the expression of a biomarker associated with the disorder; (7) relief, to some extent, of one or more symptoms associated with a disorder; (8) increase in the length of disease-free presentation following treatment; or (9) decreased mortality at a given point of time following treatment.

### Infectious Diseases

The methods, components and compositions of the present invention may be used to diagnose, prevent, treat and/or manage infectious diseases. Infectious diseases, also known as transmissible diseases or communicable diseases, are caused by invasion and multiplication of agents in the body. Infection agents are species typically not present within the body and may be, but are not limited to, viruses, bacteria, prions, nematodes, fungus, parasites or arthropods. Additionally, an infection or symptoms associated with an infection may be caused by one or more toxins produced by such agents. Humans, and other mammals, react to infections with an innate immune system response, often involving an inflammation. The illnesses and symptoms involved with infections vary according to the infectious agent. Many infections may be subclinical without presenting any definite or observable symptoms, whereas some infections cause severe symptoms, require hospitalization or may be life-threatening. Some infections are localized, whereas some may overcome the body through blood circulation or lymphatic vessels. Some infections have long-term effects on wellbeing of infected individuals.

Infectious agents may be transmitted to humans via different routes. For example, infection agents may be transmitted by direct contact with an infected human, an infected animal, or an infected surface. Infections may be transmitted by direct contact with bodily fluids of an infected human or an animal, e.g. blood, saliva, sweat, tears, mucus, female ejaculate, semen, vomit or urine. For example, infection may be transmitted by a fecal-oral route, referring to an infected person shedding the virus in fecal particles which then enters to person's mouth causing infection. The fecal-oral route is especially common transmission route in environments with poor sanitation and hygiene. Non-limiting examples of agents transmitted by the fecal-oral route include bacteria, e.g. shigella, Salmonella typhi and Vibrio Cholerae, virus, e.g. norovirus, rotavirus, enteroviruses, and hepatitis A, fungi, e.g. Entamadeba histolytica, parasites, tape worms, transmitted by contaminated food or beverage, leading to food poisoning or gastroenteritis. Infections may be transmitted by a respiratory route, referring to agents that are spread through the air. Typical examples include agents spread as small droplets of liquid or as aerosols, e.g. respiratory droplets expelled from the mouth and nose while coughing and sneezing. Typical examples of respiratory transmitted diseases include the common cold mostly implicated to rhinoviruses, influenza caused by influenza viruses, respiratory tract infections caused by e.g. respiratory syncytial virus (RSV). Infections may be transmitted by a sexual transmission route. Examples of common sexually transmitted infections include e.g. human immunodeficiency virus (HIV) causing acquired immune deficiency syndrome (AIDS), chlamydia caused by Neisseria gonorrhoeae bacteria, fungal infection Candidiasis caused by Candida yeast, and Herpes Simplex disease caused by herpes simplex virus. Infections may be transmitted by an oral transmission route, e.g. by kissing or sharing a drinking glass. A common infection transmitted by oral transmission is an infectious mononucleosis caused by Epstein-Barr virus. Infections may be transmitted by a vertical transmission, also known as “mother-to-child transmission,” from mother to an embryo, fetus or infant during pregnancy or childbirth. Examples of infection agents that may be transmitted vertically include HIV, chlamydia, rubella, Toxoplasma gondii, and herpes simplex virus. Infections may be transmitted by an iatrogenic route, referring to a transmission by medical procedures such as injection (contaminated reused needles and syringes), or transplantation of infected material, blood transfusions, or infection occurring during surgery. For example, methicillin-resistant Staphylococcus aureus (MRSA), which may cause several severe infections, may be transmitted via iatrogenic route during surgery. Infections may also be transmitted by vector-borne transmission, where a vector may be an organism transferring the infection agents from one host to another. Such vectors may be triatomine bugs, e.g. trypanosomes, parasites, animals, arthropods including e.g. mosquitos, flies, lice, flees, tick and mites or humans. Non-limiting examples of mosquito-borne infections include Dengue fever, West Nile virus related infections, Yellow fever and Chikungunya fever. Non-limiting examples of parasite-borne diseases include malaria, Human African trypanosomiasis and Lyme disease. Non-limiting examples of diseases spread by humans or mammals include HIV, Ebola hemorrhagic fever and Marburg fever.

Traditionally infectious diseases are treated with medications and/or good supportive care. Medical prevention, treatment and/or management of bacterial infections may include administration of antibiotics. Antibiotics may inhibit the colonization of bacteria or kill the bacteria. Antibiotics include e.g. penicillins, cephalosporins, macrolides, fluoroquinolones, sulfonamides, tetracyclines, and aminoglycosides. Antibiotics may be specific to a certain bacteria or act against broad spectrum of bacteria. Some types of bacteria are especially susceptible to antibiotics, whereas some bacteria are more resistant. Development of bacterial strain mutations that are resistant to antibiotics is an increasing concern. Methicillin-resistant Staphylococcus aureus (MRSA), vancomycin-resistant Enterococcus (VRE), multi-drug-resistant Mycobacterium tuberculosis (MDR-TB) and Klebsiella pneumoniae carbapenemase-producing bacteria (KPC) are examples of bacteria that are resistant to most general antibiotics. Due to the emerging resistance, unnecessary administration and overdosing of antibiotics should be avoided. Medical prevention, treatment and/or management of viral infections may include administration of antiviral medications. Antiviral medications may be specific to a certain bacteria or act against a broad spectrum of viruses. Currently antiviral medications are available for e.g. HIV, influenza, hepatitis B and C. Medical prevention, treatment and/or management of viral infections may include administration of antifungal medication. Antifungal medication kills or prevents the growth of fungi. Types of antifungal medications include e.g. imidazoles, triazoles and thiazoles, allylamines, and echinocandins. Development of antifungal medication capable of targeting fungal cells without affecting human cells is a challenge due to the similarities of human and fungal cell on the molecular level. Typically, medical treatment is combined with good supportive care, which includes provision of fluids, bed rest, medication to relieve pain and lower fever, supportive alternative medicine such as vitamins, antioxidants and other supplements important for wellbeing of patients.

Antibody therapies for infectious diseases have also been developed. Examples of commercial therapeutic antibodies include raxibacumab (developed by Cambridge Antibody Technology and Human Genome Sciences) which is an antibody for the prophylaxis and treatment of inhaled anthrax, SHIGAMAB™ (developed by Bellus Health Inc.) is a monoclonal antibody for treatment of Shiga toxin induced hemolytic uremic syndrome, and actoxumab and bezlotoxumab (developed by Medarex Inc. and the University of Massachusetts Medical School) are commercial human monoclonal antibodies targeting C. difficile toxin A and toxin B, respectively.

Infectious diseases and/or infection related diseases, disorders, and/or conditions that may be treated by methods, components and compositions of the present invention include, but are not limited to, 14-day measles, 5-day fever, acne, acquired immunodeficiency syndrome (AIDS), acrodermatitis chronica atrophicans (ACA), acute hemorrhagic conjunctivitis, acute hemorrhagic cystitis, acute rhinosinusitis, adult T-cell leukemia-lymphoma (ATLL), African sleeping sickness, alveolar hydatid, amebiasis, amebic meningoencephalitis, anaplasmosis, anthrax, arboviral, ascariasis, aseptic meningitis, Athlete's foot, Australian tick typhus, avian Influenza, babesiosis, bacillary angiomatosis, bacterial meningitis, bacterial vaginosis, balanitis, balantidiasis, Bang's disease, Barmah Forest virus, bartonellosis, bat lyssavirus, Bay sore, Baylisascaris, beaver fever, beef tapeworm, bejel, biphasic meningoencephalitis, black bane, black death, black piedra, Blackwater fever, blastomycosis, blennorrhea of the newborn, blepharitis, boils, Bornholm disease, Borrelia miyamotoi disease, botulism, boutonneuse fever, Brazilian purpuric fever, break bone fever, brill, bronchiolitis, bronchitis, brucellosis, bubonic, bubonic plague, bullous impetigo, Burkholderia mallei, Burkholderia pseudomallei, burly ulcers mycoburuli ulcers, Busse-Buschke disease, California group encephalitis, campylobacteriosis, candidiasis, canefield fever, canicola fever, capillariasis, carate, carbapenem-resistant enterobacteriaceae (CRE), Carrion's disease, cat scratch fever, cave disease, central Asian hemorrhagic fever, Central European tick, cervical cancer, Chagas disease, cancroid, Chicago disease, chickenpox, Chiclero's ulcer, chikungunya fever, chlamydial, cholera, chromoblastomycosis, ciguatera, clap, clonorchiasis, Clostridium difficile, Clostridium perfringens, coccidioidomycosis fungal, coenurosis, colorado tick fever, condyloma accuminata, condyloma lata, Congo fever, Congo hemorrhagic fever virus, conjunctivitis, cowpox, crabs, Crimean disease, croup, crypto, cryptococcosis, cryptosporidiosis, cutaneous larval migrans, cyclosporiasis, cystic hydatid, cysticercosis, cystitis, Czechoslovak tick, d68 (EV-d68), dacryocytitis, dandy fever, darling's disease, deer fly fever, dengue fever types 1, 2, 3, and 4, desert rheumatism, devil's grip, diphasic milk fever, diphtheria, disseminated intravascular coagulation, dog tapeworm, donovanosis, dracontiasis, dracunculosis, duke's disease, dum dum disease, Durand-Nicholas-Favre disease, dwarf tapeworm, E. coli, eastern equine encephalitis, Ebola hemorrhagic fever, Ebola virus disease (EVD), ectothrix, ehrlichiosis, encephalitis, endemic relapsing fever, endemic syphilis, endophthalmitis, endothrix, enterobiasis, enterotoxin—B poisoning (staph food poisoning), enterovirus, epidemic keratoconjunctivitis, epidemic relapsing fever, epidemic typhus, epiglottitis, epsilon toxin, erysipelis, erysipeloid, erysipelothricosis, erythema chronicum migrans, erythema infectiosum, erythema marginatum, erythema multiforme, erythema nodosum, erythema nodosum leprosum, erythrasma, espundia, eumycotic mycetoma, European blastomycosis, exanthem subitum, eyeworm, Far-Eastern tick, fascioliasis, fievre boutonneuse, fifth disease, Filatow-Dukes' disease, fish tapeworm, Fitz-Hugh-Curtis syndrome—perihepatitis, flinders island spotted fever, flu, folliculitis, four corners disease, frambesia, francis disease, furunculosis, gas gangrene, gastroenteritis, genital herpes, genital warts, German measles, Gerstmann-Straussler-Scheinker (GSS), giardiasis, Gilchrist's disease, gingivitis, gingivostomatitis, glanders, glandular fever, gnathostomiasis, gonococcal, gonorrhea, granuloma inguinale, guinea worm, haemophilus influenza disease, hamburger disease, Hansen's disease, Hantaan disease, Hantaan-Korean hemorrhagic fever, hantavirus pulmonary syndrome (HPS), hard chancre, hard measles, Haverhill fever, head and body lice, heartland fever, helicobacterosis, hemolytic uremic syndrome (HUS), hepatitis A, hepatitis B, hepatitis C, hepatitis D, hepatitis E, herpangina, herpes-genital, herpes labialis, herpes-neonatal, hidradenitis, histoplasmosis, histoplasmosis, his-werner disease, hiv, hookworm s, hordeola, HTLV-associated myelopathy (HAM), human granulocytic ehrlichiosis, human monocytic ehrlichiosis, human papillomarivus (HPV), human pulmonary syndrome, human pulmonary syndrome (HPS), human T-cell lymphotropic virus (HTLV), hydatid cyst, hydrophobia, impetigo, including congenital, inclusion conjunctivitis, infantile diarrhea, infectious mononucleosis, infectious myocarditis, infectious pericarditis, influenza, isosporiasis, Israeli spotted fever, Japanese encephalitis, jock itch, jorge lobo disease, jungle yellow fever, Junin Argentinian hemorrhagic fever, kala azar, Kaposi's sarcoma, keloidal blastomycosis, keratoconjunctivitis, kuru, Kyasanur forest disease, lacrosse encephalitis, lassa hemorrhagic fever, legionellosis, legionnaires disease, legionnaire's pneumonia, Lemierre's syndrome, lemming fever, leprosy, leptospirosis, listeria, listeriosis, liver fluke, lobo's mycosis, lock jaw, lockjaw, loiasis, louping ill, Ludwig's angina, lung fluke, Lyme disease, lymphogranuloma venereum (LGV), Machupo Bolivian hemorrhagic fever, Madura foot, mal del pinto, malaria, malignant pustule, Malta fever, Marburg hemorrhagic fever, masters disease, maternal sepsis, measles, Mediterranean spotted fever, melioidosis, meningitis, meningococcal disease, Middle East Respiratory Syndrome (MERS), methicillin-resistant Staphylococcus aureus (MRSA), milker's nodule, molluscum contagiosum, moniliasis, monkeypox, mononucleosis, mononucleosis-like syndrome, Montezuma's revenge, morbilli, mucormycosis, multiple organ dysfunction syndrome (MODS), multiple-system atrophy (MSA), mumps, murine typhus, Murray Valley encephalitis (MVE), mycoburuli ulcers, mycotic vulvovaginitis, myositis, Nanukayami fever, necrotizing fasciitis, necrotizing fasciitis-type 1, necrotizing fasciitis-type 2, negishi, new world spotted fever, nocardiosis, nongonococcal urethritis, non-polio enterovirus, norovirus, North American blastomycosis, North Asian tick typhus, Norwalk virus, Norwegian itch, O'hara disease, Omsk hemorrhagic fever, onchoceriasis, onychomycosis, opisthorchiasis, opthalmia neonatorium, oral hairy leukoplakia, orf, oriental sore, oriental spotted fever, ornithosis, Oroya fever, otitis externa, otitis media, pannus, paracoccidioidomycosis, paragonimiasis, parainfectious, paralytic shellfish poisoning, paronychia, parotitis, parrot fever, pediculosis, peliosis hepatica, pelvic inflammatory disease, pertussis, phaeohyphomycosis, pharyngoconjunctival fever, piedra, pigbel, pink eye conjunctivitis, pinta, pinworm, pitted keratolysis, pityriasis versicolor, plague, pleurodynia, pneumococcal disease, pneumocystis pneumonia, pneumocystosis, pneumonia, polio, poliomyelitis, polycystic hydatid, Pontiac fever, pork tapeworm, Posada-Wernicke disease, postanginal septicemia, Powassan, progressive multifocal leukoencephalopathy (PML), progressive rubella panencephalitis, prostatitis, pseudomembranous colitis, psittacosis, puerperal fever, pustular rash diseases, pyelonephritis, pylephlebitis, q-fever, quinsy, quintana fever, rabbit fever, rabies, raccoon roundworm, rat bite fever, rat tapeworm, Reiter syndrome, relapsing fever, respiratory syncytial virus (RSV), rheumatic fever, rhodotorulosis, ricin poisoning, rickettsia pox, rickettsiosis, Rift valley fever, ringworm, Ritter's disease, river blindness, rocky mountain spotted fever, rose handler's disease, rose rash of infants, roseola, Ross river fever, rotavirus, roundworm s, rubella, rubeola, Russian spring, salmonellosis gastroenteritis, San Joaquin valley fever, Sao Paulo encephalitis, Sao Paulo fever, scabies infestation, scalded skin syndrome, scalded skin syndrome, scarlatina, scarlet fever, schistosomiasis, scombroid, scrub typhus, sennetsu fever, sepsis, septic shock, severe acute respiratory syndrome, severe acute respiratory syndrome (SARS), shiga toxigenic Escherichia coli, shigella, shigellosis gastroenteritis, shinbone fever, shingles, shipping fever, siberian tick typhus, sinusitis, sixth disease, slapped cheek disease, sleeping sickness, small pox, smallpox, snail fever, soft chancre, southern tick associated rash illness, sparganosis, Spelunker's disease, sporadic typhus, sporotrichosis, spotted fever, spring, St. Louis encephalitis, staphylococcal food poisoning, staphylococcal, strep. throat, streptococcal disease, streptococcal toxic-shock syndrome, strongyloiciasis, stye, subacute sclerosing panencephalitis (SSAPE), sudden acute respiratory syndrome, sudden rash, swimmer's ear, swimmer's itch, swimming pool conjunctivitis, sylvatic yellow fever, syphilis, systemic inflammatory response syndrome (SIRS), tabes dorsalis, taeniasis, taiga encephalitis, tanner's disease, tapeworm s, temporal lobe encephalitis, tertiary syphilis, tetani, tetanus, threadworm s, thrush, tick, tick typhus, tinea barbae, tinea capitis, tinea corporis, tinea cruris, tinea manuum, tinea nigra, Tinea pedis, tinea unguium, tinea versicolor, torulopsosis, torulosis, toxic shock syndrome, toxoplasmosis, transmissible spongioform, traveler's diarrhea, trench fever 5, trichinellosis, trichomoniasis, trichomycosis axillaris, trichuriasis, tropical spastic paraparesis (TSP), trypanosomiasis, tuberculosis (TB), tularemia, typhoid fever, typhus fever, ulcus molle, undulant fever, urban yellow fever, urethritis, vaginitis, vaginosis, valley fever, vancomycin intermediate (VISA), vancomycin resistant (VRSA), varbuncle, varicella, variola, varrion's disease, venezuelan equine encephalitis, Verruga peruana, vibrio, Vibrio cholerae, vibriosis, vincent's disease or trench mouth, viral conjunctivitis, viral meningitis, viral meningoencephalitis, viral rash, visceral larval migrans, vomito negro, vulvovaginitis, warts, Waterhouse, Weil's disease, West Nile fever, Western equine encephalitis, Whipple's disease, whipworm, white piedra, whitlow, Whitmore's disease, whooping cough, winter diarrhea, wolhynia fever, wool sorters' disease, yaws, yellow fever, yersinosis, zahorsky's disease, zika virus disease, zoster, zygomycosis, acute bacterial rhinosinusitis, lobomycosis, and/or any other infectious diseases, disorders or conditions.

### Preventative Applications

In some embodiments, the delivery vehicles comprising payloads described herein may be used to prevent disease or stabilize the progression of disease.

In some embodiments, the delivery vehicles comprising payloads described herein may be used as a prophylactic to prevent a disease or disorder in the future.

In some embodiments, the delivery vehicles comprising payloads described herein may be used to halt further progression of a disease or disorder.

### Vaccine

In some embodiments, the delivery vehicles comprising payloads described herein may be used as, and/or in a manner similar to that of a vaccine. As used herein, a “vaccine” is a biological preparation that improves immunity to a particular disease or infectious agent.

In some embodiments, the delivery vehicles comprising payloads described herein may be used as, and/or in a manner similar to that of a vaccine for a therapeutic area such as, but not limited to, cardiovascular, CNS, dermatology, endocrinology, oncology, immunology, respiratory, and anti-infective.

In some embodiments, the delivery vehicles comprising payloads described herein may be used as a vaccine to diagnose, prevent, treat and/or manage a foodborne illness.

In some embodiments, the delivery vehicles comprising payloads described herein may be used as a vaccine to diagnose, prevent, treat and/or manage gastroenteritis.

In some embodiments, the delivery vehicles comprising payloads described herein may be used as a vaccine to diagnose, prevent, treat and/or manage influenza.

In some embodiments, the delivery vehicles comprising payloads described herein may be used as a prophylactic to diagnose, prevent, treat and/or manage HIV.

In some embodiments, the delivery vehicles comprising payloads described herein may be used as a prophylactic to diagnose, prevent, treat and/or manage coronavirus.

In some embodiments, the delivery vehicles comprising payloads described herein may be used as a prophylactic to diagnose, prevent, treat and/or manage COVID-19.

In some embodiments, the delivery vehicles comprising payloads described herein may be used as a prophylactic to diagnose, prevent, treat and/or manage polio.

In some embodiments, the delivery vehicles comprising payloads described herein may be used as a prophylactic to diagnose, prevent, treat and/or manage tetanus.

In some embodiments, the delivery vehicles comprising payloads described herein may be used as a prophylactic to diagnose, prevent, treat and/or manage Hepatitis A.

In some embodiments, the delivery vehicles comprising payloads described herein may be used as a prophylactic to diagnose, prevent, treat and/or manage Hepatitis B.

In some embodiments, the delivery vehicles comprising payloads described herein may be used as a prophylactic to diagnose, prevent, treat and/or manage Hepatitis C.

In some embodiments, the delivery vehicles comprising payloads described herein may be used as a prophylactic to diagnose, prevent, treat and/or manage Rubella.

In some embodiments, the delivery vehicles comprising payloads described herein may be used as a prophylactic to diagnose, prevent, treat and/or manage Hib (Haemophilus influenzae type b).

In some embodiments, the delivery vehicles comprising payloads described herein may be used as a prophylactic to diagnose, prevent, treat and/or manage Measles.

In some embodiments, the delivery vehicles comprising payloads described herein may be used as a prophylactic to diagnose, prevent, treat and/or manage Pertussis (Whooping Cough).

In some embodiments, the delivery vehicles comprising payloads described herein may be used as a prophylactic to diagnose, prevent, treat and/or manage Pneumococcal Disease.

In some embodiments, the delivery vehicles comprising payloads described herein may be used as a prophylactic to diagnose, prevent, treat and/or manage Rotavirus.

In some embodiments, the delivery vehicles comprising payloads described herein may be used as a prophylactic to diagnose, prevent, treat and/or manage Mumps.

In some embodiments, the delivery vehicles comprising payloads described herein may be used as a prophylactic to diagnose, prevent, treat and/or manage Chickenpox.

In some embodiments, the delivery vehicles comprising payloads described herein may be used as a prophylactic to diagnose, prevent, treat and/or manage Diphtheria.

### Cancers

In some embodiments, the delivery vehicles comprising payloads described herein may be used for treating cancer. According to the present disclosure, cancer embraces any disease or malady characterized by uncontrolled cell proliferation, e.g., hyperproliferation. Cancers may be characterized by tumors, e.g., solid tumors or any neoplasm.

In some embodiments, the delivery vehicles comprising payloads of the present disclosure have been found to inhibit cancer and/or tumor growth. They may also reduce, including cell proliferation, invasiveness, and/or metastasis, thereby rendering them useful for the treatment of a cancer.

In some embodiments, the delivery vehicles comprising payloads of the present disclosure may be used to prevent the growth of a tumor or cancer, and/or to prevent the metastasis of a tumor or cancer. In some embodiments, compositions of the present teachings may be used to shrink or destroy a cancer.

In some embodiments, the delivery vehicles comprising payloads provided herein are useful for inhibiting proliferation of a cancer cell. In some embodiments, the delivery vehicles comprising payloads provided herein are useful for inhibiting cellular proliferation, e.g., inhibiting the rate of cellular proliferation, preventing cellular proliferation, and/or inducing cell death. In general, the delivery vehicles comprising payloads as described herein can inhibit cellular proliferation of a cancer cell or both inhibiting proliferation and/or inducing cell death of a cancer cell.

The cancers treatable by methods of the present teachings generally occur in mammals. Mammals include, for example, humans, non-human primates, dogs, cats, rats, mice, rabbits, ferrets, guinea pigs horses, pigs, sheep, goats, and cattle. In various embodiments, the cancer is lung cancer, breast cancer, e.g., mutant BRCA1 and/or mutant BRCA2 breast cancer, non-BRCA-associated breast cancer, colorectal cancer, ovarian cancer, pancreatic cancer, colorectal cancer, bladder cancer, prostate cancer, cervical cancer, renal cancer, leukemia, central nervous system cancers, myeloma, and melanoma.

### Diagnostics

In some embodiments, the delivery vehicles comprising payloads described herein may be used for diagnostic purposes or as diagnostic tools for any of the aforementioned diseases or disorders.

In some embodiments, the delivery vehicles comprising payloads described herein may be used to detect a biomarker for disease diagnosis.

In some embodiments, the delivery vehicles comprising payloads described herein may be used for diagnostic imaging purposes, e.g., MRI, PET, CT or ultrasound.

### VII. Kits and Devices

### Kits

The disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

In some embodiments, the present disclosure provides kits for modulating the expression of genes in vitro or in vivo, comprising nucleic acid vaccine compositions of the present disclosure or a combination of nucleic acid vaccine compositions of the present disclosure, nucleic acid vaccine compositions modulating other genes, siRNAs, miRNAs or other oligonucleotide molecules.

The kit may further comprise packaging and instructions and/or a delivery agent to form a formulation, e.g., for administration to a subject in need of treatment using the nucleic acid vaccine compositions described herein. The delivery agent may comprise a saline, a buffered solution, a lipidoid, a dendrimer or any suitable delivery agent.

In one non-limiting example, the buffer solution may include sodium chloride, calcium chloride, phosphate and/or EDTA. In another non-limiting example, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose (See U.S. Pub. No. 20120258046; herein incorporated by reference in its entirety). In yet another non-limiting example, the buffer solutions may be precipitated or it may be lyophilized. The amount of each component may be varied to enable consistent, reproducible higher concentration saline or simple buffer formulations. The components may also be varied in order to increase the stability of nucleic acid vaccine compositions in the buffer solution over a period of time and/or under a variety of conditions.

### Devices

The present disclosure provides for devices which may incorporate nucleic acid vaccine compositions of the present disclosure. These devices can contain a stable formulation available to be immediately delivered to a subject in need thereof, such as a human patient.

Non-limiting examples of the devices include a pump, a catheter, a needle, a transdermal patch, a pressurized olfactory delivery device, electroporation devices, iontophoresis devices, multi-layered microfluidic devices. The devices may be employed to deliver nucleic acid vaccine compositions of the present disclosure according to single, multi- or split-dosing regiments. The devices may be employed to deliver nucleic acid vaccine compositions of the present disclosure across biological tissue, intradermal, subcutaneously, or intramuscularly. More examples of devices suitable for delivering oligonucleotides are disclosed in International Publication WO 2013/090648, the contents of which are incorporated herein by reference in their entirety.

### VIII. Definitions

At various places in the present specification, substituents of compounds of the present disclosure are disclosed in groups or in ranges. It is specifically intended that the present disclosure include each and every individual subcombination of the members of such groups and ranges.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Adjuvant: As used herein, the term “adjuvant” means a substance that enhances a subject's immune response to an antigen. The nucleic acid vaccines described herein may optionally comprise one or more adjuvants.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antigen: As defined herein, the term “antigen” refers to a composition, for example, a substance or agent which causes an immune response in an organism, e.g., causes the immune response of the organism to produce antibodies against the substance or agent in particular, which provokes an adaptive immune response in an organism. Antigens can be any immunogenic substance including, in particular, proteins, polypeptides, polysaccharides, nucleic acids, lipids and the like. Exemplary antigens are derived from infectious agents. Such agents can include parts or subunits of infectious agents, for example, coats, coat components, e.g., coat protein or polypeptides, surface components, e.g., surface proteins or polypeptides, capsule components, cell wall components, flagella, fimbriae, and/or toxins or toxoids) of infectious agents, for example, bacteria, viruses, and other microorganisms. Certain antigens, for example, lipids and/or nucleic acids are antigenic, preferably, when combined with proteins and/or polysaccharides.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety which is capable of or maintains at least two functions. The functions may affect the same outcome or a different outcome. The structure that produces the function may be the same or different.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, a polynucleotide described herein may be considered biologically active if even a portion of the polynucleotides is biologically active or mimics an activity considered biologically relevant.

Chimera: As used herein, “chimera” is an entity having two or more incongruous or heterogeneous parts or regions.

Compound: As used herein, the term “compound,” is meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Controlled Release: As used herein, the term “controlled release” refers to a pharmaceutical composition or compound release profile that conforms to a particular pattern of release to effect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of a polynucleotide to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Encoded protein cleavage signal: As used herein, “encoded protein cleavage signal” refers to the nucleotide sequence which encodes a protein cleavage signal.

Engineered: As used herein, embodiments of the nucleic acid vaccines are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Exosome: As used herein, “exosome” is a vesicle secreted by mammalian cells or a complex involved in RNA degradation.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least a polynucleotide of a nucleic acid vaccine and a delivery agent.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75% 80%, 85%, 90%, 95%, or 99% identical or similar. The term “homologous” necessarily refers to a comparison between at least two sequences (polynucleotide or polypeptide sequences). Two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between polynucleotide molecules (e.g. DNA molecules and/or RNA molecules) and/or between polypeptide molecules.

Calculation of the percent identity of two polynucleotide sequences, for example, can be performed by aligning the two sequences for optimal comparison purposes (e.g., gaps can be introduced in one or both of a first and a second nucleic acid sequences for optimal alignment and nonidentical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M, ed., Oxford University Press, N.Y., 1988; Biocomputing: Informatics and Genome Projects, Smith, D. W., ed., Academic Press, N.Y, 1993; Sequence Analysis in Molecular Biology, von Heinje, G., Academic Press, 1987; Computer Analysis of Sequence Data, Part I, Griffin, A. M, and Griffin, H. G., eds., Humana Press, N.J., 1994; and Sequence Analysis Primer, Gribskov, M. and Devereux, J., eds., M Stockton Press, N.Y, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM 120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al, J. Molec. Biol., 215, 403 (1990)).

Infectious Agent: As used herein, the phrase “infectious agent” means an agent capable of producing an infection in an organism, for example, in an animal. An infectious agent may refer to any microorganism, virus, infectious substance, or biological product that may be engineered as a result of biotechnology, or any naturally occurring or bioengineered component of any such microorganism, virus, infectious substance, or biological product, can cause emerging and contagious disease, death or other biological malfunction in a human, an animal, a plant or another living organism.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein, a “linker” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker may be of sufficient length as to not interfere with incorporation into a nucleic acid sequence.

Modified: As used herein “modified” refers to a changed state or structure of a molecule described herein. Molecules may be modified in many ways including chemically, structurally, and functionally.

Mucus: As used herein, “mucus” refers to the natural substance that is viscous and comprises mucin glycoproteins.

Naturally occurring: As used herein, “naturally occurring” means existing in nature without artificial aid.

Neutralizing antibody: As used herein, a “neutralizing antibody” refers to an antibody which binds to its antigen and defends a cell from an antigen or infectious agent by neutralizing or abolishing any biological activity it has.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic Acid Vaccine: As used herein, “nucleic acid vaccine” refers to a vaccine or vaccine composition which includes a nucleic acid or nucleic acid molecule (e.g., a polynucleotide) encoding an antigen (e.g., an antigenic protein or polypeptide.) In exemplary embodiments, a nucleic acid vaccine includes a ribonucleic (“RNA”) polynucleotide, ribonucleic acid (“RNA”) or ribonucleic acid (“RNA”) molecule. Such embodiments can be referred to as ribonucleic acid (“RNA”) vaccines.

Off-target: As used herein, “off target” refers to any unintended effect on any one or more target, gene, or cellular transcript.

Open reading frame: As used herein, the term “open reading frame” or “ORF” refers to a continuous polynucleotide sequence, for example, a DNA sequence or RNA sequence (e.g., an mRNA sequence), comprising a start codon, a subsequent region comprising a plurality of amino acid-encoding codons, and a terminal stop codon, wherein the region comprising the plurality of amino acid-encoding codons contains no stop codons.

Operably linked: As used herein, the phrase “operably linked” refers to a functional connection between two or more molecules, constructs, transcripts, entities, moieties or the like.

Part: As used herein, a “part” or “region” of a polynucleotide is defined as any portion of the polynucleotide which is less than the entire length of the polynucleotide.

Peptide: As used herein, “peptide” is less than or equal to 50 amino acids long, e.g., about 5, 10, 15, 20, 25, 30, 35, 40, 45, or 50 amino acids long.

Paratope: As used herein, a “paratope” refers to the antigen-binding site of an antibody.

Patient: As used herein, “patient” refers to a subject who may seek or be in need of treatment, requires treatment, is receiving treatment, will receive treatment, or a subject who is under care by a trained professional for a particular disease or condition.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Beige et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound described herein wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidi-none (NMP), dimethyl sulfoxide (DMSO), N,N′-dimethyl-formamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Polypeptide per unit drug (PUD): As used herein, a PUD or product per unit drug, is defined as a subdivided portion of total daily dose, usually 1 mg, pg, kg, etc., of a product (such as a polypeptide) as measured in body fluid or tissue, usually defined in concentration such as pmol/mL, mmol/mL, etc. divided by the measure in the body fluid.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease. An “immune prophylaxis” refers to a measure to produce active or passive immunity to prevent the spread of disease.

Protein cleavage site: As used herein, “protein cleavage site” refers to a site where controlled cleavage of the amino acid chain can be accomplished by chemical, enzymatic or photochemical means.

Protein cleavage signal: As used herein “protein cleavage signal” refers to at least one amino acid that flags or marks a polypeptide for cleavage.

Protein of interest: As used herein, the terms “proteins of interest” or “desired proteins” include those provided herein and fragments, mutants, variants, and alterations thereof.

Purified: As used herein, “purify,” “purified,” “purification” means to make substantially pure or clear from unwanted components, material defilement, admixture or imperfection.

Repeated transfection: As used herein, the term “repeated transfection” refers to transfection of the same cell culture with a polynucleotide a plurality of times. The cell culture can be transfected at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times at least 18 times, at least 19 times, at least 20 times, at least 25 times, at least 30 times, at least 35 times, at least 40 times, at least 45 times, at least 50 times or more.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event.

Similarity: As used herein, the term “similarity” refers to the overall relatedness between polymeric molecules, e.g., between polynucleotide molecules (e.g., DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition may be administered, e.g., for experimental, diagnostic, prophylactic, and/or therapeutic purposes. Typical subjects include animals (e.g., mammals such as mice, rats, rabbits, non-human primates, and humans).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules described herein may be chemical or enzymatic.

Vaccine: As used herein, a vaccine is a compound or composition which comprises at least one polynucleotide encoding at least one antigen.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr. period. It may be administered as a single unit dose.

Transfection: As used herein, the term “transfection” refers to methods to introduce exogenous nucleic acids into a cell. Methods of transfection include, but are not limited to, chemical methods, physical treatments and cationic lipids or mixtures.

Translation: As used herein “translation” is the process by which a polynucleotide molecule is processed by a ribosome or ribosomal-like machinery, e.g., cellular or artificial, to produce a peptide or polypeptide.

Transcription: As used herein “transcription” is the process by which a polynucleotide molecule is processed by a polymerase or other enzyme to produce a polynucleotide, e.g., an RNA polynucleotide.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. Treatment may be administered to a subject who does not exhibit signs of a disease, infection, disorder, and/or condition and/or to a subject who exhibits only early signs of a disease, infection, disorder, and/or condition for the purpose of decreasing the risk of developing pathology associated with the disease, infection, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vaccine: As used herein, the phrase “vaccine” refers to a biological preparation that improves immunity in the context of a particular disease, disorder or condition.

Viral protein: As used herein, the phrase “viral protein” means any protein originating from a virus.

The term “alkyl” refers to the radical of saturated aliphatic groups, including straight-chain alkyl groups (i.e., linear chain alkyl groups), branched-chain alkyl groups, cycloalkyl (alicyclic) groups, alkyl-substituted cycloalkyl groups, and cycloalkyl-substituted alkyl groups.

In some embodiments, a straight chain or branched chain alkyl has 30 or fewer carbon atoms in its backbone (e.g., C1-C30 for straight chains, C3-C30 for branched chains), 20 or fewer, 12 or fewer, or 7 or fewer. Likewise, in some embodiments cycloalkyls have from 3-10 carbon atoms in their ring structure, e.g., have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Example of substituents are defined in more detail further below. In some embodiments, these substituents can include, but are not limited to, halogen, hydroxyl, carbonyl (such as a carboxyl, e.g., acetoxy, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, alkyl (e.g., C1-C14 alkyl), alkenyl (e.g., C2-C10 alkenyl), heterocyclyl, aralkyl, or an aromatic or heteroaromatic moiety.

Unless the number of carbons is otherwise specified, “lower alkyl” as used herein means an alkyl group, as defined above, but having from one to ten carbons, or from one to six carbon atoms in its backbone structure. Likewise, “lower alkenyl” and “lower alkynyl” have similar chain lengths. In some embodiments, alkyl groups are lower alkyls. In some embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The term “alkylthio” refers to an alkyl group, as defined above, having a sulfur radical attached thereto. In some embodiments, the “alkylthio” moiety is represented by one of —S-alkyl, —S-alkenyl, and —S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

The terms “alkenyl” and “alkynyl”, refer to unsaturated aliphatic groups analogous in length to the alkyls described above, but that contain at least one double or triple bond respectively. In some embodiments, the alkenyl and alkynyl groups can have from 1 to 8, e.g., from 1 to 3, double bonds or triple bonds, respectively. Alkenyl and alkynyl groups can be unsubstituted or substituted with one or more substituents defined herein, such as the substituents that are optionally present on the alkyl groups, as defined above.

The terms “alkoxyl” or “alkoxy” as used herein refers to an alkyl group, as defined above, having an oxygen radical attached thereto. Representative alkoxyl groups include methoxy, ethoxy, propyloxy, and tert-butoxy. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. “Aryloxy” can be represented by —O-aryl and “heteroaryloxy” can be represented by —O-heteroaryl, wherein aryl and heteroaryl are as defined below. The alkoxy, heteroaryloxy and aryloxy groups can be substituted as described above for alkyl.

The terms “amine” and “amino” are art-recognized and refer to both unsubstituted and substituted amino groups, e.g., a moiety that can be represented by the general formula:

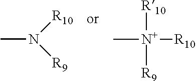

wherein R₉, R₁₀, and R′₁₀ each independently represent a hydrogen, an alkyl, an alkenyl, —(CH₂)ₘ—R₈ or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In still other embodiments, the term “amino” does not encompass amido groups, e.g., wherein one of R₉ and R₁₀ represents a carbonyl. In additional embodiments, R₉ and R₁₀ (and optionally R′₁₀) each independently represent a hydrogen, an alkyl or cycloalkyl, an alkenyl or cycloalkenyl, or alkynyl. Thus, the term “alkylamino” as used herein means an amino group, as defined above, having a substituted (as described above for alkyl) or unsubstituted alkyl attached thereto, i.e., at least one of R₉ and R₁₀ is an alkyl group.

The term “amido” is art-recognized as an amino-substituted carbonyl and includes a moiety that can be represented by the general formula:

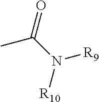

wherein R₉ and R₁₀ are as defined above.

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles”, “heteroaryl” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “aralkyl,” as used herein, refers to an alkyl group substituted with an aryl group (e.g., an aromatic or heteroaromatic group).

The term “carbocycle,” as used herein, refers to an aromatic or non-aromatic ring in which each atom of the ring is carbon.

“Heterocycle” or “heterocyclic,” as used herein, refers to a cyclic radical attached via a ring carbon or nitrogen of a monocyclic or bicyclic ring containing 3-10 ring atoms, for example, from 5-6 ring atoms, consisting of carbon and one to four heteroatoms each selected from the group consisting of non-peroxide oxygen, sulfur, and N(Y) wherein Y is absent or is H, O, (C₁-C₁₀) alkyl, phenyl or benzyl, and optionally containing 1-3 double bonds and optionally substituted with one or more substituents. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, and —CN.

The term “carbonyl” is art-recognized and includes such moieties as can be represented by the general formula:

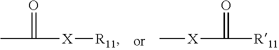

wherein X is a bond or represents an oxygen or a sulfur, and R₁₁ represents a hydrogen, an alkyl, a cycloalkyl, an alkenyl, a cycloalkenyl, or an alkynyl, R′₁₁ represents a hydrogen, an alkyl, a cycloalkyl, an alkenyl, a cycloalkenyl, or an alkynyl. Where X is an oxygen and R₁₁ or R′₁₁ is not hydrogen, the formula represents an “ester”. Where X is an oxygen and R₁₁ is as defined above, the moiety is referred to herein as a carboxyl group, and particularly when R₁₁ is a hydrogen, the formula represents a “carboxylic acid”. Where X is an oxygen and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “heteroatom” as used herein means an atom of any element other than carbon or hydrogen. Examples of heteroatoms are boron, nitrogen, oxygen, phosphorus, sulfur and selenium. Other useful heteroatoms include silicon and arsenic.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, for example, 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation, for example, by rearrangement, cyclization, or elimination.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

In various embodiments, the substituent is selected from alkoxy, aryloxy, alkyl, alkenyl, alkynyl, amido, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amido, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amido, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester (e.g, acetoxy), heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from alkyl, alkenyl, cyano, halogen, hydroxyl, acetoxy, and nitro.

The term “with any possible isomerism” used in relation to any chemical group mentioned herein, such as alkyl, alkenyl, or alkynyl groups for instance, means that such groups can have any possible isomeric structure, including linear, branched, and/or cyclic structures.

### EXAMPLES

In the following examples, abbreviations may be used and are defined in Table 2.

TABLE 2
Abbreviations and their definitions.

| Abbreviation | Description |
|---|---|
| ¹H | proton |
| ¹³C | carbon-13 |
| ° C. | degrees Celsius |
| Calcd | calculated |
| cm⁻¹ | wave numbers |
| d | day(s); doublet (spectral) |
| DCC | dicyclohexylcarbodiimide |
| DCM | dichloromethane |
| DCU | dicyclohexyl urea |
| dd | doublet of doublets (spectral) |
| DIBAL-H | diisobutylaluminum hydride |
| DMP | dess-Martin periodinane |
| DMF | dimethylformamide |
| DMAP | dimethylaminopyridine |
| DMSO | dimethylsulfoxide |
| dt | doublet of triplets (spectral) |
| dtt | doublet of triplets of triplets (spectral) |
| equiv | equivalent(s) |
| EtOAc | ethyl acetate |
| FT-IR | Fourier-transform infrared spectroscopy |
| g | gram(s) |
| h | hour(s) |
| Hex | Hexane |
| HRMS | high resolution mass spectrometry |
| HPLC | high performance liquid chromatography |
| Hz | hertz |
| J | coupling constant |
| L | liter(s) |
| LCMS | liquid chromatography coupled with mass spectrometry |
| LNP | lipid nanoparticle |
| M | molar |
| m | multiplet (spectral) |
| mg | milligram(s) |
| min | minute(s) |
| mL | milliliter(s) |
| mmol | millimole(s) |
| mol | mole(s) |
| mRNA | messenger RNA |
| m/z | mass to charge ratio |
| NBS | N-bromosuccinimide |
| nm | nanometer(s) |
| NMR | nuclear magnetic resonance |
| ppm | parts per million |
| Ph | Phenyl |
| q | quartet (spectral) |
| qt | quartet of triplet |
| quint | quintet |
| R_{f} | retention factor (in chromatography) |
| Rt | retention time |
| RT | room temperature |
| s | singlet (spectral) |
| sept | septet (spectral) |
| t | triplet (spectral) |
| TEA | triethylamine |
| THF | tetrahydrofuran |
| tt | triplet of triplet |
| wt. | weight |
| δ | chemical shift |

### Example 1: Methods of Making the Lipids

The lipids of the present disclosure may be prepared using any convenient methodology. In a rational approach, the lipids are constructed from their individual components. The components can be covalently bonded to one another through functional groups, as is known in the art, where such functional groups may be present on the components or introduced onto the components using one or more steps, e.g., oxidation reactions, reduction reactions, cleavage reactions and the like. Functional groups that may be used in covalently bonding the components together to produce the lipids: hydroxy, sulfhydryl, amino, and the like. Where necessary and/or desired, certain moieties on the components may be protected using blocking groups, as is known in the art, see, e.g., Green & Wuts, Protective Groups in Organic Synthesis (John Wiley & Sons) (1991).

Alternatively, the lipids can be produced using known combinatorial methods to produce large libraries of potential lipids which may then be screened for identification of a lipid with desired functionalities.

Disulfide Lipids with Two Branches

As a non-limiting example, Compound 100 may be prepared according to the following synthesis scheme. Other disulfide lipids with two branches may be prepared in similar synthetic steps.

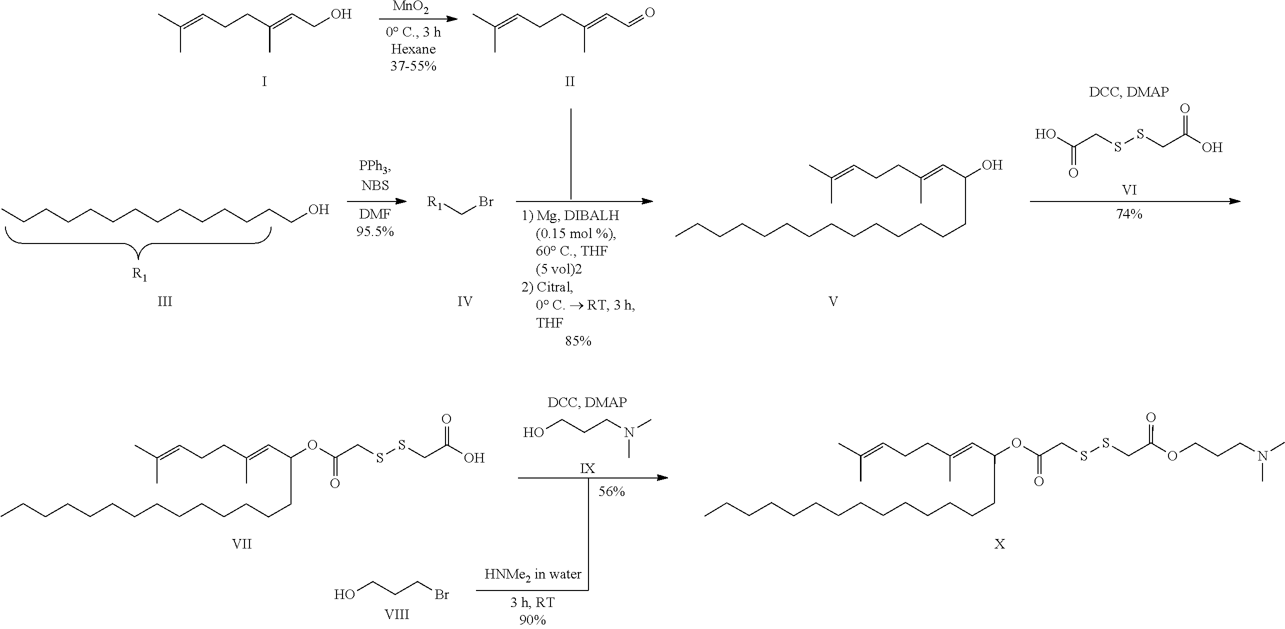

### Step 1

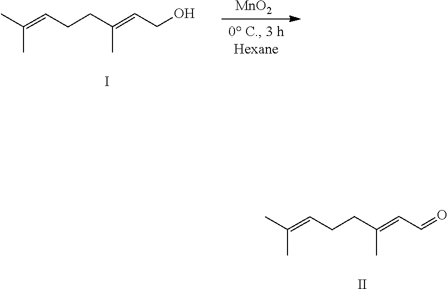

Manganese dioxide (40 g, 4 equiv) was added to the flask under Ar atmosphere, later geraniol (I) (3.85 g, 1 equiv) in dry hexane was added at 0° C. and left to stir for 3 hours. The solution was filtered through a celite pad and concentrated under vacuum. Column chromatography was performed using 10% EtOAc:Hex to afford compound II. ¹H NMR (401 MHz, CDCl₃) δ 10.00 (d, J=8.1 Hz, 1H), 5.89 (dd, J=8.0, 2.4 Hz, 1H), 5.13-5.03 (m, 1H), 2.28-2.18 (m, 4H), 2.17 (d, J=1.3 Hz, 3H), 1.69 (d, J=1.4 Hz, 3H), 1.61 (d, J=1.4 Hz, 3H).

### Step 2

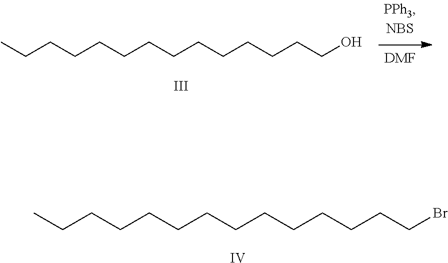

The typical procedure as applied to the preparation of product IV involves the addition of N-bromosuccinimide (1.64 g, 2 equiv) followed by triphenylphosphine (2.46 g, 2 equiv) to a cooled solution of the alcohol (III) (1 g, 1 equiv) in anhydrous DMF and heating the mixture to 50° C. for 20 min. Methyl alcohol is added to quench excess reagent, followed by n-butanol to aid in the removal of DMF under reduced pressure. After concentration, the product was isolated from the by-products (succinimide and triphenylphosphine oxide) by addition of ether followed by aqueous extraction. Purification by column chromatography afforded compound IV. ¹H NMR (401 MHz, CDCl₃) δ 3.42 (t, J=6.9 Hz, 2H), 1.86 (p, J=6.8 Hz, 2H), 1.43 (p, J=7.1 Hz, 3H), 1.37-1.22 (m, 19H), 0.89 (t, J=6.3 Hz, 3H).

### Step 3

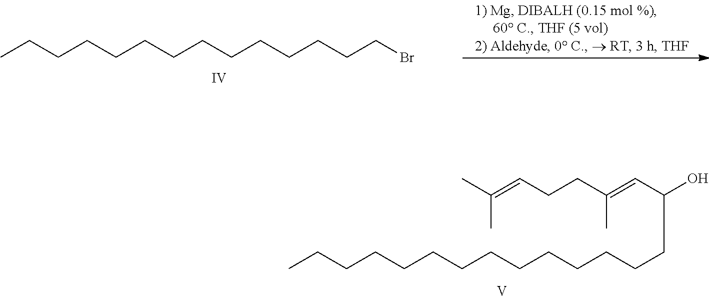

Magnesium (0.3 g, 1.8 equiv, 0.01 mol) was activated, grinded and added into the flask under argon atmosphere. 1 volume of THF added (3 mL) and DIBAL-H was added. 1-bromotetradecane (IV) (2 g, 2 mL, 1.2 equiv, 8 mmol) was dissolved in 5 mL of THF and one half of the solution was added to the magnesium turnings. The reaction is exothermic, and the solution turned gray. Second part of the solution added, and reaction was left for 1 h to stir at RT. Gray solution was formed and upon cooling Grignard reagent precipitated. (E)-3,7-dimethylocta-2,6-dienal (II) (1 g, 1 equiv, 7 mmol) in THF (5 mL) was added with precipitate dissolving upon addition and solution vigorously warming up. After 1 h reaction quenched with NH₄Cl and extracted with EtOAc. The organic phase was washed with brine and dried over sodium sulfate, filtered and concentrated to give a residue. The residue was purified by column chromatography to give compound V as a yellow oil. ¹H NMR (600 MHz, CDCl₃) δ 5.17 (dq, J=8.7, 1.3 Hz, 1H), 5.09 (t, J=1.5 Hz, 1H), 4.36 (q, J=8.9 Hz, 1H), 2.11 (dt, J=14.1, 7.0 Hz, 2H), 2.05-2.00 (m, 2H), 1.69 (s, 6H), 1.26 (s, 30H), 0.89 (t, J=7.0 Hz, 3H).

### Step 4

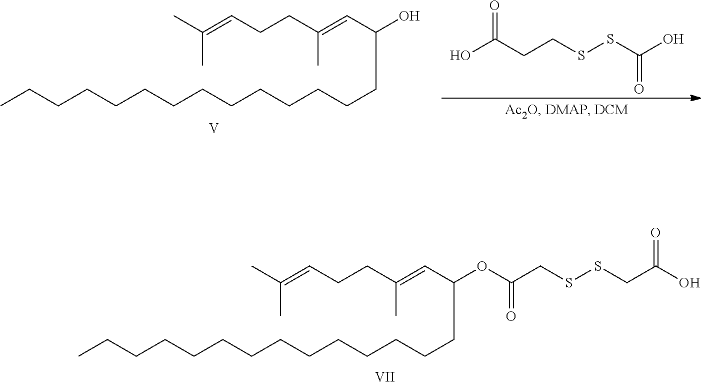

A solution of 2,2′-disulfanediyldiacetic acid (VI) (1.34 g, 2.74 equiv, 7.35 mmol) and acetic anhydride (20.2 mL, 80 equiv, 214 mmol) was stirred for 2 h at 25° C. The solution was then evaporated to dryness under vacuum, with addition of toluene three times. To the residue was added dichloromethane (2 mL), (E)-2,6-dimethyldocosa-2,6-dien-8-ol (V, 940 mg, 1 equiv, 2.68 mmol) and a catalytic amount of DMAP (32.8 mg, 0.1 equiv, 268 μmol). The reaction mixture was stirred at room temperature for 5 min. The product (VII) was purified by silica gel column chromatography, eluting with a solution of hexane, ethyl acetate and acetic acid. ¹H NMR (401 MHz, CDCl₃) δ 5.54 (dt, J=9.2, 6.7 Hz, 1H), 5.16-5.02 (m, 2H), 3.62 (s, 2H), 3.57 (d, J=1.1 Hz, 2H), 2.16-1.98 (m, 4H), 1.74 (s, 3H), 1.68 (s, 4H), 1.61 (s, 3H), 1.29 (t, J=6.6 Hz, 26H), 0.89 (t, 3H).

### Step 5

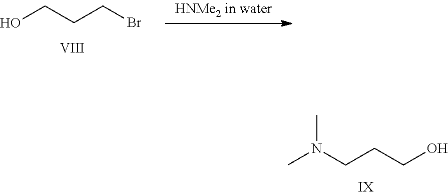

Trimethylenebromohydrin (VIII) (4 g, 3 mL, 1 equiv, 0.03 mol) was added dropwise to dimethylamine (14 mL, 40% Wt, 4 equiv, 0.11 mol) in water. DCM (40 mL) was added, and the mixture was washed with a 5% w/w solution of NaHCO₃ (3×10 mL) followed by brine (10 mL). The phases were separated, and the organic phase dried over MgSO₄. After filtration and elimination of the solvent by distillation at atmospheric pressure, the product was obtained as a yellowish oil (IX, 135 mg). No further purification was performed. ¹H NMR (600 MHz, CDCl₃) δ 3.83-3.77 (m, 2H), 2.57-2.52 (m, 2H), 2.26 (s, 6H), 1.73-1.66 (m, 2H).

### Step 6

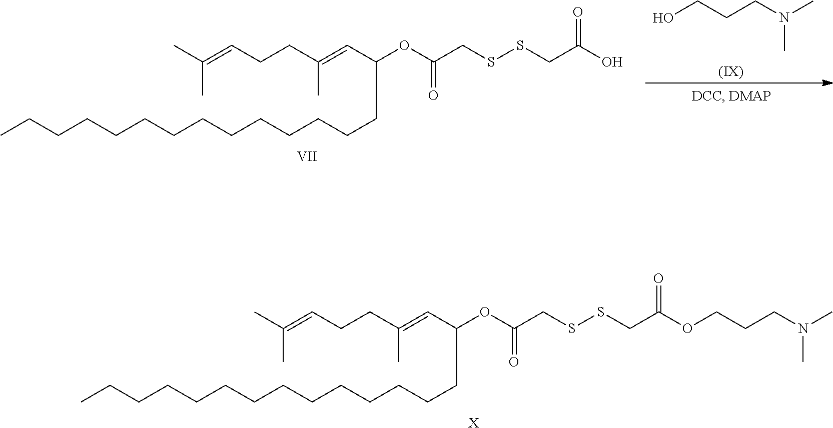

(E)-2-((2-((2,6-dimethyldocosa-2,6-dien-8-yl)oxy)-2-oxoethyl)disulfaneyl)acetic acid (VII) (809 mg, 1 equiv, 1.57 mmol) and dichloromethane (10 mL) were stirred in an ice-water bath. After 5 min, 3-(dimethylamino)propan-1-ol (IX) (279 μL, 1.5 equiv, 2.36 mmol) was added into the solution. Later addition of dicyclohexylmethanediimine (486 mg, 1.5 equiv, 2.36 mmol) and N,N-dimethylpyridin-4-amine (19.2 mg, 0.1 equiv, 157 μmol) was done. The solution was stirred for a further 2 h, filtered to remove DCU and purified by silica gel column chromatography (eluent: chloroform and 1% methanol). The product (X, Compound 100) was obtained as an orange oil with a 56% yield.

Compound 100: ¹H NMR (400 MHz, CDCl₃) δ 5.53 (dt, J=9.3, 6.8 Hz, 1H), 5.18-5.03 (m, 2H), 4.21 (t, J=6.6 Hz, 2H), 3.58 (s, 2H), 3.55 (d, J=1.3 Hz, 2H), 2.36 (d, J=7.3 Hz, 2H), 2.24 (s, 6H), 2.15-2.05 (m, 2H), 2.05-1.99 (m, 2H), 1.84 (d, J=6.8 Hz, 2H), 1.73 (d, J=1.3 Hz, 3H), 1.68 (d, J=1.4 Hz, 4H), 1.61 (d, J=1.3 Hz, 3H), 1.27 (s, 24H), 0.92-0.84 (t, J=6.6 Hz, 3H). ¹³C NMR (151 MHz, CDCl₃) δ 169.29, 168.58, 141.01, 131.69, 123.77, 123.15, 73.18, 63.94, 55.86, 45.10, 41.79, 41.75, 41.32, 39.52, 34.83, 31.90, 29.68, 29.63, 29.59, 29.54, 29.44, 29.33, 26.54, 26.52, 26.26, 25.66, 25.04, 22.66, 17.68, 16.84, 14.09. FT-IR 2924, 2856, 1738 cm⁻¹. Anal. Calcd; C, 66.06; H, 10.25; N, 2.33; S, 10.69. Found; C, 65.94; H, 10.36; N, 2.67; S, 9.34. HRMS (m/z) Calcd. For C₃₃H₆₁NS₂O₄ 600.4115; Found 600.4120.

Compound 105 was prepared according to the method used to prepare Compound 100. The following intermediate VIIa has been characterized after step 4:

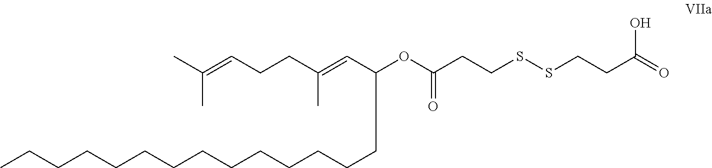 [SMILES: CCCCCCCCCCCCCCC(/C=C(\C)CCC=C(C)C)OC(=O)CCSSCCC(=O)O]

Compound VIIa: ¹H NMR (400 MHz, CDCl₃) δ 5.51 (dt, J=9.2, 6.7 Hz, 1H), 5.13-5.02 (m, 2H), 2.99-2.87 (m, 4H), 2.84-2.76 (m, 2H), 2.75-2.66 (m, 2H), 2.15-1.98 (m, 6H), 1.72 (s, 3H), 1.68 (s, 3H), 1.61 (s, 3H), 1.26 (s, 24H), 0.88 (t, J=6.5 Hz, 3H).

Then, compound 105 was obtained at the end of step 6.

Compound 105: ¹H NMR (600 MHz, CDCl₃) δ 5.54-5.46 (m, 1H), 5.13-5.01 (m, 2H), 4.16 (t, J=6.4 Hz, 2H), 2.95-2.88 (m, 4H), 2.72 (td, J=14.4, 7.2 Hz, 4H), 2.37 (t, J=7.2 Hz, 2H), 2.25 (s, 6H), 2.08 (t, J=5.6 Hz, 2H), 2.04-2.00 (m, 2H), 1.87-1.79 (m, 2H), 1.72 (s, 3H), 1.68 (s, 3H), 1.60 (s, 3H), 1.26 (s, 26H), 0.88 (t, J=6.8 Hz, 3H). ¹³C NMR (101 MHz, CDCl₃) δ 171.6, 171.0, 140.8, 140.5, 131.9, 131.7, 124.3, 123.9, 123.8, 123.5, 72.2, 71.9, 63.2, 62.9, 56.2, 56.2, 45.5, 39.5, 35.2, 34.9, 34.5, 34.5, 34.1, 34.0, 33.4, 33.2, 32.5, 31.9, 29.7, 29.7, 29.7, 29.6, 29.6, 29.5, 29.5, 29.4, 27.0, 27.0, 26.6, 26.3, 25.7, 25.3, 25.1, 23.3, 22.7, 21.0, 17.7, 17.6, 16.8, 14.1; FT-IR 2922, 1732, 1236, 1169 cm⁻¹; LCMS: Rt=3.105 min, [M+H]⁺=628.9; HPLC: Rt=2.660 min, purity: 95.2%.

Compound 106 can be prepared according to the method used to prepare Compound 100. At the end of step 3, the following intermediate Vi has been characterized:

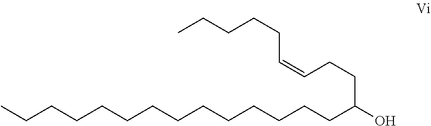 [SMILES: CCCCC/C=C\CCC(O)CCCCCCCCCCCCCC]

Compound Vi: ¹H NMR (400 MHz, CDCl₃) δ 5.47-5.32 (m, 2H), 3.71-3.55 (m, 1H), 2.25-2.09 (m, 2H), 2.08-2.02 (m, 2H), 1.56-1.21 (m, 35H), 0.94-0.85 (m, 6H).

Then, intermediate Vi can be reacted according to step 4 to form the compound VIIi:

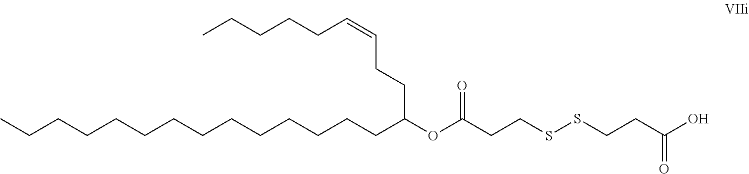 [SMILES: CCCCC/C=C\CCC(CCCCCCCCCCCCCC)OC(=O)CCSSCCC(=O)O]

Compound VIIi: ¹H NMR (400 MHz, CDCl₃) δ 5.43-5.26 (m, 2H), 4.91 (m, 1H), 2.93 (m, 4H), 2.84-2.77 (m, 2H), 2.76-2.68 (m, 2H), 2.09-1.95 (m, 4H), 1.64-1.49 (m, 4H), 1.35-1.20 (m, 30H), 0.88 (m, 6H).

Compound VIIi can also been prepared according to an alternative approach to step 4, represented as the following step 4i.

### Step 4i

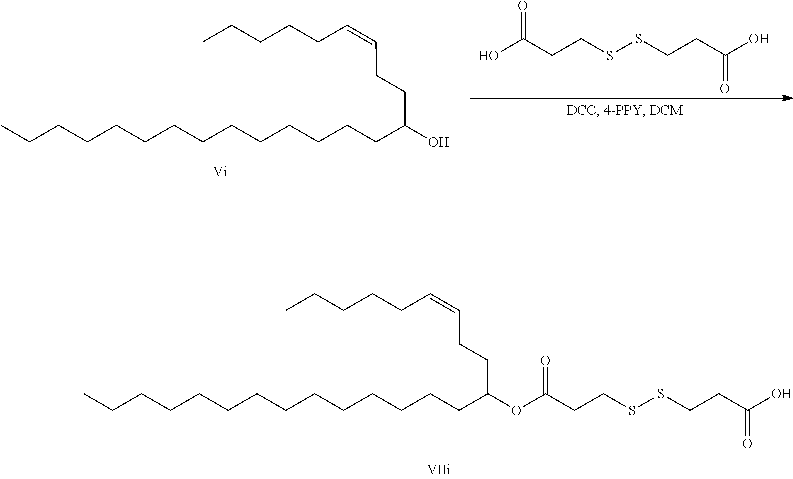 [SMILES: CCCCC/C=C\CCC(O)CCCCCCCCCCCCCC ; O=C(O)CCSSCCC(=O)O ; CCCCC/C=C\CCC(CCCCCCCCCCCCCC)OC(=O)CCSSCCC(=O)O]

To a solution of (Z)-tetracos-6-en-10-ol (Vi) (5 g, 14.18 mmol, 1 equiv),3-(2-carboxyethyl-disulfanyl)propanoic acid (11.93 g, 56.72 mmol, 4 equiv) in dichloromethane (50 mL) was added dicyclohexylcarbodiimide (4.39 g, 21.27 mmol, 4.30 mL, 1.50 equiv) and 4-pyrrolidin-1-ylpyridine (525.35 mg, 3.54 mmol, 0.25 equiv). The mixture was stirred at 20° C. for 15 h. The reaction mixture was filtered, the filtrate was concentrated to afford a residue. The residue was purified by column chromatography (SiO₂, petroleum ether/ethyl acetate=10/1 to 4/1) to give 3-[[3-[1-[(Z)-non-3-enyl]pentadecoxy]-3-oxo-propyl]disulfanyl]propanoic acid (VIIi, 2.3 g, 4.22 mmol, 29.77% yield, 95% purity) as a colorless oil.

Then, compound 106 was obtained at the end of step 6.

Compound 106: ¹H NMR (600 MHz, CDCl₃) δ 5.42-5.26 (m, 2H), 4.91 (m, 1H), 4.18-4.12 (m, 2H), 2.96-2.90 (m, 4H), 2.77-2.69 (m, 4H), 2.37 (t, J=7.4 Hz, 2H), 2.25 (s, 6H), 2.05-1.96 (m, 4H), 1.85-1.80 (m, 2H), 1.62-1.50 (m, 4H), 1.33-1.21 (m, 30H), 0.88 (dt, J=3.4, 6.8 Hz, 6H). ¹³C NMR (101 MHz, CDCl₃) δ 171.7, 171.4, 130.7, 128.5, 74.7, 63.1, 56.1, 45.3, 35.0, 34.1, 33.2, 31.9, 29.7, 29.7, 29.5, 29.4, 27.2, 23.2, 22.7, 22.6, 14.1. FT-IR: 2922, 1732, 1169 cm⁻¹; LCMS: Rt=3.147 min, [M+H]+=631.2; HPLC: Rt=3.560 min, purity: 99.0%.

Disulfide Lipids with Three Branches

As a non-limiting example, Compound 109 may be prepared according to the following synthesis scheme. Other disulfide lipids with three branches may be prepared in similar synthetic steps.

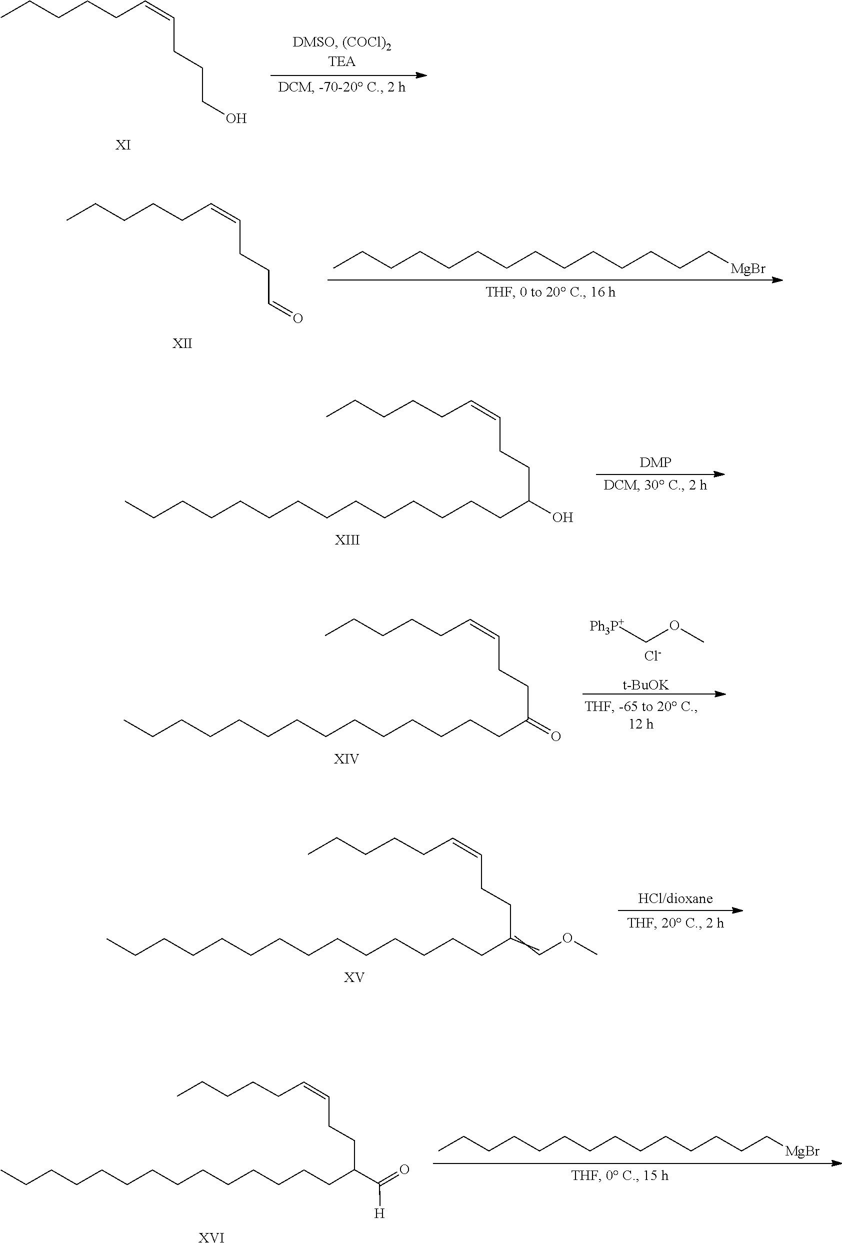

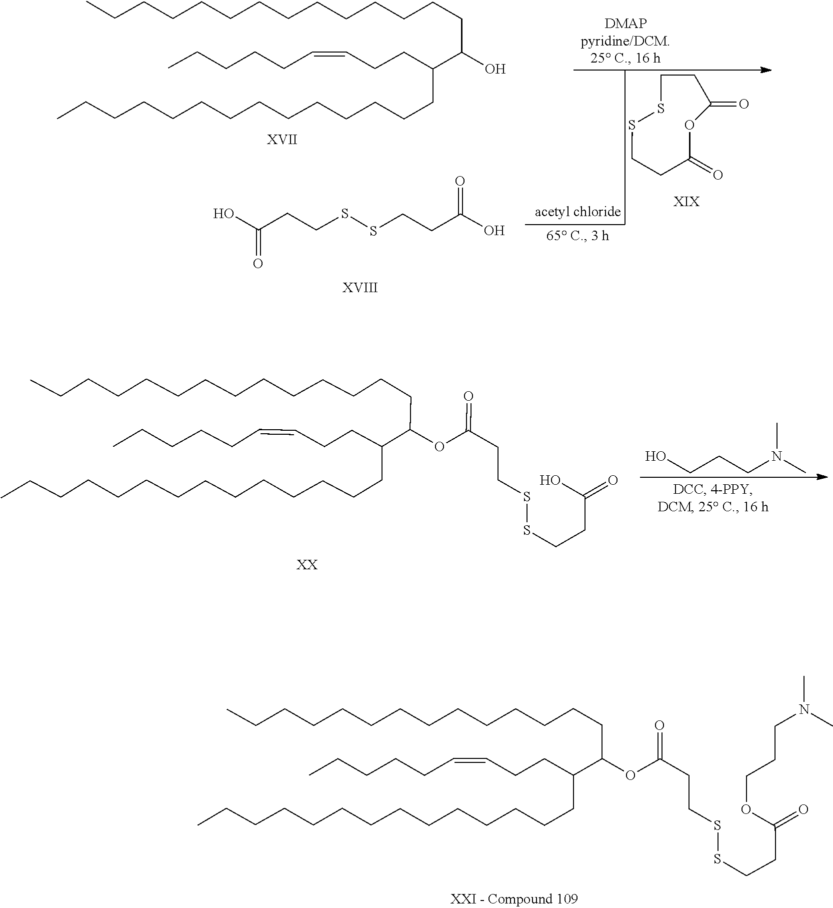

### Step 1

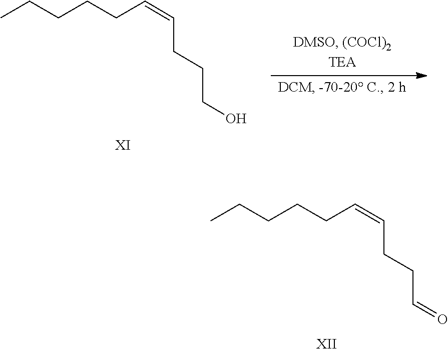

To a solution of oxalyl dichloride (60.9 g, 480 mmol, 42.0 mL, 1.50 equiv) in dichloromethane (500 mL) at −78° C. was added dropwise a solution of dimethylsulfoxide (75.0 g, 960 mmol, 75.0 mL, 3.00 equiv). The mixture was stirred at −78° C. for 0.5 hours. (Z)-dec-4-en-1-ol XI (50.0 g, 320 mmol, 1.00 equiv) was added dropwise to the mixture, stirred at −78° C. for 0.5 hours. Triethylamine (130 g, 1.28 mol, 178 mL, 4.00 equiv) was added dropwise to the mixture and stirred at −78° C. for 0.5 hours, after that the mixture was warmed up to room temperature and stirred at 20° C. for 0.5 hours. The reaction mixture was partitioned between hydrochloric acid (1.00 M in water) (2 L) and dichloromethane (1 L). The organic phase was separated, washed with dichloromethane (500 mL), dried over sodium sulfate, filtered and concentrated under reduced pressure to give a residue. The residue was purified by column chromatography (SiO₂, Petroleum ether/Ethyl acetate=100/0 to 10/1). (Z)-dec-4-enal (XII, 48.0 g, 249 mmol, 77.8% yield, 80.0% purity) as a yellow oil. ¹H NMR: (400 MHz, CDCl₃) δ 9.70 (t, J=1.4 Hz, 1H), 5.44-5.20 (m, 2H), 2.46-2.35 (m, 2H), 2.34-2.24 (m, 2H), 2.02-1.90 (m, 2H), 1.33-1.12 (m, 6H), 0.82 (t, J=6.8 Hz, 3H).

### Step 2

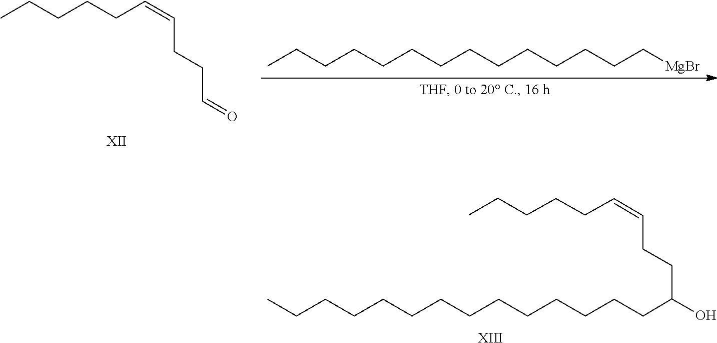

Magnesium (8.51 g, 350 mmol, 1.80 equiv) in tetrahydrofuran (120 mL) was added to the flask at nitrogen atmosphere and then diisobutylaluminum hydride (1.00 M in tetrahydrofuran, 2.90 mL, 0.0150 equiv) was added to the mixture. 1-bromotetradecane (64.7 g, 233 mmol, 69.6 mL, 1.20 equiv) was dissolved in tetrahydrofuran (150 mL) and one half the solution was added to the magnesium turnings. Exothermic reaction observed, and solution turned gray. Second part of the solution added, and reaction was left for 1 hour to stir at 60° C. Gray solution was formed and upon cooling Grignard reagent precipitated. (Z)-dec-4-enal (XII, 30.0 g, 194 mmol, 1.00 equiv) in tetrahydrofuran (150 mL) was added at 0° C. with precipitate dissolving upon addition and solution vigorously warming up. The mixture was stirred at 20° C. for 16 hours. The mixture was quenched with ammonium chloride (600 mL), extracted with ethyl acetate (100 mL×3), the organic phase was washed with brine (300 mL), dried over sodium sulfate, filtered and concentrated to give a residue. The residue was purified by flash silica gel chromatography (ISCO®; 660 g SepaFlash® Silica Flash Column, Eluent of from 0 to 1% Ethyl acetate/Petroleum ether gradient at 100 mL/min). Petroleum ether/Ethyl acetate=20/1, R_{f}=0.400. (Z)-tetracos-6-en-10-ol (XIII, 53.0 g, 150 mmol, 77.3% yield) was obtained as a white solid. ¹H NMR: (400 MHz, CDCl₃) δ 5.47-5.32 (m, 2H), 3.68-3.56 (m, 1H), 2.24-2.09 (m, 2H), 2.05 (q, J=6.4 Hz, 2H), 1.59-1.47 (m, 3H), 1.39-1.25 (m, 32H), 0.89 (dt, J=3.2, 6.8 Hz, 6H).

### Step 3

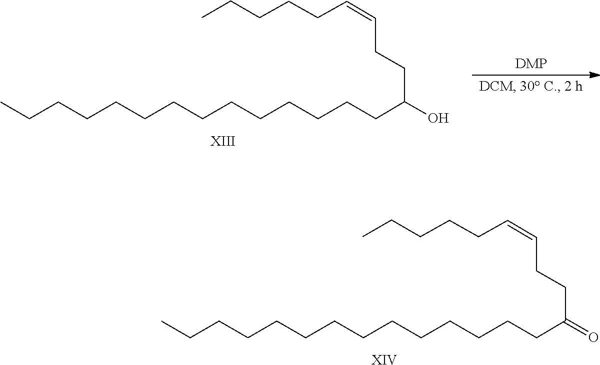

To a solution of (Z)-tetracos-6-en-10-ol (XIII, 44.5 g, 126 mmol, 1.00 equiv) in dichloromethane (450 mL) was added Dess-Martin periodinane (107.0 g, 252 mmol, 78.1 mL, 2.00 equiv) portion-wise at a temperature of about 20˜30° C., and the resulting mixture was stirred at 30° C. for 2 hours. The mixture was filtered and the filtrate was quenched with water (500 mL) and basified with saturated sodium bicarbonate (500 mL) and saturated sodium sulfite (500 mL), the organic phase was separated and the water layer was extracted with dichloromethane (100 mL×3), the collected layers were dried over anhydrous sodium sulfate, filtered and concentrated in vacuum to give a residue. The residue was purified by column chromatography (SiO₂, n-Heptane/Ethyl acetate=1/0 to 5/1). Petroleum ether/Ethyl acetate=20/1, R_{f}=0.600. (Z)-tetracos-6-en-10-one (XIV, 35.0 g, 99.8 mmol, 79.1% yield) was obtained as a yellow oil. ¹H NMR: (400 MHz, CDCl₃) δ 5.44-5.20 (m, 2H), 2.45-2.33 (m, 4H), 2.28 (q, J=7.2 Hz, 2H), 2.01 (q, J=7.2 Hz, 2H), 1.55 (t, J=7.2 Hz, 2H), 1.37-1.22 (m, 28H), 0.87 (dt, J=2.0, 6.8 Hz, 6H).

### Step 4

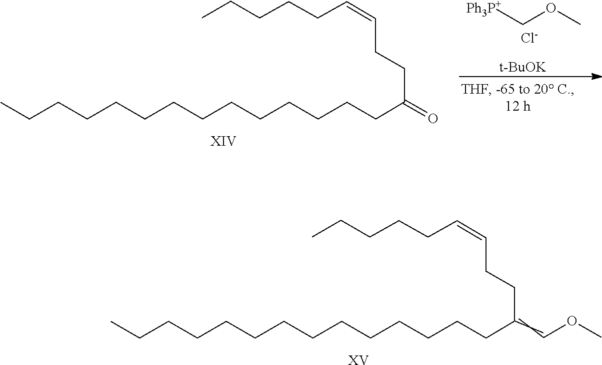

To a suspension of (methoxymethyl)triphenylphosphonium chloride (51.3 g, 150 mmol, 1.50 equiv) in dry tetrahydrofuran (150 mL) stirred at a temperature of about −65˜−55° C. was added potassium tert-butoxide (1.00 M in tetrahydrofuran, 150 mL, 1.50 equiv) dropwise. The reaction mixture was allowed to stir at a temperature of about −10˜0° C. for 30 min, then cooled to −65° C., after which (Z)-tetracos-6-en-10-one (XIV, 35.0 g, 99.8 mmol, 1.00 equiv) was added. The reaction mixture was allowed to warm to 25° C. and stirred for 11.5 hours. The mixture was quenched with water (250 mL) and ethyl acetate (250 mL), the aqueous solution was extracted with ethyl acetate (250 mL×3), then the collected layers were washed with water (500 mL) and brine (500 mL), dried over anhydrous sodium sulfate, filtered and concentrated in vacuum to give a residue. The crude residue was used directly without further purification. (6Z)-10-(methoxymethylene)tetracos-6-ene (XV, 38.0 g, crude) was obtained as a yellow oil.

### Step 5

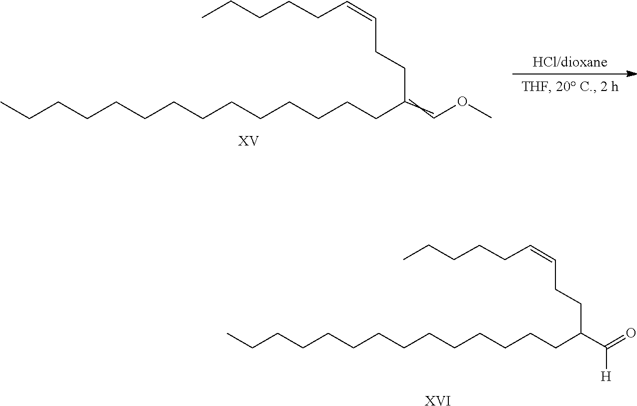

To a solution of (6Z)-10-(methoxymethylene)tetracos-6-ene (XV, 37.0 g, 97.71 mmol, 1.00 equiv) in dichloromethane (200 mL) was added hydrochloric acid (4.00 M in dioxane, 24.4 mL, 1.00 equiv) at 25° C., the resulting mixture was stirred at 25° C. for further 2 hrs. The mixture was quenched with saturated sodium sulfate (100 mL) and diluted with dichloromethane (200 mL), the organic phase was separated and the aqueous solution was extracted with dichloromethane (100 mL×3), the collected layers were washed with brine (300 mL) and dried over anhydrous sodium sulfate, filtered and concentrated in vacuum to give a residue. The crude residue was used without further purification. (Z)-2-(non-3-en-1-yl)hexadecanal (XVI, 30.0 g, 82.3 mmol, 84.2% yield) was obtained as a yellow oil. ¹H NMR: (400 MHz, CDCl₃) δ 9.59 (d, J=3.2 Hz, 1H), 5.48-5.24 (m, 2H), 2.27 (dtt, J=3.2, 5.4, 8.0 Hz, 1H), 2.11-1.94 (m, 4H), 1.76-1.58 (m, 2H), 1.55-1.40 (m, 2H), 1.39-1.25 (m, 30H), 0.89 (dt, J=2.8, 6.8 Hz, 6H).

### Step 6

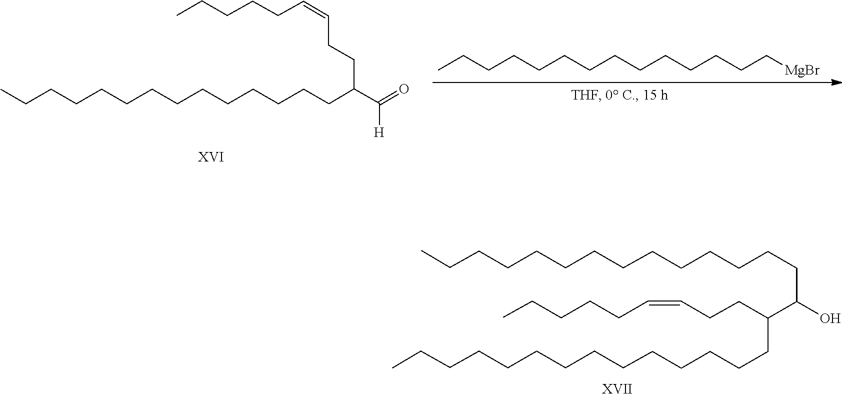

Magnesium (720 mg, 29.6 mmol, 1.80 equiv) in tetrahydrofuran (24 mL) was added to the flask at nitrogen atmosphere and then diisobutylaluminum hydride (1.00 M in tetrahydrofuran, 247 μL, 0.0150 equiv) was added to the mixture. 1-bromotetradecane (5.47 g, 19.8 mmol, 5.90 mL, 1.20 equiv) was dissolved in tetrahydrofuran (30 mL) and one half the solution was added to the magnesium turnings. Exothermic reaction observed, and solution turned gray. Second part of the solution added, and reaction was left for 1 hour to stir at 60° C. Gray solution was formed and upon cooling Grignard reagent precipitated. (Z)-2-(non-3-en-1-yl)hexadecanal (XVI, 6.00 g, 16.5 mmol, 1.00 equiv) in tetrahydrofuran (30.0 mL) was added at 0° C. with precipitate dissolving upon addition and solution vigorously warming up. The mixture was stirred at 20° C. for 12 hrs. The mixture was quenched with ammonium chloride (100 mL), extracted with ethyl acetate (200 mL×3), the organic phase was washed with brine (100 mL), dried over sodium sulfate, filtered and concentrated to give the residue. The residue was purified by flash silica gel chromatography (ISCO®; 120 g SepaFlash® Silica Flash Column, Eluent from 0 to 3% Ethyl acetate/Petroleum ether gradient @100 mL/min). Petroleum ether/Ethyl acetate=20/1, R_{f}=0.400. (Z)-16-(non-3-en-1-yl)triacontan-15-ol (XVII, 3.50 g, 6.22 mmol, 37.8% yield) was obtained as a yellow oil. ¹H NMR: (400 MHz, CDCl₃) δ 5.48-5.26 (m, 2H), 3.65-3.58 (m, 1H), 3.36 (d, J=0.8 Hz, 1H), 2.14-1.92 (m, 4H), 1.55-1.14 (m, 61H), 0.95-0.78 (m, 9H).

### Step 7

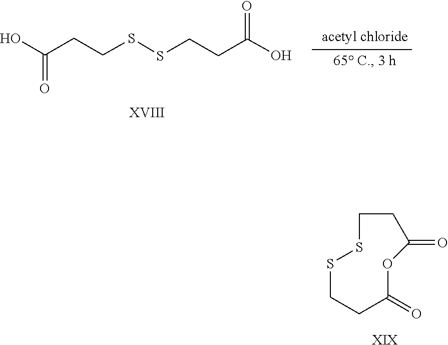

A mixture of 3-(2-carboxyethyldisulfanyl)propanoic acid (XVIII, 10.0 g, 47.6 mmol, 1.00 equiv) in acetyl chloride (50 mL) was stirred at 65° C. for 3 hours under nitrogen. The reaction mixture was concentrated in vacuum to afford the product of 1,5,6-oxadithionane-2,9-dione (XIX, 9.00 g, crude) which was used in the next step without further purification.

### Step 8

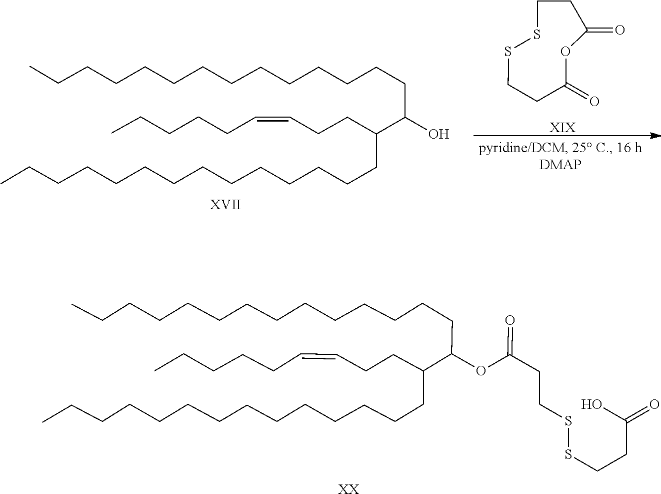

A solution of 1,5,6-oxadithionane-2,9-dione (XIX, 7.38 g, 38.36 mmol, 10.0 equiv) and 4-dimethylaminopyridine (421 mg, 3.45 mmol, 0.900 equiv) in pyridine (15 mL) and dichloromethane (30 mL) was stirred at 25° C. for 0.5 hrs, then 16-[(Z)-non-3-enyl]triacontan-15-ol (XVII, 2.70 g, 3.84 mmol, 80.0% purity, 1.00 equiv) was added, and the reaction mixture was stirred at 25° C. for 11.5 hrs. The reaction mixture was diluted with dichloromethane (50 mL) and washed with hydrochloric acid (2.00 M in water) (20 mL×2). The organic phases were washed with brine (15 mL), dried over anhydrous sodium sulfate, filtered and concentrated in vacuum to give a residue. The residue was purified by silica gel chromatography (SiO₂, Petroleum ether:Ethyl acetate=5:1) to afford the product of 3-[[3-[2-[(Z)-non-3-enyl]-1-tetradecyl-hexadecoxy]-3-oxo-propyl]disulfanyl]propanoic acid (XX, 1.50 g, 1.99 mmol, 51.87% yield) as a yellow oil. ¹H NMR: (400 MHz, CDCl₃) δ 5.43-5.31 (m, 2H), 4.99 (td, J=4.0, 8.0 Hz, 1H), 2.98-2.89 (m, 4H), 2.85-2.79 (m, 2H), 2.76-2.69 (m, 2H), 2.08-1.99 (m, 4H), 1.58-1.47 (m, 3H), 1.32-1.17 (m, 58H), 0.93-0.87 (m, 9H).

### Step 9

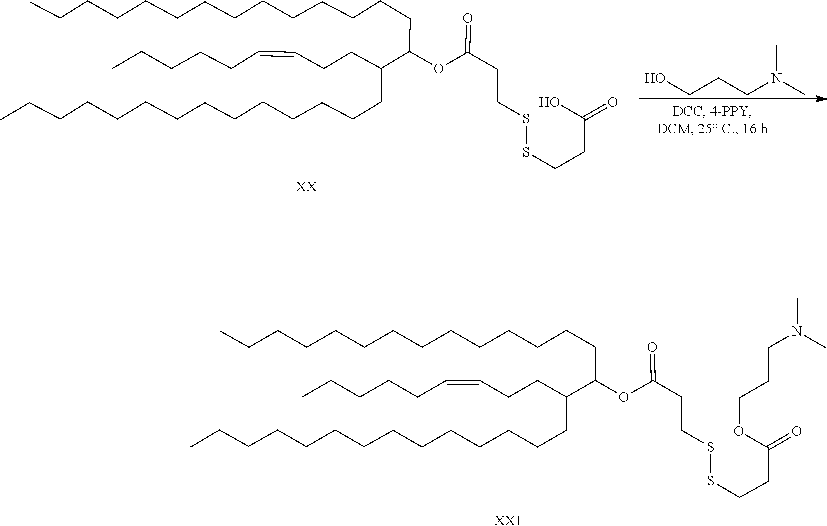

A solution of 3-[[3-[2-[(Z)-non-3-enyl]-1-tetradecyl-hexadecoxy]-3-oxo-propyl]disulfanyl]propanoic acid (XX, 1.50 g, 1.99 mmol, 1.00 equiv), 3-(dimethylamino)-propan-1-ol (246 mg, 2.38 mmol, 279 uL, 1.20 equiv), dicyclohexylmethanediimine (450.8 mg, 2.18 mmol, 442 uL, 1.10 eq) and 4-(pyrrolidin-1-yl)-pyridine (58.9 mg, 397 umol, 0.200 equiv) in dichloromethane (15 mL) was stirred at 25° C. for 12 hrs. The mixture was filtered and the filtrate was concentrated in vacuum to give a residue. The residue was purified by silica gel chromatography (Dichloromethane:Methanol=10:1) to afford the product of 3-(dimethylamino)propyl-3-[[3-[2-[(Z)-non-3-enyl]-1-tetradecyl-hexadeoxy]-3-oxo-propyl]disulfanyl]propanoate (XXI, Compound 109, 1.20 g, 1.36 mmol, 68.7% yield, 95.6% purity) as a yellow oil.

Compound 109: ¹H NMR: (400 MHz, CDCl₃) δ 5.46-5.25 (m, 2H), 4.98 (td, J=4.0, 8.0 Hz, 1H), 4.18 (t, J=6.0 Hz, 2H), 2.99-2.90 (m, 4H), 2.77-2.64 (m, 4H), 2.41 (t, J=8.0 Hz, 2H), 2.28 (s, 6H), 2.09-1.82 (m, 10H), 1.33-1.18 (m, 57H), 0.93-0.86 (m, 9H). ¹³C NMR (100 MHz, CDCl₃) 171.7, 171.4, 130.3, 129.5, 63.1, 56.1, 45.3, 40.6, 34.4, 34.1, 31.9, 29.7, 29.7, 29.4, 27.3, 26.7, 25.1, 22.7, 22.6, 14.1, 14.1. FT-IR: 2920, 2850, 1458, 1234 cm⁻¹; LCMS: R_{f}=2.723 min, [M+H]⁺=814.4; HPLC: R_{f}=3.123 min

### Example 2: Lipids Stability

The lipids stability was evaluated by ¹H NMR at −20° C. and at room temperature. The results are reported in Table 3. “+” means the lipid is stable, “Minor” means the main characteristic peaks of the lipid are present, no major change of the ¹H NMR spectrum is observed, but some extra peaks appearance observed. “Major” means main the characteristic peaks of the lipid underwent transformation, some peaks can be missing or decreased in intensity, impurity peaks either increased in size or represent main peaks, as observed on ¹H NMR. “n.d.” means “not determined”.

TABLE 3
Lipids stability characterized by ¹H NMR at −20° C. and room temperature

| | | | Room Temperature | | | | | |
|---|---|---|---|---|---|---|---|---|
| | At −20° C., | | 1 | 2 | 3 | 4 | 2 | 3 |
| Lipid | months | | week | weeks | weeks | weeks | months | months |
| 100 | 3 | + | + | Minor | Minor | Minor | Major | Major |
| 105 | 3 | + | + | + | + | + | + | + |
| 106 | 3 | + | + | + | + | + | + | n.d. |
| 109 | 3 | + | + | + | + | + | + | n.d. |

### Example 3: Surface pKa Values of LNPs

The surface pKa values of LNPs were determined as follows. Briefly, solutions of 100 mM citrate buffer at, 100 mM Tris-HCl and 50 mM sodium bicarbonate-sodium hydroxide buffer were titrated to pH values varying by 1 from 3.0 to 11.0 and aliquoted into a clear 96-well plate. LNPs and 2-(p-toluidinyl)naphthalene-6-sulfonic acid (TNS) were diluted into these solutions to obtain a final concentration of 20 μM lipid and 6 μM TNS, respectively. Fluorescence intensity was read on a Glowmax discover plate reader at an excitation of 322 nm and an emission of 430 nm. pKa values were calculated as the pH corresponding to 50% LNP protonation, assuming minimum and maximum fluorescence values corresponded to zero and 100% protonation, respectively.

TABLE 4
surface pKa values measured using the 6-(p-Toluidino)-
2-naphthalenesulfonyl chloride (TNS) assay

| LNPs Formed with the Following | |
|---|---|
| Ionizable Lipids | Surface pKa |
| 100 | 7.5 |
| 105 | 7.7 |
| 106 | 8.2 |
| 109 | 7.0 |

### Example 4: Methods of Making the Delivery Vehicles

The delivery vehicles such as LNPs of the present disclosure may be prepared using any convenient methodology. In one non-limiting example, the LNPs are formed by mixing equal volumes of lipids dissolved in alcohol with oligonucleotide payloads dissolved in a citrate buffer by an impinging jet process.

The lipid solution contains a cationic lipid compound of the present disclosure, a helper lipid, a neutral lipid and a PEGylated lipid. The payload to total lipid ratio is approximately 1:30-40 (wt/wt) depending on molecular weight of ionizable lipid. The LNPs are formed by mixing equal volumes of lipid solution in ethanol with oligonucleotide payloads dissolved in a citrate buffer by an impinging jet process through a mixing device. The mixed LNP solution is held at room temperature for 0-24 hrs prior to a dilution step.

The solution is then concentrated and diafiltered with suitable buffer by ultrafiltration or dialysis process using membranes. The final product is sterile filtered and stored at −20 or −80° C.

In another non-limiting example, the LNPs are prepared using an alcohol dilution method. An mRNA solution is prepared by dissolving mRNA in a buffered solution. A lipid solution is prepared by dissolving an ionizable disulfide lipid according to the present disclosure, a neutral lipid (i.e., phospholipid), a helper lipid (i.e., cholesterol) and a conjugated lipid (i.e. PEGylated lipid) in absolute ethanol. The mRNA and lipid solutions were mixed, prior to a dilution step. The resulting LNPs were then concentrated and submitted to buffer exchange.

### Example 5: Characterization of the Delivery Vehicles

Selected ionizable lipids were screened to prepare LNPs formulated with 3-GP mRNA (internal control vaccine mRNA). Physicochemical parameters recorded for LNPs prepared using ionizable lipids from the present application (compounds 100, 105, 106 and 109) are summarized in Table 5 (all results are from duplicate measurements).

TABLE 5
Physicochemical parameters of LNPs with 3-GP mRNA

| | LNPs-100-01 | LNPs-105-01 | LNPs-106-01 | LNPs-109-01 |
|---|---|---|---|---|
| Ionizable Lipid | 100 | 105 | 106 | 109 |
| Size (nm) | 60.45 | 57.85 | 84.69 | 65.66 |
| | 59.65 | 57.94 | 87.91 | 65.07 |
| PDI | 0.1943 | 0.1803 | 0.1654 | 0.2087 |
| | 0.1732 | 0.1552 | 0.1616 | 0.2296 |
| Zeta Potential | −7.002 | 2.929 | 2.847 | −0.02492 |
| (mV) | −5.305 | 3.551 | 3.348 | −2.142 |
| EE (%) | 92.5 | 98.6 | 94.7 | 98.8 |
| Concentration - | 221.0 | 183.1 | 211.8 | 204.4 |
| ribo (ug/mL) | | | | |
| HPLC Purity (%) | 98.10/98.06 | 96.81/96.77 | 98.36/98.36 | 98.37/98.43 |
| Concentration - | 263/261 | 224/225 | 274/273 | 256/257 |
| HPLC (ug/mL) | | | | |
| pH | 7.89 | 7.54 | 7.52 | 7.63 |
| Osmolality | 653 | 608/527 | 603/599 | 557 |
| (mOsm) | 619 | 57.85 | 84.69 | 635 |

Various conditions were screened in order to study LNPs for mRNA delivery (3-GP mRNA) using compound 105 as the ionizable lipid. Formulations that were used in order to obtain the LNPs were obtained by preparing LNPs with an ionizable disulfide lipid according to the present disclosure, a neutral lipid (e.g., phospholipid), a helper lipid (e.g., cholesterol), a conjugated lipid (e.g., PEGylated lipid), optionally an additional cationic lipid and excipients. LNPs obtained were characterized in term of particle size, polydispersity index (PDI) and percentage of mRNA encapsulation as summarized in Table 6.

TABLE 6
Characterization of LNPs with 3-GP mRNA

| | Ionizable | | | |
|---|---|---|---|---|
| | Lipid | Size (nm) | PDI | Encapsulation (%) |
| LNPs-105-02 | 105 | 60 | 0.18 | 0.25 |
| LNPs-105-03 | 105 | 55 | 0.14 | 0.28 |
| LNPs-105-04 | 105 | 66 | 0.16 | 0.24 |
| LNPs-105-05 | 105 | 57 | 0.17 | 0.26 |

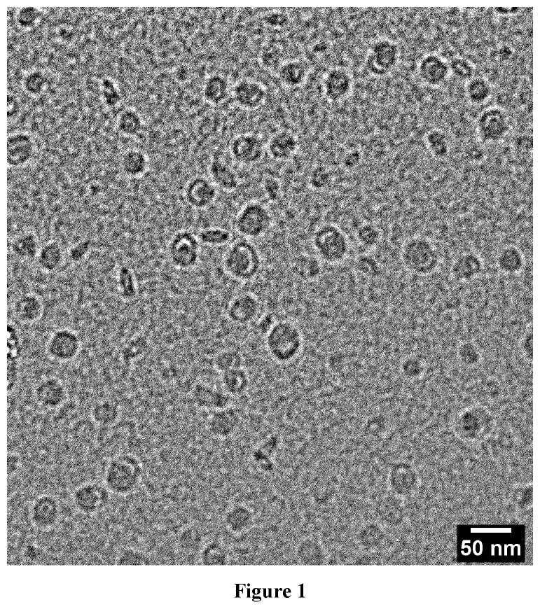

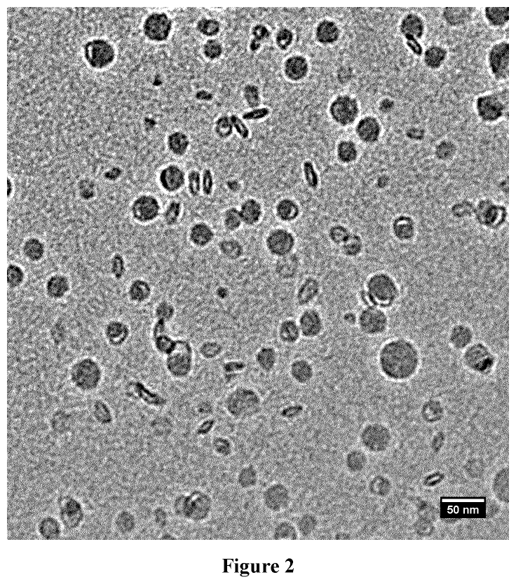

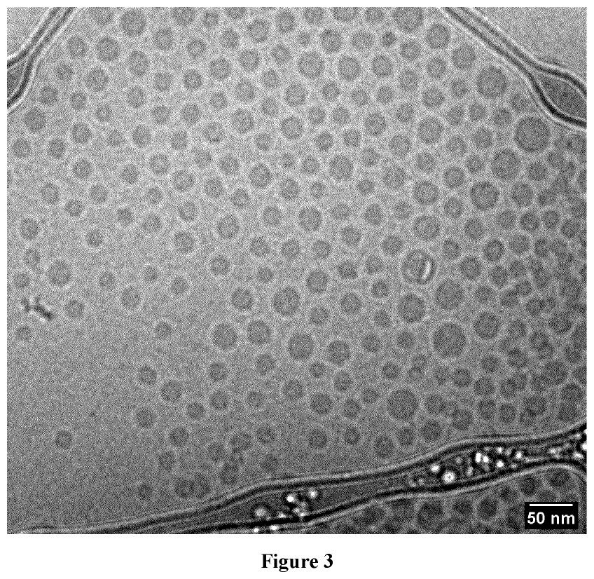

Particle size and PDI of LNPs were measured from TEM-images (FIGS. 1-3). The LNPs formulations were mixed by slow rotation of the container. Aliquots were pipetted out for TEM sample preparation.

Cryo-TEM specimen preparation: Cryo-TEM specimen of each formulation was prepared using plunge freezing method. Briefly, one drop of formulation was placed on lacey carbon film supported TEM grids which were pre-treated with glow discharge; then excess solution was blotted away from the TEM grid before being rapidly plunged into liquid ethane (−180° C.); the frozen samples on TEM grids were transferred and stored at liquid nitrogen temperature before imaging.

Cryo-TEM imaging: While imaging the lipid nanoparticles in TEM, the temperature of specimen was kept at −180° C. All Cryo-TEM images were obtained on JEOL2200FS TEM with an in-column Omega energy filter and running at accelerating voltage of 200 kV. A 10 eV energy slit and defocusing were applied for contrast enhancement. The magnifications were optimized for reasonable resolution and particle numbers in the field of view. 10 to 15 cryo-TEM images of LNPs for each sample were taken from different areas across the TEM grids.

### In Vivo Activity of LNPs

LNPs prepared using compounds 100, 105, 106 and 109 respectively were evaluated for specific CD8 T-cells expansion in mice where the payload is 3-GP mRNA (comprising epitopes encoding gp33/34). The LNPs were effective at expanding both gp33 and gp34 specific T-cells thereby demonstrating that the LNPs are effective at delivering, initiating expression and antigen processing of gp33 and gp34.

### EQUIVALENTS AND SCOPE

Those skilled in the art will recognize, or be able to ascertain using no more than routine experimentation, many equivalents to the specific embodiments described herein. The scope is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments, to the tenth of the unit of the lower limit of the range, unless the context clearly dictates otherwise.

In addition, it is to be understood that any particular embodiment that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions described herein (e.g., any therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A compound of Formula (I): 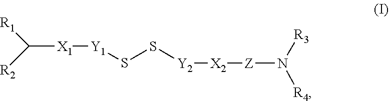
or a pharmaceutically acceptable salt thereof, wherein
X1 and X2 are independently an optionally substituted ester, amino, amido or either group,
Y1 and Y2 are independently a bond or an optionally substituted C1-C10 alkyl group with any possible isomerism,
Z is an optionally substituted C1-C10 alkyl group with any possible isomerism,
R₁ and R₂ are independently an optionally substituted C8-C20 alkyl, C8-C20 alkenyl, or C8-C20 alkynyl group with any possible isomerism, and
R₃ and R₄ are independently H, an optionally substituted C1-C4 alkyl group, or an optionally substituted C1-C4 alkoxyl group.

2. The compound of claim 1, or the pharmaceutically acceptable salt thereof, wherein
X1 and X2 independently represent an ester bond —CO—O— or —O—CO—,
Y1 and Y2 are independently an optionally substituted linear or branched C1-C10 alkyl group,
Z is an optionally substituted linear or branched C1-C10 alkyl group,
R₁ and R₂ are independently a linear or branched C8-C20 alkyl, a linear or branched C8-C20 alkenyl, or a linear or branched C8-C20 alkynyl group, wherein the C8-C20 alkyl is optionally substituted with a linear or branched C2-C12 alkenyl group, and
R₃ and R₄ are independently an optionally substituted C1-C4 alkyl group.

3. The compound of claim 1 or 2, or the pharmaceutically acceptable salt thereof, wherein
X1 represents an ester bond —CO—O— and X2 represents an ester bond —O—CO—, or X1 represents an ester bond —O—CO— and X2 represents an ester bond —CO—O—,
Y1 and Y2 independently represent a linear or branched C1-C10 alkyl group,
Z is a linear or branched C1-C10 alkyl group,
R₁ and R₂ are different and independently represent a linear or branched C8-C20 alkyl, or a linear or branched C8-C20 alkenyl, wherein the C8-C20 alkyl is optionally substituted with a linear or branched C2-C12 alkenyl group, and
R₃ and R₄ are independently a C1-C4 alkyl group, wherein the C1-C4 alkyl group is optionally substituted with halogen, hydroxyl, acetoxy, alkoxycarbonyl, formyl, acyl, thiocarbonyl, alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, an aromatic moiety or an heteroaromatic moiety.

4. The compound of any one of claims 1 to 3, or the pharmaceutically acceptable salt thereof, wherein
X1 represents an ester bond —CO—O— and X2 represents an ester bond —O—CO—, or X1 represents an ester bond —O—CO— and X2 represents an ester bond —CO—O—,
Y1 and Y2 independently represent a linear or branched C1-C10 alkyl group,
Z is a linear or branched C1-C10 alkyl group,
R₁ and R₂ are different and independently represent a linear or branched C8-C20 alkyl, or a linear or branched C8-C20 alkenyl, wherein the C8-C20 alkyl is optionally substituted with a linear or branched C2-C12 alkenyl group, and
R₃ and R₄ are independently a C1-C4 alkyl group, wherein the C1-C4 alkyl group is optionally substituted with hydroxyl or acetoxy.

5. The compound of any one of claims 1 to 4, or the pharmaceutically acceptable salt thereof, wherein
X1 represents an ester bond —CO—O— and X2 represents an ester bond —O—CO—, or X1 represents an ester bond —O—CO— and X2 represents an ester bond —CO—O—,
Y1 and Y2 independently represent a linear or branched C1-C4 alkyl group,
Z is a linear or branched C1-C10 alkyl group,
R₁ and R₂ are different and independently represent a linear or branched C8-C20 alkyl, or a linear or branched C8-C20 alkenyl, wherein the C8-C20 alkyl is optionally substituted with a linear C2-C12 alkenyl group, and
R₃ and R₄ are independently a C1-C4 alkyl group, wherein the C1-C4 alkyl group is optionally substituted with hydroxyl or acetoxy.

6. The compound of any one of claims 1 to 5, or the pharmaceutically acceptable salt thereof, wherein Y1 and Y2 are identical.

7. The compound of any one of claims 1 to 6, or the pharmaceutically acceptable salt thereof, wherein Z is a linear or branched C1-C4 alkyl group.

8. The compound of any one of claims 1 to 7, or the pharmaceutically acceptable salt thereof, wherein R₁ and R₂ are different and independently represent a linear or branched C8-C18 alkyl, or C8-C18 alkenyl, wherein the C8-C18 alkyl is optionally substituted with a C6-C10 alkenyl group, the alkenyl groups independently comprising one or two double bonds.

9. The compound of any one of claims 1 to 8, or the pharmaceutically acceptable salt thereof, wherein R₃ and R₄ are independently a C1-C2 alkyl group, wherein the C1-C2 alkyl group is optionally substituted with hydroxyl or acetoxy.

10. The compound of any one of claims 1 to 9, or the pharmaceutically acceptable salt thereof, wherein R₃ and R₄ are identical.

11. The compound of claim 1, wherein the compound has a structure of Formula (I1), (I2), (I3), (I4) or (I5): 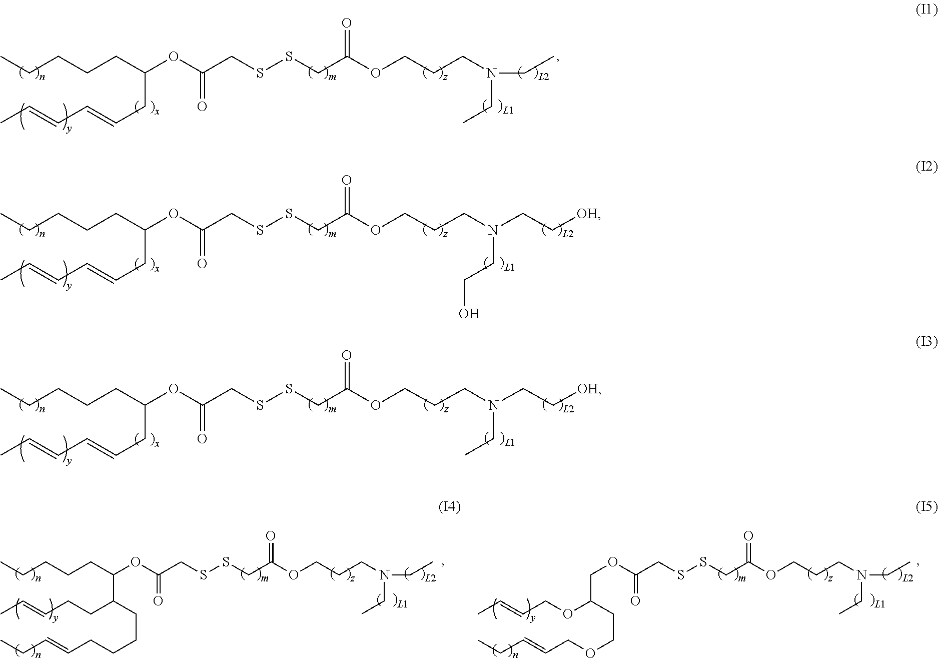
or a pharmaceutically acceptable salt thereof, and wherein n is a number from 4 to 16; x is a number from 1 to 15; y is a number from 1 to 8; m is a number from 1 to 10; z is a number from 0 to 8;
and L1 and L2 are independently a number from 0 to 3.

12. The compound of claim 1, wherein the compound has a structure of Formula (I6): 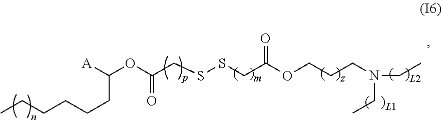
a pharmaceutically acceptable salt thereof, and wherein n is a number from 3 to 16; m and p are each independently a number from 1 to 10; z is a number from 0 to 8; and L1 and L2 are independently a number from 0 to 3; and A is a linear or branched C4-C20 alkyl, a linear or branched C4-C20 alkenyl, or a linear or branched C4-C20 alkynyl group.

13. The compound of claim 12, or a pharmaceutically acceptable salt thereof, wherein n is a number from 1 to 16; m and p are independently a number from 1 to 3; z is a number from 0 to 2;
L1 and L2 are 0; and A is a linear or branched C5-C20 alkyl, a linear or branched C5-C20 alkenyl, or a linear or branched C5-C20 alkynyl group.

14. The compound of claim 12 or 13, wherein the compound has a structure of Formula (I6a) or (I6b), or a pharmaceutically acceptable salt thereof, 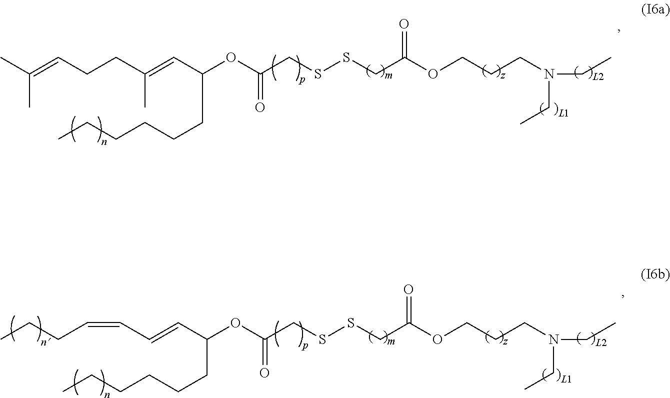
wherein m, n, p, z, L1 and L2 are as defined in claims 12 or 13, and n′ is a number from 1 to 14.

15. The compound of claim 1, wherein the compound has a structure of Formula (I7): 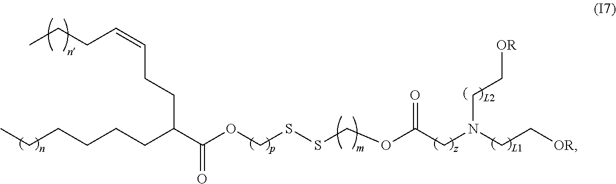
or a pharmaceutically acceptable salt thereof, wherein n and n′ are each independently a number from 1 to 16; m and p are each independently a number from 1 to 10; z is a number from 1 to 8; L1 and L2 are independently a number from 0 and 3; and R is H or —COCH₃.

16. The compound of claim 15, or the pharmaceutically acceptable salt thereof, wherein n and n′ are each independently a number from 1 to 16; m and p are each independently a number from 1 to 3; z is 1 or 2; L1 and L2 are 1; and R is H or —COCH₃.

17. The compound of claim 1, wherein the compound has a structure of Formula (I8): 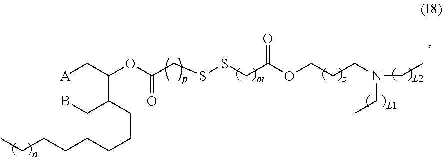
or a pharmaceutically acceptable salt thereof, wherein m and p are independently a number from 1 to 10; n is a number from 1 to 16; z is a number from 0 to 8; L1 and L2 are independently a number from 0 to 3; and A and B are independently a linear or branched C4-C20 alkyl, a linear or branched C4-C20 alkenyl, or a linear or branched C4-C20 alkynyl group.

18. The compound of claim 17, or the pharmaceutically acceptable salt thereof, wherein m and p are independently a number from 1 to 3; n is a number from 1 to 16; z is a number from 0 to 2; L1 and L2 are 0; A is a linear or branched C5-C20 alkyl, or a linear or branched C5-C20 alkenyl; and
B is a linear or branched C5-C20 alkenyl group.

19. The compound of claim 17 or 18, wherein the compound has a structure of Formula (I8a) 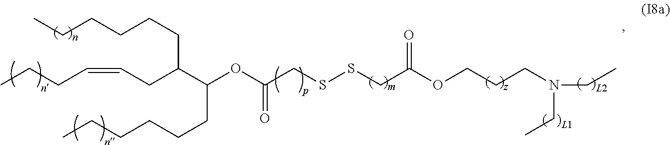
or a pharmaceutically acceptable salt thereof, wherein m, n, p, z, L1 and L2 are as defined in claims 17 or 18, n′ is a number from 1 to 14 and n″ is a number from 1 to 14.

20. The compound of claim 1, wherein the compound has a structure of Formula (I9): 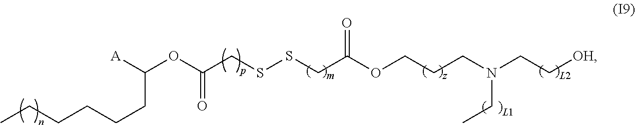
or a pharmaceutically acceptable salt thereof, and wherein n is a number from 1 to 16; m and p are independently a number from 1 to 10; z is a number from 0 to 8; L1 and L2 are independently a number from 0 to 3; and A is a linear or branched C4-C20 alkyl, a linear or branched C4-C20 alkenyl, or a linear or branched C4-C20 alkynyl group.

21. The compound of claim 20, or the pharmaceutically acceptable salt thereof, wherein n is a number from 1 to 16; m and p are independently a number from 1 to 3; z is a number from 0 to 2;
L1 is 0; L2 is 1; and A is a linear or branched C5-C20 alkenyl group.

22. A compound selected from the group consisting of Compounds 100, 101, 102, 103, 104, 105, 106, 107, 108, 109, 110, 111 and 112 of Table 1, or a pharmaceutically acceptable salt thereof.

23. The compound of any one of claims 1 to 22, wherein the compound is in the form of any enantiomer and/or any diastereoisomer thereof, or any mixture thereof.

24. A nanoparticle comprising at least one compound of any one of claims 1 to 23 or the pharmaceutically acceptable salt thereof.

25. A construct comprising the nanoparticle of claim 24 and a cargo, wherein the cargo is a small molecule, an antibody, a polynucleotide, or a polypeptide.

26. A vaccine comprising the nanoparticle of anyone of claim 24.

27. A method of vaccinating a subject against an infectious agent comprising
a) contacting a subject with the vaccine of claim 26, and
b) eliciting an immune response.

28. The method of claim 27, wherein the infectious agent is Campylobacter jejuni, Clostridium difficile, Entamoeba histolytica, enterotoxin B, Norwalk virus or norovirus, Helicobacter pylori, rotavirus, candida yeast, coronavirus including SARS-CoV, SARS-CoV-2 and MERS-CoV, Enterovirus 71, Epstein-Barr virus, Gram-Negative Bacteria including Bordetella, Gram-Positive Bacteria including Clostridium tetani, Francisella tularensis, Streptococcus bacteria and Staphylococcus bacteria, and Hepatitis, Human Cytomegalovirus, Human Immunodeficiency Virus, Human Papilloma Virus, Influenza, John Cunningham Virus, Mycobacterium, Poxviruses, Pseudomonas Aeruginosa, Respiratory Syncytial Virus, Rubella virus, Varicella zoster virus, Chikungunya virus, Dengue virus, Rabies virus, Trypanosoma cruzi and/or Chagas disease, Ebola virus, Plasmodium falciparum, Marburg virus, Japanese encephalitis virus, St. Louis encephalitis virus, West Nile Virus, Yellow Fever virus, Bacillus anthracis, Botulinum toxin, Ricin, or Shiga toxin and/or Shiga-like toxin.

29. A method of treating cancer of a subject in need thereof, comprising administering the nanoparticle of claim 24.

30. The method of claim 29, wherein the cancer is lung cancer, breast cancer, colorectal cancer, ovarian cancer, pancreatic cancer, colorectal cancer, bladder cancer, prostate cancer, cervical cancer, renal cancer, leukemia, central nervous system cancers, myeloma, or melanoma.

31. The vaccine of claim 26 for use in eliciting an immune response in a subject against an infectious agent.

32. The vaccine for use of claim 31, wherein the infectious agent is Campylobacter jejuni, Clostridium difficile, Entamoeba histolytica, enterotoxin B, Norwalk virus or norovirus, Helicobacter pylori, rotavirus, candida yeast, coronavirus including SARS-CoV, SARS-CoV-2 and MERS-CoV, Enterovirus 71, Epstein-Barr virus, Gram-Negative Bacteria including Bordetella, Gram-Positive Bacteria including Clostridium Tetani, Francisella Tularensis, Streptococcus bacteria and Staphylococcus bacteria, and Hepatitis, Human Cytomegalovirus, Human Immunodeficiency Virus, Human Papilloma Virus, Influenza, John Cunningham Virus, Mycobacterium, Poxviruses, Pseudomonas Aeruginosa, Respiratory Syncytial Virus, Rubella virus, Varicella zoster virus, Chikungunya virus, Dengue virus, Rabies virus, Trypanosoma cruzi and/or Chagas disease, Ebola virus, Plasmodium falciparum, Marburg virus, Japanese encephalitis virus, St. Louis encephalitis virus, West Nile Virus, Yellow Fever virus, Bacillus anthracis, Botulinum toxin, Ricin, or Shiga toxin and/or Shiga-like toxin.

33. The nanoparticle of claim 24 for use in treating cancer of a subject in need thereof.

34. The nanoparticle for use of claim 33, wherein the cancer is lung cancer, breast cancer, colorectal cancer, ovarian cancer, pancreatic cancer, colorectal cancer, bladder cancer, prostate cancer, cervical cancer, renal cancer, leukemia, central nervous system cancers, myeloma, or melanoma.
